(19) United States
(12) Patent Application Publication
(10) Pub. No.: US 2025/0136592 A1
(43) Pub. Date: May 1, 2025

(54) C-LINKED INHIBITORS OF ENL/AF9 YEATS

(71) Applicant: Bridge Medicines, New York, NY (US)

(72) Inventors: Tammy Ladduwahetty, London (GB); Joseph P. Vacca, Telford, PA (US); Sébastien L. Degorce, Hertfordshire (GB); Bradley Sherborne, Hertfordshire (GB); Tanweer A. Khan, Bridgewater, NJ (US); David John Huggins, New York, NY (US); Nigel Liverton, Harlyesville, PA (US)

(21) Appl. No.: 18/560,663

(22) PCT Filed: May 10, 2022
(86) PCT No.: PCT/US22/28516

§ 371 Date: Nov. 13, 2023

Related U.S. Application Data
(60) Provisional application No. 63/188,426, filed on May 13, 2021.

(51) Int. Cl. C07D 471/04; A61K 31/4184; A61K 31/423; A61K 31/437; A61K 31/4439; A61K 31/444; A61K 31/454; A61K 31/4545; A61K 31/4709; A61K 31/4725; A61K 31/501; A61K 31/502; A61K 31/506; A61K 31/517; A61K 31/538; A61K 45/06; C07D 401/14; C07D 403/14; C07D 413/14; C07D 519/00

(52) U.S. Cl. CPC C07D 471/04; A61K 31/4184; A61K 31/423; A61K 31/437; A61K 31/4439; A61K 31/444; A61K 31/454; A61K 31/4545; A61K 31/4709; A61K 31/4725; A61K 31/501; A61K 31/502; A61K 31/506; A61K 31/517; A61K 31/538; A61K 45/06; C07D 401/14; C07D 403/14; C07D 413/14; C07D 519/00

(57) ABSTRACT

Compounds of Formula I and pharmaceutical compositions comprising compounds of Formula I are disclosed. Methods for treating acute leukemias using the compounds of Formula I and pharmaceutical compositions comprising the same are also disclosed.

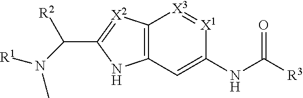

### CROSS REFERENCE TO RELATED APPLICATION

This application is a national phase application under 35 U.S.C. § 371 of International Application No. Patent Application No. PCT/US2022/028516, which claims priority to U.S. Provisional Patent Application No. 63/188,426, filed May 13, 2021. The contents of each of the above-identified applications is hereby fully incorporated herein by reference.

### FIELD OF THE INVENTION

The present application relates generally to compounds that inhibit ENL/AF9 YEATS and therapeutic methods of using such compounds. The compounds and methods find use in treating a variety of different diseases, including blood cancers such as leukemia.

### BACKGROUND OF THE INVENTION

The epigenome is an ensemble of chemical compounds contiguous to the DNA, responsible for the modification of the genome without altering the DNA sequences. It is dynamically regulated by chemical changes of DNA, RNA, and histones, around which DNA is packaged. It has been demonstrated that mutations in genes encoding epigenetic regulators plays a role in acute myeloid leukemia (AML) pathogenesis (Shih A H, Abdel-Wahab O, Patel J P, et al. “The role of mutations in epigenetic regulators in myeloid malignancies.” Nat. Rev. Cancer 2012; 12:599-612).

ENL is a chromatin reader protein possessing an amino-terminal YEATS domain (named for the first-discovered members of the family: Yaf9, ENL, AF9, Taf14, Sas5) and a disordered carboxy-terminal protein-protein interaction (PPI) interface. YEATS are a family of histone acetyllysine readers that act as effectors by allowing chromatin to be more accessible to RNA polymerase and transcriptional factors. Erb, et al. reported that a disproportionate number of leukemia proto-oncogenes and dependencies have ENL at their promoters (Erb, M. A. et al., “Transcription control by the ENL YEATS domain in acute leukaemia,” Nature 543, 270-274 (2017). Wan, et al. found that ENL binds to acetylated histone H3, and then colocalizes with H3K27 and H3K9ac on the promoters of genes essential for leukemia, and that ENL is required for AML maintenance (Wan L., et al. “ENL links histone acetylation to oncogenic gene expression in acute myeloid leukaemia,” Nature 2017; 543:265-9).

Given ENL's role in proliferation of leukemias, inhibitors of the YEATS domain of ENL are potential targets for treatment of blood cancers. For instance, Moustakim, et al. described small molecule inhibitors of ENL YEATS domain (Moustakim, M., et al., “Discovery of an MLLT1/3 YEATS Domain Chemical Probe,” Angew. Chem. Int. Ed. 2018, 57, 16302-16307). Moustakim's inhibitors compound contains a cyclic, nitrogenous heterocycle connected through a nitrogen atom to methylene group attached to a benzimidazole core. However, there remains a need for improved inhibitors useful for treating blood cancers.

### SUMMARY OF THE INVENTION

The invention is directed to compounds, pharmaceutical compositions, and methods for inhibiting YEATS/ENL and thereby treating various cancers, particularly blood cancers such as leukemia.

In a first aspect, the present invention relates to compounds of Formula I:

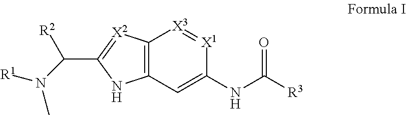

wherein:
X¹, X², and X³ are independently chosen from N and CH;
R¹ and R² are chosen from:
   (a) R¹ and R² taken together form a pyrrolidine or piperidine; and
   (b) R¹ and R² are methyl;
R³ is a fused bicycle selected from:
   (a) a fused 5,6 bicyclic heterocycle, optionally substituted with one or more C₁-C₆ alkyl;
   (b) a fused 6,5 bicyclic heterocycle, optionally substituted with one or more of the following: C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl; and
   (c) a fused 6,6 bicyclic heterocycle, optionally substituted with one or more of the following: C₁-C₆ alkyl, C₁-C₆ alkoxy, halogen, oxo, and NHR⁵, wherein R⁵ is chosen from hydrogen and C₁-C₆ alkyl.

In a second aspect, the present invention relates to pharmaceutical composition comprising a compound of Formula I and one or more pharmaceutically acceptable carriers. The pharmaceutical compositions can further comprise one or more therapeutic agents. Exemplary therapeutic agents include Bcl-2 inhibitors, cyclin-dependent kinase 4 and 6 (CDK 4/6) inhibitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators and chemotherapeutic agents.

In a third aspect, the present invention relates to methods of treating acute leukemias comprising administering a therapeutically effective amount of a compound of Formula I or a pharmaceutical composition comprising the same to a subject in need thereof. The acute leukemia can be acute lymphoblastic leukemia (ALL) or acute myelogenous leukemia (AML).

### DETAILED DESCRIPTION OF THE INVENTION

### I. Definitions

As used herein, “acyl” refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself is called “oxo”.

As used herein, the term “alkyl” includes linear or branched hydrocarbon structures. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below, e.g., C₁-C₁₀ alkyl, C₁-C₈ alkyl and C₁-C₆ alkyl.

As used herein, “aryl” and “heteroaryl” mean (i) a phenyl group (or benzene) or a monocyclic 5- or 6-membered heteroaromatic ring containing 1-4 heteroatoms selected from O, N, or S; (ii) a bicyclic 9- or 10-membered aromatic or heteroaromatic ring system containing 0-4 heteroatoms selected from O, N, or S; or (iii) a tricyclic 13- or 14-membered aromatic or heteroaromatic ring system containing 0-5 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

As used herein, “arylalkyl” refers to a substituent in which an aryl residue is attached to the parent structure through alkyl. Examples are benzyl, phenethyl and the like. “Heteroarylalkyl” refers to a substituent in which a heteroaryl residue is attached to the parent structure through alkyl. In one embodiment, the alkyl group of an arylalkyl or a heteroarylalkyl is an alkyl group of from 1 to 6 carbons. Examples include, e.g., pyridinylmethyl, pyrimidinylmethyl and the like.

As used herein, “C₁ to C₂₀ hydrocarbon” or “C₁ to C₂₀ hydrocarbyl” (as a substituent) includes alkyl, cycloalkyl, polycycloalkyl, alkenyl, alkynyl, aryl and combinations thereof. Examples include cyclopropylmethyl, benzyl, phenethyl, cyclohexylmethyl, camphoryl and naphthylethyl. Hydrocarbon refers to any substituent comprised of hydrogen and carbon as the only elemental constituents. Cycloalkyl is a subset of hydrocarbyl and includes cyclic hydrocarbon groups of from 3 to 8 carbon atoms. Examples of cycloalkyl groups include c-propyl, c-butyl, c-pentyl, norbornyl and the like.

“Alkoxy” or “alkoxyl” refers to groups of from 1 to 8 carbon atoms of a straight, branched or cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy.

As used herein, “carbocycle” is includes ring systems in which the ring atoms are all carbon but of any oxidation state. Thus (C₃-C₈) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

As used herein, the term “therapeutically effective amount” refers to any amount of a compound of the present invention or any other pharmaceutically active agent which, as compared to a corresponding a patient who has not received such an amount of the compound of the present invention or the other pharmaceutically active agent, results in improved treatment, healing, prevention, or amelioration of a disease, disorder, or side effect, or a decrease in the rate of advancement of a disease or disorder.

As used herein, the term “fused bicycles” refers to bicyclic carbocycles and bicyclic heterocycles in which each ring (a carbocycle or heterocycle) shares two adjacent atoms with another ring (a carbocycle or heterocycle). Each ring of the fused carbocycle can be selected from non-aromatic or aromatic rings. In preferred embodiments, the aromatic ring, such as phenyl, may be fused to another aromatic ring. In other embodiments, the aromatic ring may be fused to a non-aromatic ring, for example, cyclohexane, cyclopentane, or cyclohexene. Exemplary fused bicycles include 6,6; 6,5; and 5,6 fused bicyclic systems, wherein each number indicates the number of atoms in each ring. The fused bicycle can be substituted at any one or more position where it can have a hydrogen atom. The fused bicycle is bonded to the parent structure at the first numbered ring, e.g., the “6” ring of a fused 6,5 bicycle.

As used herein, “heterocycle” means a cycloalkyl or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Non-limiting examples of heteroaromatic rings include furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, benzothiazole, triazole, tetrazole, pyridine, quinoline, isoquinoline, pyrazine, quinoxaline, acridine, pyrimidine, quinazoline, pyridazine, cinnoline, phthalazine, and triazine. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygen atoms, as well as other heteroatoms. A sulfur heterocycle is a heterocycle containing at least one sulfur in the ring; it may contain additional sulfur atoms, as well as other heteroatoms. Oxygen heteroaryl is a subset of oxygen heterocycle; non-limiting examples include furan and oxazole. Sulfur heteroaryl is a subset of sulfur heterocycle; examples include thiophene and thiazine. A nitrogen heterocycle is a heterocycle containing at least one nitrogen in the ring; it may contain additional nitrogen atoms, as well as other heteroatoms. Non-limiting examples include piperidine, piperazine, morpholine, pyrrolidine and thiomorpholine. Nitrogen heteroaryl is a subset of nitrogen heterocycle; non-limiting examples include pyridine, pyrrole and thiazole.

Bicyclic nitrogenous heterocycles include (1) fused bicycles such as octahydrocyclopenta[c]pyrrole; (2) azaspirohexanes, heptanes and octanes, such as 6-oxa-2-azaspiro[3.4]octane, 2,6-diazaspiro[3.4]octane, 2-azaspiro[3.3]heptane, 2-oxa-6-azaspiro[3.3]heptane, and 7-oxa-2-azaspiro[3.5]nonane; and (3) an azabicycloalkane: 8-azabicyclo[3.2.1]octane. In the compounds described herein, these bicyclic nitrogenous heterocycles may be attached to the carbon bearing R³ via carbon.

As used herein, the term “optionally substituted” may be used interchangeably with “unsubstituted or substituted.” The term “substituted” refers to the replacement of one or more hydrogen atoms in a specified group with a specified radical. For example, substituted aryl, heterocyclyl etc. refer to aryl or heterocyclyl wherein one or more hr atoms in each residue are replaced with halogen, haloalkyl, alkyl, acyl, alkoxyalkyl, hydroxyloweralkyl, carbonyl, phenyl, heteroaryl, benzenesulfonyl, hydroxy, loweralkoxy, haloalkoxy, oxaalkyl, carboxy, alkoxycarbonyl [—C(═O)O-alkyl], carboxamido [—C(═O)NH₂], alkylaminocarbonyl [—C(═O)NH-alkyl], cyano, acetoxy, nitro, amino, alkylamino, dialkylamino, dialkylaminoalkyl, dialkylaminoalkoxy, (cycloalkyl)alkyl, heterocyclyl, heterocyclylalkyl, alkylaminoalkyl, heterocyclylaminoalkyl, heterocyclylalkylaminoalkyl, cycloalkylaminoalkyl, cycloalkylalkylaminoalkyl, arylaminoalkyl, and arylalkylaminoalkyl, mercapto, alkylthio, alkylsulfinyl, benzyl, heterocyclyl, phenoxy, benzyloxy, heteroaryloxy, aminosulfonyl, amidino, guanidino, and ureido. (C₁₋₆) hydrocarbyl, —SO₂alkyl, —SO₂NH₂, or —SO₂NHalkyl.

As used herein, “oxaalkyl” refers to alkyl residues in which one or more carbons (and their associated hydrogens) have been replaced by oxygen. Examples include methoxypropoxy, 3,6,9-trioxadecyl and the like. Alkoxy is a subset of oxaalkyl in which the carbon at the point of attachment is replaced by oxygen. The term oxaalkyl is intended as it is understood in the art [see Naming and Indexing of Chemical Substances for Chemical Abstracts, published by the American Chemical Society, 196, but without the restriction of 127 (a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Non-limiting examples include ethylaminoethyl and methylthiopropyl.

As used herein, “solvate” refers to a compound of Formula I in the solid state, wherein molecules of a suitable solvent are incorporated in the crystal lattice along with the compound of Formula I. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

As used herein, the term “subject” or “subject in need thereof” are used interchangeably herein. These terms refer to a patient who has been diagnosed with the underlying disorder to be treated. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder.

### II. Compounds

The present invention provides compounds of Formula I:

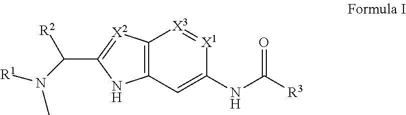

wherein:
X¹, X², and X³ are independently chosen from N and CH;
R¹ and R² are chosen from:
   (a) R¹ and R² taken together form a pyrrolidine or piperidine; and
   (b) R¹ and R² are methyl;
R³ is a fused bicycle selected from:
   (a) a fused 5,6 bicyclic heterocycle, optionally substituted with one or more C₁-C₆ alkyl;
   (b) a fused 6,5 bicyclic heterocycle, optionally substituted with one or more of the following: C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl; and
(c) a fused 6,6 bicyclic heterocycle, optionally substituted with one or more of the following: C₁-C₆ alkyl, C₁-C₆ alkoxy, halogen, oxo, and NHR⁵, wherein R⁵ is chosen from hydrogen and C₁-C₆ alkyl.

In one aspect, the compounds are pyrrolo[3,2-c]pyridines of Formula II:

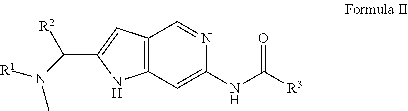

wherein R¹, R² and R³ are as defined above for Formula I.

In a particular embodiment, R¹ and R² of Formula II are methyl and the compounds are of Formula IIa:

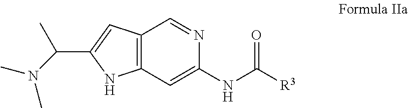

wherein R³ is defined as above for Formula I.

In another particular embodiment, R¹ and R² of Formula II together form a pyrrolidine and the compounds are of Formula IIb:

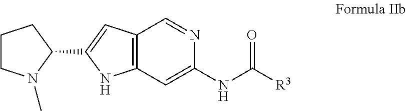

wherein R³ is defined as above for Formula I.

In certain sub-embodiments of Formula IIb, R³ is a fused 5,6 bicyclic heterocycle, optionally substituted with one or more C₁-C₆ alkyl (i.e., one or more R⁶). In all the embodiments described hereinbelow, R⁶ can be a substituent on any ring position of the fused heterocycle. An exemplary fused 5,6 bicyclic heterocycle is the following:

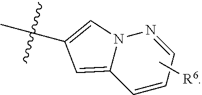

In other sub-embodiments of Formula IIb, R³ is a fused 6,5 bicyclic heterocycle, optionally substituted with one or more R⁶ (e.g., one or more of the following): C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl). Exemplary fused 6,5 bicyclic heterocycles include the following:

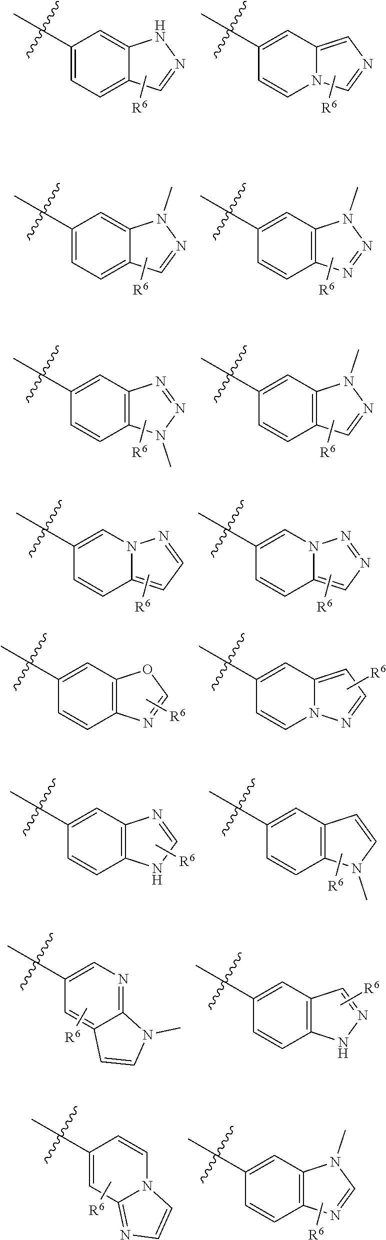

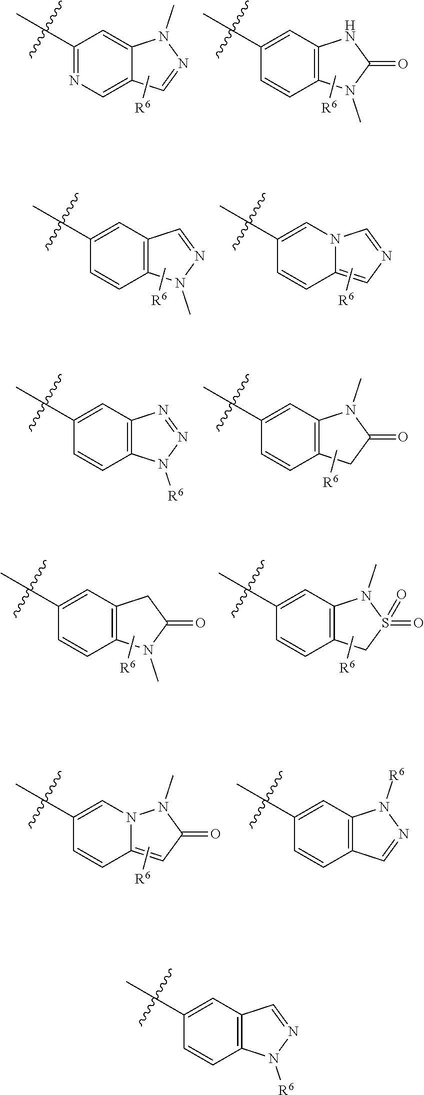

In still other sub-embodiments of Formula IIb, R³ is a fused 6,6 bicyclic heterocycle, optionally substituted with one or more of the following (i.e., one or more R⁶): C₁-C₆ alkyl, C₁-C₆ alkoxy, halogen, oxo, and NHR₅, wherein R⁵ is chosen from hydrogen and C₁-C₆ alkyl. Exemplary fused 6,6 bicyclic heterocycles include the following:

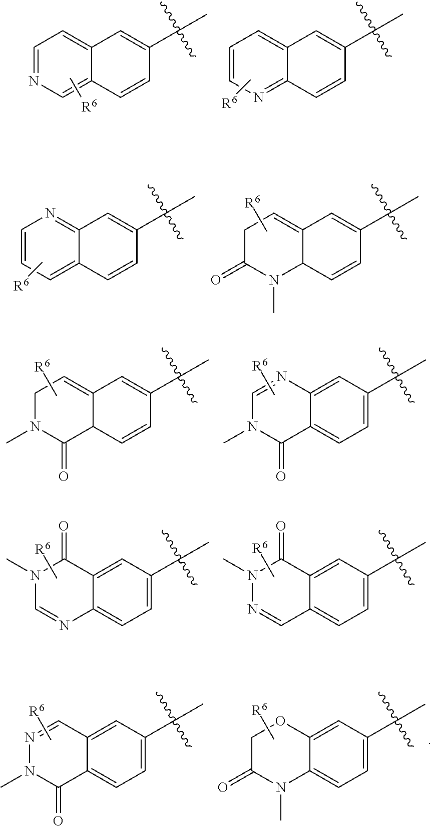

In a particular embodiment, R¹ and R² of Formula II together form a piperidine and the compounds are of Formula IIc:

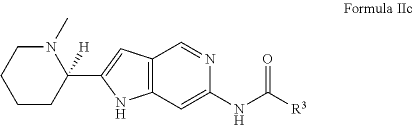

wherein R³ is defined as above for Formula I.

In certain sub-embodiments of Formula IIc, R³ is a fused 5,6 bicyclic heterocycle, optionally substituted with one or more C₁-C₆ alkyl (i.e., one or more R⁶). In all the embodiments described hereinbelow, R⁶ can be a substituent on any ring position of the fused heterocycle. An exemplary fused 5,6 bicyclic heterocycle is the following:

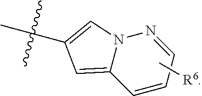

In other sub-embodiments of Formula IIc, R³ is a fused 6,5 bicyclic heterocycle, optionally substituted with one or more R⁶ (e.g., one or more of the following): C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl. Exemplary fused 6,5 bicyclic heterocycles include the following:

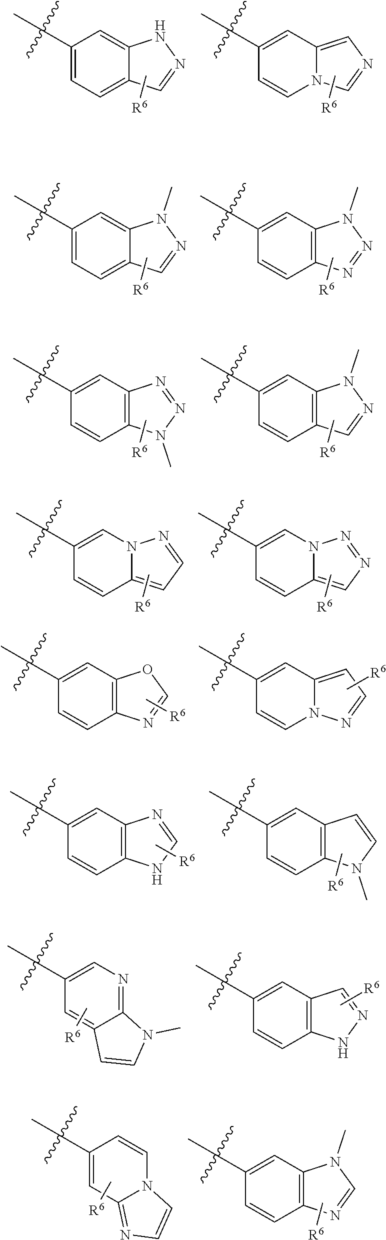

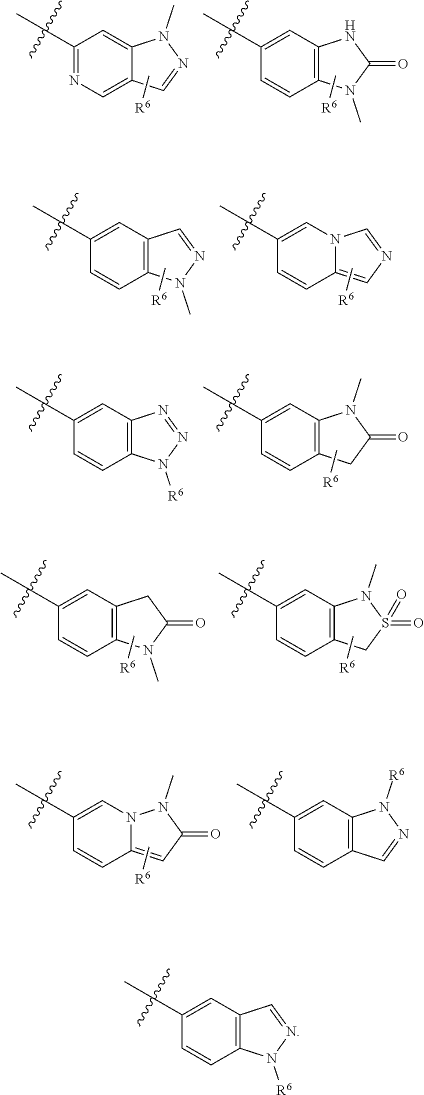

In still other sub-embodiments of Formula IIc, R³ is a fused 6,6 bicyclic heterocycle, optionally substituted with one or more of the following (i.e., one or more R⁶): C₁-C₆ alkyl, C₁-C₆ alkoxy, halogen, oxo, and NHR⁵, wherein R⁵ is chosen from hydrogen and C₁-C₆ alkyl. Exemplary fused 6,6 bicyclic heterocycles include the following:

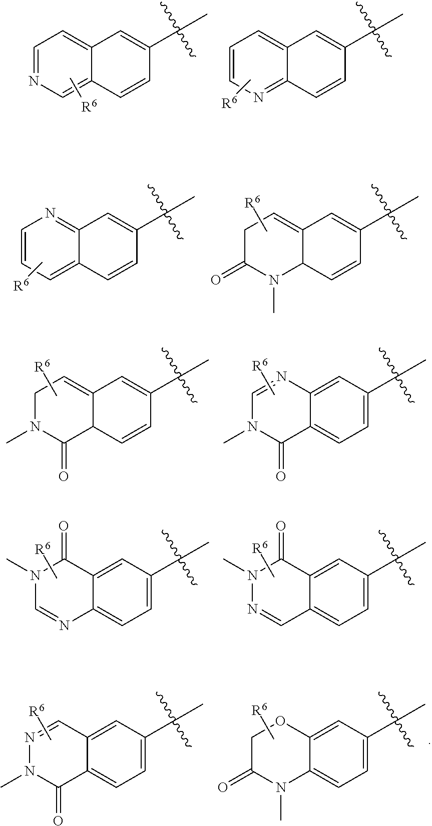

In another aspect, the compounds are 1H-pyrrolo[3,2-b]pyridines of Formula II:

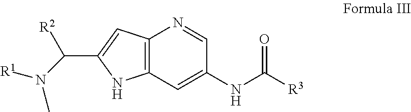

wherein R¹, R² and R³ are as defined above for Formula I.

In a particular embodiment, R¹ and R² of Formula III together form a pyrrolidine and the compounds are of Formula IIIa:

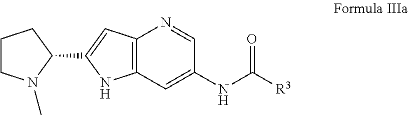

wherein R³ is defined as above for Formula I.

In certain sub-embodiments of Formula IIIa, R³ is a fused 6,5 bicyclic heterocycle, optionally substituted with one or more of the following (i.e., one or more R⁶): C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl. Exemplary fused 6,5 bicyclic heterocycles include the following:

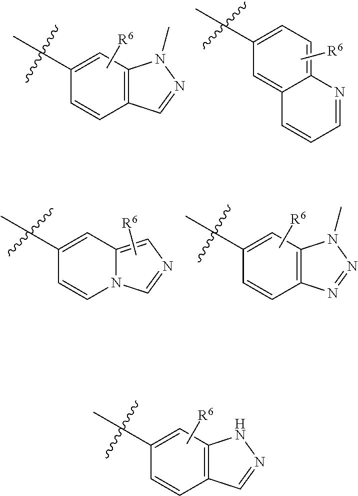

In other sub-embodiments of Formula IIIa, R³ is a fused 6,6 bicyclic heterocycle, optionally substituted with one or more of the following (i.e., one or more R⁶): C₁-C₆ alkyl, C₁-C₆ alkoxy, halogen, and NHR⁵, wherein R⁵ is chosen from hydrogen C₁-C₆ alkyl. An exemplary fused 6,6 bicyclic heterocycles include the following:

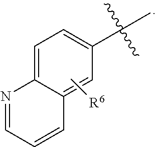

In still another aspect, the compounds are benzimidazoles of Formula IV:

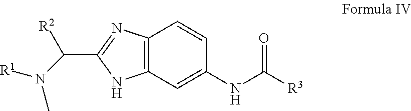

wherein R¹, R² and R³ are as defined above for Formula I.

In one particular embodiment, R¹ and R² of Formula IV together form a piperidine and the compounds are of Formula IVa:

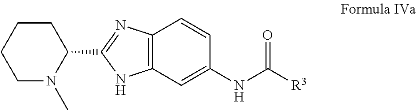

wherein R³ is defined as above for Formula I.

In another particular embodiment, R¹ and R² of Formula IV together form a pyrrolidine and the compounds are of Formula IVb:

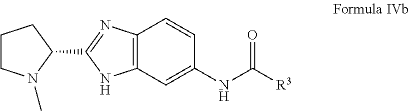

wherein R³ is defined as above for Formula I.

In certain sub-embodiments of Formula IVb, R³ is a fused 6,5 bicyclic heterocycle, optionally substituted with one or more of the following (i.e., one or more R⁶): C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl. Exemplary fused 6,5 bicyclic heterocycles include the following:

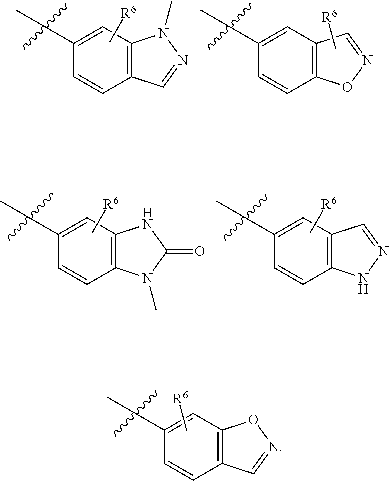

In a further sub-embodiment, the compounds are of Formula IVb′:

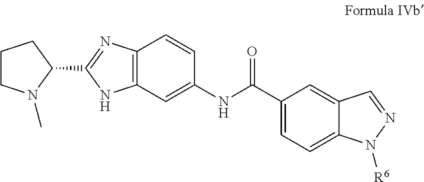

wherein R⁶ is as described above. In a particular embodiment, R⁶ is a heterocycle.

In other sub-embodiments of Formula IVb, R³ is a fused 6,6 bicyclic heterocycle, optionally substituted with one or more of the following (i.e., one or more R⁶): C₁-C₆ alkyl, C₁-C₆ alkoxy, halogen, and NHR⁵, wherein R⁵ is chosen from hydrogen C₁-C₆ alkyl. Exemplary fused 6,6 bicyclic heterocycles include the following:

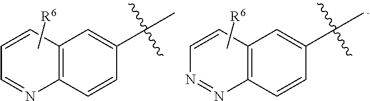

In yet another aspect, the compounds are of Formula V:

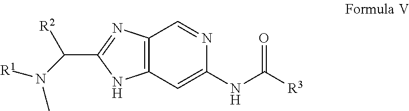

wherein R¹, R² and R³ are as defined above for Formula I.

In a particular embodiment, R¹ and R² of Formula V together form a pyrrolidine and the compounds are of Formula Va:

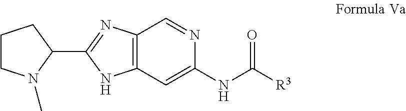

wherein R³ is defined as above for Formula I.

In certain sub-embodiments of Formula Va, R³ is a fused 6,5 bicyclic heterocycle, optionally substituted with one or more of the following (i.e., one or more R⁶): C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl. An exemplary fused 6,5 bicyclic heterocycles includes the following:

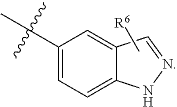

In a yet another aspect, the compounds are of Formula VI:

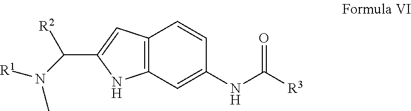

wherein R¹, R² and R³ are as defined above for Formula I.

In a particular embodiment, R¹ and R² of Formula VI together form a pyrrolidine and the compounds are of Formula VIa:

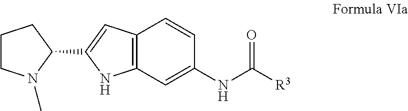

wherein R³ is defined as above for Formula I.

In certain sub-embodiments of Formula IVa, R³ is a fused 6,6 bicyclic heterocycle, optionally substituted with one or more of the following (i.e., one or more R⁶): C₁-C₆ alkyl, C₁-C₆ alkoxy, halogen, and NHR⁵, wherein R⁵ is chosen from hydrogen C₁-C₆ alkyl. An exemplary fused 6,6 bicyclic heterocycles include the following:

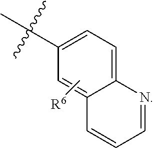

As used herein, “a compound”-unless expressly further limited—is intended to include salts of that compound. Thus, for example, the recitation “a compound of Formula I” as depicted above, would include salts:

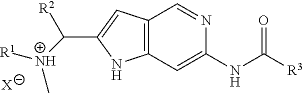

in which X is any counterion. In a particular embodiment, the term “compound of Formula I” refers to the compound or a pharmaceutically acceptable salt thereof. The term “pharmaceutically acceptable salt” refers to salts prepared from pharmaceutically acceptable non-toxic acids or bases including inorganic acids and bases and organic acids and bases. When the compounds of the present invention are basic, as they usually would be, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. Further pharmaceutically acceptable salts include, when appropriate, nontoxic ammonium cations and carboxylate, sulfonate and phosphonate anions attached to alkyl having from 1 to 20 carbon atoms.

Unless otherwise stated or depicted, structures depicted herein are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the invention.

In certain embodiments, the compound has a R stereochemical configurations at the chiral center of Formula I. Compounds having R stereochemistry generally show higher activity than the corresponding S enantiomer. In other embodiments, the compound has a S stereochemical configurations at the chiral center of Formula I.

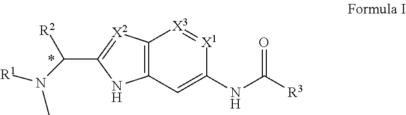

Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### III. Pharmaceutical Compositions

The present invention also provides pharmaceutical compositions comprising at least one compound described herein (including pharmaceutically acceptable salts and solvates thereof).

A pharmaceutical composition comprises at least one compound described herein and one or more pharmaceutically acceptable excipients. Exemplary excipients include, but are not limitated to, including, but not limited to, one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opaquing agents, glidants, processing aids, colorants, sweeteners, taste-masking agents, perfuming agents, flavoring agents, diluents, polishing agents, polymer matrix systems, plasticizers and other known additives to provide an elegant presentation of the drug or aid in the manufacturing of a medicament or pharmaceutical product comprising a composition of the present inventions. Examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams &Wilkins, 2000; and Rowe, Raymond C. Handbook of Pharmaceutical Excipients. Chicago, Pharmaceutical Press, 2005.

Non-limiting examples of excipients include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), hydroxypropyl cellulose, titanium dioxide, talc, calcium carbonate (e.g., granules or powder), microcrystalline cellulose, powdered cellulose, dextrates, kaolin, silicic acid, sorbitol, starch, pre-gelatinized starch, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, a syloid silica gel (AEROSIL200, manufactured by W. R. Grace Co. of Baltimore, MD), a coagulated aerosol of synthetic silica (marketed by Degussa Co. of Plano, TX), CAB-O-SIL (a pyrogenic silicon dioxide product sold by Cabot Co. of Boston, MA), colorants and mixtures thereof.

The pharmaceutical compositions can optionally include one or more additional therapeutic agents.

Additional therapeutic agents include Bcl-2 inhibitors, cyclin-dependent kinase 4 and 6 (CDK 4/6) inhibitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators, and chemotherapeutic agents.

The standard of care for AML and ALL is currently chemotherapy with a chemotherapeutic agent. Exemplary chemotherapeutic agents include, but are not limited to, daunorubicin, cytarabine, methotrexate, mitoxantrone, methotrexate, mafosamide and vincristine.

Targeted therapeutic agents e.g., those discussed below, can be used alone or in combination with a chemotherapeutic agent.

Exemplary Bcl-2 inhibitors include, but are not limited to, e.g. oblimersen, navitoclax and venetoclax.

Exemplary cyclin-depenent kinases 4 and 6 (CDK 4/6) inhibitors include, but are not limited to, palbociclib, ribociclib and abemaciclib.

Epigenetic modulators include, but are not limited to, menin-histone methyltransferase MLL (i.e., menin-MLL) inhibitors, FLT3 inhibitors, P-TEFb inhibitors, histone methyltransferase inhibitors (e.g., DOTIL and EZH2 inhibitors), bromodomain and extra-terminal domain (BET) inhibitors and dihydroorotate dehydrogenase (DHODH) inhibitors.

Exemplary FLT3 inhibitors include, but are not limited to, sorafenib, lestaurtinib, sunitinib, tandutinib, quizartinib, midostaurin, gilteritinib, crenolanib, cabozantinib and ponatinib.

Combinations of epigenetic modulators, e.g., menin-MLL inhibitors and FLT3 inhibitors, are also contemplated as these have shown enhanced apotosis induction in AML models.

In one embodiment, the additional therapeutic agents comprise a combination of at least one Bcl-2 inhibitor and at least one FLT3 inhibitor.

Exemplary DNA methyltransferase inhibitors include, but are not limited to, azacytidine and decitabine.

Exemplary HDAC inhibitors include, but are not limited to, panobinostat and vorinostat.

ExemplarymTOR inhibitors include, but are not limited to, everolimus.

Exemplary glucocorticoids include, but are not limited to, dexamethasone and prednisolone.

Exemplary mutant isocitrate dehydrogenase inhibitors include, but are are not limited to, ivosidenib (IDH1) and enasidenib (IDH2).

In one embodiment, the additional therapeutic agents comprise a combination of at least one isocitrate dehydrogenase inhibitor and at least one CDK 4/6 inhibitor.

### IV. Methods of Use

The present invention also relates to methods of using at least one compound described herein or a pharmaceutical composition described herein to suppress oncogene expression in a cell. In one embodiment, a method of suppressing oncogene expression in a cell comprises exposing the cell to at least one compound described herein. The present invention also relates to methods of using at least one compound described herein or a pharmaceutical composition described herein to treat an acute leukemia. In one embodiment, a method of treating an acute leukemia comprises administering a therapeutically effective amount of at least one compound described herein to a subject in need thereof.

Acute leukemias are rapidly progressing leukemia characterized by replacement of normal bone marrow by blast cells of a clone arising from malignant transformation of a hematopoietic cell. The acute leukemias include acute lymphoblastic leukemia (ALL) and acute myelogenous leukemia (AML). ALL often involves the CNS, whereas acute monoblastic leukemia involves the gums, and AML involves localized collections in any site (granulocytic sarcomas or chloromas).

In one embodiment, the acute leukemia is ALL. ALL is the most common malignancy in children, with a peak incidence from ages 3 to 5 years. It also occurs in adolescents and has a second, lower peak in adults. Typical treatment emphasizes early introduction of an intensive multidrug regimen, which may include prednisone, vincristine, anthracycline or asparaginase. Other drugs and combinations are cytarabine and etoposide, and cyclophosphamide. Relapse usually occurs in the bone marrow but may also occur in the CNS or testes, alone or concurrent with bone marrow. Although second remissions can be induced in many children, subsequent remissions tend to be brief.

In another embodiment, the acute leukemia is AML. The incidence of AML increases with age; it is the more common acute leukemia in adults. AML may be associated with chemotherapy or irradiation (secondary AML). Remission induction rates are lower than with ALL, and long-term disease-free survival reportedly occurs in only 20 to 40% of patients. Treatment differs most from ALL in that AML responds to fewer drugs. The basic induction regimen includes cytarabine; along with daunorubicin or idarubicin. Some regimens include 6-thioguanine, etoposide, vincristine, and prednisone. Clinical aspects of AML are reviewed by C. A. Schiffer and R. M. Stone in Cancer Medicine, Ed. David W. Kufe et al, 6th Edition, B. C. Decker, 2003.

This French, American, and British (FAB) classification has been developed to diagnose and classify acute myeloid leukemia. The diagnosis of acute myeloid leukemia requires that myeloblasts constitute 30% (or 20% based on a recent World Health Organization (WHO) classification system) or more of bone marrow cells or circulating white blood cells. The hematologic properties of the disease define the various subtypes described below. The FAB nomenclature (M1 through M7) classifies the subtypes of acute myeloid leukemia according to the normal marrow elements that the blasts most closely resemble. The following list includes both the FAB classifications as well as additional classes recognized by the WHO.
Acute myeloid leukemia, minimally differentiated (MO)
Acute myeloid leukemia without maturation (M1)
Acute myeloid leukemia with maturation (M2)
Acute myeloid leukemia with maturation with t (8;21)
Acute promyelocytic leukemia (M3)
Hypergranular type
Microgranular type
Acute myelomonocytic leukemia (M4)
Acute myelomonocytic leukemia with increased marrow eosinophils (M4E0)
Acute Monocytic Leukemia (M5)
Acute monoblastic leukemia (M5a)
Acute monocytic leukemia with maturation (M5b)
Erythroleukemia Erythroid/myeloid) (M6a)
Pure erythroid malignancy (M6b)
Acute megakaryoblastic leukemia (M7)
Acute megakaryoblastic leukemia associated with t (1;22)
Acute basophilic leukemia
Acute myelofibrosis (acute myelodysplasia with myelofibrosis)
Acute leukemia and transient myeloproliferative disorder in Down's Syndrome Hypocellular acute myeloid leukemia
Myeloid sarcoma

In one embodiment, a method of treating a subtype of AML listed above comprises administering a therapeutically effective amount of at least one compound described herein to a subject in need thereof.

The at least one compound used in the present methods can be provided in the form of a pharmaceutical composition described hereinabove.

Routes of administration include enteral, such as oral; and parenteral, such as intravenous, intra-arterial, intramuscular, intranasal, rectal, intraperitoneal, subcutaneous and topical routes.

For parenteral administration, the active compounds may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agents. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active compounds may be combined with one or more solid inactive ingredients for the preparation of tablets, capsules, pills, powders, granules or other suitable oral dosage forms. For example, the active compounds may be combined with at least one excipient such as fillers, binders, humectants, disintegrating agents, solution retarders, absorption accelerators, wetting agents, absorbents or lubricating agents.

The specific doses of the active compound(s) employed in the composition and methods of the invention to obtain therapeutic benefit will, of course, be determined by the particular circumstances of the individual patient. Such circumstances include the size, weight, age and sex of the patient, the nature and stage of the disease, the aggressiveness of the disease, and the route of administration.

For the compounds described herein, the preferred daily dose is in the range of about 1 to about 10,000 mg, more preferably from about 5 to about 5,000 mg, still more preferably about 10 to about 3,000, most preferably about 50 to about 1,000, for example. In certain embodiments, the preferred daily dose is in the range of about 50 mg to about 4,000 mg, about 100 mg to about 3,000 mg, about 500 to about 2,000 or about 750 mg to about 1,500 mg. In other embodiments, the preferred daily dose is in the range of 2,000 mg to about 10,000 mg, about 3,000 to about 9,000 mg, about 4,000 mg to about 8,000 mg, or about 4,500 to about 7,500 mg.

A dose may be administered one to four times a day, e.g., once a day, as required to provide therapeutic benefit. In certain embodiments, a therapeutic compound of the invention is administered intravenously, either as a one-time dose or as part of a scheduled dosing regimen that may be spread out over several days, weeks, or months. The compounds of the invention may also be administered by periodic injection, as needed to obtain a therapeutic benefit.

The methods described herein can further comprise administration of an additional therapeutic agent, e.g., Bcl-2 inhibitors, cyclin-dependent kinase 4 and 6 (CDK 4/6) inhibitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators, and chemotherapeutic agents. The additional therapeutic agent can be administered either simultaneously or sequentially with the compounds described herein. In some embodiments administration of a compound described herein and additional therapeutic agent can produce a synergistic effect.

### EXAMPLES

The following compounds have been prepared, isolated and characterized using the methods disclosed herein. They demonstrate a partial scope of the invention and are not meant to be limiting of the scope of the invention.

The compounds of the present invention were prepared by methods well known in the art of synthetic organic chemistry. During synthetic sequences it was sometimes necessary or desirable to protect sensitive or reactive groups on any of the molecules concerned. This was achieved by means of conventional protecting groups, such as those described in T. W. Greene and P. G. M. Wuts Greene's Protective Groups in Organic Synthesis, Fourth edition, John Wiley and Sons, 2006. The protecting groups were removed at a convenient subsequent stage using methods well known in the art.

All reactions were performed under a dry atmosphere of nitrogen unless otherwise specified. Indicated reaction temperatures refer to the reaction bath, while room temperature (rt) is noted as 25° C. Commercial grade reagents and anhydrous solvents were used as received from vendors and no attempts were made to purify or dry these components further. Removal of solvents under reduced pressure was accomplished with a Buchi rotary evaporator at approximately 28 mm Hg pressure using a Teflon-linked KNf vacuum pump. Flash column chromatography was carried out using a Teledyne Isco CombiFlash Companion unit with RediSep Rf silica gel columns. Proton NMR spectra were obtained on a 300 MHz and 400 MHz Bruker Nuclear Magnetic Resonance Spectrometer. Chemical shifts (δ) are reported in parts per million (ppm) and coupling constants (J) values are given in Hz, with the following spectral pattern designations: s, singlet; d, doublet; t, triplet; q, quartet; dd, doublet of doublet; m, multiplet; brs, broad singlet. Tetramethylsilane was used as an internal reference. Mass spectroscopic analysis were performed using positive and negative mode electron spray ionization (ESI) on an Agilent 1200 system. High pressure liquid chromatography (HPLC) purity analysis was performed using a Varian Pro Star HPLC system with a binary solvent system A and B using a gradient elution [A, H₂O with 0.0284% NH₄OAc and 0.0116% Acetic acid; B, CH₃CN] and flow rate=1 mL/min, with PDA Scan for UV detection. The following Varian Pro Star HPLC method was used to establish compound purity:

### Intermediate 1: (R)-2-(1-methylpyrrolidin-2-yl)-1H-benzo[d]imidazol-5-amine

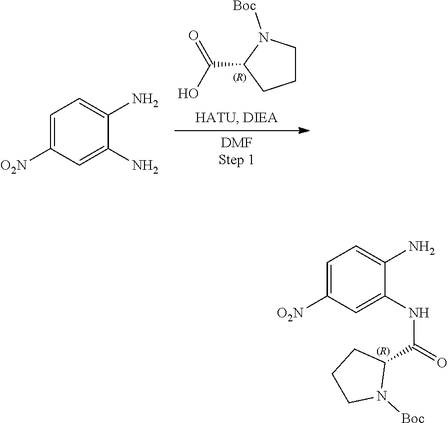

Into a 500 mL round-bottom flask purged and maintained with an inert atmosphere of nitrogen, was placed 2-amino-4-nitroaniline (20.00 g, 133.06 mmol, 1.00 equiv), (2R)-1-methylpyrrolidine-2-carboxylic acid (18.55 g, 143.64 mmol, 1.10 equiv), HATU (59.56 g, 156.72 mmol, 1.20 equiv), DIEA (67.48 g, 552.4 mmol, 4.00 equiv), DCM (200.00 mL). The resulting solution was stirred for 2 hr at 25° C. The resulting mixture was concentrated. This resulted in 46 g (crude) of (2R)-N-(2-amino-5-nitrophenyl)-1-methylpyrrolidine-2-carboxamide as a brown solid.

LC-MS: (ES, m/z): [M+1]⁺=351.

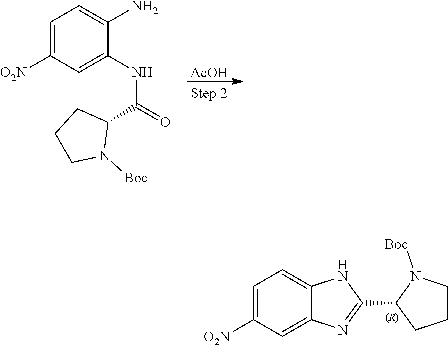

Into a 1000-mL 3-necked round-bottom flask purged and maintained with an inert atmosphere of nitrogen, was placed tert-butyl (2R)-2-[(2-amino-5-nitrophenyl)carbamoyl]pyrrolidine-1-carboxylate (46.00 g, 131.29 mmol, 1.00 equiv), AcOH (460 mL). The resulting solution was stirred for 48 hr at 60° C. The resulting mixture was concentrated and quenched by the addition of 500 mL of water. The resulting solution was extracted with 3×100 mL of ethyl acetate and the organic layers combined. The resulting mixture was washed with 3×100 of brine. The resulting mixture was concentrated. The residue was applied onto a silica gel column with ethyl acetate/petroleum ether (1:2). This resulted in 32 g (73.3%) of tert-butyl (2R)-2-(5-nitro-1H-1,3-benzodiazol-2-yl) pyrrolidine-1-carboxylate as a brown solid.

LC-MS: (ES, m/z): [M+1]⁺=333.

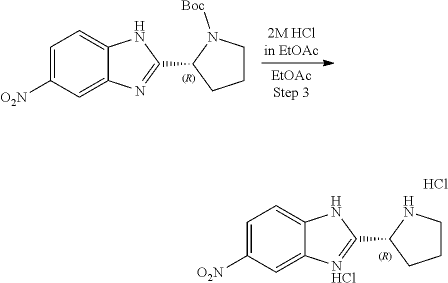

Into a 500-mL 3-necked round-bottom flask purged and maintained with an inert atmosphere of nitrogen, was placed tert-butyl (2R)-2-(5-nitro-1H-1,3-benzodiazol-2-yl) pyrrolidine-1-carboxylate (32.00 g, 96.281 mmol, 1.00 equiv). To the above HCl (g) in EA (320 mL, 5605.6 mmol, 58.22 equiv) was introduced in at 25° C. The resulting solution was stirred for 2 hr at 25° C. The solids were collected by filtration. This resulted in 26 g (crude) of 5-nitro-2-[(2R)-pyrrolidin-2-yl]-1H-1,3-benzodiazole hydrochloride as a yellow solid.

LC-MS: (ES, m/z): [M+1]⁺=233.

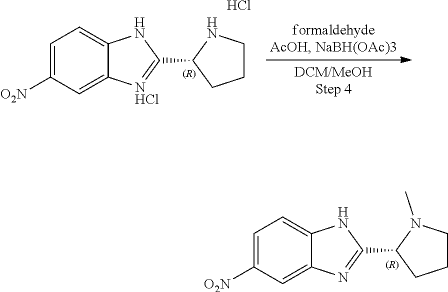

Into a 250-mL 3-necked round-bottom flask purged and maintained with an inert atmosphere of nitrogen, was placed 5-nitro-2-[(2R)-pyrrolidin-2-yl]-1H-1,3-benzodiazole dihydrochloride (9.00 g, 29.493 mmol, 1.00 equiv), formaldehyde (8.86 g, 294.93 mmol, 10.00 equiv), DCM (90.00 mL), MeOH (45.00 mL), NaBH(OAc)₃ (62.51 g, 294.93 mmol, 10.00 equiv). The resulting solution was stirred for 2 hr at 25° C. The reaction was then quenched by the addition of 400 ml of water/ice. The resulting solution was extracted with 3×200 mL of ethyl acetate and the organic layers combined and concentrated. The residue was applied onto a silica gel column with ethyl acetate/petroleum ether (1:1). This resulted in 6.3 g (86.74%) of 2-[(2R)-1-methylpyrrolidin-2-yl]-5-nitro-1H-1,3-benzodiazole as a brown solid.

LC-MS: (ES, m/z): [M+1]⁺=247.

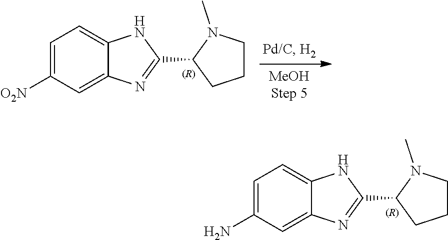

Into a 1000-mL round-bottom flask, was placed 2-[(2R)-1-methylpyrrolidin-2-yl]-5-nitro-1H-1,3-benzodiazole (5.00 g, 20.30 mmol, 1.00 equiv), Pd/C (432.00 mg, 4.050 mmol, 0.20 equiv), methanol (500.00 mL). To the above H₂ (g) was introduced in at 25° C. The resulting solution was stirred for 1 overnight at 25° C. The solids were filtered out. The resulting mixture was concentrated. This resulted in 4.55 g (91.1%) of 2-[(2R)-1-methylpyrrolidin-2-yl]-1H-1,3-benzodiazol-5-amine as a light brown oil.

LC-MS: [M+1]⁺=217.

¹H-NMR (300 MHz, Methanol-d₄, ppm)>7.28 (dd, J=8.4, 0.6 Hz, 1H), 6.87 (dd, J=2.1, 0.6 Hz, 1H), 6.71 (dd, J=8.4, 2.1 Hz, 1H), 3.54-3.41 (m, 1H), 3.20 (td, J=8.7, 7.5, 2.4 Hz, 1H), 2.49-2.36 (m, 1H), 2.35-2.22 (m, 4H), 2.07-1.85 (m, 3H).

### Intermediate 2: (R)-2-(1-methylpyrrolidin-2-yl)-1-((2-(trimethylsilyl) ethoxy)methyl)-1H-pyrrolo[3,2-c]pyridin-6-amine

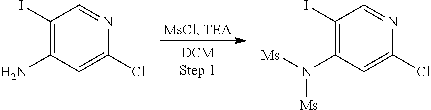 [SMILES: Nc1cc(Cl)ncc1I ; "MsCl, TEA" ; CS(=O)(=O)N(c1cc(Cl)ncc1I)S(C)(=O)=O]

Into a 20-L 4-necked round-bottom flask purged and maintained with an inert atmosphere of nitrogen, was placed 2-chloro-5-iodopyridin-4-amine (490.0 g, 1.90 mol, 1.00 equiv), TEA (974 g, 9.60 mol, 5.00 equiv), DCM (12.30 L). This was followed by the addition of a solution of MsCl (882 g, 7.70 mol, 4.00 equiv) in DCM (7.4 L) dropwise with stirring at 0-5° C. The resulting solution was stirred for 6 hr at 0-10° C. The pH value of the solution was adjusted to 7-8 with NaHCO₃ (1 mol/L). The resulting solution was extracted with 3×5 L of dichloromethane and the organic layers combined and dried over anhydrous sodium sulfate and concentrated. This resulted in 935 g (94.6%) of N-(2-chloro-5-iodopyridin-4-yl)-N-methanesulfonylmethanesulfonamide as yellow oil.

LC-MS: (ES, m/z): [M+1]⁺=411.

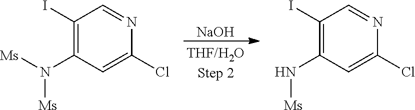 [SMILES: CS(=O)(=O)N(c1cc(Cl)ncc1I)S(C)(=O)=O ; "NaOH" ; CS(=O)(=O)Nc1cc(Cl)ncc1I]

Into a 10-L 4-necked round-bottom flask purged and maintained with an inert atmosphere of nitrogen, was placed N-(2-chloro-5-iodopyridin-4-yl)-N-methanesulfonylmethanesulfonamide (935.0 g, 2.28 mol, 1.00 equiv), THF (4.70 L), H₂O (4.70 L), NaOH (455 g, 11.4 mol, 5.00 equiv). The resulting solution was stirred for 16 hr at room temperature. The resulting mixture was concentrated. The pH value of the solution was adjusted to 3-4 with citric acid (1 mol/L). The solids were collected by filtration. This resulted in 438 g (57.9%) of N-(2-chloro-5-iodopyridin-4-yl) methanesulfonamide as a white solid.

LC-MS (ES, m/z): [M+1]⁺=333.

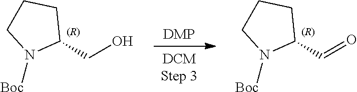

Into a 10-L 4-necked round-bottom flask purged and maintained with an inert atmosphere of nitrogen, was placed tert-butyl (2R)-2-(hydroxymethyl) pyrrolidine-1-carboxylate (530.00 g, 2.6 mol, 1.00 equiv), DCM (5.30 L), DMP (1340 g, 3.16 mol, 1.20 equiv). The resulting solution was stirred for 6 hr at room temperature. The resulting solution was diluted with 5.3 L of H₂O. The resulting solution was extracted with 3×10 L of ethyl acetate and the organic layers combined. The resulting mixture was washed with 3×5 L of NaS₂O₃ (aq.) and 3×5 L of NaHCO₃ (aq.). The resulting mixture was washed with 3×10 L of Brine. The mixture was dried over anhydrous sodium sulfate and concentrated. This resulted in 415 g (79.09%) of tert-butyl (2R)-2-formylpyrrolidine-1-carboxylate as yellow oil.

LC-MS: (ES, m/z): [M+1]⁺=200.

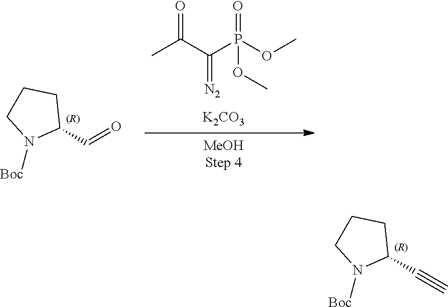

Into a 10-L 4-necked round-bottom flask purged and maintained with an inert atmosphere of nitrogen, was placed K₂CO₃ (348 g, 2.5 mol, 1.20 equiv), methanol (4.15 L), tert-butyl (2R)-2-formylpyrrolidine-1-carboxylate (415.00 g, 2.09 mol, 1.00 equiv), dimethyl (1-diazo-2-oxopropyl)phosphonate (600 g, 3.1 mol, 1.50 equiv). The resulting solution was stirred for 16 hr at room temperature. The resulting solution was diluted with 4 L of H₂O. The resulting solution was extracted with 3×4 L of petroleum ether and the organic layers combined and dried over anhydrous sodium sulfate and concentrated. This resulted in 297 g (73.03%) of tert-butyl (2R)-2-ethynylpyrrolidine-1-carboxylate as yellow oil.

LC-MS: (ES, m/z): [M+1]⁺=196.

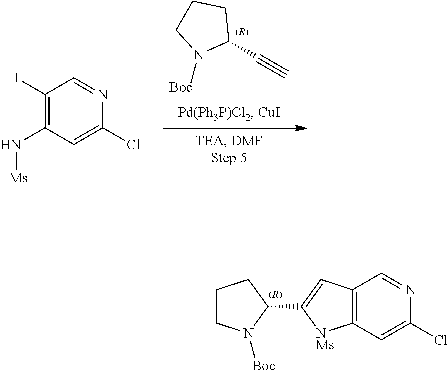

Into a 10-L 4-necked round-bottom flask purged and maintained with an inert atmosphere of nitrogen, was placed N-(2-chloro-5-iodopyridin-4-yl) methanesulfonamide (438.00 g, 1.32 mol, 1.00 equiv), TEA (533 g, 5.27 mol, 4.00 equiv), dimethylformamide (4.40 L), tert-butyl (2R)-2-ethynylpyrrolidine-1-carboxylate (283 g, 1.45 mol, 1.10 equiv), Pd(PPh₃)₂Cl₂ (46 g, 0.066 mol, 0.05 equiv), CuI (25 g, 0.13 mol, 0.10 equiv). The resulting solution was stirred for 6 hr at 55° C. The resulting solution was diluted with 4.4 L of H₂O. The resulting solution was extracted with 3×4.4 L of ethyl acetate and the organic layers combined. The resulting mixture was washed with 3×4.4 L of brine. The mixture was dried over anhydrous sodium sulfate and concentrated. The residue was applied onto a silica gel column with ethyl acetate/petroleum ether (1:5). This resulted in 363 g (68.9%) of tert-butyl (2R)-2-[6-chloro-1-methanesulfonylpyrrolo[3,2-c]pyridin-2-yl]pyrrolidine-1-carboxylate as a white solid.

LC-MS: (ES, m/z): [M+1]⁺=400.

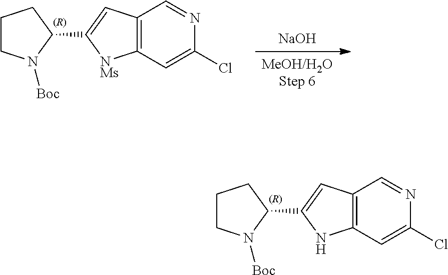

Into a 10-L 4-necked round-bottom flask purged and maintained with an inert atmosphere of nitrogen, was placed tert-butyl (2R)-2-[6-chloro-1-methanesulfonylpyrrolo[3,2-c]pyridin-2-yl]pyrrolidine-1-carboxylate (363.00 g, 0.91 mol, 1.00 equiv), MeOH (2.50 L), H₂O (1.10 L), NaOH (109 g, 2.72 mol, 3.00 equiv). The resulting solution was stirred for 16 hr at room temperature. The resulting mixture was concentrated. The solids were collected by filtration. This resulted in 259 g (88.67%) of tert-butyl (2R)-2-[6-chloro-1H-pyrrolo[3,2-c]pyridin-2-yl]pyrrolidine-1-carboxylate as a white solid.

LC-MS: (ES, m/z): [M+1]⁺=322.

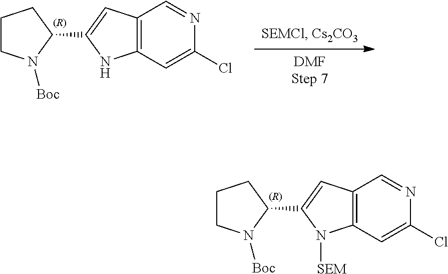

Into a 5-L 4-necked round-bottom flask purged and maintained with an inert atmosphere of nitrogen, was placed tert-butyl (2R)-2-[6-chloro-1H-pyrrolo[3,2-c]pyridin-2-yl]pyrrolidine-1-carboxylate (259.0 g, 0.80 mol, 1.00 equiv), Cs₂CO₃ (787 g, 2.4 mol, 3.00 equiv), DMF (2.60 L). This was followed by the addition of SEMCl (161 g, 0.97 mol, 1.20 equiv) dropwise with stirring at 0-5° C. The resulting solution was stirred for 6 hr at room temperature. The resulting solution was diluted with 2.6 L of H₂O. The resulting solution was extracted with 3×2.6 L of ethyl acetate and the organic layers combined. The resulting mixture was washed with 3×2 L of brine. The mixture was dried over anhydrous sodium sulfate and concentrated. The solids were collected by filtration. This resulted in 248 g (68.2%) of tert-butyl (2R)-2-(6-chloro-1-[[2-(trimethylsilyl)ethoxy]methyl]pyrrolo[3,2-c]pyridin-2-yl) pyrrolidine-1-carboxylate as a white solid.

LC-MS: (ES, m/z): [M+1]⁺=452.

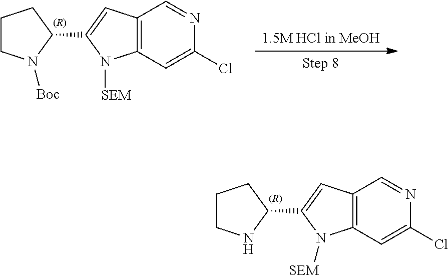

Into a 10-L 4-necked round-bottom flask purged and maintained with an inert atmosphere of nitrogen, was placed tert-butyl (2R)-2-(6-chloro-1-[[2-(trimethylsilyl)ethoxy]methyl]pyrrolo[3,2-c]pyridin-2-yl) pyrrolidine-1-carboxylate (248.00 g, 0.55 mol, 1.00 equiv), MeOH (2.40 L), HCl (1.5 M) in MeOH (1.20 L). The resulting solution was stirred for 12 hr at room temperature. The resulting mixture was concentrated. The resulting solution was diluted with 2.5 L of H₂O. The pH value of the solution was adjusted to 7-8 with NaHCO₃ (1 mol/L). The resulting solution was extracted with 3×2.5 L of dichloromethane and the organic layers combined and concentrated. The residue was applied onto a silica gel column with ethyl acetate/petroleum ether (1:2). This resulted in 177 g (91.7%) of (2R)-2-(6-chloro-1-[[2-(trimethylsilyl)ethoxy]methyl]pyrrolo[3,2-c]pyridin-2-yl) pyrrolidine as a white solid.

LC-MS (ES, m/z): [M+1]⁺=352.

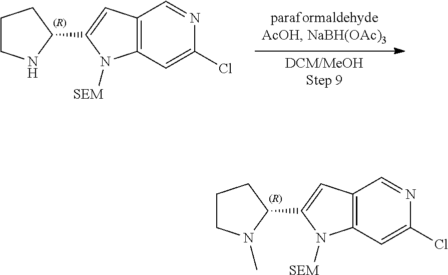

Into a 10-L 4-necked round-bottom flask purged and maintained with an inert atmosphere of nitrogen, was placed (2R)-2-(6-chloro-1-[[2-(trimethylsilyl)ethoxy]methyl]pyrrolo[3,2-c]pyridin-2-yl) pyrrolidine (177.00 g, 0.5 mol, 1.00 equiv), DCM (3.50 L), MeOH (1.77 L), paraformaldehyde (453 g, 5 mol, 10.00 equiv), NaBH(OAc)₃ (640 g, 3 mol, 6.00 equiv). The resulting solution was stirred for 12 hr at room temperature. The pH value of the solution was adjusted to 8-9 with NaHCO₃ (1 mol/L). The solids were filtered out. The resulting solution was extracted with 3×1.7 L of dichloromethane and the organic layers combined and concentrated. The residue was applied onto a silica gel column with ethyl acetate/petroleum ether (1:3). This resulted in 129 g (70.1%) of (2R)-2-(6-chloro-1-[[2-(trimethylsilyl)ethoxy]methyl]pyrrolo[3,2-c]pyridin-2-yl)-1-methylpyrrolidine as yellow oil.

LC-MS: (ES, m/z): [M+1]⁺=366.

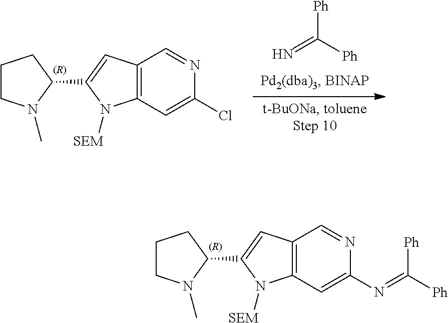

Into a 5-L 4-necked round-bottom flask purged and maintained with an inert atmosphere of nitrogen, was placed (2R)-2-(6-chloro-1-[[2-(trimethylsilyl)ethoxy]methyl]pyrrolo[3,2-c]pyridin-2-yl)-1-methylpyrrolidine (129.00 g, 0.35 mol, 1.00 equiv), toluene (2.60 L), BINAP (22 g, 0.035 mol, 0.10 equiv), t-BuONa (101 g, 1.06 mol, 3.00 equiv), Pd₂(dba)₃ (16 g, 0.017 mol, 0.05 equiv), diphenylmethanimine (192 g, 1.06 mol, 3.00 equiv). The resulting solution was stirred for 16 hr at 110° C. The resulting solution was diluted with 2.6 L of EA. The resulting mixture was washed with 3×1 L of brine. The mixture was dried over anhydrous sodium sulfate and concentrated. The residue was applied onto a silica gel column with THF/PE (1:3). This resulted in 131 g (72.8%) of N-[2-[(2R)-1-methylpyrrolidin-2-yl]-1-[[2-(trimethylsilyl)ethoxy]methyl]pyrrolo[3,2-c]pyridin-6-yl]-1,1-diphenylmethanimine as yellow oil.

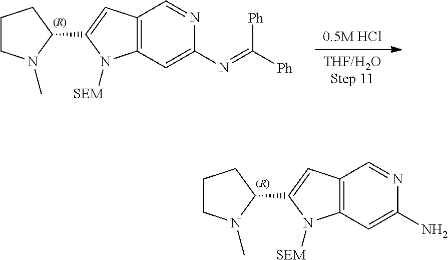

Into a 10-L 4-necked round-bottom flask purged and maintained with an inert atmosphere of nitrogen, was placed N-[2-[(2R)-1-methylpyrrolidin-2-yl]-1-[[2-(trimethylsilyl)ethoxy]methyl]pyrrolo[3,2-c]pyridin-6-yl]-1,1-diphenylmethanimine (131.00 g, 0.26 mol, 1.00 equiv), THF (6.50 L), H₂O (1.10 L), HCl (0.5 M) (88 g, 1.28 mol, 5.00 equiv). The resulting solution was stirred for 12 hr at room temperature. The resulting solution was diluted with 2.6 L of H₂O. The resulting solution was extracted with 3×1 L of dichloromethane and the aqueous layers combined. The pH value of the solution was adjusted to 8-9 with NaHCO₃ (1 mol/L). The resulting solution was extracted with 3×2 L of dichloromethane and the organic layers combined and dried over anhydrous sodium sulfate and concentrated. The residue was applied onto a silica gel column with THF/PE (1:1). This resulted in 53.1 g (59.74%) of 2-[(2R)-1-methylpyrrolidin-2-yl]-1-[[2-(trimethylsilyl)ethoxy]methyl]pyrrolo[3,2-c]pyridin-6-amine as brown oil.

LC-MS: (ES, m/z): [M+1]⁺=347.

¹H-NMR: (300 MHz, CD₃OD. ppm): δ 8.18 (d, J=1.0 Hz, 1H), 6.68 (d, J=1.0 Hz, 1H), 6.46 (s, 1H), 5.61-5.48 (m, 2H), 3.61-3.49 (m, 3H), 3.23 (t, J=7.9 Hz, 1H), 2.46-2.35 (m, 2H), 2.33 (s, 3H), 2.02-1.84 (m, 3H), 0.90 (dd, J=8.8, 7.4 Hz, 2H).

### Intermediate 3: 2-(1-methylpyrrolidin-2-yl)-1-((2-(trimethylsilyl) ethoxy)methyl)-1H-imidazo[4,5-c]pyridin-6-amine

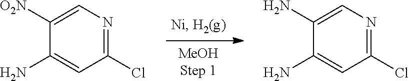 [SMILES: Nc1cc(Cl)ncc1[N+](=O)[O-] ; "MeOHStep 1" ; "Ni, H2(g)" ; Nc1cnc(Cl)cc1N]

Into a 250-mL round-bottom flask, was placed 2-chloro-5-nitropyridin-4-amine (5.00 g, 28.8 mmol, 1.00 equiv), methanol (100.00 mL), Raney Ni (1.69 g, 28.808 mmol, 1.00 equiv). To the above H₂ (g) (5 atm) was introduced in at 25° C. The resulting solution was stirred for 1 overnight at 25° C. The solids were filtered out. The resulting mixture was concentrated. This resulted in 4 g (96.7%) of 6-chloropyridine-3,4-diamine as a brown solid.

LC-MS (ES, m/z): [M+1]⁺=144.

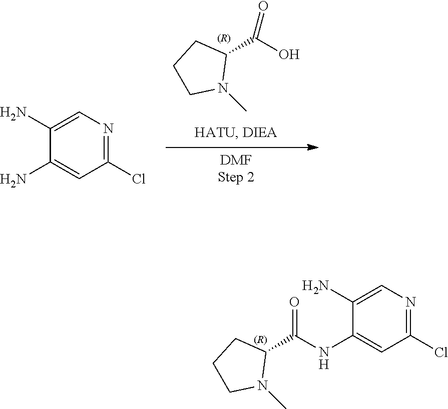

Into a 40-mL round-bottom flask purged and maintained with an inert atmosphere of nitrogen, was placed 6-chloropyridine-3,4-diamine (1.00 g, 6.97 mmol, 1.00 equiv), 1-methylpyrrolidine-2-carboxylic acid (0.99 g, 7.67 mmol, 1.10 equiv), DMF (10.00 mL), HATU (3.18 g, 8.36 mmol, 1.20 equiv), DIEA (3.60 g, 27.86 mmol, 4.00 equiv). The resulting solution was stirred for 1 overnight at 25° C. The reaction was then quenched by the addition of 40 mL of water/ice. The resulting solution was extracted with 4×20 mL of ethyl acetate and the organic layers combined. The resulting mixture was washed with 3×20 mL of brine. The resulting mixture was concentrated. This resulted in 4 g (crude) of N-(5-amino-2-chloropyridin-4-yl)-1-methylpyrrolidine-2-carboxamide as a brown solid.

LC-MS (ES, m/z): [M+1]⁺=255.

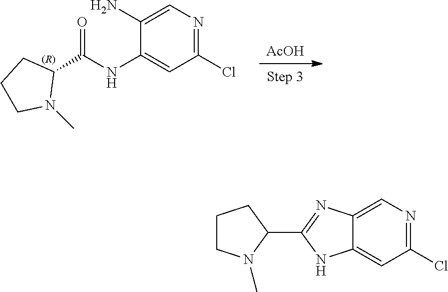

Into a 250-mL round-bottom flask purged and maintained with an inert atmosphere of nitrogen, was placed (2R)—N-(5-amino-2-chloropyridin-4-yl)-1-methylpyrrolidine-2-carboxamide (4.00 g, 1 equiv), acetic acid (80.00 mL). The resulting solution was stirred for 48 hr at 120° C. The resulting mixture was concentrated. The residue was applied onto a silica gel column with THF/PE (1:5) . . . . This resulted in 1.7 g (32%) of 2-[6-chloro-1H-imidazo[4,5-c]pyridin-2-yl]-1-methylpyrrolidine as a brown solid.

LC-MS: (ES, m/z): [M+1]⁺=237.

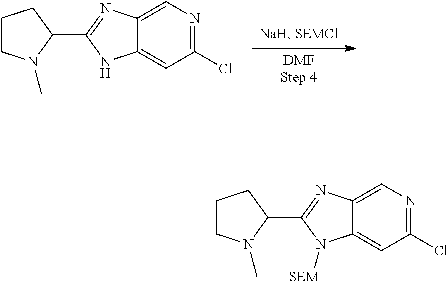

Into a 50-mL 3-necked round-bottom flask purged and maintained with an inert atmosphere of nitrogen, was placed 2-[6-chloro-1H-imidazo[4,5-c]pyridin-2-yl]-1-methylpyrrolidine (1.00 g, 4.225 mmol, 1.00 equiv), DMF (15.00 mL). NaH (0.20 g, 8.450 mmol, 2.00 equiv) was added and the resulting solution was stirred for 30 min at 0° C. [2-(chloromethoxy)ethyl]trimethylsilane (0.70 g, 4.225 mmol, 1.00 equiv) was added and the resulting solution was allowed to react, with stirring, for an additional 90 min at 0° C. The reaction was then quenched by the addition of 40 mL of water/ice. The resulting solution was extracted with 3×20 mL of ethyl acetate and the organic layers combined. The resulting mixture was washed with 3×20 mL of brine. The resulting mixture was concentrated. The residue was applied onto a silica gel column with PE/THF (1:2). This resulted in 350 mg (22.6%) of 2-(6-chloro-1-[[2-(trimethylsilyl)ethoxy]methyl]imidazo[4,5-c]pyridin-2-yl)-1-methylpyrrolidine as brown oil.

LC-MS: (ES, m/z): [M+1]⁺=367.

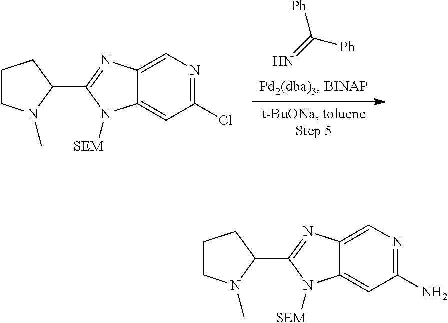

Into a 50 mL round-bottom flask were added 2-(6-chloro-1-{[2-(trimethylsilyl)ethoxy]methyl}imidazo[4,5-c]pyridin-2-yl)-1-methylpyrrolidine (350 mg, 0.954 mmol, 1.00 equiv), diphenylmethanimine (518.57 mg, 2.862 mmol, 3.0 equiv), BINAP (118.78 mg, 0.191 mmol, 0.2 equiv), tris((1E,4E)-1,5-diphenylpenta-1,4-dien-3-one) trichloromethane dipalladium (98.72 mg, 0.095 mmol, 0.1 equiv), t-BuONa (274.98 mg, 2.862 mmol, 3.0 equiv) and toluene (10 mL, 93.989 mmol, 98.55 equiv) at room temperature. The resulting mixture was stirred for 5 h at 110 degrees C. under nitrogen atmosphere. The mixture was allowed to cool down to room temperature. The reaction was quenched by the addition of water/ice (60 mL) at room temperature. The aqueous layer was extracted with CH₂Cl₂ (3×30 mL). The resulting mixture was concentrated under reduced pressure. The residue was dissolved in THF (25 ml) and HCl (0.5M) (10 mL) was added into the solution. The resulting mixture was stirred for 10 h at room temperature. The resulting mixture was concentrated under reduced pressure to remove THF. The aqueous layer was extracted with EtOAc (3×10 mL). The aqueous layer was basified to pH 8 with saturated NaHCO₃ (aq.). The aqueous layer was extracted with CH₂Cl₂ (3×40 mL). The resulting mixture was concentrated under reduced pressure. The residue was purified by silica gel column chromatography, eluted with PE/EA (1:9) to afford 2-(1-methylpyrrolidin-2-yl)-1-((2-(trimethylsilyl) ethoxy)methyl)-1H-imidazo[4,5-c]pyridin-6-amine (180 mg, 53%) as a brown solid.

LC-MS: (ES, m/z): [M+1]⁺=348.

Intermediate 4: (R)-2-(1-methylpiperidin-2-yl)-1-((2-(trimethylsilyl) ethoxy)methyl)-1H-pyrrolo[3,2-c]pyridin-6-amine

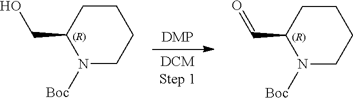

Into a 50-mL 3-necked round-bottom flask, was placed tert-butyl (2R)-2-(hydroxymethyl)piperidine-1-carboxylate (5.00 g, 23.22 mmol, 1.00 equiv), Dess-Martin periodinane (19.70 g, 46.45 mmol, 2.00 equiv), DCM (20.00 mL). The resulting solution was stirred for 3 hr at room temperature. The reaction was then quenched by the addition of Na₂S₂O₃ (aq). The resulting solution was extracted with 2×50 mL of dichloromethane and the organic layers combined and concentrated. This resulted in 4.0 g (80.8%) of tert-butyl (2R)-2-formylpiperidine-1-carboxylate as brown oil.

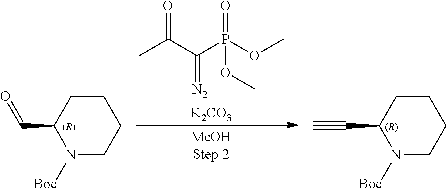

Into a 50-mL 3-necked round-bottom flask, was placed tert-butyl (2R)-2-formylpiperidine-1-carboxylate (4.00 g, 18.755 mmol, 1.00 equiv), K₂CO₃ (3.11 g, 22.506 mmol, 1.20 equiv), MeOH (12.00 mL). This was followed by the addition of a solution of dimethyl (1-diazo-2-oxopropyl)phosphonate (5.40 g, 0.028 mmol, 1.50 equiv) in MeOH (6 mL) dropwise with stirring at 0° C. The resulting solution was stirred for 6 hr at room temperature. The resulting solution was extracted with 2×50 mL of petroleum ether and the organic layers combined and concentrated. This resulted in 2 g (51%) of tert-butyl (2R)-2-ethynylpiperidine-1-carboxylate as yellow oil.

GC-MS: (ES, m/z): [M−81]=128.

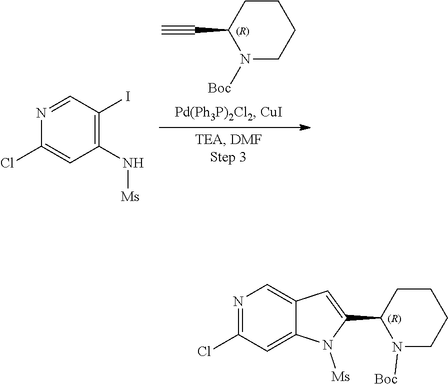

Into a 50-mL 3-necked round-bottom flask purged and maintained with an inert atmosphere of nitrogen, was placed N-(2-chloro-5-iodopyridin-4-yl) methanesulfonamide (700.0 mg, 2.1 mmol, 1.00 equiv), tert-butyl (2R)-2-ethynylpiperidine-1-carboxylate (881.11 mg, 4.210 mmol, 2.00 equiv), CuI (40.09 mg, 0.211 mmol, 0.10 equiv), TEA (852.02 mg, 8.420 mmol, 4.00 equiv), DMF (10.00 mL), Pd(PPh₃)₂Cl₂ (295.5 mg, 0.421 mmol, 0.20 equiv). The resulting solution was stirred for 2 hr at 55° C. The solids were filtered out. The resulting mixture was concentrated. The residue was applied onto a silica gel column with ethyl acetate/petroleum ether (1:10). This resulted in 540 mg (62%) of tert-butyl (2R)-2-[6-chloro-1-methanesulfonylpyrrolo[3,2-c]pyridin-2-yl]piperidine-1-carboxylate as a brown solid.

LC-MS: (ES, m/z): [M+H]=414

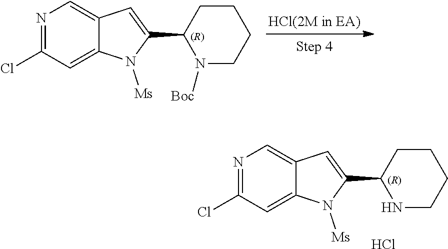

Into a 50-mL round-bottom flask, was placed tert-butyl (2R)-2-[6-chloro-1-methanesulfonylpyrrolo[3,2-c]pyridin-2-yl]piperidine-1-carboxylate (430.00 mg), HCl (gas) in ethyl acetate (10.00 mL). The resulting solution was stirred for 6 hr at room temperature. The resulting mixture was concentrated. This resulted in 380 mg of (2R)-2-[6-chloro-1-methanesulfonylpyrrolo[3,2-c]pyridin-2-yl]piperidine hydrochloride as a brown solid.

LC-MS: (ES, m/z): [M+H−HCl]=314.

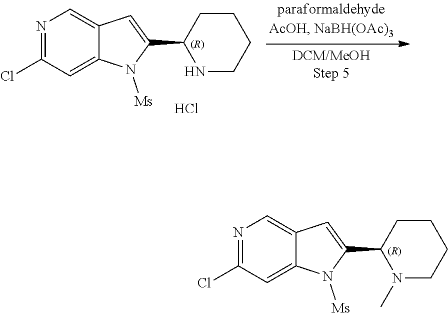

Into a 100-mL 3-necked round-bottom flask, was placed (2R)-2-[6-chloro-1-methanesulfonylpyrrolo[3,2-c]pyridin-2-yl]piperidine hydrochloride (380.00 mg, 1.089 mmol, 1.00 equiv), DCM (20.00 mL), MeOH (10.00 mL), paraformaldehyde (488.63 mg, 5.43 mmol, 5.00 equiv), NaBH(OAc)₃ (2299.37 mg, 10.85 mmol, 10.00 equiv). The resulting solution was stirred for 12 hr at room temperature. The reaction was then quenched by the addition of 20 mL of water. The resulting solution was extracted with 2×30 mL of dichloromethane and the organic layers combined and concentrated. The residue was applied onto a silica gel column with ethyl acetate/petroleum ether (1:1). This resulted in 201 mg (56.5%) of (2R)-2-[6-chloro-1-methanesulfonylpyrrolo[3,2-c]pyridin-2-yl]-1-methylpiperidine as a white solid.

LC-MS: (ES, m/z): [M+H]=328.

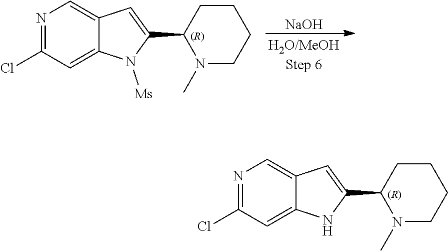

Into a 50-mL round-bottom flask, was placed (2R)-2-[6-chloro-1-methanesulfonylpyrrolo[3,2-c]pyridin-2-yl]-1-methylpiperidine (185.00 mg, 0.564 mmol, 1.00 equiv), NaOH (67.71 mg, 0.000 mmol, 3.00 equiv), H₂O (1.00 mL), MeOH (5.00 mL). The resulting solution was stirred for 2 hr at room temperature. The resulting solution was extracted with 2×20 mL of ethyl acetate and the organic layers combined and concentrated. This resulted in 120 mg (85.2%) of (2R)-2-[6-chloro-1H-pyrrolo[3,2-c]pyridin-2-yl]-1-methylpiperidine as a brown solid.

LC-MS: (ES, m/z): [M+H]=250.

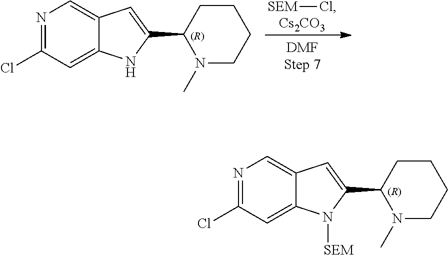

Into a 50-mL 3-necked round-bottom flask, was placed (2R)-2-[6-chloro-1H-pyrrolo[3,2-c]pyridin-2-yl]-1-methylpiperidine (120.0 mg, 0.480 mmol, 1.00 equiv), Cs₂CO₃ (469.7 mg, 1.44 mmol, 3.00 equiv), DMF (5.00 mL), SEM-Cl (120.16 mg, 0.720 mmol, 1.50 equiv). The resulting solution was stirred for 2 hr at room temperature. The solids were filtered out. The resulting solution was extracted with 2×20 mL of ethyl acetate and the organic layers combined and concentrated. The residue was applied onto a silica gel column with ethyl acetate/petroleum ether (1:2). This resulted in 85 mg (46.6%) of (2R)-2-(6-chloro-1-[[2-(trimethylsilyl) ethoxy]methyl]pyrrolo[3,2-c]pyridin-2-yl)-1-methylpiperidine as light brown oil.

LC-MS: (ES, m/z): [M+H]=380.

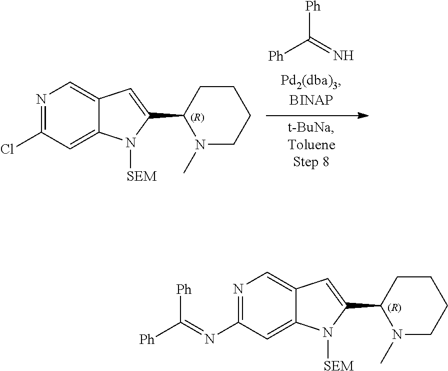

Into a 8-mL sealed tube purged and maintained with an inert atmosphere of nitrogen, was placed 2-(6-chloro-1-[[2-(trimethylsilyl)ethoxy]methyl]-octahydropyrrolo[3,2-c]pyridin-2-yl)-1-methylpiperidine (80.00 mg, 0.206 mmol, 1.00 equiv), benzenemethanimine (112.09 mg, 0.618 mmol, 3.00 equiv), t-BuONa (59.43 mg, 0.618 mmol, 3.00 equiv), toluene (3.00 mL), Pd₂(dba)₃·CHCl₃ (23.71 mg, 0.041 mmol, 0.20 equiv), BINAP (51.35 mg, 0.082 mmol, 0.40 equiv). The resulting solution was stirred for 5 hr at 100° C. The resulting mixture was concentrated. This resulted in 100 mg (crude) of N-[2-[(2R)-1-methylpiperidin-2-yl]-1-[[2-(trimethylsilyl)ethoxy]methyl]pyrrolo[3,2-c]pyridin-6-yl]-1,1-diphenylmethanimine as brown oil.

LC-MS: (ES, m/z): [M+H]=525.

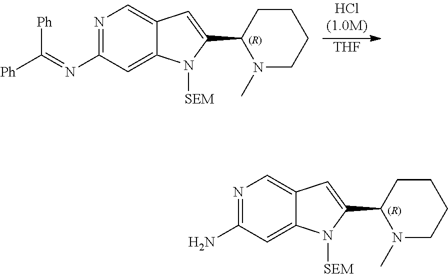

Into a 50-mL round-bottom flask, was placed N-[2-[(2R)-1-methylpiperidin-2-yl]-1-[[2-(trimethylsilyl)ethoxy]methyl]pyrrolo[3,2-c]pyridin-6-yl]-1,1-diphenylmethanimine (100.00 mg, 0.191 mmol, 1.00 equiv), THF (5.00 mL), HCl (5.00 mL). The resulting solution was stirred for 16 hr at room temperature. The resulting solution was extracted with 2×20 mL of ethyl acetate and the organic layers combined and concentrated. The residue was applied onto a silica gel column with ethyl acetate/petroleum ether (1:1). This resulted in 44 mg (34% for two steps) of 2-[(2R)-1-methylpiperidin-2-yl]-1-[[2-(trimethylsilyl)ethoxy]methyl]pyrrolo[3,2-c]pyridin-6-amine as a brown solid. The product was further purified by SFC with the following conditions (Column: Lux 5 um Amylose-1, 5*25 cm, 10 um; Mobile Phase A: CO₂, Mobile Phase B: IPA (0.5% 2M NH₃-MeOH); Flow rate: 160 mL/min; Gradient: isocratic 40% B; Column Temperature (° C.): 35; Back Pressure (bar): 100; Wave Length: 220 nm: RT1 (min): 4.47; RT2 (min): 5.89; Sample Solvent: ACN; Injection Volume: 2 mL and the major enantiomer collected to obtain material >98% ee.

LC-MS: (ES, m/z): [M+H]=361

LC-MS: (ES, m/z): [M+H]=361

¹H-NMR (300 MHz, Methanol-d₄, ppm): δ 8.23-8.17 (m, 1H), 6.68 (s, 1H), 6.47 (s, 1H), 3.56 (t, J=8.2 Hz, 2H), 3.08 (d, J=11.9 Hz, 1H), 2.24 (d, J=14.1 Hz, 1H), 2.15 (s, 3H), 1.97-1.68 (m, 5H), 1.48 (d, J=10.5 Hz, 1H), 1.17 (d, J=6.2 Hz, 3H), 0.91 (t, J=8.1 Hz, 2H), −0.22 (s, 9H).

### Intermediate 5: 2-[(2R)-1-methylpyrrolidin-2-yl]-1-{[2-(trimethylsilyl)ethoxy]methyl}pyrrolo[3,2-b]pyridin-6-amine

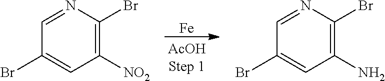

Into a 2 L 4-necked round-bottom flask were added 2,5-dibromo-3-nitropyridine (60 g, 212.85 mmol, 1.00 equiv) and acetic acid (900 mL, 70.41 equiv) at room temperature. To the stirred solution was added iron (71.32 g, 1277 mmol, 6.0 equiv) in portions at room temperature under nitrogen atmosphere. The resulting mixture was stirred for 6 hr at room temperature. The reaction was quenched by the addition of water/ice (2 L) at room temperature. The precipitated solids were collected by filtration and washed with water (3×300 mL). This resulted in 2,5-dibromopyridin-3-amine (60 g, crude) as a brown solid.

LC-MS (ES, m/z): [M+1]⁺=251.

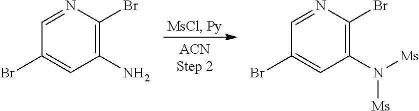 [SMILES: Nc1cc(Br)cnc1Br ; "MsCl, Py" ; CS(=O)(=O)N(c1cc(Br)cnc1Br)S(C)(=O)=O]

Into a 3 L 4-necked round-bottom flask were added 2,5-dibromopyridin-3-amine (60 g, 238.2 mmol, 1.00 equiv), acetonitrile (ACN, 1200 mL), and pyridine (56.52 g, 714.5 mmol, 3.0 equiv) at room temperature. To a stirred solution were added methanesulfonyl chloride (81.8 g, 714.5 mmol, 3.0 equiv) dropwise at 0° C. under nitrogen atmosphere. The resulting mixture was stirred for 6 hr at room temperature. The reaction was quenched by the addition of water/ice (2000 mL) at room temperature. The resulting mixture was extracted with CH₂Cl₂ (2×1000 mL). The combined organic layers were washed with brine (3×500 mL), dried over anhydrous Na₂SO₄. After filtration, the filtrate was concentrated under reduced pressure. This resulted in N-(2,5-dibromopyridin-3-yl)-N-methanesulfonylmethanesulfonamide (78.5 g, 80.76%) as a brown solid which was used directly in the next step.

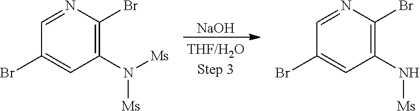

Into a 5 L 4-necked round-bottom flask were added N-(2,5-dibromopyridin-3-yl)-N-methanesulfonylmethanesulfonamide (78.5 g, 192.4 mmol, 1.00 equiv), tetrahydrofuran (2400 mL, 173 equiv), water (471 mL, 136 equiv) and NaOH (46.16 g, 1154.2 mmol, 6.0 equiv) at room temperature. The resulting mixture was stirred for 16 hr at room temperature under nitrogen atmosphere. The aqueous layer was extracted with CH₂Cl₂ (3×1500 mL). The resulting mixture was concentrated under reduced pressure. The residue was purified by silica gel column chromatography, eluted with PE/EA (1:1) afford N-(2,5-dibromopyridin-3-yl) methanesulfonamide (55.1 g, 86.8%) as a brown solid.

LC-MS (ES, m/z): [M−1]⁻=327.

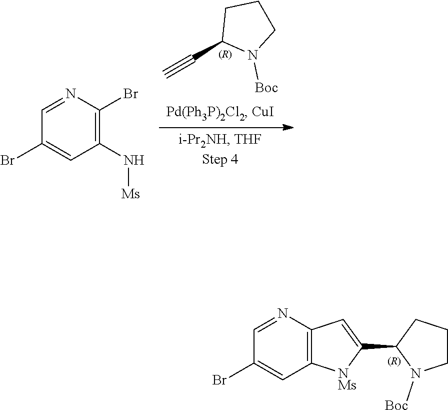

Into a 2 L 4-necked round-bottom flask were added N-(2,5-dibromopyridin-3-yl) methanesulfonamide (29 g, 87.9 mmol, 1.00 equiv), tert-butyl (2R)-2-ethynylpyrrolidine-1-carboxylate (22.31 g, 114.2 mmol, 1.3 equiv), copper (I) iodide (1.67 g, 8.8 mmol, 0.1 equiv), Pd(PPh₃)₂Cl₂, diisopropylamine (71.14 g, 703 mmol, 8.0 equiv) and tetrahydrofuran (850 mL) at room temperature. The resulting mixture was stirred for 2 hr at 50° C. under nitrogen atmosphere. The mixture was allowed to cool down to room temperature. The reaction was quenched by the addition of sat. NH₄Cl (aq.) (1500 mL) at room temperature. The aqueous layer was extracted with EtOAc (3×600 mL). The resulting mixture was concentrated under reduced pressure. The residue was purified by silica gel column chromatography, eluted with PE/EA (1:1) to afford tert-butyl (2R)-2-{6-bromo-1-methanesulfonylpyrrolo[3,2-b]pyridin-2-yl}pyrrolidine-1-carboxylate (18.5 g, 47.4%) as a brown solid.

LC-MS (ES, m/z): [M+1]⁺⁻=444.

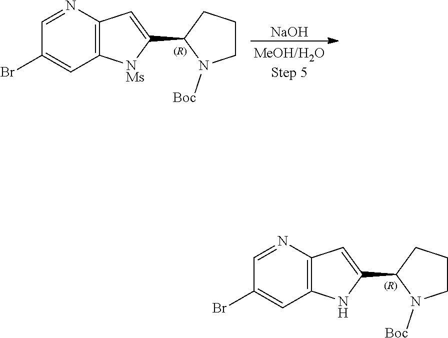

Into a 500 mL 3-necked round-bottom flask were added tert-butyl (2R)-2-{6-bromo-1-methanesulfonylpyrrolo[3,2-b]pyridin-2-yl}pyrrolidine-1-carboxylate (18.7 g, 42.1 mmol, 1.00 equiv), H₂O (56 mL), MeOH (130 mL) and NaOH (5.1 g, 126.3 mmol, 3.0 equiv) at room temperature. The resulting mixture was stirred for 16 hr at room temperature under nitrogen atmosphere. The resulting mixture was diluted with water (250 mL). The aqueous layer was extracted with CH₂Cl₂ (3×200 mL). The resulting mixture was concentrated under reduced pressure. This resulted in tert-butyl (2R)-2-{6-bromo-1H-pyrrolo[3,2-b]pyridin-2-yl}pyrrolidine-1-carboxylate (14.6 g, 94.7%) as a brown solid.

LC-MS (ES, m/z): [M+1]⁺=366.

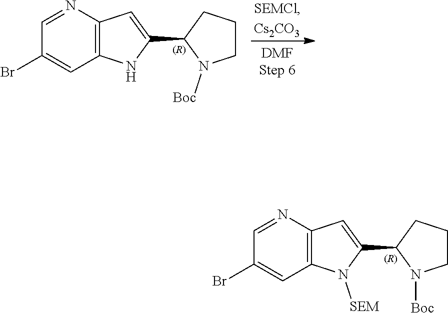

Into a 500 mL 3-necked round-bottom flask were added tert-butyl (2R)-2-{6-bromo-1H-pyrrolo[3,2-b]pyridin-2-yl}pyrrolidine-1-carboxylate (14.6 g, 40 mmol, 1.00 equiv), N,N-dimethylformamide (220 mL) and Cs₂CO₃ (39.1 g, 119.6 mmol, 3.0 equiv) at room temperature. To the above mixture was added [2-(chloromethoxy)ethyl]trimethylsilane (6.65 g, 39.86 mmol, 1.0 equiv) dropwise over 20 min at room temperature. The resulting mixture was stirred for additional 5.5 hr at room temperature. The reaction was quenched with water/ice (600 mL) at room temperature. The aqueous layer was extracted with EtOAc (3×300 mL). The resulting mixture was concentrated under reduced pressure. The residue was purified by silica gel column chromatography, eluted with PE/EA (3:1) to afford tert-butyl (2R)-2-(6-bromo-1-{[2-(trimethylsilyl)ethoxy]methyl}pyrrolo[3,2-b]pyridin-2-yl) pyrrolidine-1-carboxylate (14.6 g, 73.8%) as a brown oil.

LC-MS (ES, m/z): [M+1]⁺=496.

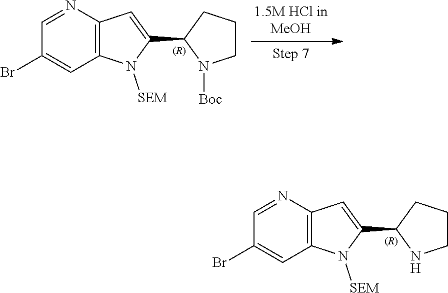

Into a 500 mL 3-necked round-bottom flask were added tert-butyl (2R)-2-(6-bromo-1-{[2-(trimethylsilyl)ethoxy]methyl}pyrrolo[3,2-b]pyridin-2-yl) pyrrolidine-1-carboxylate (14.6 g, 29.4 mmol, 1.00 equiv) and HCl in MeOH (200 mL, 1.5 mol/L) at room temperature. The resulting mixture was stirred for 12 hr at room temperature under nitrogen atmosphere. The reaction was quenched with sat. NaHCO₃ (aq.) (600 mL) at room temperature. The mixture/residue was basified to pH 8 with saturated NaHCO₃. The aqueous layer was extracted with EtOAc (3×300 mL). The resulting mixture was concentrated under reduced pressure. This resulted in (2R)-2-(6-bromo-1-{[2-(trimethylsilyl)ethoxy]methyl}pyrrolo[3,2-b]pyridin-2-yl) pyrrolidine (9.2 g, 78.9%) as a brown oil.

LC-MS (ES, m/z): [M+1]⁺⁻=396.

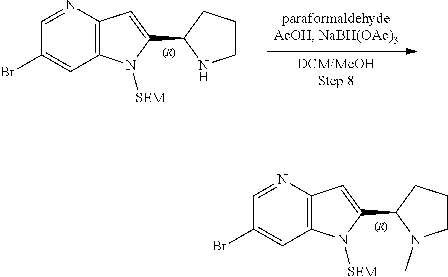

To a stirred solution of (2R)-2-(6-bromo-1-{[2-(trimethylsilyl)ethoxy]methyl}pyrrolo[3,2-b]pyridin-2-yl) pyrrolidine (2.3 g, 5.8 mmol, 1.00 equiv) and paraformaldehyde (2.61 g, 58.02 mmol, 10 equiv), acetic acid (0.35 g, 5.8 mmol, 1.0 equiv) in methanol (46 mL), DCM (23 mL) was added NaBH(OAc)₃ (11.07 g, 52.218 mmol, 9.0 equiv) in portions at room temperature under nitrogen atmosphere. The resulting mixture was stirred for 12 hr at room temperature under nitrogen atmosphere. The reaction was quenched by the addition of water/ice (200 mL) at room temperature. The mixture/residue was basified to pH 8 with NaHCO₃. The resulting mixture was filtered, the filter cake was washed with CH₂Cl₂ (30 mL). The resulting mixture was extracted with CH₂Cl₂ (3× 60 mL). The combined organic layers were washed with brine (2×40 mL), dried over anhydrous Na₂SO₄. After filtration, the filtrate was concentrated under reduced pressure. This resulted in (2R)-2-(6-bromo-1-{[2-(trimethylsilyl)ethoxy]methyl}pyrrolo[3,2-b]pyridin-2-yl)-1-methylpyrrolidine (1.93 g, 81%) as a brown oil.

LC-MS (ES, m/z): [M+1]⁺⁻=410.

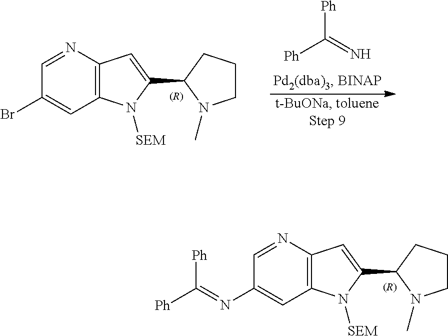

Into a 100 mL 3-necked round-bottom flask were added (2R)-2-(6-bromo-1-{[2-(trimethylsilyl)ethoxy]methyl}pyrrolo[3,2-b]pyridin-2-yl)-1-methylpyrrolidine (1.93 g, 4.7 mmol, 1.00 equiv), diphenylmethanimine (2.56 g, 14.1 mmol, 3.0 equiv), BINAP (0.59 g, 0.940 mmol, 0.2 equiv), Pd₂(dba)₃ (0.43 g, 0.47 mmol, 0.1 equiv), sodium tert-butoxide (1.36 g, 14.1 mmol, 3.0 equiv) and toluene (50 mL) at room temperature. The resulting mixture was stirred for 16 hr at 110° C. under nitrogen atmosphere. The mixture was allowed to cool down to room temperature. The reaction was quenched with water/ice at room temperature. The aqueous layer was extracted with CH₂Cl₂ (3×40 mL). The resulting mixture was concentrated under reduced pressure. This resulted in N-{2-[(2R)-1-methylpyrrolidin-2-yl]-1-{[2-(trimethylsilyl)ethoxy]methyl}pyrrolo[3,2-b]pyridin-6-yl}-1,1-diphenylmethanimine (5.5 g, crude) as a brown oil.

LC-MS (ES, m/z): [M+1]⁺⁻=511.

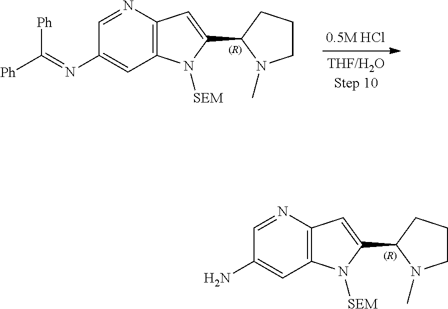

Into a 1000 mL 3-necked round-bottom flask were added N-{2-[(2R)-1-methylpyrrolidin-2-yl]-1-{[2-(trimethylsilyl)ethoxy]methyl}pyrrolo[3,2-b]pyridin-6-yl}-1,1-diphenylmethanimine (5.5 g, 10.8 mmol, 1.00 equiv), HCl (55 mL) (1 mol/L) and tetrahydrofuran (275 mL) at room temperature. The resulting mixture was stirred for 12 hr at room temperature under nitrogen atmosphere. The resulting mixture was concentrated under reduced pressure to remove THF. The aqueous layer was extracted with EtOAc (3×50 mL). The aqueous layer basified to pH 8 with NaHCO₃. The aqueous layer was extracted with CH₂Cl₂ (3×40 mL). The combined organic layers were washed with brine (3×40 mL), dried over anhydrous Na₂SO₄. After filtration, the filtrate was concentrated under reduced pressure. This resulted in 2-[(2R)-1-methylpyrrolidin-2-yl]-1-{[2-(trimethylsilyl)ethoxy]methyl}pyrrolo[3,2-b]pyridin-6-amine (1.4 g, 37.5%) as a brown oil.

LC-MS (ES, m/z): [M+1]⁺=347.

¹H-NMR (400 MHZ, Methanol-d₄, ppm) δ 7.92 (d, J=2.3 Hz, 1H), 7.27 (d, J=2.3 Hz, 1H), 6.51 (s, 1H), 5.64-5.47 (m, 3H), 3.61-3.50 (m, 2H), 3.28-3.19 (m, 1H), 2.34 (m, 4H), 2.03-1.85 (m, 5H), 0.93-0.85 (m, 2H), −0.04 (s, 9H).

### Intermediate 6: (R)-2-(1-methylpyrrolidin-2-yl)-1H-indol-6-amine

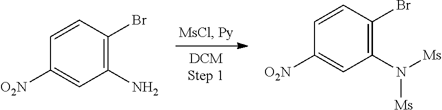 [SMILES: Nc1cc([N+](=O)[O-])ccc1Br ; CS(=O)(=O)N(c1cc([N+](=O)[O-])ccc1Br)S(C)(=O)=O]

Into a 250 mL 3-necked round-bottom flask were added benzenamine, 2-bromo-5-nitro-(10 g, 46.078 mmol, 1.00 equiv), pyridine (80 mL) and DCM (120 mL) at room temperature. To the above mixture was added methanesulfonyl chloride (21.1 g, 184.3 mmol, 4 equiv) dropwise at 0° C. The resulting mixture was stirred for overnight at room temperature. The reaction was quenched by the addition of NaHCO₃ (aq, 500 mL) at room temperature. The resulting mixture was separated and the aqueous layer was extracted with DCM (2×100 mL). The combined organic layers were washed with brine (1×400 mL), dried over anhydrous Na₂SO₄. After filtration, the filtrate was concentrated under reduced pressure. This resulted in N-(2-bromo-5-nitrophenyl)-N-methanesulfonylmethanesulfonamide (17 g, crude) as a brown solid. The crude product was used in the next step directly without further purification.

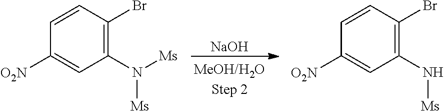

Into a 500 mL round-bottom flask were added N-(2-bromo-5-nitrophenyl)-N-methanesulfonylmethanesulfonamide (17 g, 45.6 mmol, 1.00 equiv) and THF (280 mL) at room temperature. To the above mixture was added a solution of NaOH (11 g, 275.020 mmol, 6.04 equiv) in H₂O (140 mL). The resulting mixture was stirred for overnight at room temperature. The resulting mixture was concentrated under vacuum. The resulting mixture was diluted with water (250 mL). The mixture was acidified to pH 3 with HCl (aq.). The precipitated solids were collected by filtration and washed with water (2×100 mL). The resulting solid was dried under infrared light. This resulted in N-(2-bromo-5-nitrophenyl) methanesulfonamide (11 g, 81.8%) as a brown solid.

¹H-NMR (300 MHz, DMSO-d₆, ppm) δ 9.85 (s, 1H), 8.21 (d, J=3 Hz, 1H), 8.0 (s, 2H), 3.16 (s, 3H).

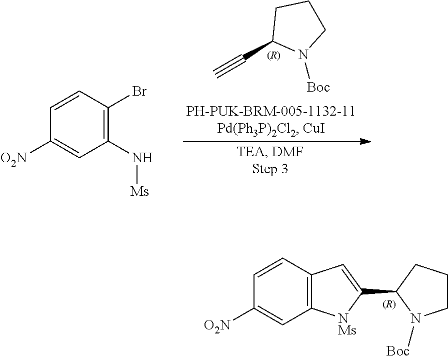

Into a 500 mL round-bottom flask were added N-(2-bromo-5-nitrophenyl) methanesulfonamide (10 g, 34 mmol, 1.00 equiv), tert-butyl (2R)-2-ethynylpyrrolidine-1-carboxylate (7.94 g, 40.66 mmol, 1.2 equiv) and TEA (27.43 g, 271.07 mmol, 8.00 equiv) at room temperature. The resulting mixture was bubbled with N₂ for 10 min, then Pd(PPh₃)₂Cl₂ (2.38 g, 3.389 mmol, 0.1 equiv) and CuI (1.29 g, 6.78 mmol, 0.2 equiv) were added. The resulting mixture was stirred for overnight at 70° C. under nitrogen atmosphere. The reaction was quenched by the addition of sat. NH₄Cl (aq.) (75 mL) at 0° C. The precipitated solids were collected by filtration and washed with water (2×100 mL). The residue was dissolved in ethyl acetate (500 mL). The resulting mixture was dried over anhydrous Na₂SO₄. After filtration, the filtrate was concentrated under reduced pressure. The residue was purified by silica gel column chromatography, eluted with PE/EA (3:1) to afford tert-butyl (2R)-2-(1-methanesulfonyl-6-nitroindol-2-yl) pyrrolidine-1-carboxylate (9 g, 64.9%) as a yellow solid.

LC-MS (ES, m/z): [M+H−56+41]⁺=395.

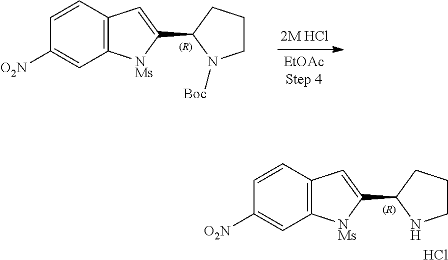

Into a 100 mL round-bottom flask were added tert-butyl (2R)-2-(1-methanesulfonyl-6-nitroindol-2-yl) pyrrolidine-1-carboxylate (620 mg, 1.514 mmol, 1.00 equiv), DCM (13.00 mL, 204.466 mmol, 135.05 equiv), and HCl (2M in EA) (7.57 mL, 15.140 mmol, 10 equiv) at room temperature. The resulting mixture was stirred for overnight at room temperature. The resulting mixture was concentrated under vacuum. This resulted in 1-methanesulfonyl-6-nitro-2-[(2R)-pyrrolidin-2-yl]indole hydrochloride (480 mg, 91.7%) as a yellow solid. The crude was used in the next step directly without further purification.

LC-MS (ES, m/z): [M+1]⁺=310.

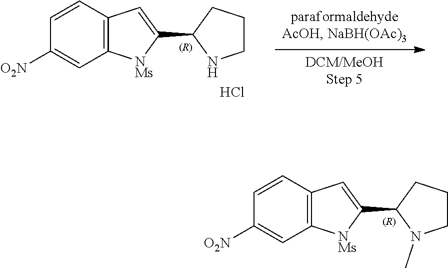

Into a 500 mL 3-necked round-bottom flask were added 1-methanesulfonyl-6-nitro-2-[(2R)-pyrrolidin-2-yl]indole hydrochloride (10 g, 28.9 mmol, 1.00 equiv), DCM (200 mL), MeOH (100 mL), and paraformaldehyde (10.42 g, 115.7 mmol, 4 equiv) at room temperature. To the above mixture was added NaBH(OAc)₃ (36.77 g, 173.5 mmol, 6 equiv) in 3 portions over 1.5 hr at room temperature. The resulting mixture was stirred for additional overnight at room temperature. The reaction was quenched by the addition of NaHCO₃ (sat.) (400 mL) at room temperature. The aqueous layer was extracted with DCM (3×100 mL). The combined organic layers were dried over anhydrous Na₂SO₄. After filtration, the filtrate was concentrated under reduced pressure. The residue was purified by silica gel column chromatography, eluted with PE/EA (1:9) to afford 1-methanesulfonyl-2-[(2R)-1-methylpyrrolidin-2-yl]-6-nitroindole (5.7 g, 61%) as a yellow solid.

LC-MS (ES, m/z): [M+1]⁺=324.

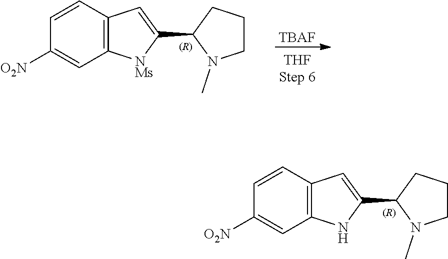

Into a 250 mL 3-necked round-bottom flask were added 1-methanesulfonyl-2-[(2R)-1-methylpyrrolidin-2-yl]-6-nitroindole (5.7 g, 17.63 mmol, 1.00 equiv), THF (90 mL) and TBAF (23.04 g, 88.1 mmol, 5 equiv) at room temperature. The resulting mixture was stirred for overnight at room temperature. The resulting mixture was concentrated under reduced pressure. The residue was dissolved in DCM (20 mL). The residue was purified by silica gel column chromatography, eluted with DCM/MeOH (25:1) to afford 2-[(2R)-1-methylpyrrolidin-2-yl]-6-nitro-1H-indole (3.2 g, 74%) as a yellow solid.

LC-MS (ES, m/z): [M+1]⁺=246.

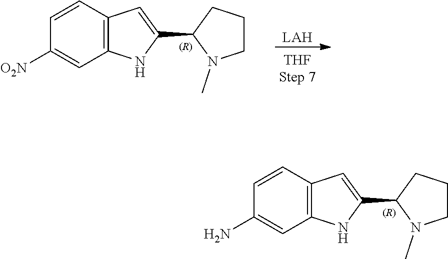

Into a 8 mL vial were added LAH (154.74 mg, 4.080 mmol, 10 equiv) and THF (1 mL) at room temperature. To the above mixture was added a mixture of 2-[(2R)-1-methylpyrrolidin-2-yl]-6-nitro-1H-indole (100 mg, 0.408 mmol, 1.00 equiv) in THF (2 mL) dropwise at 80° C. The resulting mixture was stirred for additional 2 hr at 80° C. under nitrogen atmosphere. The reaction was quenched by the addition of Na₂SO₄·10H₂O (2 g) at 0° C. The resulting mixture was filtered, the filter cake was washed with THF (3×2 mL). The filtrate was concentrated under reduced pressure. This resulted in (R)-2-(1-methylpyrrolidin-2-yl)-1H-indol-6-amine (82 mg, 57.4%) as a black solid. The crude product was used immediately in the next step directly without further purification.

LC-MS (ES, m/z): [M+1]⁺=216.

### Acid Intermediates:

Acid 1: 3-(pyridin-4-yl)benzo[d]isoxazole-6-carboxylic acid

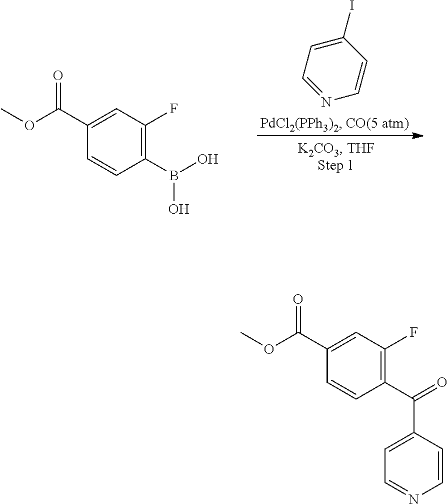

Into a 50-mL pressure reactor, was placed 4-iodopyridine (1.20 g, 5.854 mmol, 1.00 equiv), 2-fluoro-4-(methoxycarbonyl)phenylboronic acid (2.32 g, 0.012 mmol, 2.00 equiv), bis(triphenylphosphine) palladium chloride (0.41 g, 0.585 mmol, 0.10 equiv), potassium carbonate (3.26 g, 0.023 mmol, 4.00 equiv), tetrahydrofuran (12.00 mL), CO (5 atm). The resulting solution was stirred for 16 h overnight at 80° C. The reaction mixture was cooled to 25° C. with an ice/salt bath. The solids were filtered out. The resulting mixture was concentrated. The residue was applied onto a silica gel column with ethyl acetate/petroleum ether (1:1). This resulted in 600 mg (39.5%) of methyl 3-fluoro-4-(pyridine-4-carbonyl)benzoate as a brown solid.

LC-MS (ES, m/z): [M+1]⁺=260.

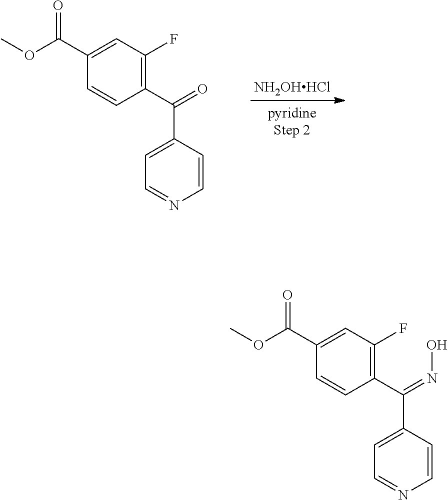

Into a 50-mL round-bottom flask purged and maintained with an inert atmosphere of nitrogen, was placed methyl 3-fluoro-4-(pyridine-4-carbonyl)benzoate (600.00 mg, 2.31 mmol, 1.00 equiv), NH₂OH:HCl (7.00 mg, 0.101 mmol, 0.04 equiv), pyridine (7.50 mL). The resulting solution was stirred for 3 hr at 115° C. The reaction was then quenched by the addition of 40 mL of ice/water. The resulting solution was extracted with 3×20 mL of ethyl acetate and the organic layers combined. The resulting mixture was washed with 3×20 mL of brine. The resulting mixture was concentrated. This resulted in 420 mg (66.2%) of methyl 3-fluoro-4-[(1Z)-(hydroxyimino) (pyridin-4-yl)methyl]benzoate as a light yellow solid.

LC-MS (ES, m/z): [M+1]⁺=275.

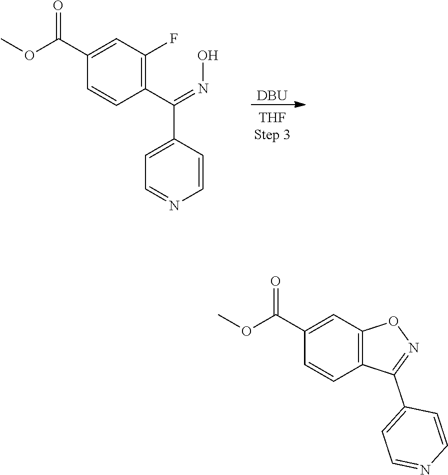

Into a 50-mL round-bottom flask purged and maintained with an inert atmosphere of nitrogen, was placed methyl 3-fluoro-4-[(1Z)-(hydroxyimino) (pyridin-4-yl)methyl]benzoate (400.00 mg, 1.459 mmol, 1.00 equiv), tetrahydrofuran (8 mL), DBU (1102.03 mg, 4.376 mmol, 3.00 equiv). The resulting solution was stirred for 8 hr at 65° C. The reaction was then quenched by the addition of 30 mL of water/ice. The resulting solution was extracted with 3×10 mL of ethyl acetate and the organic layers combined. The resulting mixture was washed with 3×10 mL of aq of citric acid (5%). The resulting mixture was concentrated. The residue was applied onto a silica gel column with ethyl acetate/hexane (1:1). This resulted in 160 mg (43.2%) of methyl 3-(pyridin-4-yl)-1,2-benzoxazole-6-carboxylate as a light yellow solid.

LC-MS (ES, m/z): [M+1]⁺=255.

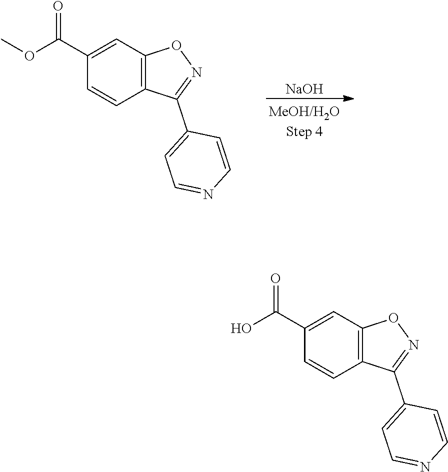 [SMILES: COC(=O)c1ccc2c(-c3ccncc3)noc2c1 ; "NaOH" ; O=C(O)c1ccc2c(-c3ccncc3)noc2c1]

Into a 8-mL vial purged and maintained with an inert atmosphere of nitrogen, was placed methyl 3-(pyridin-4-yl)-1,2-benzoxazole-6-carboxylate (160.00 mg, 0.629 mmol, 1.00 equiv), MeOH (4.00 mL), H₂O (1.00 mL), sodium hydroxide (50.34 mg, 1.259 mmol, 2.00 equiv). The resulting solution was stirred for 12 hr at 25° C. The pH value of the solution was adjusted to pH=3 with HCl (37%). The resulting mixture was concentrated. This resulted in 220 mg (with NaCl) of 3-(pyridin-4-yl)-1,2-benzoxazole-6-carboxylic acid as an off-white solid.

LC-MS (ES, m/z): [M+1]⁺=241.

Acid 2: 3-acetylimidazo[1,5-a]pyridine-7-carboxylic acid

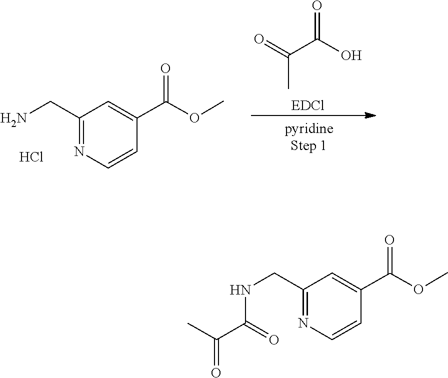

Into a 50-mL round-bottom flask, was placed methyl 2-(aminomethyl)pyridine-4-carboxylate hydrochloride (700.00 mg, 3.454 mmol, 1.00 equiv), pyruvic acid (608.40 mg, 2.00 equiv), pyridine (2.00 mL), EDCI (1324.43 mg, 6.908 mmol, 2.00 equiv). The resulting solution was stirred for 5 hr at room temperature. The resulting mixture was concentrated. The residue was applied onto a silica gel column with ethyl acetate/petroleum ether (1:1). This resulted in 400 mg (49%) of methyl 2-[(2-oxopropanamido)methyl]pyridine-4-carboxylate as a yellow solid.

LC-MS−: (ES, m/z): [M+H]=237

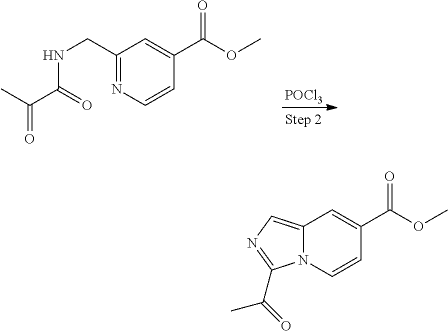

Into a 50-mL round-bottom flask, was placed methyl 2-[(2-oxopropanamido)methyl]pyridine-4-carboxylate (340.00 mg, 1.44 mmol), POCl₃ (5.00 mL). The resulting solution was stirred for 10 hr at room temperature. The resulting mixture was concentrated. The reaction was then quenched by the addition of water/ice. The resulting solution was extracted with 2×20 mL of ethyl acetate and the organic layers combined and concentrated. The residue was applied onto a silica gel column with ethyl acetate/petroleum ether (1:1). This resulted in 50 mg (16%) of methyl 3-acetylimidazo[1,5-a]pyridine-7-carboxylate as an off-white solid.

LC-MS: (ES, m/z): [M+H]=219.

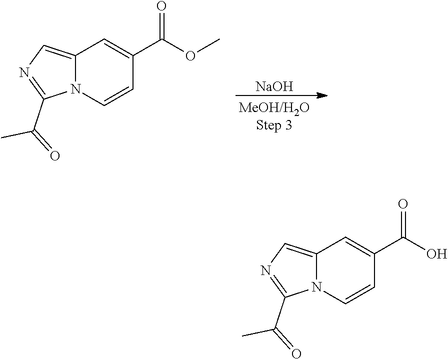 [SMILES: COC(=O)c1ccn2c(C(C)=O)ncc2c1 ; "NaOH" ; CC(=O)c1ncc2cc(C(=O)O)ccn12]

Into an 8-mL sealed tube, was placed methyl 3-acetylimidazo[1,5-a]pyridine-7-carboxylate (50.00 mg, 0.229 mmol, 1.00 equiv), NaOH (18.33 mg, 2.00 equiv), H₂O (1.00 mL), MeOH (5.00 mL). The resulting solution was stirred for 16 hr at room temperature. The resulting mixture was concentrated. The crude product was purified by Prep-HPLC with the following conditions (Prep-HPLC-003): Column, SunFire Prep C18 OBD Column, 19*150 mm, 5 nm, mobile phase, Water (0.05% HCl) and ACN (15% Phase B up to 45% in 7 min); Detector, uv 254 nm. This resulted in 30 mg (64.12%) of 3-acetylimidazo[1,5-a]pyridine-7-carboxylic acid as a white solid.

LC-MS: (ES, m/z): [M+H]=205

Acid 3: 1-methyl-1H-pyrazolo[4,3-c]pyridine-6-carboxylic acid

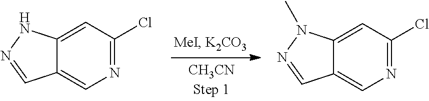

Into a 100-mL 3-necked round-bottom flask purged and maintained with an inert atmosphere of nitrogen, was placed 6-chloro-1H-pyrazolo[4,3-c]pyridine (2.00 g, 13.023 mmol, 1.00 equiv), methyl iodide (2.77 g, 0.020 mmol, 1.5 equiv), acetonitrile (40.00 mL), potassium carbonate (3.63 g, 0.026 mmol, 2.0 equiv). The resulting solution was stirred for 6 hr at 60° C. The solids were filtered out. The reaction was then quenched by the addition of 100 mL of water/ice. The resulting solution was extracted with 3×50 mL of ethyl acetate. The resulting mixture was washed with 3×50 mL of brine. The resulting mixture was concentrated. The residue was applied onto a silica gel column with ethyl acetate/hexane (1:5). This resulted in 1 g (45.8%) of 6-chloro-1-methylpyrazolo[4,3-c]pyridine as an off-white solid.

LC-MS: (ES, m/z): [M+1]⁺=168.

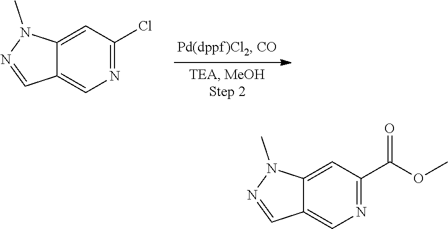

Into a 50-mL sealed tube, was placed 6-chloro-1-methylpyrazolo[4,3-c]pyridine (700.00 mg, 4.177 mmol, 1.00 equiv), TEA (1267.89 mg, 12.530 mmol, 3.00 equiv), MeOH (20.00 mL), Pd(dppf)Cl₂ (305.60 mg, 0.418 mmol, 0.1 equiv), CO (5 atm). The resulting solution was stirred for 3 hr at 130° C. The resulting mixture was concentrated. The residue was applied onto a silica gel column with ethyl acetate/hexane (1:3). This resulted in 750 mg (94%) of methyl 1-methylpyrazolo[4,3-c]pyridine-6-carboxylate as a brown solid.

LC-MS (ES, m/z): [M+1]⁺=192.

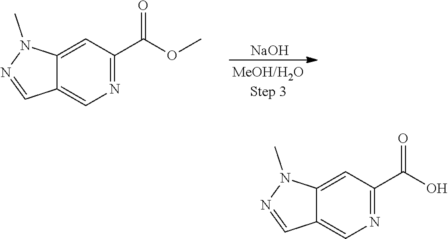

Into a 50-mL round-bottom flask purged and maintained with an inert atmosphere of nitrogen, was placed methyl 1-methylpyrazolo[4,3-c]pyridine-6-carboxylate (750.00 mg, 3.923 mmol, 1.00 equiv), MeOH (8.00 mL), H₂O (2.00 mL), sodium hydroxide (313.80 mg, 7.846 mmol, 2.00 equiv). The resulting solution was stirred for 16 hr at 25° C. The reaction was then quenched by the addition of 15 mL of water/ice. The pH value of the solution was adjusted to 3 with HCl (37%). The resulting solution was extracted with 3×10 mL of dichloromethane and the organic layers combined and concentrated. This resulted in 500 mg (72%) of 1-methylpyrazolo[4,3-c]pyridine-6-carboxylic acid as a light yellow solid.

LC-MS: (ES, m/z): [M+1]⁺=178.

Acid 4: 5-fluoro-3-methylbenzo[d]isoxazole-6-carboxylic acid

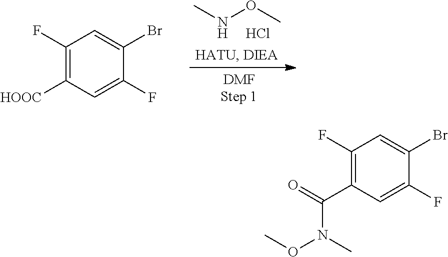

Into a 250-mL 3-necked round-bottom flask purged and maintained with an inert atmosphere of nitrogen, was placed 4-bromo-2,5-difluorobenzoic acid (4.50 g, 19 mmol, 1.00 equiv), N,O-dimethylhydroxylamine hydrochloride (2.22 g, 22.784 mmol, 1.20 equiv), dimethylformamide (90 mL), HATU (10.83 g, 28.481 mmol, 1.50 equiv), DIEA (9.82 g, 75.948 mmol, 4.00 equiv). The resulting solution was stirred for 6 hr at 25° C. The reaction was then quenched by the addition of 300 ml of water/ice. The resulting solution was extracted with 3×80 mL of ethyl acetate The resulting mixture was washed with 3×80 mL of brine. The resulting mixture was concentrated. This resulted in 4.47 g (84.1%) of 4-bromo-2,5-difluoro-N-methoxy-N-methylbenzamide as a brown solid.

LC-MS (ES, m/z): [M+1]⁺=280.

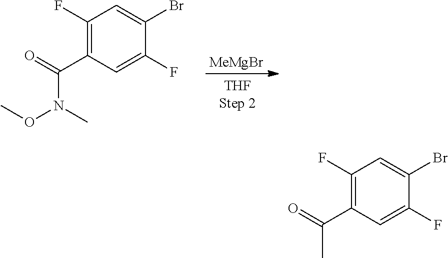

Into a 250-mL 3-necked round-bottom flask purged and maintained with an inert atmosphere of nitrogen, was placed 4-bromo-2,5-difluoro-N-methoxy-N-methylbenzamide (4.43 g, 15.82 mmol, 1.00 equiv), tetrahydrofuran (86 mL), methylmagnesium bromide (3 mol/L in Et₂O) (15.82 mL, 47.45 mmol, 3.00 equiv) was added and the resulting solution was stirred for 30 min at −78° C. The resulting solution was allowed to react, with stirring, for an additional 2.5 hr at 25° C. The reaction was then quenched by the addition of 300 mL of water/ice. The resulting solution was extracted with 3×80 mL of ethyl acetate and the organic layers combined. The resulting mixture was washed with 3×80 mL of brine. The resulting mixture was concentrated. This resulted in 2.1 g (56.49%) of 1-(4-bromo-2,5-difluorophenyl) ethanone as a dark yellow solid.

LC-MS (ES, m/z): [M+1]⁺=235.

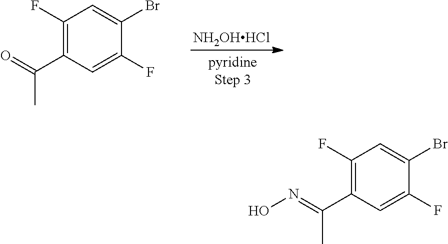

Into a 40-mL vial purged and maintained with an inert atmosphere of nitrogen, was placed 1-(4-bromo-2,5-difluorophenyl) ethanone (500.00 mg, 2.13 mmol, 1.00 equiv), pyridine (10.00 mL), NH₂OH:HCl (1035 mg, 14.889 mmol, 7.00 equiv). The resulting solution was stirred for 3 hr at 105° C. The reaction was then quenched by the addition of 30 mL of HCl. (10%). The resulting solution was extracted with 4×8 mL of ethyl acetate concentrated. This resulted in 500 mg (94.00%) of (E)-N-[1-(4-bromo-2,5-difluorophenyl)ethylidene]hydroxylamine as a light yellow solid.

LC-MS (ES, m/z): [M+1]⁺=250.

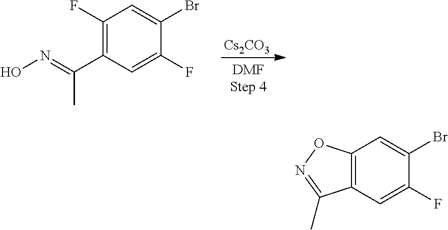

Into a 40-mL vial purged and maintained with an inert atmosphere of nitrogen, was placed (E)-N-[1-(4-bromo-2,5-difluorophenyl)ethylidene]hydroxylamine (460.00 mg, 1.840 mmol, 1.00 equiv), dimethylformamide (10.00 mL), cesium carbonate (2405 mg, 7.360 mmol, 4.00 equiv). The resulting solution was stirred for 2 hr at 70° C. The reaction was then quenched by the addition of 30 mL of water/ice. The resulting solution was extracted with 3×10 mL of ethyl acetate. The resulting mixture was washed with 3×10 mL of brine. The resulting mixture was concentrated. This resulted in 350 mg (82.70%) of 6-bromo-5-fluoro-3-methyl-1,2-benzoxazole as a dark brown solid.

LC-MS (ES, m/z): [M+1]⁺=230.

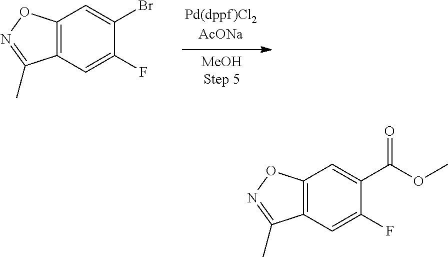

Into a 50-mL pressure reactor, was placed 6-bromo-5-fluoro-3-methyl-1,2-benzoxazole (320.00 mg, 1.39 mmol, 1.00 equiv), methanol (10.00 mg), sodium acetate (342.35 mg, 4.173 mmol, 3.0 equiv), Pd(dppf)Cl₂ (101.79 mg, 0.139 mmol, 0.10 equiv), CO (20 atm). The resulting solution was stirred for 4 hr at 80° C. The resulting mixture was concentrated. The residue was applied onto a silica gel column with ethyl acetate/hexane (1:1). This resulted in 53 mg (18.2%) of methyl 5-fluoro-3-methyl-1,2-benzoxazole-6-carboxylate as a light brown solid.

LC-MS (ES, m/z): [M+1]⁺=210.

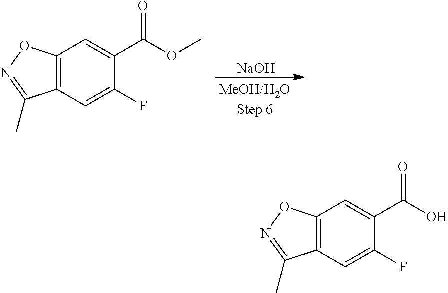

Into a 8-mL round-bottom flask purged and maintained with an inert atmosphere of nitrogen, was placed methyl 5-fluoro-3-methyl-1,2-benzoxazole-6-carboxylate (53.00 mg, 0.253 mmol, 1.00 equiv), MeOH (2.00 mg), H₂O (0.50 mg), sodium hydroxide (20.27 mg, 0.507 mmol, 2.00 equiv). The resulting solution was stirred for 12 hr at 25° C. The pH value of the solution was adjusted to 3 with HCl (37%). The resulting solution was extracted with 3×3 mL of dichloromethane concentrated. This resulted in 45 mg (91%) of 5-fluoro-3-methyl-1,2-benzoxazole-6-carboxylic acid as an off-white solid.

LC-MS (ES, m/z): [M−1]−=194.

Acid 5: 7-fluoro-2-methylquinoline-6-carboxylic acid

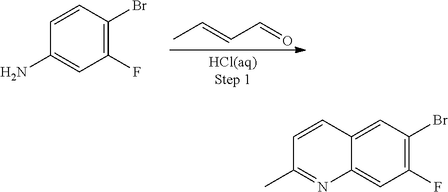

Into a 250-mL 3-necked round-bottom flask purged and maintained with an inert atmosphere of nitrogen, was placed 4-bromo-3-fluoroaniline (5.50 g, 28.95 mmol, 1.00 equiv), HCl (37%) (110.00 mL), crotonaldehyde (5.07 g, 72.335 mmol, 2.50 equiv). The resulting solution was stirred for 5 hr at 110° C. The pH value of the solution was adjusted to 9 with NaOH (10%). The resulting solution was extracted with 3×150 mL of dichloromethane and the organic layers combined and concentrated. The residue was applied onto a silica gel column with ethyl acetate/petroleum ether (1:1). This resulted in 1.5 g (21.6%) of 6-bromo-7-fluoro-2-methylquinoline as a brown solid.

LC-MS (ES, m/z): [M+1]⁺=240.

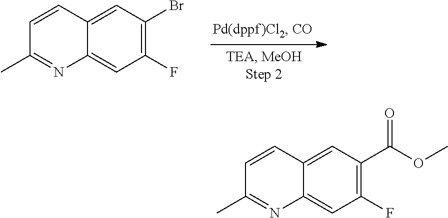

Into a 50-mL sealed tube, was placed 6-bromo-7-fluoro-2-methylquinoline (600.00 mg, 2.5 mmol, 1.00 equiv), TEA (758.7 mg, 7.498 mmol, 3.00 equiv), MeOH (20.00 mL), Pd(dppf)Cl₂ (182.9 mg, 0.250 mmol, 0.1 equiv), CO (5 atm). The resulting solution was stirred for 3 hr at 130° C. The resulting mixture was concentrated. The residue was applied onto a silica gel column with ethyl acetate/hexane (1:3). This resulted in 480 mg (87.6%) of methyl 7-fluoro-2-methylquinoline-6-carboxylate as a brown solid.

LC-MS (ES, m/z): [M+1]⁺=220.

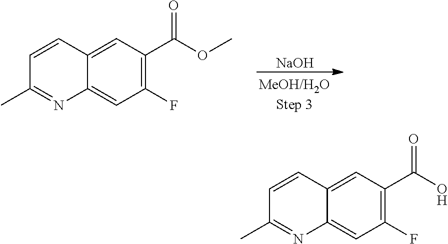

Into a 50-mL round-bottom flask, was placed methyl 7-fluoro-2-methylquinoline-6-carboxylate (480.00 mg, 2.19 mmol, 1.00 equiv), MeOH (4.00 mL), H₂O (1.00 mL), sodium hydroxide (175.16 mg, 4.38 mmol, 2.00 equiv). The resulting solution was stirred for 16 hr at 25° C. The pH value of the solution was adjusted to 3 with HCl (10%). The resulting mixture was concentrated. The residue was dissolved in 20 mL of MeOH/DCM=1:4. The solids were filtered out. The filtrate was concentrated. This resulted in 390 mg (86.8%) of 7-fluoro-2-methylquinoline-6-carboxylic acid as an off-white solid.

LC-MS (ES, m/z): [M+1]⁺=206.

Acid 6: 7-fluoro-3-methylbenzo[d]isoxazole-6-carboxylic acid

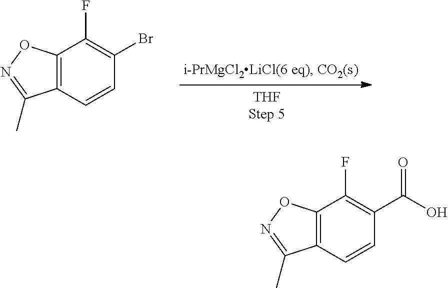

Into a 25-mL 2-necked round-bottom flask purged and maintained with an inert atmosphere of nitrogen, was placed 6-bromo-7-fluoro-3-methyl-1,2-benzoxazole (Prepared according to Acid 4, Step 4 using 4-bromo-2,3-difluorobenzoic acid) 150.00 mg, 0.652 mmol, 1.00 equiv), tetrahydrofuran (5 mL). isopropylmagnesium chloride lithium chloride complex (1 mol/L in THF) (3.91 mL, 3.912 mmol, 6.00 equiv) was added and the resulting solution was stirred for 30 min at −20° C. The resulting solution was allowed to react, with stirring, for an additional 2.5 hr at 25° C. Then the resulting solution was poured into CO₂ (s). The reaction was then quenched by the addition of 20 mL of water/ice. The resulting solution was extracted with 3×8 mL of ethyl acetate and the aqueous layers combined. The pH value of the solution was adjusted to 3 with HCl (37%). The resulting solution was extracted with 3×8 mL of dichloromethane concentrated. This resulted in 40 mg (31.4%) of 7-fluoro-3-methyl-1,2-benzoxazole-6-carboxylic acid as a light yellow solid.

LC-MS (ES, m/z): [M+1]⁺=196.

Acid 7: 1-methylimidazo[1,5-a]pyridine-6-carboxylic acid

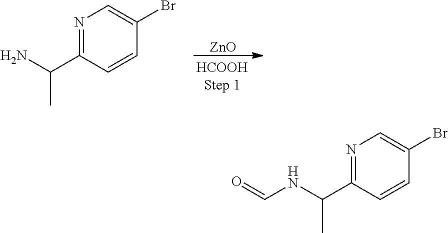

Into an 8-mL vial purged and maintained with an inert atmosphere of nitrogen, was placed 1-(5-bromopyridin-2-yl) ethanamine (2.20 g, 10.942 mmol, 1.00 equiv), zinc oxide (0.89 g, 10.942 mmol, 1.00 equiv), formic acid (6.60 mL). The resulting solution was stirred for 8 hr at 70° C. The resulting solution was diluted with 10 mL of DCM. The solids were filtered out. The resulting mixture was concentrated. The residue was applied onto a silica gel column with PE/THF (3:1). This resulted in 1.2 g (47.9%) of N-[1-(5-bromopyridin-2-yl)ethyl]formamide as colorless oil.

LC-MS (ES, m/z): [M+1]⁺=229.

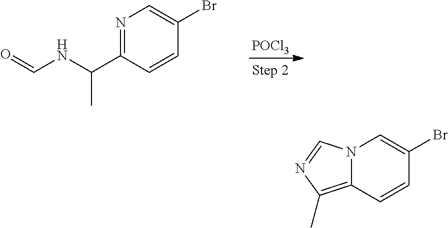

Into a 50-mL round-bottom flask purged and maintained with an inert atmosphere of nitrogen, was placed N-[1-(5-bromopyridin-2-yl)ethyl]formamide (500.00 mg, 2.18 mmol, 1.00 equiv), phosphorus oxychloride (10 mL). The resulting solution was stirred for 1 hr at 115° C. The resulting mixture was concentrated. The resulting solution was extracted with 3×10 mL of ethyl acetate and the organic layers combined. The resulting mixture was washed with 3×10 ml of brine. The resulting mixture was concentrated. This resulted in 400 mg (86.8%) of 6-bromo-1-methylimidazo[1,5-a]pyridine as a light brown solid.

LC-MS (ES, m/z): [M+1]⁺=211.

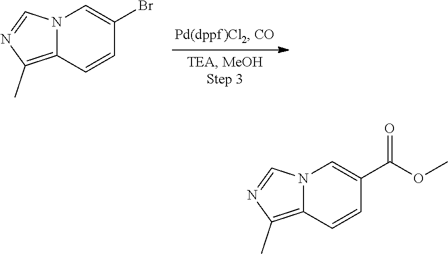

Into a 50-mL sealed tube, was placed 6-bromo-1-methylimidazo[1,5-a]pyridine (400 mg, 1.9 mmol, 1.00 equiv), Pd(dppf)Cl₂ (138.67 mg, 0.190 mmol, 0.1 equiv), TEA (575.32 mg, 5.685 mmol, 3.0 equiv), MeOH (20.00 mL), CO (5 atm). The resulting solution was stirred for 16 hr at 120° C. The resulting mixture was concentrated. The residue was applied onto a silica gel column with ethyl acetate/petroleum ether (1:3). This resulted in 1-methylimidazo[1,5-a]pyridine-6-carboxylic acid (300 mg, 90%) as a brown solid.

LC-MS (ES, m/z): [M+1]⁺=191.

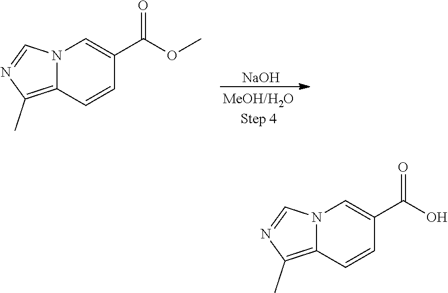

Into a 50-mL round-bottom flask purged and maintained with an inert atmosphere of nitrogen, was placed methyl 1-methylimidazo[1,5-a]pyridine-6-carboxylate (300 mg, 1.577 mmol, 1.00 equiv), sodium hydroxide (126.17 mg, 3.154 mmol, 2.0 equiv), MeOH (4 mL, 98.8 mmol, 62.6 equiv), H₂O (1 mL, 55.508 mmol, 35.19 equiv). The resulting solution was stirred for 16 hr at room temperature. The pH value of the solution was adjusted to 3 with HCl (37%). The resulting mixture was concentrated. This resulted in 1-methylimidazo[1,5-a]pyridine-6-carboxylic acid (450 mg, crude) as a brown solid.

LC-MS (ES, m/z): [M+1]⁺=177.

Acid 8: 3-(trifluoromethyl)-1H-indazole-5-carboxylic acid

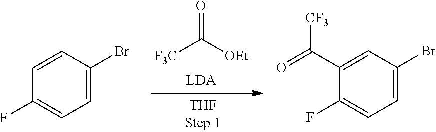 [SMILES: Fc1ccc(Br)cc1 ; CCOC(=O)C(F)(F)F ; O=C(c1cc(Br)ccc1F)C(F)(F)F]

To a stirred solution of LDA (1.86 mL, 13.72 mmol, 1.2 equiv) were added 4-bromofluorobenzene (2 g, 11.429 mmol, 1.00 equiv) in tetrahydrofuran (20 mL) dropwise at −78° C. under nitrogen atmosphere. The resulting mixture was stirred for 1 hr at −78° C. under nitrogen atmosphere. To the stirred solution was added trifluoroethyl acetate (1.95 g, 13.72 mmol, 1.2 equiv) in THF (20 mL) dropwise at −78° C. under nitrogen atmosphere. The resulting mixture was stirred for 1 hr at −78° C. under nitrogen atmosphere. The reaction was quenched by the addition of sat. NH₄Cl (aq.) (5 mL) at −78° C. The resulting mixture was diluted with water (40 mL). The aqueous layer was extracted with EtOAc (3×20 mL). The resulting mixture was concentrated under reduced pressure.

The residue was purified by silica gel column chromatography, eluted with n-hexane/EA (1:1) to afford 1-(5-bromo-2-fluorophenyl)-2,2,2-trifluoroethanone (1.5 g, 48.4%) as a light brown solid.

LC-MS (ES, m/z): [M+1]⁺=271.

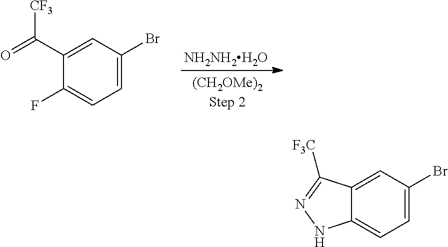

Into a 100 mL 3-necked round-bottom flask were added 1-(5-bromo-2-fluorophenyl)-2,2,2-trifluoroethanone (1.5 g, 5.54 mmol, 1.00 equiv) and 1,2-dimethoxyethane (30 mL, 332.9 mmol, 60.14 equiv), NH₂NH₂·H₂O (2.77 g, 55.4 mmol, 10 equiv) at 90° C. The resulting mixture was concentrated under reduced pressure. The reaction was quenched by the addition of water (40 mL). The resulting mixture was extracted with EtOAc (3×10 mL). The combined organic layers were washed with brine (3×10 mL), dried over anhydrous Na₂SO₄. After filtration, the filtrate was concentrated under reduced pressure. This resulted in 5-bromo-3-(trifluoromethyl)-1H-indazole (0.8 g, 54.5%) as a light brown solid.

LC-MS (ES, m/z): [M+1]⁺=265.

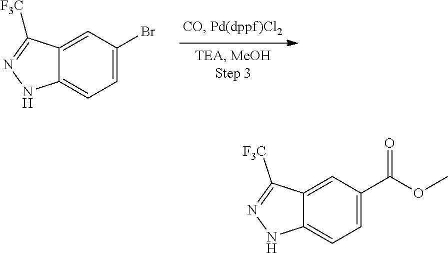 [SMILES: FC(F)(F)c1n[nH]c2ccc(Br)cc12 ; COC(=O)c1ccc2[nH]nc(C(F)(F)F)c2c1]

Into a 50-mL sealed tube, was placed 5-bromo-3-(trifluoromethyl)-1H-indazole (0.8 g, 3.018 mmol, 1.00 equiv), Pd(dppf)Cl₂ (0.44 g, 0.604 mmol, 0.2 equiv), TEA (0.92 g, 9.054 mmol, 3.0 equiv), MeOH (20.00 mL), CO (20 atm). The resulting solution was stirred for 3 hr at 130° C. The resulting mixture was concentrated. The residue was applied onto a silica gel column with ethyl acetate/petroleum ether (1:3). This resulted in methyl 3-(trifluoromethyl)-1H-indazole-5-carboxylate (0.5 g, 67.8%) as a light brown solid.

LC-MS (ES, m/z): [M+1]⁺=245

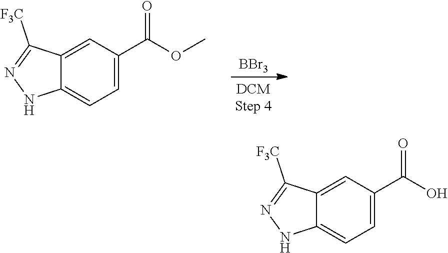

Into a 8 mL vial were added methyl 3-(trifluoromethyl)-1H-indazole-5-carboxylate (200 mg, 0.819 mmol, 1.00 equiv), DCM (4 mL, 62.920 mmol, 76.82 equiv) and boron tribromide (1 mol/L in DCM) (0.41 mL, 1.638 mmol, 2.0 equiv) at room temperature. The resulting mixture was stirred for 16 hr at room temperature under nitrogen atmosphere. The reaction was quenched by the addition of water (10 mL) at room temperature. The aqueous layer was extracted with CH₂Cl₂ (3×10 mL). The resulting mixture was concentrated under reduced pressure. This resulted in 3-(trifluoromethyl)-1H-indazole-5-carboxylic acid (110 mg, 58%) as a light brown solid.

LC-MS (ES, m/z): [M+1]⁺=231

Acid 9: 3-methylpyrazolo[1,5-a]pyridine-6-carboxylic acid

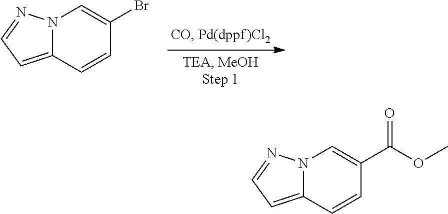

Into a 50-mL sealed tube, was placed 6-bromopyrazolo[1,5-a]pyridine (2 g, 10.150 mmol, 1.00 equiv), Pd(dppf)Cl₂ (1.49 g, 2.030 mmol, 0.2 equiv), MeOH (30 mL, 740.967 mmol, 73.00 equiv), TEA (3.08 g, 30.450 mmol, 3.0 equiv), CO (20 atm). The resulting solution was stirred for 16 hr at 80° C. The resulting mixture was concentrated. The residue was applied onto a silica gel column with ethyl acetate/petroleum ether (1:3). This resulted in methyl pyrazolo[1,5-a]pyridine-6-carboxylate (1.5 g, 83.88%) as a light brown solid.

LC-MS (ES, m/z): [M+1]⁺=177

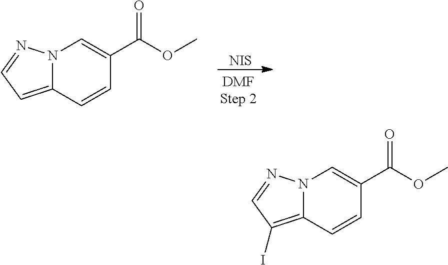

Into a 50 mL round-bottom flask were added methyl pyrazolo[1,5-a]pyridine-6-carboxylate (0.6 g, 3.406 mmol, 1.00 equiv), DMF (12 mL) and NIS (0.92 g, 4.087 mmol, 1.2 equiv) at room temperature. The resulting mixture was stirred for 3 hr at room temperature under nitrogen atmosphere. The reaction was quenched by the addition of sat. Na₂S₂O₄ (aq) (40 mL) at room temperature. The aqueous layer was extracted with EtOAc (3×10 mL). The resulting mixture was washed with 3×10 mL of brine. The resulting mixture was concentrated under reduced pressure. This resulted in methyl 3-iodopyrazolo[1,5-a]pyridine-6-carboxylate (0.9 g, 87.5%) as a light brown solid.

LC-MS (ES, m/z): [M+1]⁺=303.

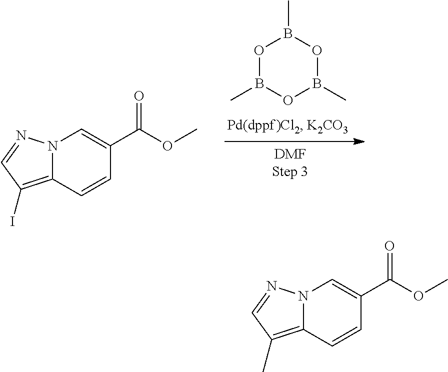

Into a 3-necked round-bottom flask were added methyl 3-iodopyrazolo[1,5-a]pyridine-6-carboxylate (0.9 g, 3.00 mmol, 1.00 equiv), trimethyl-1,3,5,2,4,6-trioxatriborinane (0.94 g, 7.5 mmol, 2.5 equiv), potassium carbonate (1.24 g, 8.937 mmol, 3.0 equiv), Pd(dppf)Cl₂ (0.22 g, 0.298 mmol, 0.1 equiv) and dimethylformamide (20 mL) at room temperature. The resulting mixture was stirred for 4 hr at 80° C. under nitrogen atmosphere. The mixture was allowed to cool down to room temperature. The reaction was quenched by the addition of water (60 mL) at room temperature. The aqueous layer was extracted with EtOAc (3×10 mL). The resulting mixture was concentrated under reduced pressure. The residue was purified by silica gel column chromatography, eluted with PE/EA (3:1) to afford methyl 3-methylpyrazolo[1,5-a]pyridine-6-carboxylate (0.32 g, 56.5%) as a light brown solid.

LC-MS (ES, m/z): [M+1]⁺=191.

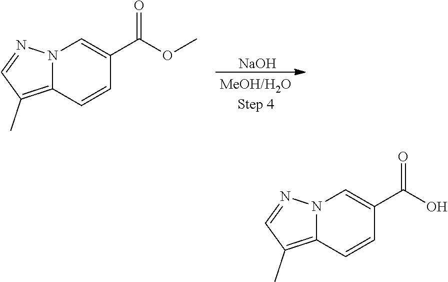

Into an 8 mL vial were added methyl 3-methylpyrazolo[1,5-a]pyridine-6-carboxylate (320 mg, 1.682 mmol, 1.00 equiv), water (1 mL, 55.509 mmol, 32.99 equiv), methanol (4 mL, 124.836 mmol, 74.20 equiv) and sodium hydroxide (134.56 mg, 3.36 mmol, 2.0 equiv) at room temperature. The resulting mixture was stirred for 16 hr at room temperature. The reaction was quenched by the addition of water/ice (10 mL) at room temperature. The mixture/residue was acidified to pH 4 with conc. HCl. The precipitated solids were collected by filtration. This resulted in 3-methylpyrazolo[1,5-a]pyridine-6-carboxylic acid (200 mg, 67.5%) as a light brown solid.

LC-MS− (ES, m/z): [M+1]⁺=177.

Acid 10: 3-(trifluoromethyl) imidazo[1,5-a]pyridine-7-carboxylic acid

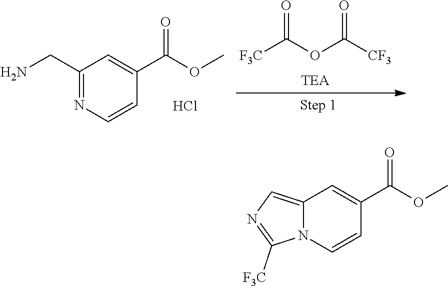

Into a 50 mL 3-necked round-bottom flask were added methyl 2-(aminomethyl)pyridine-4-carboxylate hydrochloride (500 mg, 2.5 mmol, 1.00 equiv) in THF (5 mL). To the mixture was added TEA (624.20 mg, 6.168 mmol, 2.5 equiv) at room temperature. The resulting mixture was stirred for 3 hr at 60° C. Desired product could be detected by LCMS. The resulting mixture was concentrated under vacuum. The residue was purified by silica gel column chromatography, eluted with PE/EA (1:1) to afford methyl 3-(trifluoromethyl) imidazo[1,5-a]pyridine-7-carboxylate (410 mg, 68.1%) as a brown oil.

LC-MS: (ES, m/z): [M+H]=245.

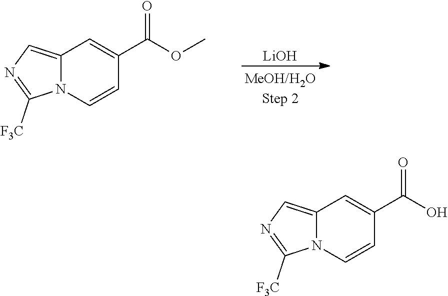 [SMILES: COC(=O)c1ccn2c(C(F)(F)F)ncc2c1 ; "LiOH" ; O=C(O)c1ccn2c(C(F)(F)F)ncc2c1]

Into a 50 mL round-bottom flask were added methyl 3-(trifluoromethyl) imidazo[1,5-a]pyridine-7-carboxylate (300 mg, 1.23 mmol, 1.00 equiv) and LiOH (58.85 mg, 2.456 mmol, 2.0 equiv) in H₂O (1 mL) and MeOH (5 mL) at room temperature. The resulting mixture was stirred for 16 hr at room temperature. The reaction was monitored by LCMS. The resulting mixture was concentrated under vacuum. The mixture was acidified to pH 5 with 1M HCl (aq). The precipitated solids were collected by filtration and dried in an oven. This resulted in 3-(trifluoromethyl) imidazo[1,5-a]pyridine-7-carboxylic acid (170 mg, 60.1%) as an off-white solid.

LC-MS: (ES, m/z): [M+H]=231.

Acid 11: 3-methylpyrazolo[1,5-a]pyridine-5-carboxylic acid

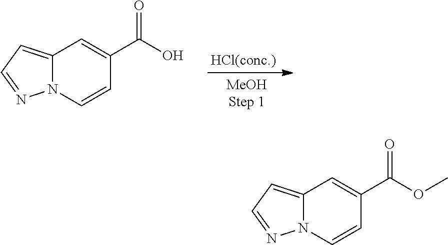

Into a 50 mL 3-necked round-bottom flask were added pyrazolo[1,5-a]pyridine-5-carboxylic acid (500 mg, 3.08 mmol, 1.00 equiv), HCl (conc.) (10 mL) and methanol (10 mL, 312.09 mmol, 101.21 equiv) at room temperature. The resulting mixture was stirred for 12 hr at 70° C. under nitrogen atmosphere. The mixture was allowed to cool down to room temperature. The mixture/residue was neutralized to pH 8 with saturated NaHCO₃ (aq.). The aqueous layer was extracted with EtOAc (3×20 mL). The resulting mixture was concentrated under reduced pressure. This resulted in methyl pyrazolo[1,5-a]pyridine-5-carboxylate (430 mg, 79.2%) as a light brown solid.

LC-MS (ES, m/z): [M+1]⁺=177.

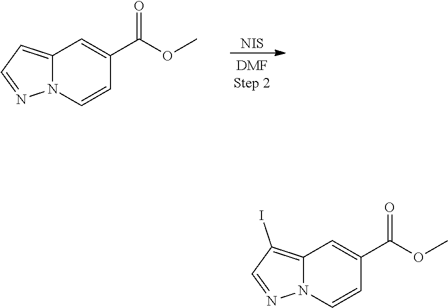

Into a 50 mL round-bottom flask were added methyl pyrazolo[1,5-a]pyridine-5-carboxylate (430 mg, 2.44 mmol, 1.00 equiv), NIS (659 mg, 2.93 mmol, 1.2 equiv), DMF (10 mL, 129.2 mmol, 52.9 equiv) at room temperature. The resulting mixture was stirred for 3 hr at room temperature under nitrogen atmosphere. The reaction was quenched by the addition of sat. Na₂S₂O₄ (aq.) (40 mL) at room temperature. The aqueous layer was extracted with EtOAc (3×10 mL). The resulting mixture was washed with 3×10 mL of brine. The resulting mixture was concentrated under reduced pressure. This resulted in methyl 3-iodopyrazolo[1,5-a]pyridine-5-carboxylate (480 mg, 65.10%) as a light brown solid.

LC-MS (ES, m/z): [M+1]⁺=303.

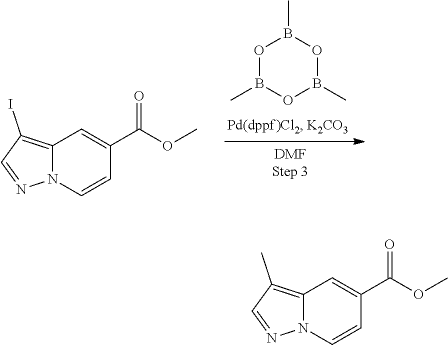

Into a mL 3-necked round-bottom flask were added methyl 3-iodopyrazolo[1,5-a]pyridine-5-carboxylate (480 mg, 1.6 mmol, 1.00 equiv), trimethyl-1,3,5,2,4,6-trioxatriborinane (598.41 mg, 4.77 mmol, 3.0 equiv), dimethylformamide (10 mL, 136.8 mmol, 86.10 equiv), Pd (dppf)Cl₂ (116.27 mg, 0.159 mmol, 0.1 equiv), K₂CO₃ (658.84 mg, 4.77 mmol, 3.0 equiv) at room temperature. The resulting mixture was stirred for 4 hr at 80° C. under nitrogen atmosphere. The mixture was allowed to cool down to room temperature. The reaction was quenched by the addition of water (60 mL) at room temperature. The aqueous layer was extracted with EtOAc (3×10 mL). The resulting mixture was concentrated under reduced pressure. The residue was purified by silica gel column chromatography, eluted with PE/EA (3:1) to afford methyl 3-methylpyrazolo[1,5-a]pyridine-5-carboxylate (220 mg, 72.8%) as a light brown solid.

LC-MS− (ES, m/z): [M+1]⁺=191

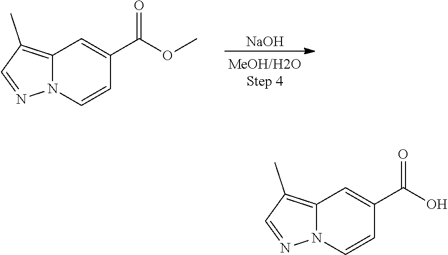

Into a 8 mL vial were added methyl methyl 3-methylpyrazolo[1,5-a]pyridine-5-carboxylate (220 mg, 1.157 mmol, 1.00 equiv), sodium hydroxide (92.53 mg, 2.314 mmol, 2.0 equiv), water (1 mL), methanol (4 mL) at room temperature. The resulting mixture was stirred for 16 hr at room temperature. The reaction was quenched by the addition of water/ice (10 mL) at room temperature. The mixture/residue was acidified to pH 4 with conc. HCl. The precipitated solids were collected by filtration. This resulted in 3-methylpyrazolo[1,5-a]pyridine-5-carboxylic acid (160 mg, 78.52%) as a light brown solid.

LC-MS: (ES, m/z): [M+1]⁺=177.

Acid 12: 1-(trifluoromethyl) imidazo[1,5-a]pyridine-6-carboxylic acid

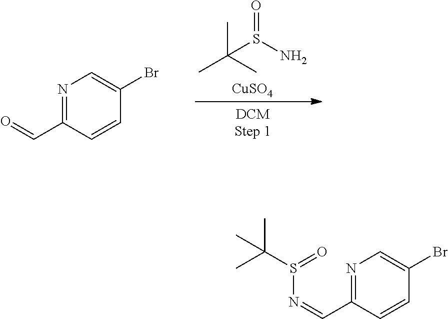

Into a 100 mL 3-necked round-bottom flask were added 5-bromopyridine-2-carbaldehyde (5 g, 26.881 mmol, 1.00 equiv) and tert-butanesulfinamide (3.26 g, 26.881 mmol, 1 equiv) in DCM (50 mL, 786.502 mmol, 29.26 equiv). To the mixture was added CuSO₄ (8.58 g, 53.762 mmol, 2 equiv) at room temperature. The resulting mixture was stirred for 1 hr at room temperature. The reaction was monitored by LCMS. The resulting mixture was filtered, the filtrate was concentrated under reduced pressure. The residue was purified by silica gel column chromatography, eluted with PE/EA (1:1) to afford N-[(1Z)-(5-bromopyridin-2-yl)methylidene]-2-methylpropane-2-sulfinamide (7 g, 90%) as a brown solid.

LC-MS: (ES, m/z): [M+H]=289.

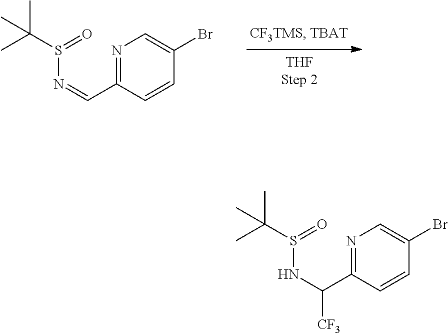 [SMILES: CC(C)(C)S(=O)/N=C\c1ccc(Br)cn1 ; CC(C)(C)S(=O)NC(c1ccc(Br)cn1)C(F)(F)F]

Into a 100 mL 2-necked round-bottom flask were added (Z)-N-((5-bromopyridin-2-yl)methylene)-2-methylpropane-2-sulfinamide 2 g, 6.916 mmol, 1.00 equiv) and tetrabutylammonium difluorotriphenylsilicate 97% (8.21 g, 15.215 mmol, 2.2 equiv) in THF (20 mL) at room temperature. To a stirred mixture was added trifluoromethyltrimethylsilane (2.36 g, 16.6 mmol, 2.4 equiv) in THF (20 mL) dropwise at −55° C. under nitrogen atmosphere. The resulting mixture was stirred for additional 1 hr at room temperature. The reaction was monitored by LCMS. The reaction was quenched by the addition of sat. NH₄Cl (aq.) (50 mL) at 0° C. The resulting mixture was extracted with EtOAc (2×50 mL). The combined organic layers were washed with brine (2×50 mL), dried over anhydrous Na₂SO₄. After filtration, the filtrate was concentrated under reduced pressure. This resulted in N-[1-(5-bromopyridin-2-yl)-2,2,2-trifluoroethyl]-2-methylpropane-2-sulfinamide (2 g) as a brown crude oil.

LC-MS: (ES, m/z): [M+H]=359.

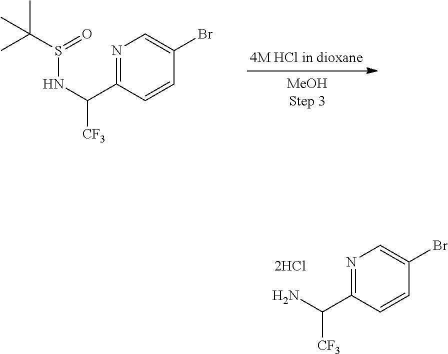 [SMILES: CC(C)(C)S(=O)NC(c1ccc(Br)cn1)C(F)(F)F ; NC(c1ccc(Br)cn1)C(F)(F)F ; "2HCl"]

Into a 50 mL round-bottom flask were added N-[1-(5-bromopyridin-2-yl)-2,2,2-trifluoroethyl]-2-methylpropane-2-sulfinamide (2 g) and HCl (gas) in 1,4-dioxane (20 mL). The resulting mixture was stirred for 2 h at room temperature. The reaction was monitored by LCMS. The resulting mixture was concentrated under vacuum. This resulted in 1-(5-bromopyridin-2-yl)-2,2,2-trifluoroethanamine dihydrochloride (1.85 g, 84% for two steps) as an off-white solid.

LC-MS: (ES, m/z): [M+H−2HCl]=255.

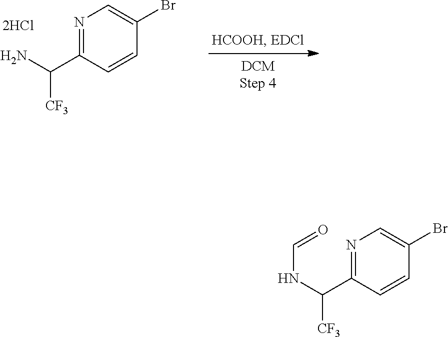 [SMILES: NC(c1ccc(Br)cn1)C(F)(F)F ; "2HCl" ; O=CNC(c1ccc(Br)cn1)C(F)(F)F]

Into a 50 mL 3-necked round-bottom flask were added 1-(5-bromopyridin-2-yl)-2,2,2-trifluoroethanamine dihydrochloride (1 g, 3.049 mmol, 1.00 equiv) and HCOOH (0.28 g, 6.098 mmol, 2 equiv) in pyridine (10 mL). Then was added EDCI (2.34 g, 12.196 mmol, 4 equiv) at room temperature. The resulting mixture was stirred for 8 hr at room temperature. The reaction was monitored by LCMS. The reaction was quenched by the addition of water (20 mL) at room temperature. The resulting mixture was extracted with EtOAc (2×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (5:1˜1:1) to afford N-[1-(5-bromopyridin-2-yl)-2,2,2-trifluoroethyl]formamide (750 mg, 86.9%) as a white solid.

LC-MS− (ES, m/z): [M+H]=283.

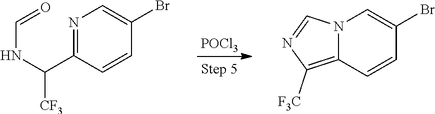

Into a 50 mL round-bottom flask were added N-[1-(5-bromopyridin-2-yl)-2,2,2-trifluoroethyl]formamide (750 mg, 2.65 mmol, 1.00 equiv) and POCl₃ (2 mL, 21.46 mmol, 8.10 equiv). The resulting mixture was stirred for 1 hr at 90° C. The reaction was monitored by LCMS. The resulting mixture was concentrated under vacuum. The residue was quenched by the addition of water (30 mL). The mixture was basified to pH 8 with saturated Na₂CO₃ (aq). The aqueous layer was extracted with EtOAc (2×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (1:1) to afford 6-bromo-1-(trifluoromethyl) imidazo[1,5-a]pyridine (653 mg, 93%) as a brown solid.

LC-MS (ES, m/z): [M+H]=265.

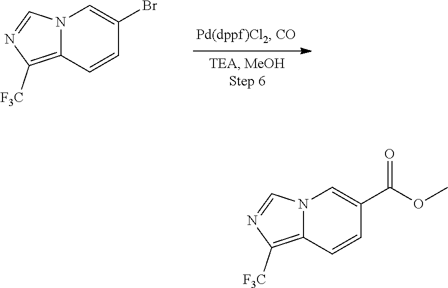 [SMILES: FC(F)(F)c1ncn2cc(Br)ccc12 ; COC(=O)c1ccc2c(C(F)(F)F)ncn2c1]

To a solution of 6-bromo-1-(trifluoromethyl) imidazo[1,5-a]pyridine (400 mg, 1.51 mmol, 1.00 equiv) and Pd(dppf)Cl₂CH₂Cl₂ (122.95 mg, 0.151 mmol, 0.1 equiv) in 10 mL MeOH was added TEA (763.60 mg, 7.545 mmol, 5 equiv) in a pressure tank. The mixture was purged with nitrogen for 1.0 min and then was pressurized to 3 MPa with carbon monoxide at 120° C. for 3 hr. The reaction mixture was cooled to room temperature and filtered to remove insoluble solids. The filtrate was concentrated under reduced pressure. The residue was purified by silica gel column chromatography, eluted with PE/EA (2:1) to afford methyl 1-(trifluoromethyl) imidazo[1,5-a]pyridine-6-carboxylate (358 mg, 97.2%) as a brown solid.

LC-MS: (ES, m/z): [M+H]=245.

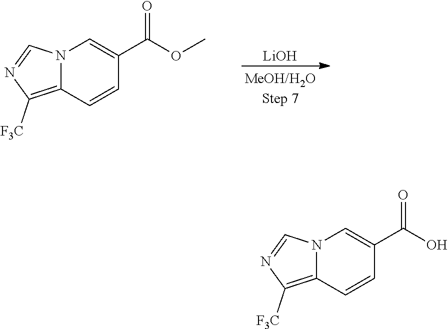 [SMILES: COC(=O)c1ccc2c(C(F)(F)F)ncn2c1 ; "LiOH" ; O=C(O)c1ccc2c(C(F)(F)F)ncn2c1]

To a solution of methyl 1-(trifluoromethyl) imidazo[1,5-a]pyridine-6-carboxylate (353 mg, 1.45 mmol, 1.00 equiv) in 10 mL MeOH and 1 mL H₂O was added LiOH (69.24 mg, 2.892 mmol, 2 equiv) in a 50 mL round-bottom flask. The resulting mixture was stirred for 16 hr at room temperature. The reaction was monitored by LCMS. The resulting mixture was concentrated under vacuum. The residue was acidified to pH 5 with citric acid. The precipitated solids were collected by filtration. The resulting solid was dried under infrared light. This resulted in 1-(trifluoromethyl) imidazo[1,5-a]pyridine-6-carboxylic acid (280 mg, 84.2%) as a white solid.

LC-MS (ES, m/z): [M+H]=231.

Acid 13: 1-methyl-3-(trifluoromethyl) indazole-6-carboxylic acid

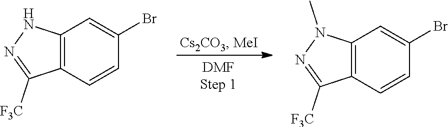

To a solution of 6-bromo-3-(trifluoromethyl)-1H-indazole (1 g, 3.773 mmol, 1.00 equiv) in 10 mL DMF was added Cs₂CO₃ (2.46 g, 7.546 mmol, 2.0 equiv) and Mel (0.70 g, 4.905 mmol, 1.3 equiv). The resulting mixture was stirred for 10 hr at room temperature. The resulting mixture was filtered. The filtrate was concentrated under reduced pressure. The residue was purified by silica gel column chromatography, eluted with PE/EA (1:1) to afford 6-bromo-1-methyl-3-(trifluoromethyl) indazole (830 mg, 78.8%) as a brown solid.

LC-MS (ES, m/z): [M+H]=278

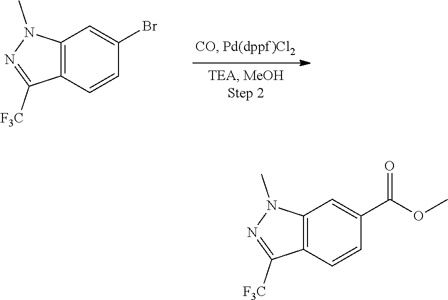 [SMILES: Cn1nc(C(F)(F)F)c2ccc(Br)cc21 ; COC(=O)c1ccc2c(C(F)(F)F)nn(C)c2c1]

To a solution of 6-bromo-1-methyl-3-(trifluoromethyl) indazole (530 mg, 1.899 mmol, 1.00 equiv) in 20 mL MeOH was added Pd(dppf)Cl₂·CH₂Cl₂ (154.72 mg, 0.190 mmol, 0.1 equiv) and TEA (960.92 mg, 9.495 mmol, 5 equiv) in a pressure tank. The mixture was purged with nitrogen for 0.5 min and then was pressurized to 3 MPa with carbon monoxide at 100° C. for 16 hr. The filtrate was concentrated under reduced pressure. The residue was purified by silica gel column chromatography, eluted with PE/EA (1:1) to afford methyl 1-methyl-3-(trifluoromethyl) indazole-6-carboxylate (437 mg, 89%) as a white solid.

LC-MS (ES, m/z): [M+H]=259

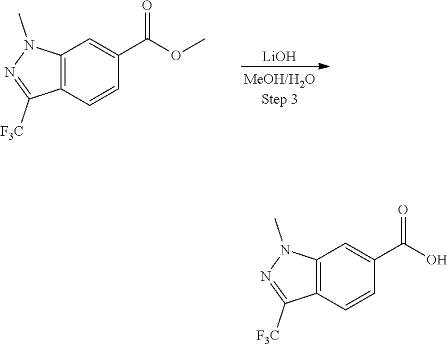 [SMILES: COC(=O)c1ccc2c(C(F)(F)F)nn(C)c2c1 ; "LiOH" ; Cn1nc(C(F)(F)F)c2ccc(C(=O)O)cc21]

To a solution of methyl 1-methyl-3-(trifluoromethyl) indazole-6-carboxylate (300 mg, 1.162 mmol, 1.00 equiv) in 10 mL MeOH was added H₂O (1 mL) and LiOH (55.65 mg, 2.324 mmol, 2 equiv) in a 50 mL round-bottom flask. The resulting mixture was stirred for 16 hr at room temperature. The reaction was monitored by LCMS. The resulting mixture was concentrated under vacuum. The residue was acidified to pH 5 with citric acid. The resulting mixture was filtered. The precipitated solids were collected by filtration. The resulting solid was dried under infrared light. This resulted in 1-methyl-3-(trifluoromethyl) indazole-6-carboxylic acid (226 mg, 79.7%) as a white solid.

LC-MS (ES, m/z): [M−H]=243.

Acid 14: 1-cyclopropylindazole-5-carboxylic acid

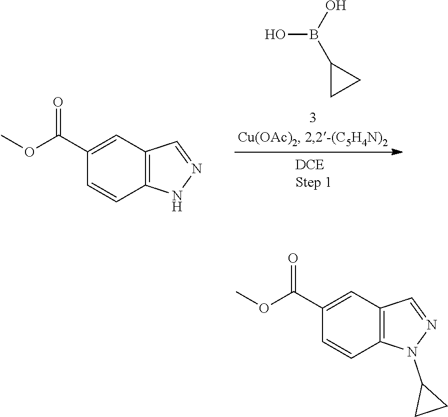 [SMILES: OB(O)C1CC1 ; COC(=O)c1ccc2[nH]ncc2c1 ; COC(=O)c1ccc2c(cnn2C2CC2)c1]

Into a 100-mL 3-necked round-bottom flask purged and maintained with an inert atmosphere of nitrogen, was placed methyl 1H-indazole-5-carboxylate (1.00 g, 5.7 mmol, 1.00 equiv), DCE (50 mL), cyclopropylboronic acid (0.98 g, 11.352 mmol, 2.00 equiv), cupric acetate (1.03 g, 5.676 mmol, 1.00 equiv), 2,2′-bipyridyl (0.89 g, 5.7 mmol, 1.00 equiv). The resulting solution was stirred for 16 hr at 70° C. in an oil bath. The resulting solution was diluted with 50 mL of NH₄Cl. The resulting solution was extracted with 2×20 mL of dichloromethane and the organic layers combined and dried over anhydrous sodium sulfate and concentrated under vacuum. The residue was applied onto a silica gel column with ethyl acetate/petroleum ether (1/8). This resulted in 600 mg (48.88%) of methyl 1-cyclopropylindazole-5-carboxylate as an off-white solid.

LC-MS: (ES, m/z): [M+H]⁺=217.

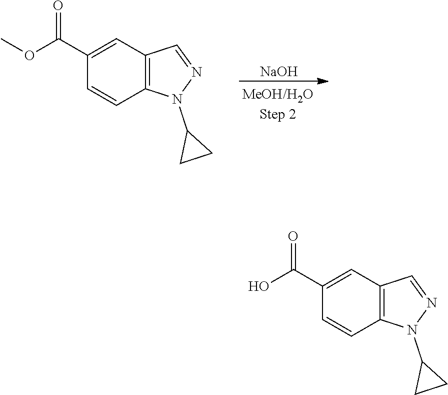 [SMILES: COC(=O)c1ccc2c(cnn2C2CC2)c1 ; "NaOH" ; O=C(O)c1ccc2c(cnn2C2CC2)c1]

Into a 40-mL vial, was placed methyl 1-cyclopropylindazole-5-carboxylate (600 mg, 2.775 mmol, 1.00 equiv), CH₃OH (12.00 mL), H₂O (4.00 mL), sodium hydroxide (550 mg, 13.88 mmol, 5.00 equiv). The resulting solution was stirred for 10 hr at 60° C. in an oil bath. The resulting mixture was concentrated under vacuum. The resulting solution was diluted with 30 mL of H₂O. The pH value of the solution was adjusted to 3 with HCl (2 mol/L). The solids were collected by filtration. This resulted in 450 mg (80.2%) of 1-cyclopropylindazole-5-carboxylic acid as an off-white solid.

LC-MS: (ES, m/z): [M+H]⁺=203.

Acid 15: 3-methyl-1-(pyridin-4-yl) indazole-5-carboxylic acid

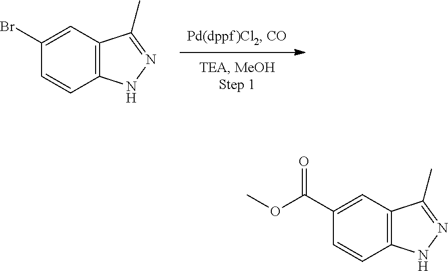

Into a 150-mL sealed tube, was placed 5-bromo-3-methyl-1H-indazole (2.00 g, 9.476 mmol, 1.00 equiv), TEA (4.79 g, 47.379 mmol, 5 equiv), MeOH (40 mL), Pd(dppf)Cl₂ CH₂Cl₂ (1.54 g, 1.895 mmol, 0.20 equiv). The resulting solution was filled with CO (30 atm) and stirred for 16 hr at 80° C. The solids were filtered out. The resulting mixture was concentrated. The residue was applied onto a silica gel column with ethyl acetate/petroleum ether (1:2). This resulted in 1.66 g (92%) of methyl 3-methyl-1H-indazole-5-carboxylate as a yellow solid.

LC-MS (ES, m/z): [M+H]=191.

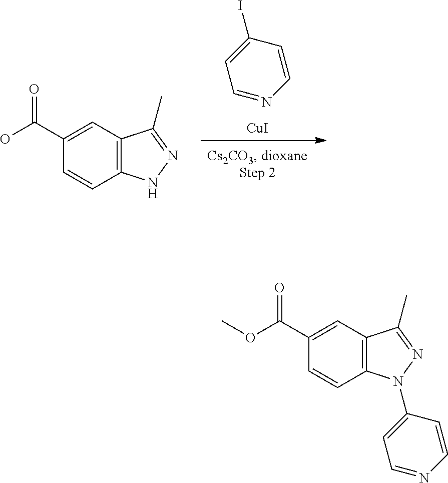

Into a 252-mL 3-necked round-bottom flask purged and maintained with an inert atmosphere of nitrogen, was placed dioxane (30.00 mL), methyl 3-methyl-1H-indazole-5-carboxylate (550.00 mg, 2.892 mmol, 1.00 equiv), 4-iodopyridine (1185.57 mg, 5.784 mmol, 2.00 equiv), (1R,2R)-cyclohexane-1,2-diamine (660.41 mg, 5.784 mmol, 2.00 equiv), CuI (550.72 mg, 2.892 mmol, 1.00 equiv), Cs₂CO₃ (2827 mg, 8.676 mmol, 3.00 equiv). The resulting solution was stirred for 24 hr at 100° C. The solids were filtered out. The resulting mixture was concentrated. The residue was applied onto a silica gel column with ethyl acetate/petroleum ether (1:2). This resulted in 770 mg (crude) of methyl 3-methyl-1-(pyridin-4-yl) indazole-5-carboxylate as a light brown solid.

LC-MS (ES, m/z): [M+H]=268.

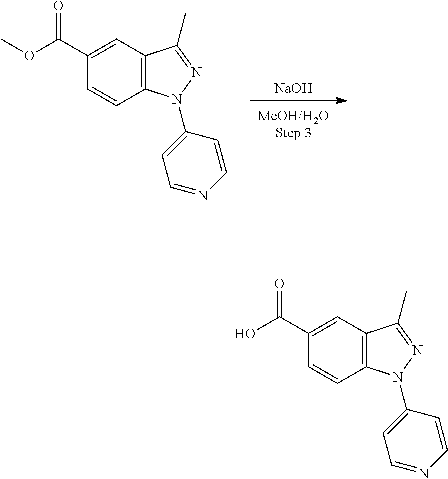 [SMILES: COC(=O)c1ccc2c(c1)c(C)nn2-c1ccncc1 ; "NaOH" ; Cc1nn(-c2ccncc2)c2ccc(C(=O)O)cc12]

Into a 50-mL round-bottom flask, was placed methyl 3-methyl-1-(pyridin-4-yl) indazole-5-carboxylate (300.00 mg, 1.122 mmol, 1.00 equiv), NaOH (89.78 mg, 2.244 mmol, 2.00 equiv), H₂O (5.00 mL), MeOH (10.00 mL). The resulting solution was stirred for 4 hr at room temperature. The resulting mixture was concentrated. The pH value of the solution was adjusted to 6 with citric acid (aq). The solids were collected by filtration. This resulted in 200 mg (70.4%) of 3-methyl-1-(pyridin-4-yl) indazole-5-carboxylic acid as a white solid.

LC-MS (ES, m/z): [M+H]=254.

Acid 16: 3-chloro-1H-indazole-5-carboxylic

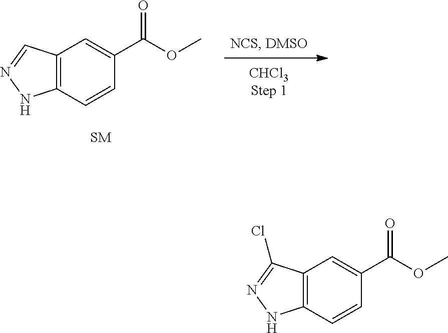

Into a 40-mL vial purged and maintained with an inert atmosphere of nitrogen, was placed methyl 1H-indazole-5-carboxylate (550.00 mg, 3.122 mmol, 1.00 equiv), CHCl₃ (11.00 mL), NCS (500.25 mg, 3.746 mmol, 1.20 equiv), DMSO (48.78 mg, 0.624 mmol, 0.20 equiv). The resulting solution was stirred for 24 hr at room temperature. The resulting solution was diluted with 30 mL of H₂O. The resulting solution was extracted with 3×30 mL of dichloromethane and the organic layers combined. The resulting mixture was washed with 2×30 mL of brine. The mixture was dried over anhydrous sodium sulfate and concentrated under vacuum. The residue was applied onto a silica gel column with ethyl acetate/petroleum ether (1/3). This resulted in 250 mg (38%) of methyl 3-chloro-1H-indazole-5-carboxylate as a brown solid.

LC-MS: (ES, m/z): [M−H]⁺=209

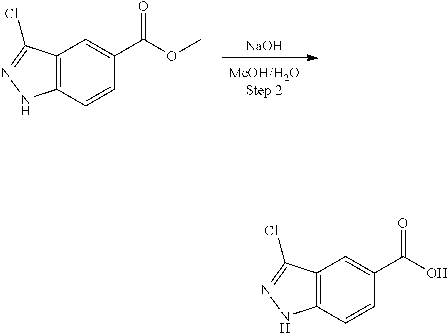

Into a 50-mL round-bottom flask, was placed methyl 3-chloro-1H-indazole-5-carboxylate (250.00 mg, 1.187 mmol, 1.00 equiv), CH₃OH (10.00 mL), H₂O (3.00 mL), sodium hydroxide (142.43 mg, 3.561 mmol, 3.00 equiv). The resulting solution was stirred for 16 hr at room temperature. The resulting solution was diluted with 20 mL of H₂O. The resulting solution was extracted with 2×20 mL of ethyl acetate and the aqueous layers combined. The pH value of the solution was adjusted to 3 with HCl (3 mol/L). The solids were collected by filtration. This resulted in 180 mg (77%) of 3-chloro-1H-indazole-5-carboxylic acid as a brown solid.

LC-MS: (ES, m/z): [M+H]⁺=197.

Acid 17: 3-fluoro-1H-indazole-5-carboxylic acid

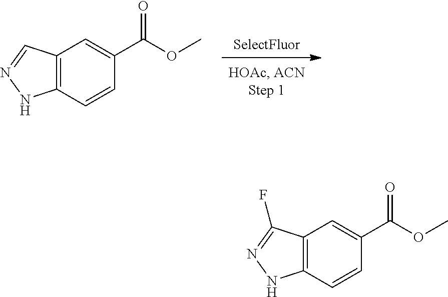

Into a 40-mL vial purged and maintained with an inert atmosphere of nitrogen, was placed methyl 1H-indazole-5-carboxylate (1.05 g, 5.960 mmol, 1.00 equiv), ACN (acetonitrile, 20.00 mL), HOAc (2.00 mL), SelectFluor (3.17 g, 8.948 mmol, 1.50 equiv). The resulting solution was stirred for 2 hr at 80° C. in an oil bath. The resulting mixture was concentrated under vacuum. The resulting solution was diluted with 30 mL of H₂O. The resulting solution was extracted with 3×20 mL of ethyl acetate and the organic layers combined. The resulting mixture was washed with 2×20 mL of brine. The mixture was dried over anhydrous sodium sulfate and concentrated under vacuum. The residue was applied onto a silica gel column with ethyl acetate/hexane (1/3). This resulted in 230 mg (20%) of methyl 3-fluoro-1H-indazole-5-carboxylate as a yellow solid.

LC-MS: (ES, m/z): [M+H]⁺=195.

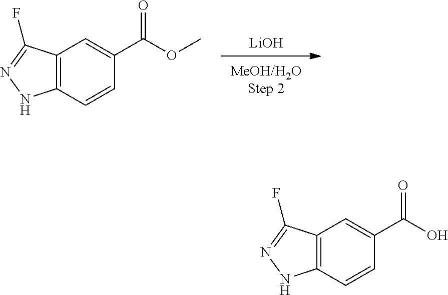

Into a 50-mL round-bottom flask, was placed methyl 3-fluoro-1H-indazole-5-carboxylate (230.00 mg, 1.185 mmol, 1.00 equiv), CH₃OH (12.00 mL), H₂O (4.00 mL), lithium hydroxide (85.11 mg, 3.554 mmol, 3.00 equiv). The resulting solution was stirred for 16 hr at room temperature. The resulting solution was diluted with 20 mL of H₂O. The resulting mixture was concentrated under vacuum. The resulting solution was extracted with 2×10 mL of ethyl acetate and the aqueous layers combined. The pH value of the solution was adjusted to 4 with HCl (1 mol/L). The solids were collected by filtration. This resulted in 150 mg (70.30%) of 3-fluoro-1H-indazole-5-carboxylic acid as a solid.

LC-MS: (ES, m/z): [M−H]⁺=179.

Acid 18: 3-methoxy-4-(methylamino) quinoline-7-carboxylic acid

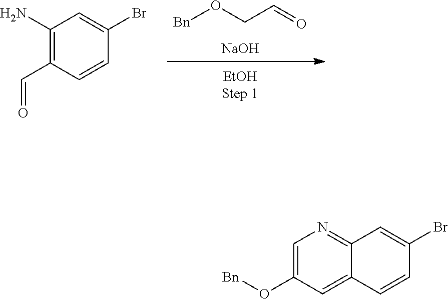

Into a 100 mL round-bottom flask were added 2-amino-4-bromobenzaldehyde (2.00 g, 10 mmol, 1.00 equiv), ethanol (40.00 mL), 2-(benzyloxy) acetaldehyde (3.45 g, 0.023 mmol, 2.3 equiv) and NaOH (2.40 g, 59.988 mmol, 6.00 equiv) at room temperature. The resulting mixture was stirred for 4 hr at 100° C. under nitrogen atmosphere. The mixture was allowed to cool down to room temperature. The resulting mixture was diluted with water (100 mL). The precipitated solids were collected by filtration and washed with water (2×30 mL). The resulting solid was dried under infrared light. This resulted in 3-(benzyloxy)-7-bromoquinoline (2.5 g, 63.7%) as a brown solid.

LC-MS (ES, m/z): [M+1]⁺=314.

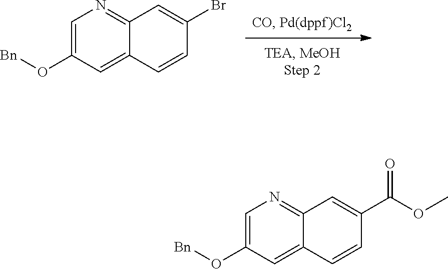 [SMILES: Brc1ccc2cc(OCc3ccccc3)cnc2c1 ; "CO, Pd(dppf)Cl2" ; COC(=O)c1ccc2cc(OCc3ccccc3)cnc2c1]

Into a 100 mL pressure reactor were added 3-(benzyloxy)-7-bromoquinoline (2.72 g, 8.66 mmol, 1.00 equiv), MeOH (60.00 mL), Pd (dppf)Cl₂ (0.63 g, 0.866 mmol, 0.10 equiv) and TEA (4.38 g, 43.287 mmol, 5.00 equiv) at room temperature. The resulting mixture was stirred for 3 hr at 100° C. under carbon monoxide (20 atm) atmosphere. The mixture was allowed to cool down to room temperature and then poured into 300 mL of water. The resulting mixture was stirred for 2 hr at room temperature. The precipitated solids were collected by filtration and washed with water (3×50 mL). The resulting solid was dried under infrared light. This resulted in methyl 3-(benzyloxy) quinoline-7-carboxylate (2.3 g, 90.6%) as a brown solid.

LC-MS (ES, m/z): [M+1]⁺=294.

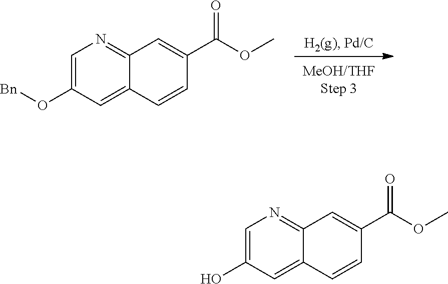 [SMILES: COC(=O)c1ccc2cc(OCc3ccccc3)cnc2c1 ; "H2(g), Pd/C" ; COC(=O)c1ccc2cc(O)cnc2c1]

Into a 1 L round-bottom flask were added methyl 3-(benzyloxy) quinoline-7-carboxylate (2.30 g, 7.841 mmol, 1.00 equiv) and MeOH (150.00 mL), THF (250.00 mL), Pd/C (0.46 g, 10% wet) at room temperature. The flask was evacuated and flushed three times with nitrogen, followed by flushing with hydrogen. The mixture was stirred 36 hr at room temperature under an atmosphere of hydrogen (balloon). The resulting mixture was filtered and the filter cake washed with MeOH (3×50 mL). The filtrate was concentrated under reduced pressure. The residue was purified by silica gel column chromatography, eluted with PE/EtOAc (1:9) to afford methyl 3-hydroxyquinoline-7-carboxylate (1 g, 62.8%) as a grey solid.

LC-MS (ES, m/z): [M+1]⁺=204.

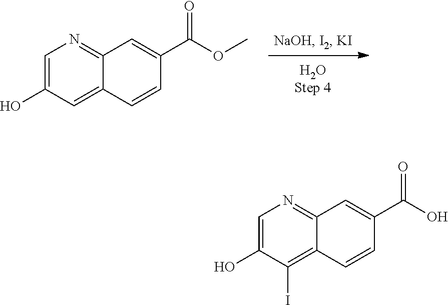

Into a 100 mL round-bottom flask were added methyl 3-hydroxyquinoline-7-carboxylate (800.00 mg, 3.937 mmol, 1 equiv) and NaOH (2 N) (16.00 mL) at room temperature. To the above mixture was added a solution of I₂ (1199.12 mg, 4.724 mmol, 1.20 equiv) and KI (20% aq) (16.00 mL, 20%) dropwise at room temperature. The resulting mixture was stirred for additional overnight at room temperature. The resulting mixture was diluted with water (50 mL). The mixture was acidified to pH 4 with acetic acid. The precipitated solids were collected by filtration and washed with water (2×10 mL). The residue was purified by reverse flash chromatography with the following conditions: column, C18 silica gel; mobile phase, ACN/0.5% TFA in water, 10% to 30% gradient in 20 min; detector, UV 254 nm. This resulted in 3-hydroxy-4-iodoquinoline-7-carboxylic acid (400 mg, 32.3%) as a yellow solid.

LC-MS (ES, m/z): [M+1]⁺=316.

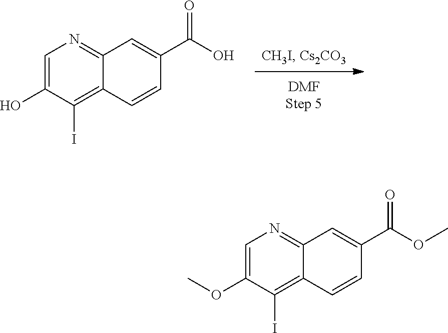

Into a 20 mL vial were added 3-hydroxy-4-iodoquinoline-7-carboxylic acid (400.00 mg, 1.27 mmol, 1.00 equiv), DMF (16.00 mL), Cs₂CO₃ (1.24 g, 3.8 mmol, 3.00 equiv) and CH₃I (450.00 mg, 3.17 mmol, 2.50 equiv) at room temperature. The resulting mixture was stirred for overnight at room temperature. The resulting mixture was filtered; the filter cake was washed with EtOAc (2×10 mL). The filtrate was washed with 2×10 mL of brine, dried over anhydrous Na₂SO₄. After filtration, the filtrate was concentrated under reduced pressure. The residue was purified by silica gel column chromatography, eluted with PE/EA (1:2) to afford methyl 4-iodo-3-methoxyquinoline-7-carboxylate (280 mg, 64.3%) as a light yellow solid.

LC-MS− (ES, m/z): [M+1]⁺=344.

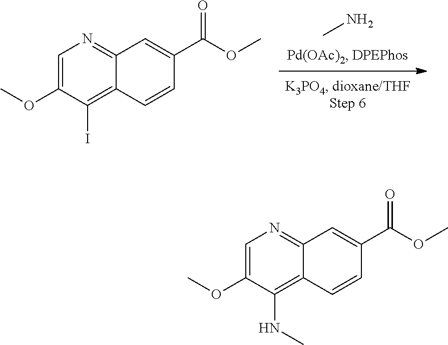

Into a 20 mL vial were added methyl 4-iodo-3-methoxyquinoline-7-carboxylate (280 mg, 0.816 mmol, 1.00 equiv), methylamine, 2M in THF (0.5 mg, 0.016 mmol, 0.02 equiv), K₃PO₄ (433.05 mg, 2.040 mmol, 2.5 equiv), [2-[2-(diphenylphosphanyl)phenoxy]phenyl]diphenylphosphane (87.90 mg, 0.163 mmol, 0.2 equiv), dioxane (10 mL, 118.041 mmol, 144.65 equiv) and Pd(OAc)₂ (18.32 mg, 0.082 mmol, 0.1 equiv) at room temperature. The resulting mixture was stirred for overnight at 90° C. under nitrogen atmosphere. The resulting mixture was diluted with EtOAc (40 mL). The resulting mixture was filtered. The filter cake was washed with EtOAc (1×10 mL). The filtrate was concentrated under reduced pressure. The residue was purified by silica gel column chromatography, eluted with PE/THF (1:4) to afford methyl 3-methoxy-4-(methylamino) quinoline-7-carboxylate (180 mg, 78.8%) as a brown solid.

LC-MS (ES, m/z): [M+1]⁺=247.

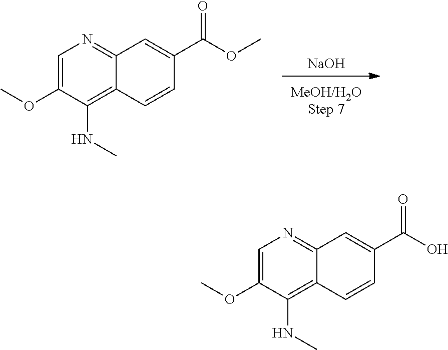

A solution of methyl 3-methoxy-4-(methylamino) quinoline-7-carboxylate (180 mg, 0.731 mmol, 1.00 equiv) and NaOH (116.94 mg, 2.924 mmol, 4 equiv) in MeOH (2 mL, 49.398 mmol, 67.58 equiv) and H₂O (2 mL, 111.017 mmol, 151.89 equiv) was stirred for 2 hr at 50° C. under nitrogen atmosphere. The mixture was acidified to pH 2-3 with dilute hydrochloric acid. The precipitated solids were collected by filtration and washed with H₂O (10 ml). This resulted in 3-methoxy-4-(methylamino) quinoline-7-carboxylic acid (100 mg, 59%) as a white solid.

LC-MS (ES, m/z): [M+1]⁺=233.

Acid 19: 1-(pyridin-2-yl) indazole-5-carboxylic acid

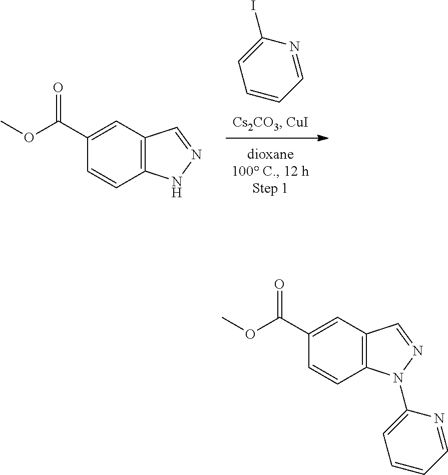

Into a 20-mL 4-necked round-bottom flask purged and maintained with an inert atmosphere of nitrogen, was placed a solution of methyl 1H-indazole-5-carboxylate (500.00 mg, 2.84 mmol, 1.00 equiv) in dioxane (10 mL), 2-iodopyridine (1163.6 mg, 5.676 mmol, 2.00 equiv), trans-1,2-diaminocyclohexane (648.17 mg, 5.68 mmol, 2.00 equiv), Cs₂CO₃ (2774 mg, 8.5 mmol, 3.00 equiv), CuI (540.51 mg, 2.84 mmol, 1.00 equiv). The resulting solution was stirred for 12 hr at 100° C. The resulting mixture was concentrated under vacuum. The residue was applied onto a silica gel column with ethyl acetate/petroleum ether (1:5). This resulted in 300 mg (41.7%) of methyl 1-(pyridin-2-yl) indazole-5-carboxylate as a yellow solid.

LC-MS (ES, m/z): [M+H]⁺=254.

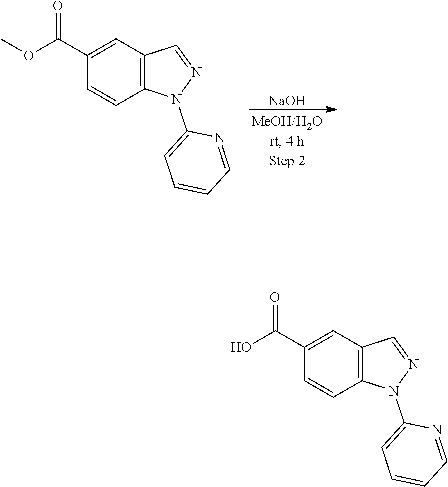 [SMILES: COC(=O)c1ccc2c(cnn2-c2ccccn2)c1 ; "NaOH" ; O=C(O)c1ccc2c(cnn2-c2ccccn2)c1]

Into a 20-mL vial, was placed a solution of methyl 1-(pyridin-2-yl) indazole-5-carboxylate (300.00 mg, 1.185 mmol, 1.00 equiv) in MeOH (5 mL), a solution of sodium hydroxide (189.51 mg, 4.740 mmol, 4.00 equiv) in H₂O (5 mL). The resulting solution was stirred for 4 hr at room temperature. The resulting mixture was concentrated. HCl (2 mol/L) was employed to adjust the pH to 2. The solids were collected by filtration. This resulted in 150 mg (52.9%) of 1-(pyridin-2-yl) indazole-5-carboxylic acid as a white solid.

Acid 20:1-(pyridin-3-yl)-1H-indazole-5-carboxylic acid

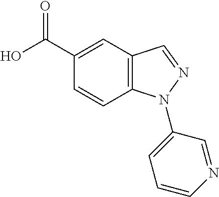 [SMILES: O=C(O)c1ccc2c(cnn2-c2cccnc2)c1]

Prepared as for 1-(pyridin-2-yl) indazole-5-carboxylic acid (Acid 19) using methyl 1H-indazole-5-carboxylate and 3-iodopyridine.

LC-MS (ES, m/z): [M+H]⁺=254.

Acid 21: 1-(pyrimidin-4-yl) indazole-5-carboxylic acid

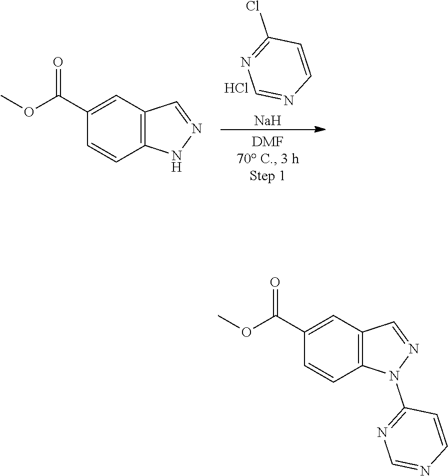

Into a 100-mL round-bottom flask purged and maintained with an inert atmosphere of nitrogen, was placed a solution of methyl 1H-indazole-5-carboxylate (500.00 mg, 2.838 mmol, 1.00 equiv) in DMF (20 mL). This was followed by the addition of NaH (81.73 mg, 2.043 mmol, 0.72 equiv, 60%) in several batches at 0° C. To this was added pyrimidine, 4-chloro-(487.57 mg, 4.257 mmol, 1.50 equiv) at 0° C. The resulting solution was stirred for 3 hr at room temperature. The reaction was then quenched by the addition of 100 ml of water/ice. The solids were collected by filtration. This resulted in 280 mg (39%) of methyl 1-(pyrimidin-4-yl) indazole-5-carboxylate as a yellow solid.

LC-MS (ES, m/z): [M+H]⁺=255

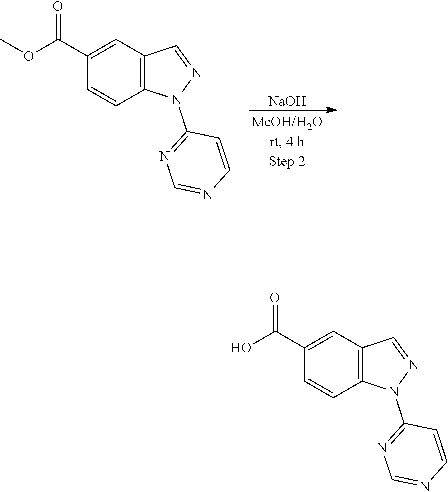 [SMILES: COC(=O)c1ccc2c(cnn2-c2ccncn2)c1 ; "NaOH" ; O=C(O)c1ccc2c(cnn2-c2ccncn2)c1]

Into a 12-mL vial purged and maintained with an inert atmosphere of nitrogen, was placed a solution of methyl 1-(pyrimidin-4-yl) indazole-5-carboxylate (280.00 mg, 1.101 mmol, 1.00 equiv) in MeOH (3 mL), a solution of sodium hydroxide (88.10 mg, 2.202 mmol, 2.00 equiv) in H₂O (2 mL). The resulting solution was stirred for 4 hr at room temperature. The resulting mixture was concentrated under vacuum. HCl (aq) (2 mol/L) was employed to adjust the pH to 2. The solids were collected by filtration. This resulted in 130 mg (49%) of 1-(pyrimidin-4-yl) indazole-5-carboxylic acid as a white solid.

LC-MS (ES, m/z): [M+H]⁺=241.

Acid 22: 1-(pyridazin-4-yl) indazole-5-carboxylic acid

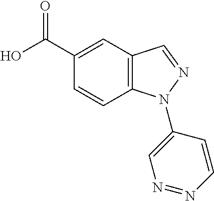 [SMILES: O=C(O)c1ccc2c(cnn2-c2ccnnc2)c1]

Prepared as for 1-(pyrimidin-4-yl) indazole-5-carboxylic acid (Acid 21) using methyl 1H-indazole-5-carboxylate and 4-bromopyridazine hydrobromide.

LC-MS: (ES, m/z): [M+H]⁺=254.

Acid 23: 3-[(tert-butoxycarbonyl) (2-methoxyethyl)amino]-1-methylindazole-6-carboxylic acid

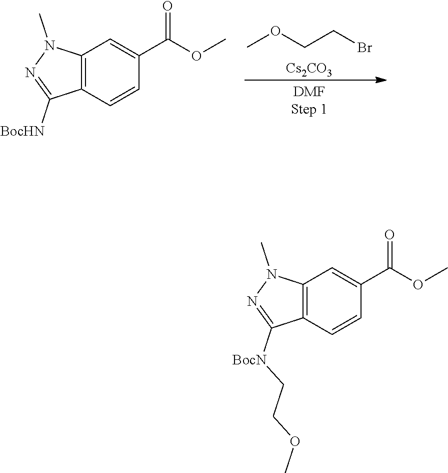

Into a 20 mL vial were added methyl 3-[(tert-butoxycarbonyl)amino]-1-methylindazole-6-carboxylate (500 mg, 1.638 mmol, 1.00 equiv), Cs₂CO₃ (1333.87 mg, 4.095 mmol, 2.5 equiv), DMF (10 mL) and 2-bromoethyl methyl ether (341.41 mg, 2.457 mmol, 1.5 equiv) at room temperature. The resulting mixture was stirred for 4 hr at 90° C. under nitrogen atmosphere. The resulting mixture was filtered, and the filter cake was washed with EtOAc (3×10 mL). The filtrate was treated with water (20 mL). The resulting mixture was separated. Then the aqueous layer was extracted with EtOAc (2×20 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (3:2) to afford methyl 3-[(tert-butoxycarbonyl) (2-methoxyethyl)amino]-1-methylindazole-6-carboxylate (480 mg, 80.7%) as a yellow oil.

LC-MS (ES, m/z): [M+1]⁺=364.

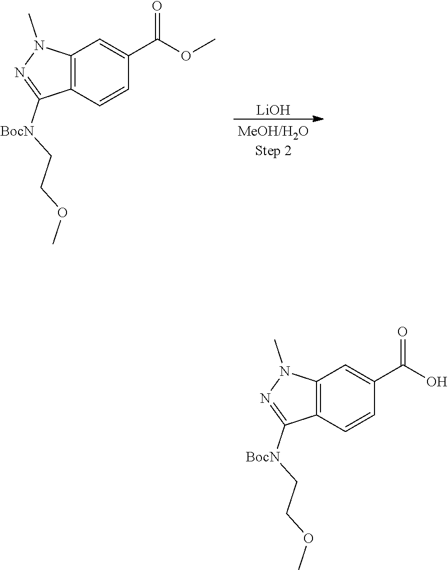 [SMILES: COCCN(C(=O)OC(C)(C)C)c1nn(C)c2cc(C(=O)OC)ccc12 ; "LiOH" ; COCCN(C(=O)OC(C)(C)C)c1nn(C)c2cc(C(=O)O)ccc12]

Into a 20 mL vial were added methyl 3-[(tert-butoxycarbonyl) (2-methoxyethyl)amino]-1-methylindazole-6-carboxylate (480 mg, 1.321 mmol, 1.00 equiv), MeOH (6 mL, 148.193 mmol, 112.20 equiv), LiOH (94.89 mg, 3.963 mmol, 3 equiv) and H₂O (2 mL, 111.017 mmol, 84.05 equiv) at room temperature. The resulting mixture was stirred for 4 hr at room temperature. The resulting mixture was concentrated under vacuum. The residue was dissolved in water (10 mL). The resulting mixture was washed with 3×10 mL of ethyl acetate. The aqueous layer was acidified to pH 6 with HCl (aq.). The mixture was allowed to cool down to 4° C. The crude product was crystallized after stay at that temperature to afford 3-[(tert-butoxycarbonyl) (2-methoxyethyl)amino]-1-methylindazole-6-carboxylic acid (130 mg, 28%) as a yellow solid.

LC-MS (ES, m/z): [M+1]⁺=350.

Acid 24: sodium 3-(oxetan-3-yl) imidazo[1,5-a]pyridine-7-carboxylate

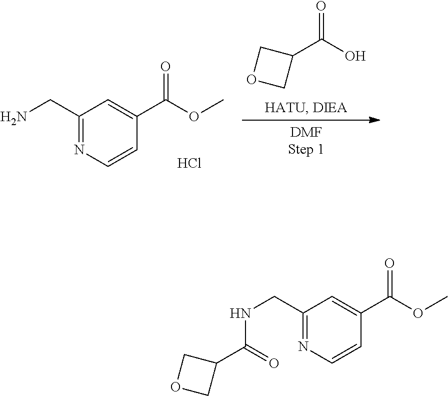

Into a 50-mL round-bottom flask purged and maintained with an inert atmosphere of nitrogen, was placed methyl 2-(aminomethyl)pyridine-4-carboxylate hydrochloride (750.00 mg, 3.7 mmol, 1.00 equiv), oxetane-3-carboxylic acid (453.42 mg, 4.441 mmol, 1.20 equiv), dimethylformamide (10.00 mL), HATU (1688.74 mg, 4.441 mmol, 1.20 equiv), DIEA (1913.39 mg, 14.805 mmol, 4.00 equiv). The resulting solution was stirred for 6 hr at room temperature. The reaction was then quenched by the addition of 30 mL of water/ice. The resulting solution was extracted with 4×10 mL of ethyl acetate and the organic layers combined and concentrated. This resulted in 400 mg (43.19%) of methyl 2-[(oxetan-3-ylformamido)methyl]pyridine-4-carboxylate as a brown solid.

LC-MS (ES, m/z): [M+1]⁺=251.

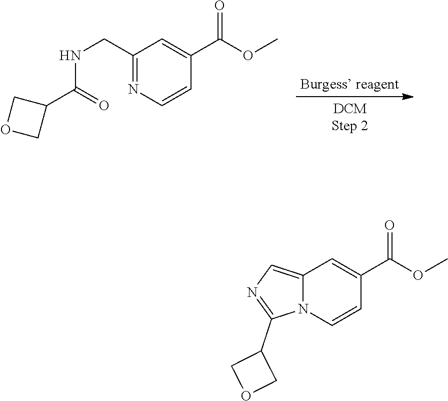 [SMILES: COC(=O)c1ccnc(CNC(=O)C2COC2)c1 ; COC(=O)c1ccn2c(C3COC3)ncc2c1]

Into a 50-mL round-bottom flask purged and maintained with an inert atmosphere of nitrogen, was placed methyl 2-[(oxetan-3-ylformamido)methyl]pyridine-4-carboxylate (400.00 mg, 1.6 mmol, 1.00 equiv), DCM (8.00 mL), Burgess reagent (1142.70 mg, 4.795 mmol, 3.0 equiv). The resulting solution was stirred for 4 hr at room temperature. The resulting mixture was concentrated. The residue was applied onto a silica gel column with THF/PE (1:3). This resulted in 140 mg (39%) of methyl (2Z)-2-(aminomethylidene)-1H-pyridine-4-carboxylate; oxetane as a light yellow solid.

LC-MS (ES, m/z): [M+1]⁺=233.

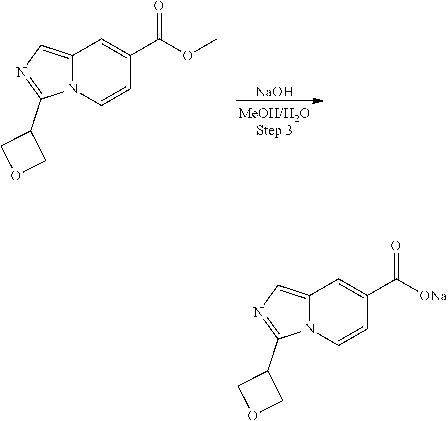

Into a 8-mL vial purged and maintained with an inert atmosphere of nitrogen, was placed methyl 3-(oxetan-3-yl) imidazo[1,5-a]pyridine-7-carboxylate (140.00 mg, 0.603 mmol, 1.00 equiv), sodium hydroxide (48.22 mg, 0.000 mmol, 2.00 equiv), MeOH (0.80 mL), H₂O (0.20 mL). The resulting solution was stirred for 16 hr at room temperature. The resulting mixture was concentrated. This resulted in 160 mg (crude) of sodium 3-(oxetan-3-yl) imidazo[1,5-a]pyridine-7-carboxylate as a light yellow solid.

LC-M (ES, m/z): [M+1]⁺=219.

Acid 25: 1-methoxyisoquinoline-6-carboxylic acid

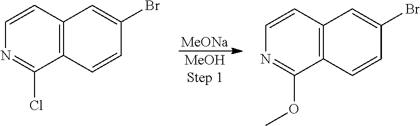

To a solution of 6-bromo-1-chloroisoquinoline (700 mg, 2.887 mmol, 1.00 equiv) in 10 mL MeOH was added MeONa (779.72 mg, 14.435 mmol, 5 equiv) in a 50 mL round-bottom flask. The resulting mixture was stirred for 23 hr at 80° C. The reaction was monitored by LCMS. The reaction was quenched by the addition of water/ice (100 mL) at room temperature. The resulting mixture was extracted with EtOAc (2×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford 6-bromo-1-methoxyisoquinoline (610 mg, 88.8%) as a yellow solid.

LC-MS (ES, m/z): [M+H]=238.

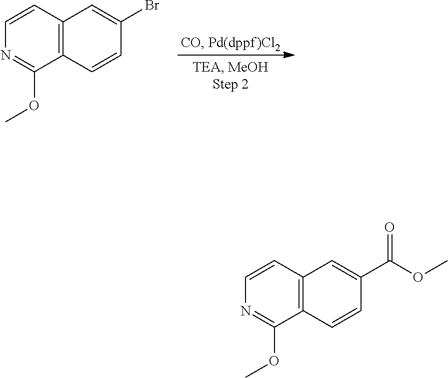 [SMILES: COc1nccc2cc(Br)ccc12 ; COC(=O)c1ccc2c(OC)nccc2c1]

To a solution of 6-bromo-1-methoxyisoquinoline (610 mg, 2.562 mmol, 1.00 equiv) in 10.0 MeOH was added TEA (1296.31 mg, 12.810 mmol, 5 equiv) and Pd(dppf)Cl₂CH₂Cl₂ (208.72 mg, 0.256 mmol, 0.1 equiv in a pressure tank. The mixture was purged with nitrogen for 0.5 min and then was pressurized to 3 MPa with carbon monoxide at 80° C. for 16 hr. The resulting mixture was concentrated under vacuum. The residue was purified by silica gel column chromatography, eluted with PE/EA (1:1) to afford methyl 1-methoxyisoquinoline-6-carboxylate (510 mg, 91.6%) as a brown solid.

LC-MS (ES, m/z): [M+H]=218.

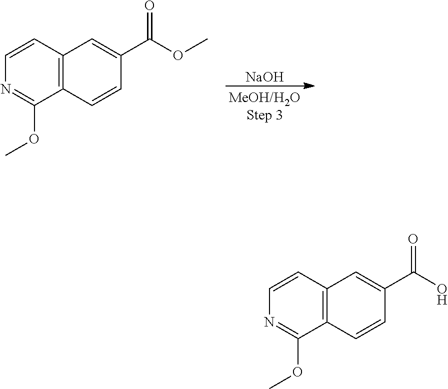

To a solution of methyl 1-methoxyisoquinoline-6-carboxylate (510 mg, 2.35 mmol, 1.00 equiv) in 10 mL MeOH and 2 mL H₂O was added NaOH (187.81 mg, 4.696 mmol, 2 equiv) in a 50 mL round-bottom flask at room temperature. The resulting mixture was stirred for 16 hr at room temperature. The reaction was monitored by LCMS. The resulting mixture was concentrated under vacuum. The residue was acidified to pH 5 with citric acid. The precipitated solids were collected by filtration. The resulting solid was dried under infrared light. This resulted in 1-methoxyisoquinoline-6-carboxylic acid (410 mg, 86%) as a white solid.

LC-MS (ES, m/z): [M+H]=204.

Acid 26: 3-methyl-1-(trifluoromethyl) imidazo[1,5-a]pyridine-6-carboxylic acid

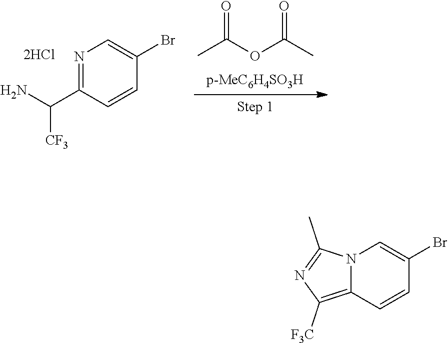

To a solution of 1-(5-bromopyridin-2-yl)-2,2,2-trifluoroethanamine dihydrochloride (850 mg, 2.592 mmol, 1.00 equiv) in acetic anhydride (10 mL, 97.954 mmol, 37.79 equiv) was added para-toluene sulfonate (892.64 mg, 5.184 mmol, 2.0 equiv). The resulting mixture was stirred for 3 hr at 100° C. The reaction was monitored by LCMS. The resulting mixture was concentrated under vacuum. The residue was purified by silica gel column chromatography, eluted with PE/EA (3:1) to afford 6-bromo-3-methyl-1-(trifluoromethyl) imidazo[1,5-a]pyridine (390 mg, 53.9%) as a brown solid.

LC-MS (ES, m/z): [M+H]=279.

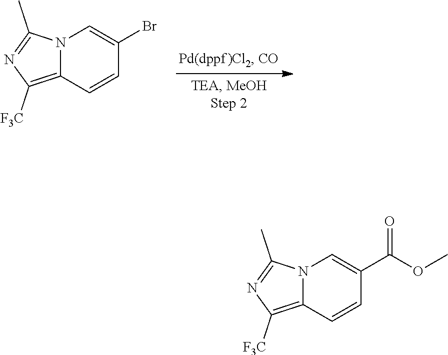 [SMILES: Cc1nc(C(F)(F)F)c2ccc(Br)cn12 ; COC(=O)c1ccc2c(C(F)(F)F)nc(C)n2c1]

To a solution of 6-bromo-3-methyl-1-(trifluoromethyl) imidazo[1,5-a]pyridine (390 mg, 1.4 mmol, 1.00 equiv) in 10 mL MeOH and TEA (707 mg, 6.99 mmol, 5 equiv) was added Pd(dppf)Cl₂·CH₂Cl₂ (113.85 mg, 0.140 mmol, 0.1 equiv) in a pressure tank. The mixture was purged with nitrogen for 0.3 min and then was pressurized to 3 MPa with carbon monoxide at 120° C. for 3 hr. The reaction mixture was cooled to room temperature and filtered to remove insoluble solids. The filtrate was concentrated under reduced pressure. The residue was purified by silica gel column chromatography, eluted with PE/EA (1:1) to afford methyl 3-methyl-1-(trifluoromethyl) imidazo[1,5-a]pyridine-6-carboxylate (290 mg, 80.4%) as a yellow solid.

LC-MS (ES, m/z): [M+H]=259.

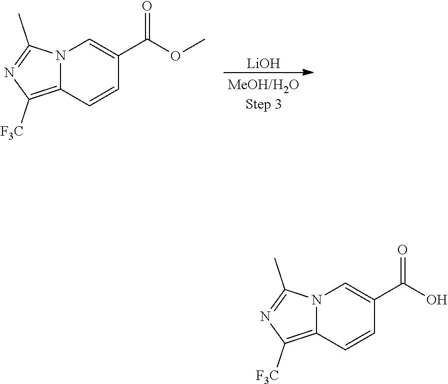

To a solution of methyl 3-methyl-1-(trifluoromethyl) imidazo[1,5-a]pyridine-6-carboxylate (290 mg, 1.123 mmol, 1.00 equiv) in 10 mL MeOH and 1 mL H₂O was added LiOH (53.79 mg, 2.246 mmol, 2 equiv) in a 50 mL round-bottom flask. The resulting mixture was stirred for 16 hr at room temperature. The reaction was monitored by LCMS. The resulting mixture was concentrated under vacuum. The residue was acidified/to pH 5 with citric acid. The precipitated solids were collected by filtration. The resulting solid was dried under infrared light. This resulted in 3-methyl-1-(trifluoromethyl) imidazo[1,5-a]pyridine-6-carboxylic acid (220 mg, 80%) as a white solid.

LC-MS (ES, m/z): [M+H]=245.

Acid 27: 3-methyl-1-(trifluoromethyl) imidazo[1,5-a]pyridine-6-carboxylic acid

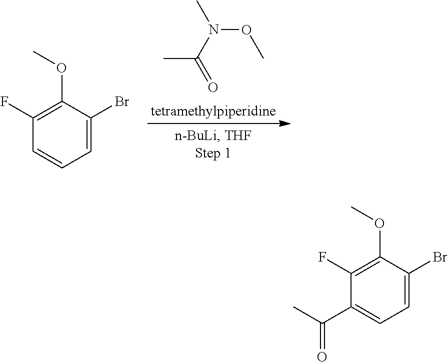

In a 100-mL 3-necked round-bottom flask, to a solution of 1-bromo-3-fluoro-2-methoxybenzene (1 g, 4.9 mmol, 1.00 equiv) in THF (20 mL) was added dropwise 2,2,6,6-tetramethylpiperidine (1.10 g, 7.803 mmol, 1.6 equiv) and n-BuLi (2.34 mL, 5.9 mmol, 1.2 equiv) at −78° C. under N₂ atmosphere. The reaction mixture was stirred at −78° C. for 15 mins. Then, N-methoxy-N-methylacetamide (1.51 g, 14.63 mmol, 3.0 equiv) was added dropwise at −78° C. and the mixture was stirred for another 1 hr. The mixture was allowed to warm to RT and stirred for 10 min. The reaction was monitored by TLC. The reaction was quenched with NH₄Cl (50 mL), and then the mixture was extracted with EtOAc (2×25 mL). The combined organic extracts were washed with brine (50 mL), dried over anhydrous Na₂SO₄, The residue was purified by silica gel column chromatography, eluted with PE/EA (5:1) to afford 1-(4-bromo-2-fluoro-3-methoxyphenyl) ethanone (1.1 g, 73%) as a brown oil.

LC-MS (ES, m/z): [M+H]=247.

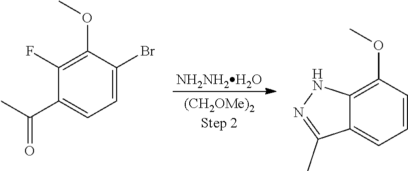

To a solution of 1-(4-bromo-2-fluoro-3-methoxyphenyl) ethanone (1.1 g, 4.45 mmol, 1.00 equiv) in 10 mL DME was added NH₂NH₂·H₂O (1 mL, 20.575 mmol, 4.62 equiv) in a 50 mL round-bottom flask. The resulting mixture was stirred for 12 hr at 100° C. The reaction was monitored by LCMS. The resulting mixture was concentrated under vacuum. The residue was purified by silica gel column chromatography, eluted with PE/EA (1:1) to afford 6-bromo-7-methoxy-3-methyl-1H-indazole (220 mg, 20.50%) as a white solid.

LC-MS (ES, m/z): [M+H]=241

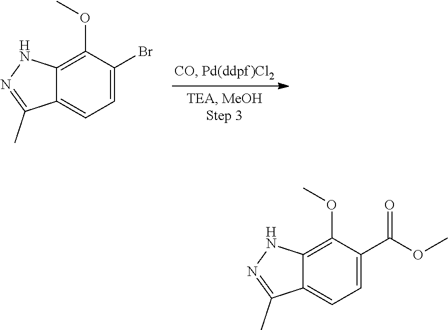 [SMILES: COc1c(Br)ccc2c(C)n[nH]c12 ; COC(=O)c1ccc2c(C)n[nH]c2c1OC]

To a solution of 6-bromo-7-methoxy-3-methyl-1H-indazole (220 mg, 0.913 mmol, 1.00 equiv) in 10 mL MeOH and TEA (461.69 mg, 4.565 mmol, 5 equiv) was added Pd(dppf)Cl₂CH₂Cl₂ (74.34 mg, 0.091 mmol, 0.1 equiv) in a pressure tank. The mixture was purged with nitrogen for 0.3 min and then was pressurized to 3 MPa with carbon monoxide at 120° C. for 4 hr. The reaction mixture was cooled to room temperature and filtered to remove insoluble solids. The filtrate was concentrated under reduced pressure. The residue was purified by silica gel column chromatography, eluted with PE/EA (3:1) to afford methyl 7-methoxy-3-methyl-1H-indazole-6-carboxylate (160 mg, 79.6%) as a brown oil.

LC-MS (ES, m/z): [M+H]=221.

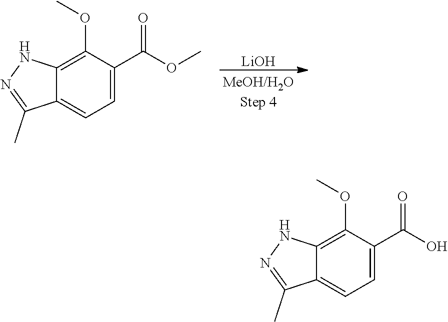

To a solution of methyl 7-methoxy-3-methyl-1H-indazole-6-carboxylate (160 mg, 0.727 mmol, 1.00 equiv) in 10 mL MeOH and 2 mL H₂O was added LiOH (34.8 mg, 1.45 mmol, 2 equiv) in a 50 mL round-bottom flask. The resulting mixture was stirred for 4 hr at room temperature. The reaction was monitored by LCMS. The resulting mixture was concentrated under vacuum. The residue was acidified to pH 5 with citric acid. The precipitated solids were collected by filtration. The resulting solid was dried under infrared light. This resulted in 3-methyl-1-(trifluoromethyl) imidazo[1,5-a]pyridine-6-carboxylic acid (88 mg, 58.7%) as a white solid.

LC-MS (ES, m/z): [M+H]=207.

Acid 28: 3-fluoro-1-methylindazole-6-carboxylic acid

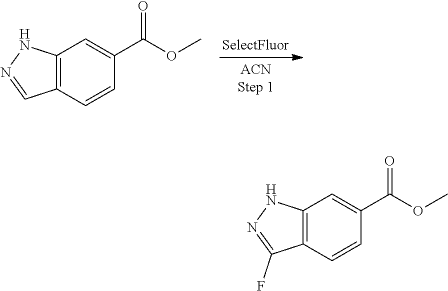

Into a 40-mL vial purged and maintained with an inert atmosphere of nitrogen, was placed methyl 1H-indazole-6-carboxylate (1.1 g, 6.244 mmol, 1.00 equiv), ACN (22 mL), and SelectFluor (2.21 g, 6.244 mmol, 1 equiv) in several batches. The resulting solution was stirred for 2 hr at 50° C. in an oil bath. The resulting mixture was concentrated under vacuum. The resulting solution was diluted with 20 mL of H₂O. The resulting solution was extracted with 3×20 mL of ethyl acetate and the organic layers combined. The resulting mixture was washed with 2×20 mL of brine. The mixture was dried over anhydrous sodium sulfate and concentrated under vacuum. The residue was applied onto a silica gel column with ethyl acetate/petroleum ether (1:1). This resulted in 300 mg (24.75%) of methyl 3-fluoro-1H-indazole-6-carboxylate as a white solid.

LC-MS: (ES, m/z): [M+H]⁺=195.

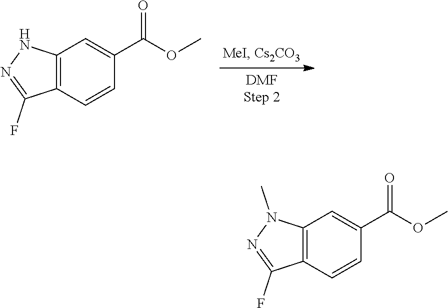

Into a 50-mL round-bottom flask, was placed methyl 3-fluoro-1H-indazole-6-carboxylate (300 mg, 1.545 mmol, 1.00 equiv), DMF (9 mL), Cs₂CO₃ (1006.83 mg, 3.090 mmol, 2 equiv), Mel (328.96 mg, 2.317 mmol, 1.5 equiv). The resulting solution was stirred for 6 hr at room temperature. The resulting solution was diluted with 30 mL of H₂O. The resulting solution was extracted with 2×20 mL of ethyl acetate and the aqueous layers combined. The resulting mixture was washed with brine 3×30 mL. The resulting mixture was concentrated under reduced pressure. This resulted in methyl 3-fluoro-1-methylindazole-6-carboxylate (200 mg, 62%) as a white solid.

LC-MS: (ES, m/z): [M+H]⁺=209.

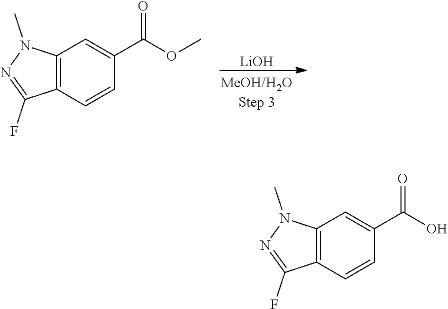

Into a 50-mL round-bottom flask, was placed methyl 3-fluoro-1-methylindazole-6-carboxylate (200 mg, 0.961 mmol, 1.00 equiv), MeOH (9 mL), H₂O (3 mL), lithium hydroxide (69.02 mg, 2.883 mmol, 3 equiv). The resulting solution was stirred for 16 hr at room temperature. The resulting mixture was concentrated under vacuum. The resulting solution was diluted with 50 mL of H₂O. The resulting solution was extracted with 2×20 mL of ethyl acetate and the aqueous layers combined. The pH value of the solution was adjusted to 3 with HCl (3 mol/L). The solids were collected by filtration. This resulted in 3-fluoro-1-methylindazole-6-carboxylic acid (150 mg, 80.4%) as a white solid.

LC-MS: (ES, m/z): [M+H]⁺=195

Acid 29: 3-methyl-[1,2,3]triazolo[1,5-a]pyridine-6-carboxylic acid

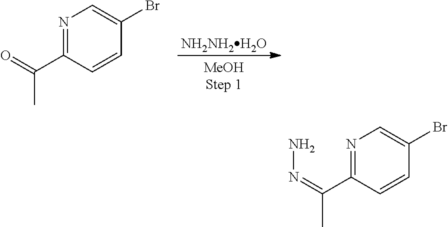

Into a 100 mL 3-necked round-bottom flask were added 1-(5-bromopyridin-2-yl) ethanone (2 g, 9.998 mmol, 1.00 equiv), methanol (40 mL, 1248.362 mmol, 124.86 equiv) and NH₂NH₂·H₂O (1.50 g, 29.994 mmol, 3.0 equiv) at room temperature. The resulting mixture was stirred for 6 hr at 60° C. under nitrogen atmosphere. The resulting mixture was concentrated under reduced pressure. This resulted in 5-bromo-2-[(1Z)-ethanehydrazonoyl]pyridine (2.1 g, 98.12%) as a light brown solid.

LC-MS (ES, m/z): [M+1]⁺=214.

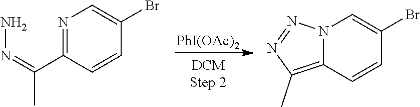

Into a 100 mL 3-necked round-bottom flask were added 5-bromo-2-[(1Z)-ethanehydrazonoyl]pyridine (2.1 g, 9.810 mmol, 1.00 equiv), DCM (40 mL, 629.202 mmol, 64.14 equiv) and iodosobenzene diacetate (3.79 g, 11.77 mmol, 1.20 equiv) at room temperature. The resulting mixture was stirred for 2 hr at room temperature under nitrogen atmosphere. The reaction was quenched by the addition of sat. sodium bisulfite (aq) (40 mL) at room temperature. The aqueous layer was extracted with CH₂Cl₂ (3×20 mL). The resulting mixture was concentrated under reduced pressure. The residue was purified by silica gel column chromatography, eluted with PE/EA (1:1) to afford 6-bromo-3-methyl-[1,2,3]triazolo[1,5-a]pyridine (1.9 g, 91.3%) as a light yellow solid.

LC-MS (ES, m/z): [M+1]⁺=212.

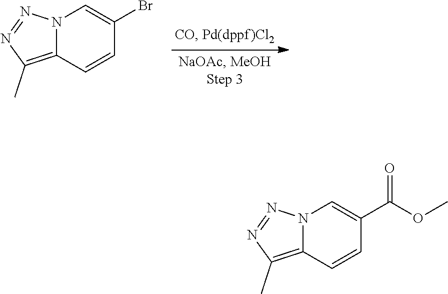

Into a 50-mL sealed tube, was placed 6-bromo-3-methyl-[1,2,3]triazolo[1,5-a]pyridine (1.9 g, 8.960 mmol, 1.00 equiv), sodium acetate (2.21 g, 26.880 mmol, 3.0 equiv), methanol (36 mL), Pd(dppf)Cl₂ (1.31 g, 1.792 mmol, 0.2 equiv), CO (20 atm). The resulting solution was stirred for 5 hr at 100° C. The resulting mixture was concentrated. The residue was applied onto a silica gel column with ethyl acetate/petroleum ether (1:1). This resulted in methyl 3-methyl-[1,2,3]triazolo[1,5-a]pyridine-6-carboxylate (1.6 g, 93.4%) as a light brown solid.

LC-MS (ES, m/z): [M+1]⁺=191.

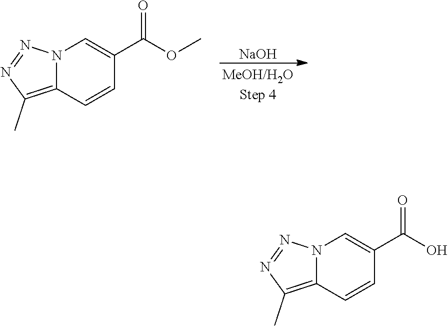

Into a 100 mL round-bottom flask were added methyl 3-methyl-[1,2,3]triazolo[1,5-a]pyridine-6-carboxylate (1.6 g, 8.369 mmol, 1.00 equiv), methanol (32 mL) and sodium hydroxide (0.67 g, 16.738 mmol, 2.00 equiv), water (8 mL) at room temperature. The resulting mixture was stirred for 16 hr at room temperature under nitrogen atmosphere. The mixture/residue was acidified to pH 4 with conc. HCl. The precipitated solids were collected by filtration. This resulted in 3-methyl-[1,2,3]triazolo[1,5-a]pyridine-6-carboxylic acid (1.3 g, 88%) as a light brown solid.

LC-MS (ES, m/z): [M+1]⁺=177.

Acid 30: 1-methylisoquinoline-6-carboxylic acid

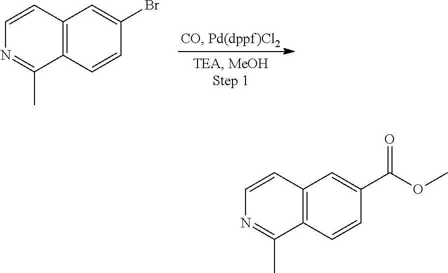

Into a 50-mL pressure reactor, was placed 6-bromo-1-methylisoquinoline (250 mg, 1.13 mmol, 1.00 equiv), CH₃OH (10 mL), Pd(dppf)Cl₂ (82.37 mg, 0.113 mmol, 0.1 equiv), TEA (455.64 mg, 4.504 mmol, 4 equiv), CO (10 atm). The resulting solution was stirred for 16 hr at 120° C. in an oil bath. The reaction mixture was cooled. The resulting mixture was concentrated under vacuum. The resulting solution was diluted with 30 mL of H₂O. The resulting solution was extracted with 3×20 mL of ethyl acetate and the organic layers combined. The resulting mixture was washed with 2×20 mL of brine. The mixture was dried over anhydrous sodium sulfate and concentrated under vacuum. The residue was applied onto a silica gel column with ethyl acetate/petroleum ether (3:1). This resulted in methyl 1-methylisoquinoline-6-carboxylate (120 mg, 53%) as an orange solid.

LC-MS: (ES, m/z): [M+H]⁺=202.

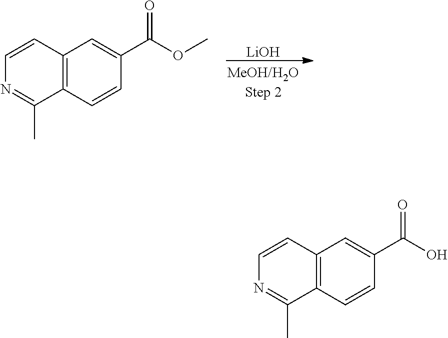

Into a 50-mL round-bottom flask, was placed methyl 1-methylisoquinoline-6-carboxylate (120 mg, 0.596 mmol, 1.00 equiv), MeOH (3 mL), H₂O (1 mL), lithium hydroxide (42.85 mg, 1.788 mmol, 3 equiv). The resulting solution was stirred for 6 hr at room temperature. The resulting mixture was concentrated under vacuum. The resulting solution was diluted with 50 mL of H₂O. The resulting solution was extracted with 2×20 mL of ethyl acetate and the aqueous layers combined. The pH value of the solution was adjusted to 3 with HCl (3 mol/L). The solids were collected by filtration. This resulted in 1-methylisoquinoline-6-carboxylic acid (80 mg, 71.7%) as an off-white solid.

LC-MS: (ES, m/z): [M+H]⁺=188.

Acid 31: 3-isopropylimidazo[1,5-a]pyridine-7-carboxylic acid

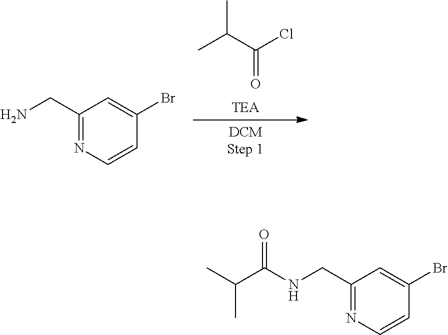

To a solution of 1-(4-bromopyridin-2-yl) methanamine (1 g, 5.35 mmol, 1.00 equiv) in 20 mL DCM was added isobutyryl chloride (0.63 g, 5.881 mmol, 1.1 equiv) and TEA (0.81 g, 8.019 mmol, 1.5 equiv) in a 100 mL 3-necked round-bottom flask. The resulting mixture was stirred for 10 hr at room temperature. The reaction was quenched by the addition of water (100 mL). The aqueous layer was extracted with CH₂Cl₂ (2×40 mL). The combined organic layers were dried over anhydrous Na₂SO₄. After filtration, the filtrate was concentrated under reduced pressure. This resulted in N-[(4-bromopyridin-2-yl)methyl]-2-methylpropanamide (1.2 g, 87.3%) as a brown solid.

LC-MS (ES, m/z): [M+H]=257.

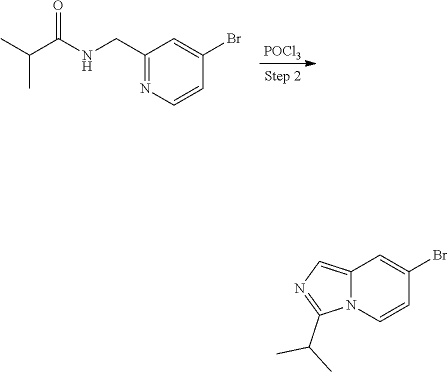

Into a 50 mL 3-necked round-bottom flask were added N-[(4-bromopyridin-2-yl)methyl]-2-methylpropanamide (1.2 g, 4.667 mmol, 1.00 equiv) and POCl₃ (10 mL, 107.284 mmol, 23 equiv). The resulting mixture was stirred for 16 hr at 100° C. The reaction was monitored by LCMS. The resulting mixture was concentrated under vacuum. The residue was basified to pH 8 with saturated Na₂CO₃ (aq.). The resulting mixture was extracted with EtOAc (2× 20 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford 7-bromo-3-isopropylimidazo[1,5-a]pyridine (902 mg, 80.8%) as a brown oil.

LC-MS (ES, m/z): [M+H]=239.

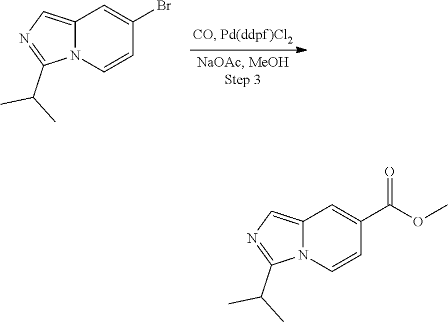 [SMILES: CC(C)c1ncc2cc(Br)ccn12 ; COC(=O)c1ccn2c(C(C)C)ncc2c1]

To a solution of 7-bromo-3-isopropylimidazo[1,5-a]pyridine (400 mg, 1.673 mmol, 1.00 equiv) in 20 mL MeOH was added Pd(dppf)Cl₂CH₂Cl₂ (136.3 mg, 0.167 mmol, 0.1 equiv) and NaOAc (686.1 mg, 8.365 mmol, 5 equiv) in a pressure tank. The mixture was purged with nitrogen for 0.2 min and then was pressurized to 3 MPa with carbon monoxide at 100° C. for 1 hr. The reaction mixture was cooled to room temperature and filtered to remove insoluble solids. The filtrate was concentrated under reduced pressure. The residue was purified by silica gel column chromatography, eluted with PE/EA (1:1) to afford methyl 3-isopropylimidazo[1,5-a]pyridine-7-carboxylate (310 mg, 84.9%) as a brown solid.

LC-MS (ES, m/z): [M+H]=219.

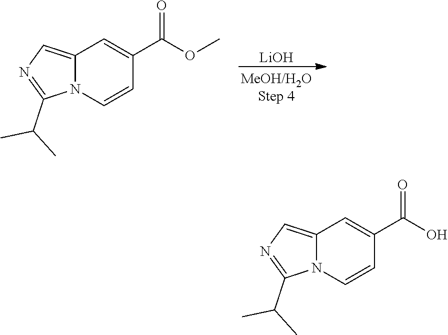

To a solution of methyl 3-isopropylimidazo[1,5-a]pyridine-7-carboxylate (310 mg, 1.42 mmol, 1.00 equiv) in 10 mL MeOH and 1 mL H₂O was added LiOH (68.03 mg, 2.84 mmol, 2 equiv) in 50 mL 3-necked round-bottom flask. The resulting mixture was stirred for 16 hr at room temperature. The reaction was monitored by LCMS. The resulting mixture was concentrated under vacuum. The residue was acidified to pH 4 with citric acid. The precipitated solids were collected by filtration and dried under infrared light. This resulted in 3-isopropylimidazo[1,5-a]pyridine-7-carboxylic acid (300 mg) as a white crude solid.

LC-MS− (ES, m/z): [M+H]=204.

Acid 32: 3-cyclopropylimidazo[1,5-a]pyridine-7-carboxylic acid

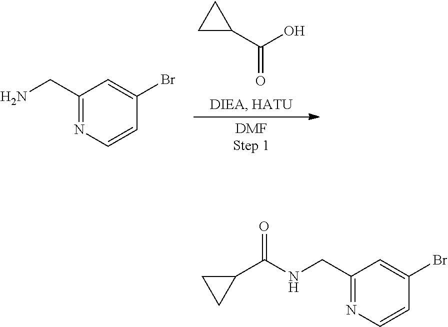 [SMILES: NCc1cc(Br)ccn1 ; O=C(O)C1CC1 ; O=C(NCc1cc(Br)ccn1)C1CC1]

Into a 100 mL 3-necked round-bottom flask were added 1-(4-bromopyridin-2-yl) methanamine (2 g, 10.693 mmol, 1.00 equiv), cyclopropanecarboxylic acid (1.20 g, 13.901 mmol, 1.3 equiv), dimethylformamide (40 mL, 547.23 mmol, 51.18 equiv), HATU (4.88 g, 12.83 mmol, 1.2 equiv) and DIEA (5.53 g, 42.77 mmol, 4.0 equiv) at room temperature. The resulting mixture was stirred for 10 hr at room temperature under nitrogen atmosphere. The reaction was quenched by the addition of water/ice (150 mL) at room temperature. The aqueous layer was extracted with EtOAc (3×60 mL). The resulting mixture was washed with 3×40 mL of brine. The resulting mixture was concentrated under reduced pressure. This resulted in N-[(4-bromopyridin-2-yl)methyl]cyclopropanecarboxamide (2.8 g, crude) as a light brown solid.

LC-MS (ES, m/z): [M+1]⁺=255.

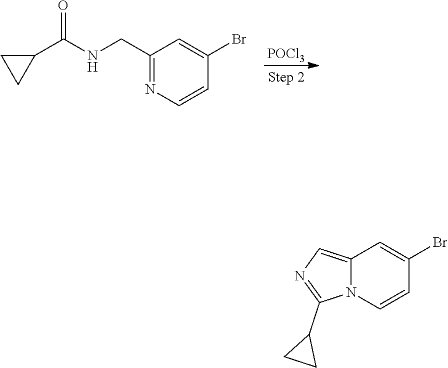

Into a 50 mL round-bottom flask were added N-[(4-bromopyridin-2-yl)methyl]cyclopropanecarboxamide (1.5 g, 5.88 mmol, 1.00 equiv) and phosphorus oxychloride (10 mL, 65.22 mmol, 11.09 equiv) at room temperature. The resulting mixture was stirred for 16 hr at 110° C. under nitrogen atmosphere. The resulting mixture was concentrated under reduced pressure. The residue was purified by silica gel column chromatography, eluted with PE/EA (3:1) to afford 7-bromo-3-cyclopropylimidazo[1,5-a]pyridine (600 mg, 43%) as a light brown oil.

LC-MS (ES, m/z): [M+1]⁺=237.

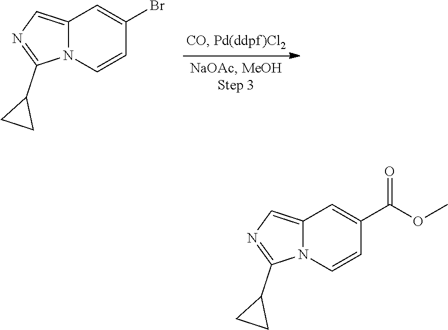 [SMILES: Brc1ccn2c(C3CC3)ncc2c1 ; COC(=O)c1ccn2c(C3CC3)ncc2c1]

Into a 50-mL sealed tube, was placed 7-bromo-3-cyclopropylimidazo[1,5-a]pyridine (600 mg, 2.531 mmol, 1.00 equiv), sodium acetate (622.8 mg, 7.593 mmol, 3.0 equiv), Pd(dppf)Cl₂ (185.16 mg, 0.253 mmol, 0.1 equiv), MeOH (20.00 mL), CO (20 atm). The resulting solution was stirred for 3 hr at 100° C. The resulting mixture was concentrated. The residue was applied onto a silica gel column with ethyl acetate/petroleum ether (1:3). This resulted in methyl 3-cyclopropylimidazo[1,5-a]pyridine-7-carboxylate (210 mg, 38%) as a light brown solid.

LC-MS (ES, m/z): [M+1]⁺=217.

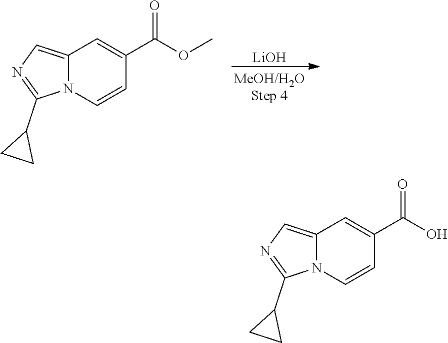

Into a 8 mL vial were added methyl 3-cyclopropylimidazo[1,5-a]pyridine-7-carboxylate (210 mg, 0.971 mmol, 1.00 equiv), methanol (4 mL, 124.836 mmol, 128.55 equiv), lithium hydroxide (46.5 mg, 1.94 mmol, 2.0 equiv), water (1 mL, 55.5 mmol, 57.16 equiv) at room temperature. The resulting mixture was stirred for 16 hr at room temperature. The reaction was quenched by the addition of water/ice (10 mL) at room temperature. The mixture/residue was acidified to pH 4 with conc. HCl. The precipitated solids were collected by filtration. This resulted in 3-cyclopropylimidazo[1,5-a]pyridine-7-carboxylic acid (305 mg, crude) as a light brown solid.

LC-MS (ES, m/z): [M+1]⁺=203.

Acid 33: 2-methylpyrrolo[1,2-b]pyridazine-6-carboxylic acid

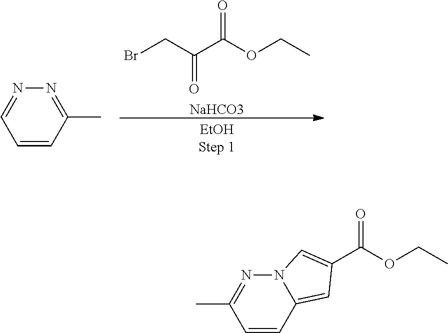

Into a 50 mL pressure reactor were added 3,6-dimethylpyridazine (2 g, 18.494 mmol, 1.00 equiv), ethyl 3-bromo-2-oxopropanoate (7.21 g, 36.99 mmol, 2.0 equiv) and sodium bicarbonate (4.66 g, 55.482 mmol, 3.0 equiv), ethanol (30 mL, 651.197 mmol, 35.21 equiv) at room temperature. The resulting mixture was stirred for 20 hr at 120° C. under nitrogen atmosphere. The resulting mixture was concentrated under reduced pressure. The residue was purified by silica gel column chromatography, eluted with PE/EA (1:1) to afford ethyl 2-methylpyrrolo[1,2-b]pyridazine-6-carboxylate (600 mg, 16%) as a light brown solid.

LC-MS (ES, m/z): [M+1]⁺=204

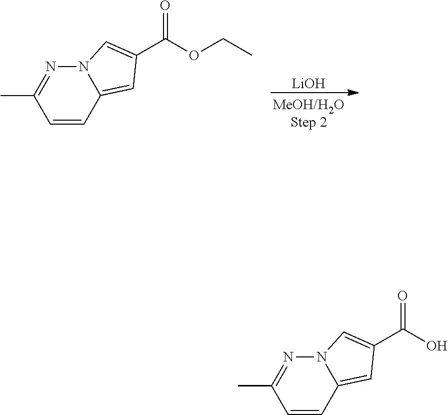

Into a 8 mL vial were added ethyl pyrrolo[1,2-b]pyridazine-6-carboxylate (600 mg, 3.2 mmol, 1.00 equiv), methanol (4 mL), lithium hydroxide (151.10 mg, 6.310 mmol, 2.0 equiv), water (1 mL, 55.5 mmol, 17.60 equiv) at room temperature. The resulting mixture was stirred for 16 hr at room temperature. The reaction was quenched by the addition of water/ice (10 mL) at room temperature. The mixture/residue was acidified to pH 4 with conc. HCl. The precipitated solids were collected by filtration. This resulted in 2-methylpyrrolo[1,2-b]pyridazine-6-carboxylic acid (335 mg, 60.3%) as a light brown solid.

LC-MS (ES, m/z): [M+1]⁺=177.

Acid 34: 1-(pyridin-4-yl) indazole-5-carboxylic acid

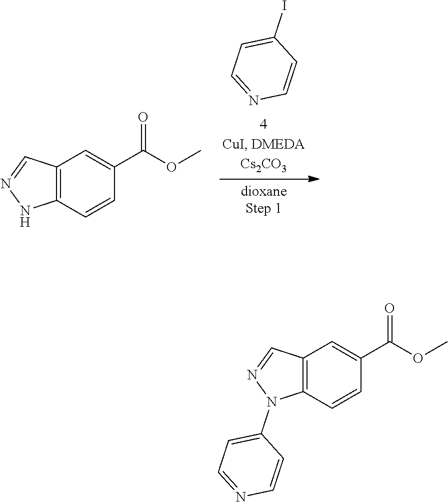 [SMILES: Ic1ccncc1 ; COC(=O)c1ccc2[nH]ncc2c1 ; COC(=O)c1ccc2c(cnn2-c2ccncc2)c1]

Into a 250-mL 3-necked round-bottom flask purged and maintained with an inert atmosphere of nitrogen, was placed methyl 1H-indazole-5-carboxylate (2.0 g, 11.35 mmol, 1.00 equiv), dioxane (40.00 mL), 4-iodopyridine (2.33 g, 11.366 mmol, 1.00 equiv), CuI (2.16 g, 11.342 mmol, 1.00 equiv), DMEDA (dimethylethylene diamine, 0.20 g, 2.269 mmol, 0.20 equiv), Cs₂CO₃ (11.10 g, 34.068 mmol, 3.00 equiv). The resulting solution was stirred for 2 days at 100° C. in an oil bath. The resulting mixture was concentrated under vacuum. The resulting solution was diluted with 40 mL of H₂O. The resulting solution was extracted with 3×20 mL of ethyl acetate and the organic layers combined. The resulting mixture was washed with 2×30 mL of brine. The mixture was dried over anhydrous sodium sulfate and concentrated under vacuum. This resulted in 1.6 g (37%) of methyl 1-(pyridin-4-yl) indazole-5-carboxylate as an off-white solid.

LC-MS: (ES, m/z): [M+H]⁺=254.

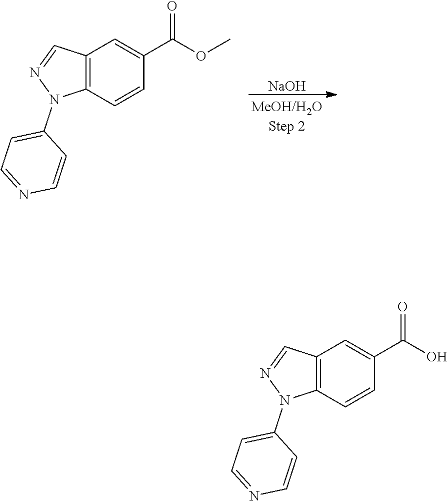 [SMILES: COC(=O)c1ccc2c(cnn2-c2ccncc2)c1 ; "NaOH" ; O=C(O)c1ccc2c(cnn2-c2ccncc2)c1]

Into a 100-mL vial, was placed methyl 1-(pyridin-4-yl) indazole-5-carboxylate (1.60 g, 6.318 mmol, 1.00 equiv), CH₃OH (32.00 mL), H₂O (10.00 mL), sodium hydroxide (1.26 g, 31.590 mmol, 5.00 equiv). The resulting solution was stirred for 16 hr at room temperature. The resulting mixture was concentrated under vacuum. The resulting solution was diluted with 30 mL of H₂O. The resulting solution was extracted with 2×30 mL of ethyl acetate and the aqueous layers combined. The pH value of the solution was adjusted to 3 with HCl (3 mol/L). The solids were collected by filtration. This resulted in 350 mg (23%) of 1-(pyridin-4-yl) indazole-5-carboxylic acid as an off-white solid.

LC-MS: (ES, m/z): [M+H]⁺=240.

Acid 35: 3-cyclopropyl-1-methylindazole-6-carboxylic acid

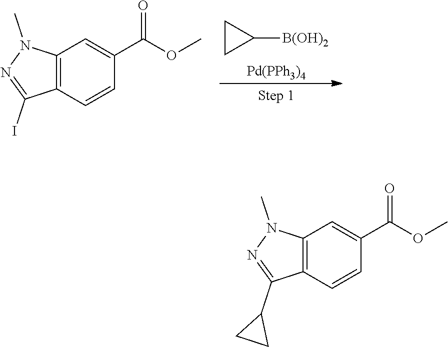

Into a 50-mL round-bottom flask purged and maintained with an inert atmosphere of nitrogen, was placed methyl 3-iodo-1-methylindazole-6-carboxylate (1.00 g, 3.16 mmol, 1.00 equiv), cyclopropylboronic acid (815 mg, 9.49 mmol, 3.00 equiv), Pd(PPh₃)₄ (365 mg, 0.316 mmol, 0.10 equiv), toluene (20.00 mL), H₂O (2.00 mL), K₃PO₄ (2.69 g, 12.656 mmol, 4.00 equiv). The resulting solution was stirred for 16 hr at 110° C. in an oil bath. The solids were filtered out. The resulting mixture was concentrated. The residue was applied onto a silica gel column with ethyl acetate/petroleum ether (1:3). This resulted in 590 mg (81%) of methyl 3-cyclopropyl-1-methylindazole-6-carboxylate as a yellow solid.

LC-MS (ES, m/z): [M+H]⁺=231.

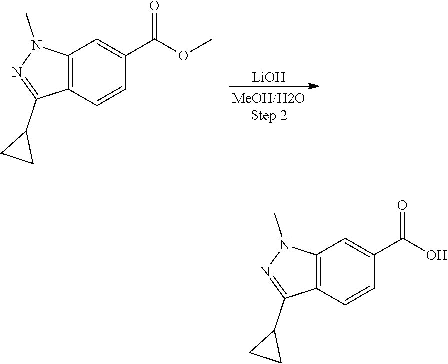 [SMILES: COC(=O)c1ccc2c(C3CC3)nn(C)c2c1 ; "LiOH" ; Cn1nc(C2CC2)c2ccc(C(=O)O)cc21]

Into a 50-mL round-bottom flask, was placed methyl 3-cyclopropyl-1-methylindazole-6-carboxylate (590.00 mg, 2.56 mmol, 1.00 equiv), LiOH (184.08 mg, 3.00 equiv), MeOH (10.00 mL), H₂O (2.00 mL). The resulting solution was stirred for 6 hr at room temperature. The resulting mixture was concentrated. The pH value of the solution was adjusted to 2˜3 with citric acid (aq). The solids were collected by filtration. This resulted in 450 mg of 3-cyclopropyl-1-methylindazole-6-carboxylic acid as a white solid.

LC-MS (ES, m/z): [M+H]⁺=217.

Acid 36: 3-cyclopropyl-1,2-benzoxazole-5-carboxylic acid

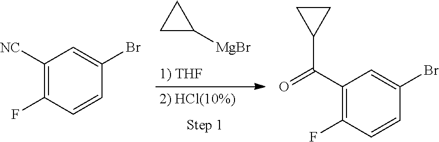

Into a 500-mL round-bottom flask purged and maintained with an inert atmosphere of nitrogen, was placed 5-bromo-2-fluorobenzonitrile (5.5 g, 27.499 mmol, 1.00 equiv), tetrahydrofuran (110.00 mL). Cyclopropylmagnesium bromide (1 mol/L in THF) (68.75 mL, 68.748 mmol, 2.50 equiv) was added and the resulting solution was stirred for 2 hr at −78° C. The resulting solution was allowed to react, with stirring, for an additional 30 min at 25° C. The reaction was then quenched by the addition of 100 mL of HCl (10%) and was stirred for an additional 6 hr. The resulting solution was extracted with 3×50 mL of ethyl acetate and the aqueous layers combined. The resulting mixture was washed with 3×50 mL of brine. The resulting mixture was concentrated. The residue was applied onto a silica gel column with ethyl acetate/hexane (1:10). This resulted in 3.3 g (49.37%) of (5-bromo-2-fluorophenyl) (cyclopropyl) methanone as an off-white solid.

LC-MS (ES, m/z): [M+1]⁺=243.

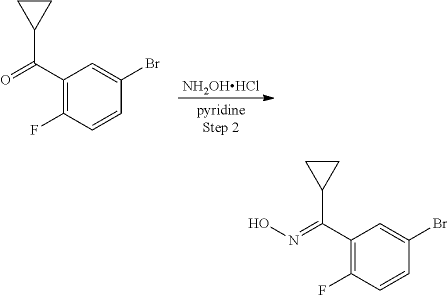

Into a 50-mL round-bottom flask purged and maintained with an inert atmosphere of nitrogen, was placed (5-bromo-2-fluorophenyl) (cyclopropyl) methanone (1.50 g, 6.17 mmol, 1.00 equiv), NH₂OH·HCl (3002 mg, 0.042 mmol, 7.00 equiv), pyridine (15.00 mL, 186.353 mmol, 30.20 equiv). The resulting solution was stirred for 3 hr at 115° C. The pH value of the solution was adjusted to 3 with HCl (1 mol/L). The resulting solution was extracted with 3×50 mL of ethyl acetate concentrated. This resulted in 1.25 g (78.5%) of (E)-N-[(5-bromo-2-fluorophenyl) (cyclopropyl)methylidene]hydroxylamine as an off-white solid.

LC-MS (ES, m/z): [M+1]⁺=258.

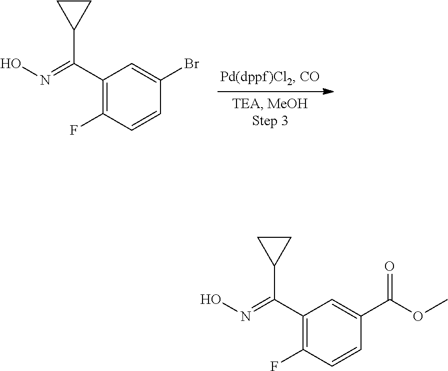 [SMILES: O/N=C(/c1cc(Br)ccc1F)C1CC1 ; COC(=O)c1ccc(F)c(/C(=N/O)C2CC2)c1]

Into a 50-mL pressure tank reactor, was placed (E)-N-[(5-bromo-2-fluorophenyl) (cyclopropyl)methylidene]hydroxylamine (1.25 g, 4.84 mmol, 1.00 equiv), TEA (1470. mg, 14.53 mmol, 3.00 equiv), Pd(dppf)Cl₂ (354 mg, 0.484 mmol, 0.10 equiv), MeOH (20.00 mL), CO (20 atm). The resulting solution was stirred for 12 hr at 80° C. The resulting mixture was concentrated. The residue was applied onto a silica gel column with ethyl acetate/hexane (1:2). This resulted in 850 mg (73.98%) of methyl 3-[(1E)-cyclopropyl(hydroxyimino)methyl]-4-fluorobenzoate as a brown solid.

LC-MS (ES, m/z): [M+1]⁺=238.

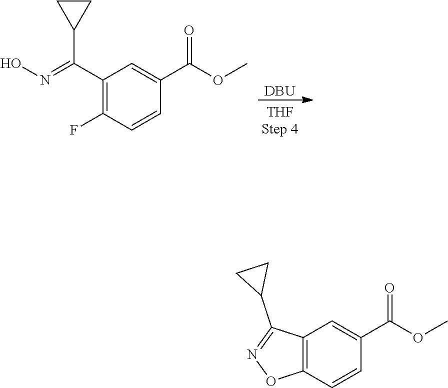

Into a 20-mL vial purged and maintained with an inert atmosphere of nitrogen, was placed methyl 3-[(1E)-cyclopropyl(hydroxyimino)methyl]-4-fluorobenzoate (450.00 mg, 1.897 mmol, 1.00 equiv), tetrahydrofuran (8 mL), DBU (1433 mg, 5.691 mmol, 3.00 equiv). The resulting solution was stirred for 12 hr at 75° C. The reaction was then quenched by the addition of 20 mL of aq of citric acid (5%). The resulting solution was extracted with 3×10 mL of ethyl acetate and the organic layers combined and concentrated. The residue was applied onto a silica gel column with ethyl acetate/hexane (1:2). This resulted in 140 mg (34%) of methyl 3-cyclopropyl-1,2-benzoxazole-5-carboxylate as an off-white solid.

LC-MS (ES, m/z): [M+1]⁺=218.

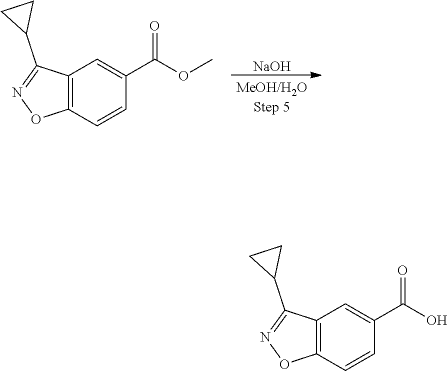

Into a 20-mL vial purged and maintained with an inert atmosphere of nitrogen, was placed methyl 3-cyclopropyl-1,2-benzoxazole-5-carboxylate (140 mg, 0.644 mmol, 1.00 equiv), H₂O (1.00 mL), methanol (4.00 mL), sodium hydroxide (51.6 mg, 1.3 mmol, 2 equiv). The resulting solution was stirred for 2 hr at 25° C. The pH value of the solution was adjusted to 3 with HCl (37%). The resulting mixture was concentrated. This resulted in 220 mg (crude) of 3-cyclopropyl-1,2-benzoxazole-5-carboxylic acid as an off-white solid.

LC-MS (ES, m/z): [M+1]⁺=204.

Acid 37: 1-methyl-2-oxo-3H-1,3-benzodiazole-5-carboxylic acid

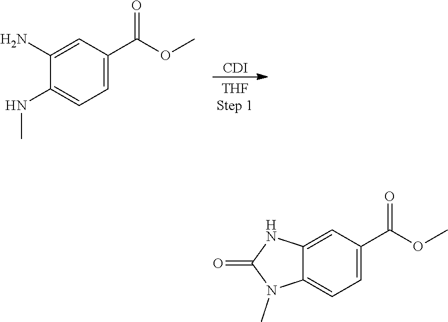

Into a 50 mL round-bottom flask were added methyl 3-amino-4-(methylamino)benzoate (600.00 mg, 3.330 mmol, 1.00 equiv), THF (20.00 mL) and CDI (1620 mg, 10 mmol, 3 equiv) at room temperature. The resulting mixture was stirred for overnight at room temperature under nitrogen atmosphere. The reaction was quenched by the addition of water (5 mL) at room temperature. The precipitated solids were collected by filtration and washed with water (3×10 mL). The resulting solid was dried under infrared light. This resulted in methyl 1-methyl-2-oxo-3H-1,3-benzodiazole-5-carboxylate (400 mg, 58.3%) as a grey solid.

LC-MS− (ES, m/z): [M+1]⁺=207

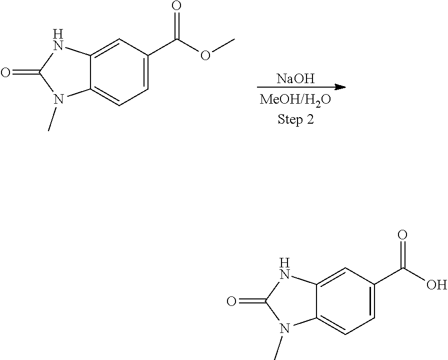

Into a 100 mL round-bottom flask were added methyl 1-methyl-2-oxo-3H-1,3-benzodiazole-5-carboxylate (700.00 mg, 3.4 mmol, 1.00 equiv), MeOH (20.00 mL), water (5.00 mL) and NaOH (543.12 mg, 13.579 mmol, 4 equiv) at room temperature. The resulting mixture was stirred for overnight at room temperature. The resulting mixture was concentrated under reduced pressure. The residue was dissolved in water (50 mL). The aqueous layer was extracted with EtOAc (3×10 mL). The aqueous phase was acidified to pH 5 with HCl (aq. 1M). The precipitated solids were collected by filtration and washed with water (2×10 mL). The resulting solid was dried under infrared light. This resulted in 1-methyl-2-oxo-3H-1,3-benzodiazole-5-carboxylic acid (500 mg, 76.6%) as a grey solid.

LC-MS (ES, m/z): [M+1]⁺=193.

Acid 38: lithio 3-methyl-1-(oxan-2-yl) indazole-5-carboxylate

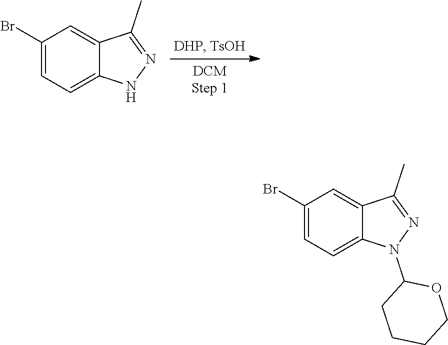 [SMILES: Cc1n[nH]c2ccc(Br)cc12 ; Cc1nn(C2CCCCO2)c2ccc(Br)cc12]

Into a 100-mL 3-necked round-bottom flask, was placed 5-bromo-3-methyl-1H-indazole (2.00 g, 9.48 mmol, 1.00 equiv), DHP (1.20 g, 14.214 mmol, 1.50 equiv), DCM (20.00 mL), TsOH (163.18 mg, 0.948 mmol, 0.10 equiv). The resulting solution was stirred for 5 hr at room temperature. The reaction was then quenched by the addition of 50 mL of water. The resulting solution was extracted with 2×50 mL of ethyl acetate and the organic layers combined and concentrated. The residue was applied onto a silica gel column with ethyl acetate/petroleum ether (1:3). This resulted in 1.6 g (57.2%) of 5-bromo-3-methyl-1-(oxan-2-yl) indazole as a white solid.

LC-MS (ES, m/z): [M+H]⁺=295.

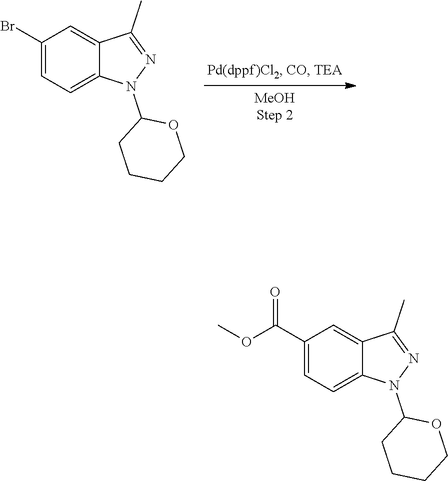 [SMILES: Cc1nn(C2CCCCO2)c2ccc(Br)cc12 ; COC(=O)c1ccc2c(c1)c(C)nn2C1CCCCO1]

Into a 100-mL pressure reactor, was placed 5-bromo-3-methyl-1-(oxan-2-yl) indazole (1.60 g, 5.420 mmol, 1.00 equiv), TEA (1.65 g, 16.260 mmol, 3.00 equiv), Pd(dppf)Cl₂ (793.22 mg, 1.084 mmol, 0.20 equiv), MeOH (20.00 mL). The flask was evacuated and flushed three times with nitrogen, followed by flushing with CO (gas). The mixture was stirred 6 hr at 60° C. under an atmosphere of CO (3 MPa). The resulting mixture was concentrated. The residue was applied onto a silica gel column with ethyl acetate/petroleum ether (1:2). This resulted in 1.25 g (84%) of methyl 3-methyl-1-(oxan-2-yl) indazole-5-carboxylate as a white solid.

LC-MS (ES, m/z): [M+H]⁺=275.

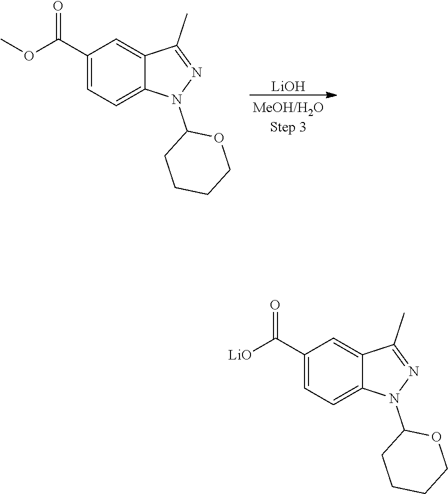

Into a 50-mL round-bottom flask, was placed methyl 3-methyl-1-(oxan-2-yl) indazole-5-carboxylate (600.0 mg, 2.19 mmol, 1.00 equiv), H₂O (2.0 mL), MeOH (10.0 mL), LiOH (157.1 mg, 6.56 mmol, 3.00 equiv). The resulting solution was stirred for 12 hr at 40° C. The resulting mixture was concentrated. This resulted in 510 mg (87.58%) of lithio 3-methyl-1-(oxan-2-yl) indazole-5-carboxylate as a white solid.

LC-MS (ES, m/z): [M+2H−Li]=275.

Acid 39: 1,3-dimethylindazole-6-carboxylic acid

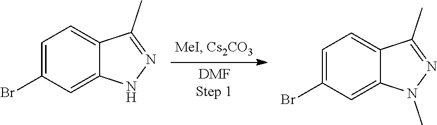

Into a 100 mL 3-necked round-bottom flask were added 6-bromo-3-methyl-1H-indazole (2.90 g, 13.74 mmol, 1.00 equiv) and DMF (60 mL), Cs₂CO₃ (8.95 g, 27.48 mmol, 2.0 equiv) at room temperature. To the above mixture was added Mel (2.34 g, 16.49 mmol, 1.2 equiv) dropwise at room temperature. The resulting mixture was stirred for additional 2 hr at room temperature. The resulting mixture was diluted with EtOAc (200 mL) and the resulting mixture was washed with 3×100 mL of brine. The resulting mixture was dried over anhydrous Na₂SO₄. After filtration, the filtrate was concentrated under reduced pressure. The residue was purified by silica gel column chromatography, eluted with PE/EtOAc (3:1) to afford 6-bromo-1,3-dimethylindazole (2.25 g, 72.75%) as a white solid, and 6-bromo-2,3-dimethylindazole (0.8 g, 26%) as a white solid.

¹H-NMR: (300 MHZ, Methanol-d₄ ppm) δ 7.72 (m, 1H), 7.60 (dd, J=8.7, 1.7 Hz, 1H), 7.23 (dd, J=8.6, 1.7 Hz, 1H), 3.95 (d, J=1.1 Hz, 3H), 2.52 (s, 3H).

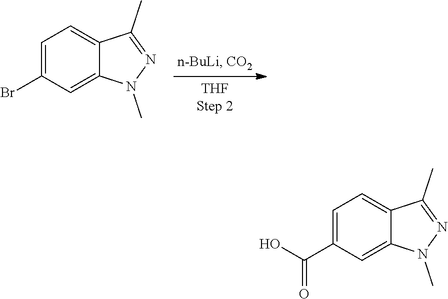

Into a 250-mL 3-necked round-bottom flask purged and maintained with an inert atmosphere of nitrogen, was placed 6-bromo-1,3-dimethylindazole under Ar at −78° C., n-BuLi (5.86 mL, 2 M soln, 1.5 equiv) was added dropwise. The resulting solution was stirred for 30 min at −78° C., followed by the slow addition of the dry ice (20.0 g). The resulting solution was stirred for 30 min at −78° C. The resulting solution was allowed to react, with stirring, for an additional 2 hr at −78° C. The reaction was then quenched by the addition of 100 ml of water/ice. The resulting solution was extracted with 2×30 mL of ethyl acetate and the aqueous layers combined. HCl (3 mol/L) was employed to adjust the pH to 3. The resulting solution was extracted with 3×30 mL of ethyl acetate dried over anhydrous sodium sulfate and concentrated under vacuum. This resulted in 1.4 g (75.3%) of 1,3-dimethylindazole-6-carboxylic acid as a white solid.

LC-MS: (ES, m/z): [M+H]⁺=191.

Acid 40: 1-[(tert-butoxycarbonyl)amino]isoquinoline-6-carboxylic acid

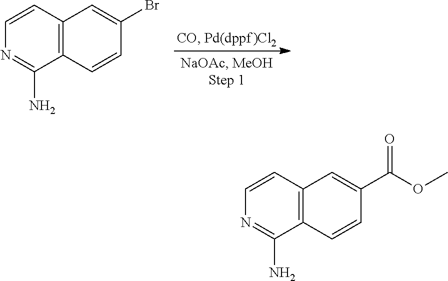

Into a 50-mL pressure tank reactor purged and maintained with an inert atmosphere of nitrogen, was placed 6-bromoisoquinolin-1-amine (1.20 g, 5.38 mmol, 1.00 equiv), CH₃OH (24.00 mL), Pd(dppf)Cl₂ (0.39 g, mmol, 0.53 mmol, 0.10 equiv), NaOAc (1.77 g, 21.58 mmol, 4.01 equiv), CO (10 atm). The resulting solution was stirred for 16 hr at 80° C. in an oil bath. The resulting mixture was concentrated under vacuum. The resulting solution was diluted with 30 mL of H₂O. The resulting solution was extracted with 3×30 mL of ethyl acetate and the organic layers combined. The resulting mixture was washed with 2×30 mL of brine. The mixture was dried over anhydrous sodium sulfate and concentrated under vacuum. The residue was applied onto a silica gel column with ethyl acetate/petroleum ether (1:1). This resulted in 0.9 g (82.7%) of methyl 1-aminoisoquinoline-6-carboxylate as a light yellow solid.

LC-MS: (ES, m/z): [M+H]⁺=203.

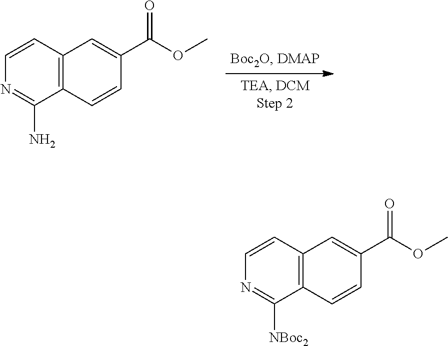

Into a 100-mL 3-necked round-bottom flask, was placed methyl 1-aminoisoquinoline-6-carboxylate (0.90 g, 4.45 mmol, 1.00 equiv), DCM (18.00 mL), BoczO (2.43 g, 11.13 mmol, 2.50 equiv), TEA (1.80 g, 17.80 mmol, 4.00 equiv), DMAP (0.05 g, 0.45 mmol, 0.10 equiv). The resulting solution was stirred for 10 hr at room temperature. The resulting solution was diluted with 30 mL of H₂O. The resulting solution was extracted with 3×30 mL of dichloromethane and the organic layers combined and dried over anhydrous sodium sulfate and concentrated under vacuum. The residue was applied onto a silica gel column with ethyl acetate/petroleum ether (1:3). This resulted in 1 g (55.8%) of methyl 1-[bis(tert-butoxycarbonyl)amino]isoquinoline-6-carboxylate as brown oil.

LC-MS: (ES, m/z): [M+H]⁺=403.

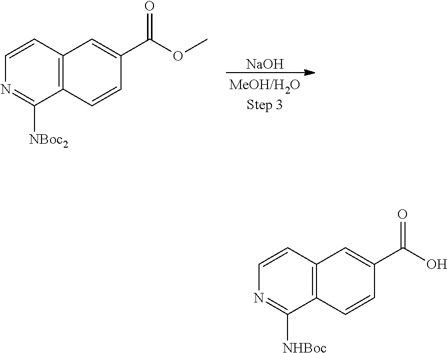

Into a 100-mL round-bottom flask, was placed methyl 1-[bis(tert-butoxycarbonyl)amino]isoquinoline-6-carboxylate (1.00 g, 2.49 mmol, 1.00 equiv), CH₃OH (20.00 mL), H₂O (7.00 mL), NaOH (0.30 g, 7.501 mmol, 3.02 equiv). The resulting solution was stirred for 16 hr at room temperature. The resulting solution was diluted with 20 mL of H₂O. The resulting mixture was concentrated under vacuum. The pH value of the solution was adjusted to 3 with HCl (3 mol/L). The resulting mixture was concentrated under vacuum. This resulted in 1 g of 1-[(tert-butoxycarbonyl)amino]isoquinoline-6-carboxylic acid as a light yellow solid.

LC-MS: (ES, m/z): [M+H]⁺=289.

Acid 41: 3-methylimidazo[1,5-a]pyridine-7-carboxylic acid

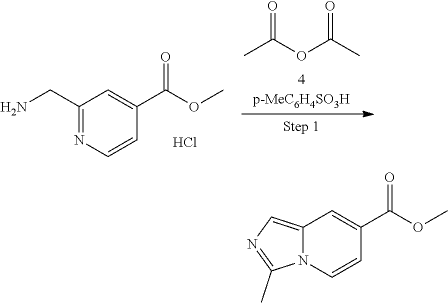

Into a 100-mL 3-necked round-bottom flask purged and maintained with an inert atmosphere of nitrogen, was placed methyl 2-(aminomethyl)pyridine-4-carboxylate hydrochloride (1.00 g, 4.9 mmol, 1.00 equiv), acetic anhydride (20.00 mL), p-toluenesulfonic acid (0.85 g, 4.936 mmol, 1.00 equiv). The resulting solution was stirred for 3 hr at 100° C. in an oil bath. The reaction mixture was cooled. The resulting solution was diluted with 40 mL of NH₃/H₂O (10%). The solids were collected by filtration. This resulted in 500 mg (53.3%) of methyl 3-methylimidazo[1,5-a]pyridine-7-carboxylate as a yellow solid.

LC-MS: (ES, m/z): [M+H]⁺=191.

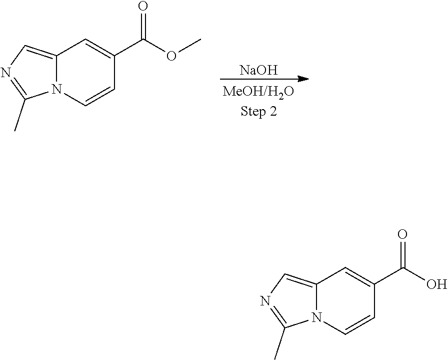

Into a 50-mL round-bottom flask, was placed methyl 3-methylimidazo[1,5-a]pyridine-7-carboxylate (500.00 mg, 2.629 mmol, 1.00 equiv), CH₃OH (10.00 mL), H₂O (3.00 mL), sodium hydroxide (315.43 mg, 7.886 mmol, 3.00 equiv). The resulting solution was stirred for 16 hr at room temperature. The resulting mixture was concentrated under vacuum. The pH value of the solution was adjusted to 3 with HCl (3M). This resulted in 300 mg (64.8%) of 3-methylimidazo[1,5-a]pyridine-7-carboxylic acid as a yellow solid.

LC-MS: (ES, m/z): [M+H]⁺=177.

Acid 42: 3-[(tert-butoxycarbonyl)(methyl)amino]-1-methylindazole-6-carboxylic acid

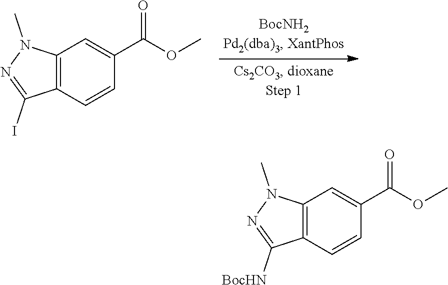 [SMILES: COC(=O)c1ccc2c(I)nn(C)c2c1 ; COC(=O)c1ccc2c(NC(=O)OC(C)(C)C)nn(C)c2c1]

Into a 50-mL round-bottom flask purged and maintained with an inert atmosphere of nitrogen, was placed methyl 3-iodo-1-methylindazole-6-carboxylate (1.00 g, 3.164 mmol, 1.00 equiv), tert-butyl carbamate (1112 mg, 0.000 mmol, 3.00 equiv), Pd₂(dba)₃ (289.69 mg, 0.316 mmol, 0.10 equiv), Xantphos (366.10 mg, 0.633 mmol, 0.20 equiv), Cs₂CO₃ (4123 mg, 12.656 mmol, 4.00 equiv), dioxane (20.00 mL). The resulting solution was stirred for 20 hr at 100° C. in an oil bath. The solids were filtered out. The resulting mixture was concentrated. The residue was applied onto a silica gel column with ethyl acetate/petroleum ether (1:2). This resulted in 850 mg of methyl 3-[(tert-butoxycarbonyl)amino]-1-methylindazole-6-carboxylate as a brown solid.

LC-MS (ES, m/z): [M+H]⁺=306.

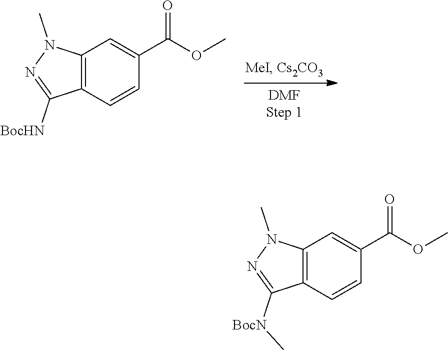 [SMILES: COC(=O)c1ccc2c(NC(=O)OC(C)(C)C)nn(C)c2c1 ; COC(=O)c1ccc2c(N(C)C(=O)OC(C)(C)C)nn(C)c2c1]

Into a 8-mL round-bottom flask purged and maintained with an inert atmosphere of nitrogen, was placed methyl 3-[(tert-butoxycarbonyl)amino]-1-methylindazole-6-carboxylate (200.00 mg, 0.66 mmol, 1.00 equiv), CH₃I (139.5 mg, 0.983 mmol, 1.50 equiv), Cs₂CO₃ (534 mg, 1.64 mmol, 2.50 equiv), DMF (3.00 mL). The resulting solution was stirred for 1 overnight at 25° C. The reaction was then quenched by the addition of 10 mL of ice/salt. The resulting solution was extracted with 3×5 mL of ethyl acetate and the organic layers combined and concentrated. The residue was applied onto a silica gel column with ethyl acetate/hexane (1:3). This resulted in 120 mg (58%) of methyl 3-[(tert-butoxycarbonyl)(methyl)amino]-1-methylindazole-6-carboxylate as a brown solid.

LC-MS (ES, m/z): [M+1]⁺=320.

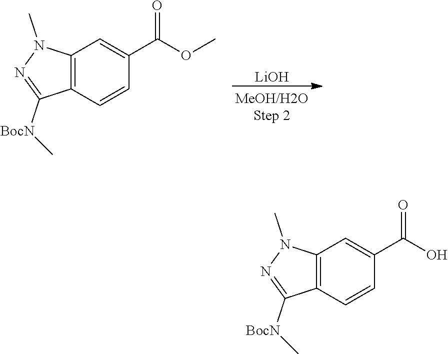 [SMILES: COC(=O)c1ccc2c(N(C)C(=O)OC(C)(C)C)nn(C)c2c1 ; "LiOH" ; CN(C(=O)OC(C)(C)C)c1nn(C)c2cc(C(=O)O)ccc12]

Into a 8-mL vial purged and maintained with an inert atmosphere of nitrogen, was placed methyl 3-[(tert-butoxycarbonyl)(methyl)amino]-1-methylindazole-6-carboxylate (120.00 mg, 0.376 mmol, 1.00 equiv), lithium hydroxide (18.00 mg, 0.75 mmol, 2.00 equiv), MeOH (1.20 mL), H₂O (0.30 mL). The resulting solution was stirred for 12 hr at 25° C. The resulting mixture was concentrated. This resulted in 110 mg (96%) of 3-[(tert-butoxycarbonyl)(methyl)amino]-1-methylindazole-6-carboxylic acid as an off-white solid.

LC-MS (ES, m/z): [M+1]⁺=306

Acid 43: 1-methylimidazo[1,5-a]pyridine-7-carboxylic acid

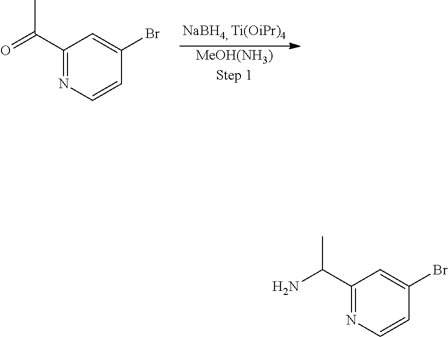

Into a 50 mL 3-necked round-bottom flask were added MeOH (14 mL, 345.785 mmol, 69.17 equiv), NH₃ (g) in MeOH (7 mL, 246.616 mmol, 49.33 equiv) and titanium isopropoxide (2.84 g, 9.998 mmol, 2.0 equiv) at room temperature. The resulting mixture was stirred for 0.5 hr at room temperature under nitrogen atmosphere. To a stirred solution/mixture was added NaBH₄ (0.28 g, 7.498 mmol, 1.5 equiv) in portions at room temperature under nitrogen atmosphere. The resulting mixture was stirred for 16 hr at room temperature under nitrogen atmosphere. The resulting mixture was concentrated under reduced pressure. The residue was purified by silica gel column chromatography, eluted with PE/THF (1:8) to afford 1-(4-bromopyridin-2-yl) ethanamine (700 mg, 69.64%) as a light brown solid.

LC-MS (ES, m/z): [M+1]⁺=201.

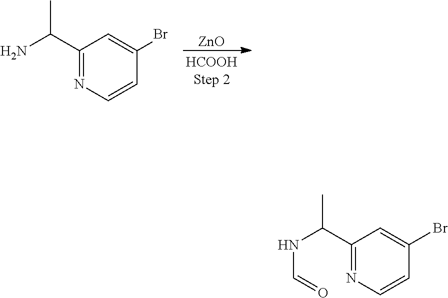

Into a 8-mL vial purged and maintained with an inert atmosphere of nitrogen, was placed 1-(4-bromopyridin-2-yl) ethanamine (700 mg, 3.481 mmol, 1.00 equiv), ZnO (311.76 mg, 3.829 mmol, 1.1 equiv), HCOOH (2.2 mL, 58.316 mmol, 16.75 equiv) The resulting solution was stirred for 8 hr at 70° C. The resulting solution was diluted with 10 mL of DCM. The solids were filtered out. The resulting mixture was concentrated. The residue was applied onto a silica gel column with DCM/MeOH (98:2). This resulted in N-[1-(4-bromopyridin-2-yl)ethyl]formamide (400 mg, 50%) as a colorless oil.

LC-MS (ES, m/z): [M+1]⁺=229.

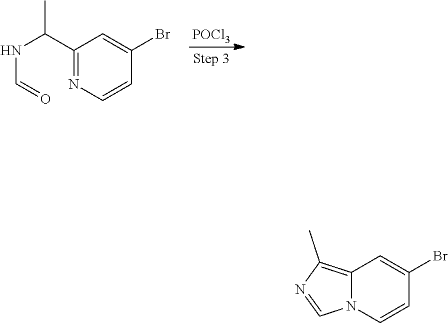

Into a 8 mL vial were added N-[1-(4-bromopyridin-2-yl)ethyl]formamide (400.00 mg, 1.746 mmol, 1.00 equiv) and POCl₃ (3.00 mL) at room temperature. The resulting mixture was stirred for 1 h at 80° C. under nitrogen atmosphere. The resulting mixture was concentrated under reduced pressure. The resulting mixture was diluted with CH₂Cl₂ (30 mL). The reaction was quenched with sat. NaHCO₃ (aq.) at room temperature. The resulting mixture was separated and the aqueous layer was extracted with CH₂Cl₂ (2× 10 mL). The combined organic layers were dried over anhydrous Na₂SO₄. After filtration, the filtrate was concentrated under reduced pressure. This resulted in 7-bromo-1-methylimidazo[1,5-a]pyridine (340 mg, 82%) as a brown solid.

LC-MS (ES, m/z): [M+1]⁺=211.

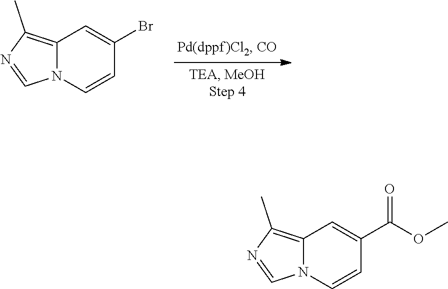

Into a 30-mL sealed tube, was placed 7-bromo-1-methylimidazo[1,5-a]pyridine (350 mg, 1.66 mmol, 1.00 equiv), Pd(dppf)Cl₂ (145.60 mg, 0.199 mmol, 0.12 equiv), TEA (503.40 mg, 4.974 mmol, 3 equiv), MeOH (10.00 mL), CO (5 atm). The resulting solution was stirred for 3 hr at 120° C. The resulting mixture was concentrated. The residue was applied onto a silica gel column with ethyl acetate/petroleum ether (1:3). This resulted in methyl 1-methylimidazo[1,5-a]pyridine-7-carboxylate (300 mg, 95%) as a brown solid.

LC-MS− (ES, m/z): [M+1]⁺=191.

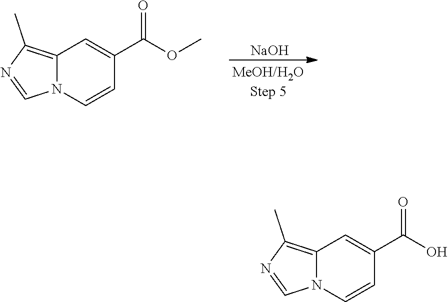

Into a 20 mL vial were added methyl 1-methylimidazo[1,5-a]pyridine-7-carboxylate (300.00 mg, 1.577 mmol, 1.00 equiv), MeOH (5.00 mL), NaOH (252.00 mg, 6.300 mmol, 3.99 equiv) and H₂O (5.00 mL) at room temperature. The resulting mixture was stirred for overnight at room temperature. The resulting mixture was concentrated under vacuum. The residue was dissolved in water (10 mL). The mixture was acidified to pH 4 with HCl (aq.). The precipitated solids were collected by filtration and washed with water (3×10 mL). The resulting solid was dried under infrared light. This resulted in 1-methylimidazo[1,5-a]pyridine-7-carboxylic acid (200 mg, 72%) as a grey solid.

LC-MS− (ES, m/z): [M+1]⁺=177.

Acid 44: 1,3-dimethylimidazo[1,5-a]pyridine-6-carboxylic acid

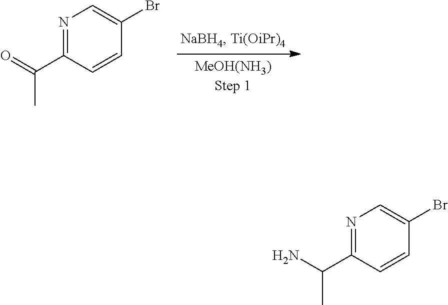

Into a 250-mL 3-necked round-bottom flask purged and maintained with an inert atmosphere of nitrogen, was placed 1-(5-bromopyridin-2-yl) ethanone (5.70 g, 28.495 mmol, 1.00 equiv), MeOH (80.00 mL), Ti(Oi-Pr)₄ (16.20 g, 56.990 mmol, 2.00 equiv). To the above NH₃ (g) in MeOH (40.00 mL) was introduced in at 25° C. The resulting solution was stirred for 1 hr at room temperature. This was followed by the addition of NaBH₄ (1617.08 mg, 42.743 mmol, 1.50 equiv) at 25° C. The resulting solution was allowed to react, with stirring, for an additional 2 hr at room temperature. The resulting mixture was concentrated. The residue was applied onto a silica gel column with THF. This resulted in 4 g (69.8%) of 1-(5-bromopyridin-2-yl) ethanamine as light yellow oil.

LC-MS (ES, m/z): [M+1]⁺=201.

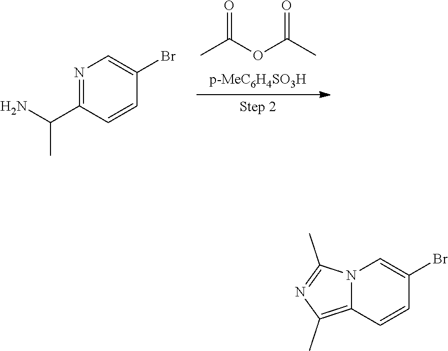

Into a 50-mL 3-necked round-bottom flask purged and maintained with an inert atmosphere of nitrogen, was placed 1-(5-bromopyridin-2-yl) ethanamine (650.0 mg, 3.23 mmol, 1.00 equiv), acetic anhydride (14 mL), p-MeC₆H₄SO₃H (556.7 mg, 3.233 mmol, 1.00 equiv). The resulting solution was stirred for 3 hr at 100° C. The resulting mixture was concentrated. The residue was applied onto a silica gel column with THF/PE (2:1). This resulted in 500 mg (68.7%) of 6-bromo-1,3-dimethylimidazo[1,5-a]pyridine as a light yellow solid.

LC-MS− (ES, m/z): [M+1]⁺=225.

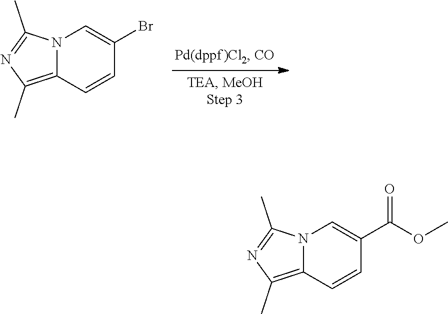

Into a 50-mL sealed tube, was placed 6-bromo-1,3-dimethylimidazo[1,5-a]pyridine (500 mg, 2.221 mmol, 1.00 equiv), Pd(dppf)Cl₂ (325 mg, 0.444 mmol, 0.2 equiv), TEA (674 mg, 6.664 mmol, 3.0 equiv), MeOH (20.00 mL), CO (20 atm). The resulting solution was stirred for 3 hr at 120° C. The resulting mixture was concentrated. The residue was applied onto a silica gel column with THF/PE (2:1). This resulted in 420 mg (92.3%) of methyl 1,3-dimethylimidazo[1,5-a]pyridine-6-carboxylate as a brown solid.

LC-MS (ES, m/z): [M+1]⁺=205.

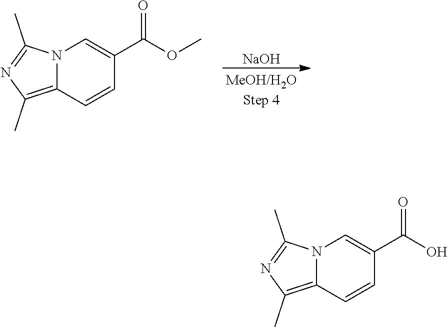

Into an 8-mL vial purged and maintained with an inert atmosphere of nitrogen, was placed methyl 1,3-dimethylimidazo[1,5-a]pyridine-6-carboxylate (420 mg, 2.057 mmol, 1.00 equiv), MeOH (4.00 mL), H₂O (1.00 mL), sodium hydroxide (164.5 mg, 0.000 mmol, 2.00 equiv). The resulting solution was stirred for 16 hr at room temperature. The pH value of the solution was adjusted to 4 with HCl (37%). The resulting mixture was concentrated. This resulted in 600 mg (crude) of 1,3-dimethylimidazo[1,5-a]pyridine-6-carboxylic acid as a brown solid.

LC-MS− (ES, m/z): [M+1]⁺=191.

Acid 45: 3,7-difluoro-1H-indazole-6-carboxylic acid

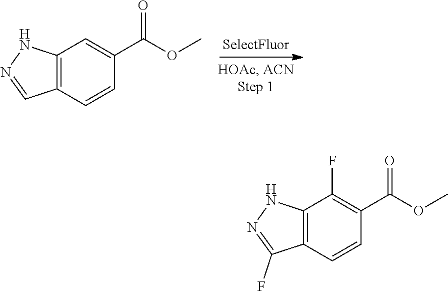

Into a 40-mL vial purged and maintained with an inert atmosphere of nitrogen, was placed methyl 1H-indazole-6-carboxylate (1.4 g, 7.947 mmol, 1.00 equiv), ACN (25 mL), HOAc (2.5 mL), and SelectFluor (8.45 g, 23.841 mmol, 3 equiv). The resulting solution was stirred for 2 hr at 85° C. in an oil bath. The resulting mixture was concentrated under vacuum. The resulting solution was diluted with 20 mL of H₂O. The resulting solution was extracted with 3×20 mL of ethyl acetate and the organic layers combined. The resulting mixture was washed with 2×20 mL of brine. The mixture was dried over anhydrous sodium sulfate and concentrated under vacuum. The residue was applied onto a silica gel column with ethyl acetate/petroleum ether (1/5). This resulted in methyl 3,7-difluoro-1H-indazole-6-carboxylate (110 mg, 6.5%) as a yellow solid.

LC-MS: (ES, m/z): [M−H]+=211

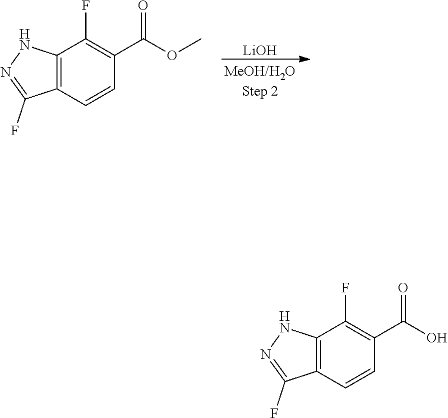

Into a 50-mL round-bottom flask, was placed methyl methyl 3,7-difluoro-1H-indazole-6-carboxylate (110 mg, 0.518 mmol, 1.00 equiv), MeOH (3 mL), H₂O (1 mL), lithium hydroxide (37.25 mg, 1.554 mmol, 3 equiv). The resulting solution was stirred for 16 hr at room temperature. The resulting mixture was concentrated under vacuum. The resulting solution was diluted with 50 mL of H₂O. The resulting solution was extracted with 2×20 mL of ethyl acetate and the aqueous layers combined. The pH value of the solution was adjusted to 3 with HCl (3 mol/L). The solids were collected by filtration. This resulted in 3-fluoro-1-methylindazole-6-carboxylic acid (150 mg, 80.4%) 3,7-difluoro-1H-indazole-6-carboxylic acid (50 mg, 48.7%) as a yellow solid.

LC-MS: (ES, m/z): [M−H]⁺=197.

Acid 46: 8-fluoro-3-methylimidazo[1,5-a]pyridine-7-carboxylic acid

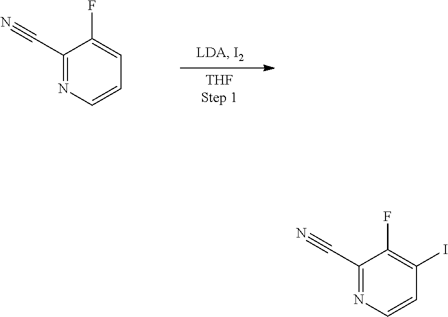 [SMILES: N#Cc1ncccc1F ; N#Cc1nccc(I)c1F]

In a 500-mL 3 necked round bottom flask, to a solution of diisopropylamine (6.22 g, 61.423 mmol, 1.5 equiv) in THF (50 mL) was added dropwise n-butyllithium solution (2.5 M in hexane, 19.6 mL) at −78° C. under N₂ atmosphere. The reaction mixture was stirred at −78° C. for 10 mins. Then a solution of 3-fluoropyridine-2-carbonitrile (5 g, 40.949 mmol, 1.00 equiv) was added dropwise and the mixture was stirred for another 20 mins. Then a solution of I₂ (11.43 g, 45.044 mmol, 1.1 equiv) in 50 mL THF was added dropwise and the mixture was stirred for another 20 mins. The reaction was quenched with sat. NH₄Cl (100 mL), and then the mixture was extracted with EtOAc (2×50 mL). The combined organic extracts were washed with brine (100 mL), dried over anhydrous Na₂SO₄, and concentrated under vacuum to yield a crude product which was directly purified by flash chromatography. This resulted in 3-fluoro-4-iodopyridine-2-carbonitrile (8.8 g, 86.7%) as a brown solid.

LC-MS: (ES, m/z): [M+H]=249

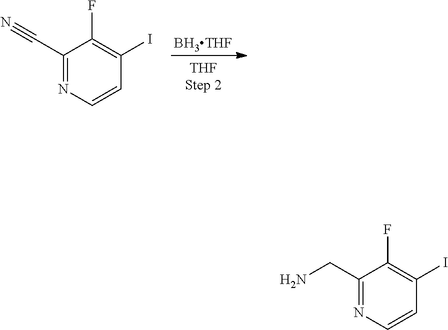 [SMILES: N#Cc1nccc(I)c1F ; NCc1nccc(I)c1F ; "BH3·THF"]

In a 500-mL 3 necked round bottom flask, to a solution of 3-fluoro-4-iodopyridine-2-carbonitrile (2.0 g, 8.065 mmol, 1.00 equiv) in THF (50 mL) was added dropwise BH₃·THF (24.19 mL, 24.195 mmol, 3.0 equiv) at room temperature under N₂ atmosphere. The reaction mixture was stirred for 16 hours at room temperature. The reaction was quenched with 1M HCl (20 mL), The mixture was basified to pH 8 with saturated NaHCO₃ (aq) and then the mixture was extracted with DCM:MeOH (4:1). The combined organic extracts were washed with brine (100 mL), dried over anhydrous Na₂SO₄, and concentrated under vacuum to yield a crude product which was directly purified by flash chromatography. This resulted in 1-(3-fluoro-4-iodopyridin-2-yl) methanamine (689 mg, 33.9%) as a brown solid.

LC-MS: (ES, m/z): [M+H]=253.

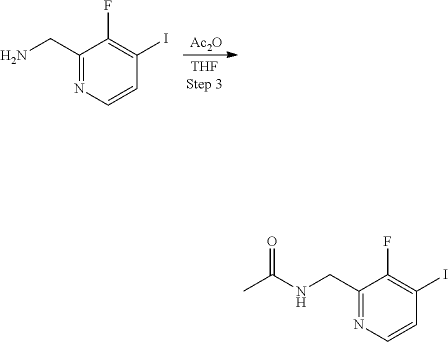

To a stirred solution/mixture of 1-(3-fluoro-4-iodopyridin-2-yl) methanamine (689 mg, 2.734 mmol, 1.00 equiv) in THF 20 mL was added Ac₂O (1395.44 mg, 13.670 mmol, 5 equiv) at room temperature. The resulting mixture was stirred for 1 hr at 70° C. The resulting mixture was concentrated under vacuum. The crude resulting mixture was used in the next step directly without further purification. This resulted in N-[(3-fluoro-4-iodopyridin-2-yl)methyl]acetamide (810 mg, crude) as a brown oil.

LC-MS: (ES, m/z): [M+H]=295.

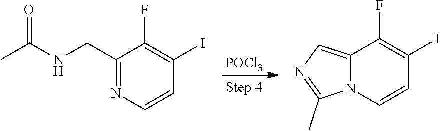

Into an 8 mL sealed tube were added N-[(3-fluoro-4-iodopyridin-2-yl)methyl]acetamide (810 mg, 1.00 equiv) and POCl₃ (5 mL) at room temperature. The resulting mixture was stirred for 16 hr at 100° C. The resulting mixture was concentrated under vacuum. The residue was basified to pH 8 with saturated NaHCO₃ (aq.). The resulting mixture was filtered. The filtrate was extracted with CH₂Cl₂ (3× 50 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (1:1) to afford 8-fluoro-7-iodo-3-methylimidazo[1,5-a]pyridine (370 mg, 17% for two steps) as a brown solid.

LC-MS: (ES, m/z): [M+H]=277.

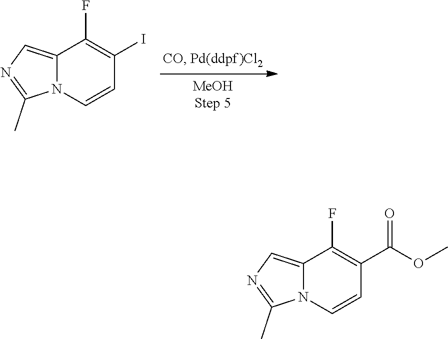

To a solution of 8-fluoro-7-iodo-3-methylimidazo[1,5-a]pyridine (370 mg, 1.340 mmol, 1.00 equiv) in 20 mL MeOH was added TEA (542.5 mg, 5.360 mmol, 4 equiv) and Pd(dppf)Cl₂CH₂Cl₂ (109.19 mg, 0.134 mmol, 0.1 equiv) in a pressure tank. The mixture was purged with nitrogen for 0.5 min and then was pressurized to 3 MPa with carbon monoxide at 100° C. for 3 hr. The reaction mixture was cooled to room temperature and filtered to remove insoluble solids. The residue was purified by silica gel column chromatography, eluted with PE/EA (3:1) to afford the methyl 8-fluoro-3-methylimidazo[1,5-a]pyridine-7-carboxylate (290 mg, 104%) as a brown solid.

LC-MS: (ES, m/z): [M+H]=209.

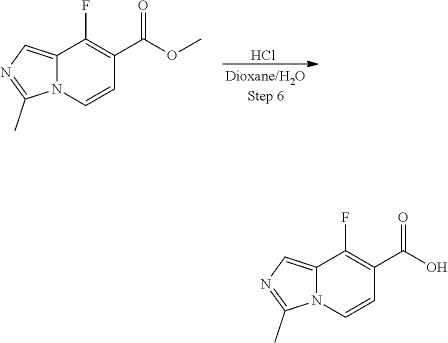

To a stirred solution of methyl 8-fluoro-3-methylimidazo[1,5-a]pyridine-7-carboxylate (120 mg) in H₂O (1 mL) and 1,4-dioxane (5 mL) was added HCl (1 mL) at room temperature. The resulting mixture was stirred for 6 hr at 90° C. The resulting mixture was concentrated under vacuum. This resulted in 8-fluoro-3-methylimidazo[1,5-a]pyridine-7-carboxylic acid (110 mg, 98%) as a brown solid.

LC-MS: (ES, m/z): [M+H]=195

Acid 47: 3-methyl-1,2,3-benzotriazole-5-carboxylic acid

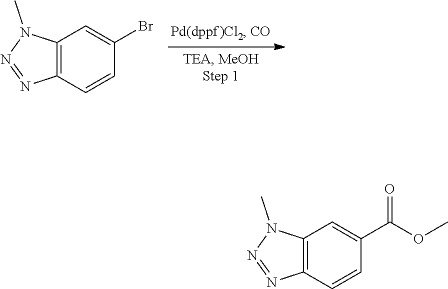

To a solution of 6-bromo-1-methyl-1,2,3-benzotriazole (800 mg, 3.773 mmol, 1.00 equiv) in CH₃OH (10 mL) was added Pd(dppf)Cl₂ (276.05 mg, 0.377 mmol, 0.1 equiv), TEA (1527.04 mg, 15.092 mmol, 4 equiv) in a pressure tank. The mixture was purged with nitrogen for 10 min and then was pressurized to 30 atm with carbon monoxide at 100° C. for 16 hr. The reaction mixture was cooled to room temperature and filtered to remove insoluble solids. The resulting mixture was extracted with EA 3×20 mL. The combined organic layers were washed with brine 3×20 mL, dried over anhydrous Na₂SO₄. After filtration, the filtrate was concentrated under reduced pressure. The residue was purified by silica gel column chromatography, eluted with THF/PE (1/1) to afford methyl 3-methyl-1,2,3-benzotriazole-5-carboxylate (600 mg, 83%) as a brown solid.

LC-MS: (ES, m/z): [M+H]⁺=192.

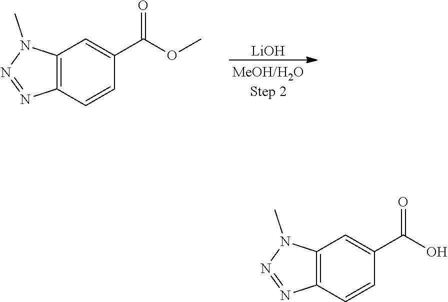

Into a 100-mL round-bottom flask, was placed methyl 3-methyl-1,2,3-benzotriazole-5-carboxylate (600 mg, 3.14 mmol, 1.00 equiv), MeOH (18 mL), H₂O (6 mL), lithium hydroxide (300.64 mg, 12.552 mmol, 4 equiv). The resulting solution was stirred for 12 hr at room temperature. The resulting mixture was concentrated under vacuum. The resulting solution was diluted with 30 mL of H₂O. The resulting solution was extracted with 2×20 mL of ethyl acetate and the aqueous layers combined. The pH value of the solution was adjusted to 3 with HCl (3 mol/L). The solids were collected by filtration. This resulted in 3-methyl-1,2,3-benzotriazole-5-carboxylic acid (450 mg, 81%) as a brown solid.

LC-MS: (ES, m/z): [M+H]⁺=178.

Acid 48: lithio 3-methyl-1-(oxan-2-yl) indazole-5-carboxylate

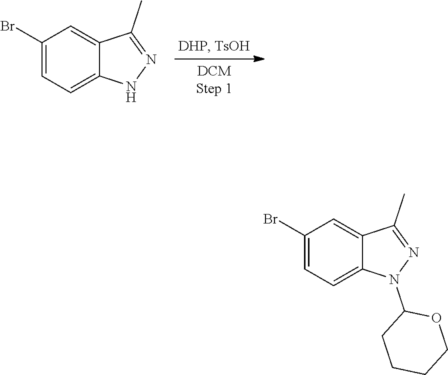 [SMILES: Cc1n[nH]c2ccc(Br)cc12 ; Cc1nn(C2CCCCO2)c2ccc(Br)cc12]

Into a 100-mL 3-necked round-bottom flask, was placed 5-bromo-3-methyl-1H-indazole (2.00 g, 9.476 mmol, 1.00 equiv), DHP (1.20 g, 14.214 mmol, 1.50 equiv), DCM (20.00 mL), TsOH (163.18 mg, 0.948 mmol, 0.10 equiv). The resulting solution was stirred for 5 hr at room temperature. The reaction was then quenched by the addition of 50 mL of water. The resulting solution was extracted with 2×50 mL of ethyl acetate and the organic layers combined and concentrated. The residue was applied onto a silica gel column with ethyl acetate/petroleum ether (1:3). This resulted in 1.6 g (57.20%) of 5-bromo-3-methyl-1-(oxan-2-yl) indazole as a white solid.

LC-MS (ES, m/z): [M+H]=295.

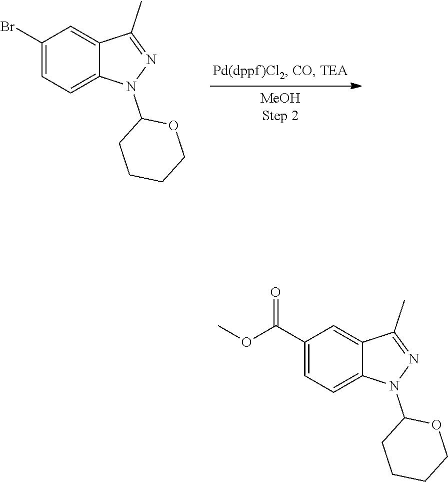 [SMILES: Cc1nn(C2CCCCO2)c2ccc(Br)cc12 ; COC(=O)c1ccc2c(c1)c(C)nn2C1CCCCO1]

Into a 100-mL pressure reactor, was placed 5-bromo-3-methyl-1-(oxan-2-yl) indazole (1.60 g, 5.420 mmol, 1.00 equiv), TEA (1.65 g, 16.260 mmol, 3.00 equiv), Pd(dppf)Cl₂ (793.22 mg, 1.084 mmol, 0.20 equiv), MeOH (20.00 mL). The flask was evacuated and flushed three times with nitrogen, followed by flushing with CO (gas). The mixture was stirred 6 hr at 60° C. under an atmosphere of CO (3 MPa). The resulting mixture was concentrated. The residue was applied onto a silica gel column with ethyl acetate/petroleum ether (1:2). This resulted in 1.25 g (84%) of methyl 3-methyl-1-(oxan-2-yl) indazole-5-carboxylate as a white solid.

LC-MS (ES, m/z): [M+H]=275.

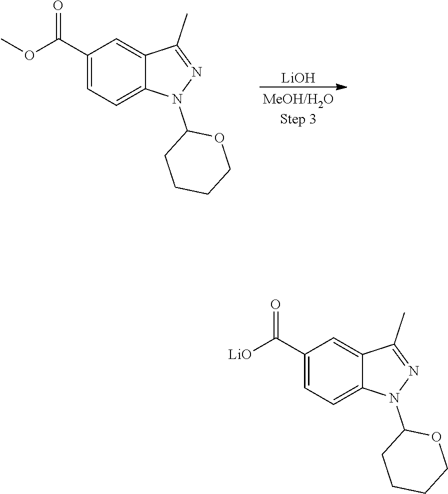

Into a 50-mL round-bottom flask, was placed methyl 3-methyl-1-(oxan-2-yl) indazole-5-carboxylate (600.00 mg, 2.187 mmol, 1.00 equiv), H₂O (2.00 mL), MeOH (10.00 mL), LiOH (157.14 mg, 6.562 mmol, 3.00 equiv). The resulting solution was stirred for 12 hr at 40° C. The resulting mixture was concentrated. This resulted in 510 mg (87.58%) of lithio 3-methyl-1-(oxan-2-yl) indazole-5-carboxylate as a white solid.

LC-MS (ES, m/z): [M+2H−Li]=275.

Acid 49: 1-cyclopropyl-1H-indazole-6-carboxylic acid

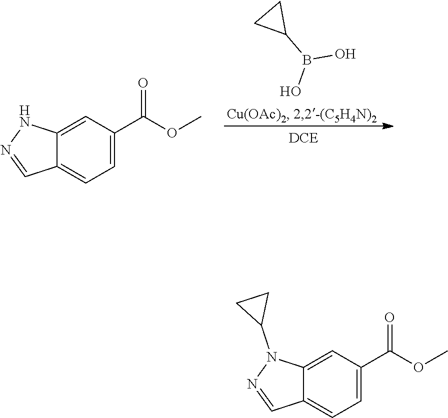

To a solution of methyl 1H-indazole-6-carboxylate (170487-40-8, 1 g, 5.7 mmol, 1 equiv) and cyclopropylboronic acid (0.98 g, 11.4 mmol, 2 equiv) in dichloroethane (20 mL) was added 2-(pyridin-2-yl)pyridine (0.89 g, 5.7 mmol, 1 equiv) and Cu(OAc)₂ (1.03 g, 5.7 mmol, 1 equiv). After stirring for 16 h at 70° C. under a nitrogen atmosphere, the resulting mixture was concentrated under reduced pressure. The residue was purified by silica gel column chromatography, eluting with ethyl acetate/petroleum ether (1:1) to afford methyl 1-cyclopropylindazole-6-carboxylate (500 mg, 41%) as yellow oil.

LC-MS: (ES, m/z): [M+H]⁺=217

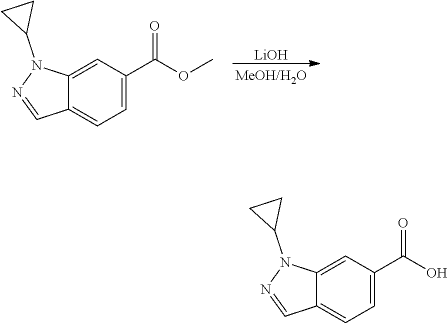

Into a 50 mL round-bottom flask was added methyl 1-cyclopropylindazole-6-carboxylate (500 mg, 2.3 mmol, 1 equiv) and lithium hydroxide (166 mg, 6.9 mmol, 3 equiv) in CH₃OH (10 mL), H₂O (3 mL) at room temperature. The resulting mixture was stirred for 10 h at room temperature. The resulting mixture was concentrated under vacuum. The resulting solution was diluted with 50 mL of H₂O. The resulting solution was extracted with 2×20 mL of ethyl acetate and the aqueous layers combined. The pH of the solution was adjusted to 3 with HCl (3M). The solids were collected by filtration. This resulted in 1-cyclopropyl-1H-indazole-6-carboxylic acid (380 mg, 81.27%) as a yellow solid.

LC-MS: (ES, m/z): [M+H]⁺=203

Acid 50: 1-methylisoquinoline-7-carboxylic acid

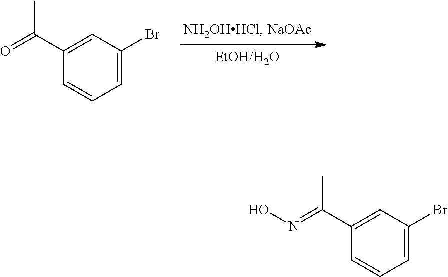

The solution of NH₂OH·HCl (5.6 g, 80.4 mmol, 1.6 equiv) and NaOAc (8.3 g, 101 mmol, 2.0 equiv) in EtOH (100 mL) and H₂O (25 mL) was stirred for 30 min at room temperature. To this mixture was added m-bromoacetophenone (10 g, 50 mmol, 1.0 equiv) in portions at room temperature, then warmed to 80° C. and stirred for more 3 h. The mixture was allowed to cool down to room temperature and concentrated under reduced pressure. The resulting mixture was diluted with water (25 mL) and extracted with EtOAc (3×80 mL). The combined organic layers were washed with brine (50 mL), dried over anhydrous Na₂SO₄. After filtration, the filtrate was concentrated under reduced pressure. The residue was purified by silica gel column chromatography, eluted with ethyl acetate/petroleum ether (5:1) to afford (E)-N-[1-(3-bromophenyl)ethylidene]hydroxylamine (10 g) as an off-white solid.

LC-MS (ES, m/z): [M+1]⁺=214

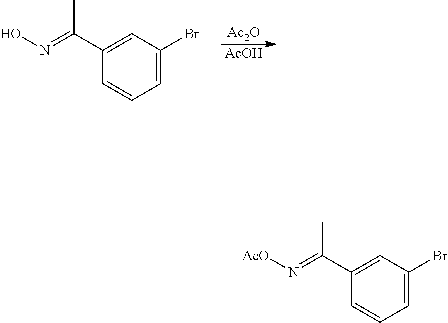

Into a 250-mL round-bottom-flask was added (E)-N-[1-(3-bromophenyl) ethylidene]hydroxylamine (10.0 g, 46.7 mmol, 1.0 equiv), acetic anhydride (50 mL) and AcOH (50 mL). The mixture was stirred for 3 h at room temperature, then concentrated under reduced pressure. The resulting solution was diluted with water (100 mL) and extracted with EtOAc (3×80 mL). The combined organic layers were washed with NaHCO₃ (5×50 mL) and brine (80 mL), dried over anhydrous Na₂SO₄. After filtration, the filtrate was concentrated under reduced pressure. The residue was purified by silica gel column chromatography, eluted with ethyl acetate/petroleum ether (5:1) to afford (E)-[1-(3-bromophenyl)ethylidene]amino acetate (7.5 g) as a light yellow solid.

LC-MS (ES, m/z): [M+Na+ACN]⁺=319

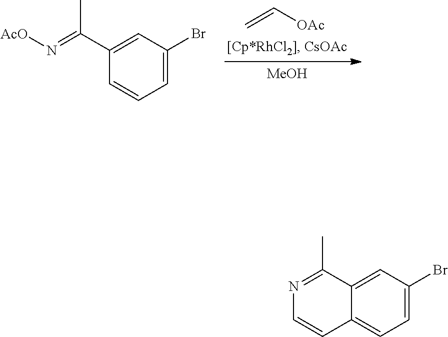

Into a 100-mL round-bottom-flask was added (E)-[1-(3-bromophenyl) ethylidene]amino acetate (5 g, 19.5 mmol, 1.0 equiv), vinyl acetate (16.8 g, 195 mmol, 10.0 equiv), CsOAc (1.1 g, 5.9 mmol, 0.3 equiv), MeOH (50 mL) and Pentamethylcyclopentadienylrhodium(III) chloride dimer (120 mg, 0.195 mmol, 0.01 equiv). The resulting mixture was stirred for 24 h at 60° C. under a nitrogen atmosphere. The mixture was allowed to cool down to room temperature and concentrated under vacuum. The residue was purified by Prep-HPLC with the following conditions: Mobile Phase A: Water (0.05% TFA), Mobile Phase B: acetonitrile. The collected solution was concentrated under vacuum to remove acetonitrile and the residue was dried by lyophilization. This resulted in 7-bromo-1-methylisoquinoline (1.8 g) as an off-white solid.

LC-MS (ES, m/z): [M+1]⁺=222

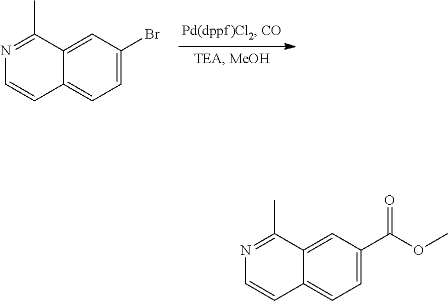

Into a 100-mL pressure vessel was added 7-bromo-1-methylisoquinoline (1.8 g, 8. mmol, 1 equiv), TEA (2.5 g, 24 mmol, 3.0 equiv), MeOH (20 mL) and Pd(dppf)Cl₂ (0.59 g, 0.81 mmol, 0.1 equiv). The resulting mixture was stirred for 4 h at 120° C. under a carbon monoxide atmosphere. The mixture was allowed to cool down to room temperature and filtered, the filter cake was washed with EtOAc (3×20 mL). The filtrate was concentrated under reduced pressure. The residue was purified by silica gel column chromatography, eluted with ethyl acetate/petroleum ether (1:2) to afford methyl 1-methylisoquinoline-7-carboxylate (1.3 g, 80%) as a light yellow solid.

LC-MS (ES, m/z): [M+1]⁺=202

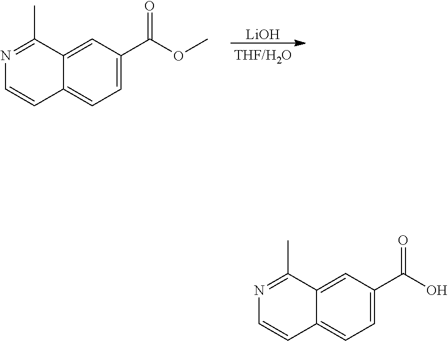

To a stirred solution of methyl 1-methylisoquinoline-7-carboxylate (1.3 g, 6.5 mmol, 1 equiv) in THF (10 mL) and H₂O (2 mL) were added lithium hydroxide (464.2 mg, 19.4 mmol, 3.0 equiv). The resulting mixture was stirred for 16 h at room temperature. The mixture was concentrated under vacuum and basified to pH 3˜4 with 2M HCl (aq). The precipitated solids were collected by filtration and washed with water (3×5 mL). This resulted in 1-methylisoquinoline-7-carboxylic acid (900 mg, 74%) as a light yellow solid.

LC-MS (ES, m/z): [M+1]⁺=188

Acid 51: 1,3-dimethyl-1H-indazole-5-carboxylic acid

Prepared according to WO2021127166, Acid F

Acid 52: 1-(pyridin-4-yl)-1H-indazole-5-carboxylic acid

Prepared according to WO2021127166, Acid AC

Acid 53: 1-(pyridin-4-yl)-1H-benzo[d][1,2,3]triazole-5-carboxylic acid

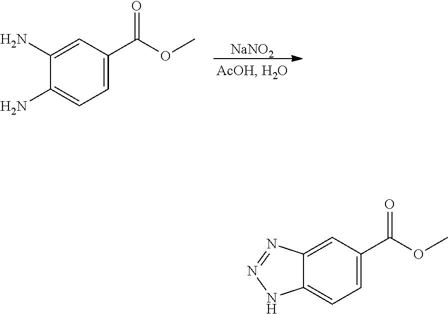

To a solution of methyl 3,4-diaminobenzoate (10.0 g, 60.2 mmol, 1.0 equiv) in AcOH (25 mL) and H₂O (45 mL), was added sodium nitrite (8.30 g, 120 mmol, 2.0 equiv) in H₂O (30 mL) dropwise at 0° C. The resulting mixture was stirred for 1 h at room temperature. The mixture was acidified to pH 8 with NaHCO₃ and extracted with EtOAc (3×80 mL). The combined organic layers were washed with brine (60 mL) and dried over anhydrous Na₂SO₄. The filtrate was concentrated under reduced pressure and the residue was purified by silica gel column chromatography, eluting with ethyl acetate/petroleum ether (1:1) to afford methyl 1H-1,2,3-benzotriazole-5-carboxylate (10.1 g) as a yellow solid.

LC-MS (ES, m/z): [M+1]⁺=178

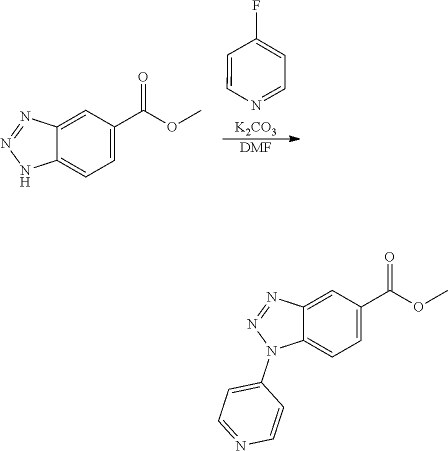

A solution of methyl 1H-1,2,3-benzotriazole-5-carboxylate (200.00 mg, 1.129 mmol, 1.0 equiv), 4-fluoropyridine (164.41 mg, 1.7 mmol, 1.5 equiv), K₂CO₃ (468 mg, 3.4 mmol, 3.0 equiv) in DMF (5 mL) was stirred for 1.5 h at 80° C. The mixture was allowed to cool down to room temperature and diluted with H₂O (20 mL). The resulting mixture was extracted with EtOAc (3×20 mL). The combined organic layers were washed with H₂O (5×20 mL) and brine (30 mL) and dried over anhydrous Na₂SO₄. After concentration under reduced pressure, the residue was purified by silica gel column chromatography, eluting with ethyl acetate/petroleum ether (1:2) to afford methyl 1-(pyridin-4-yl)-1,2,3-benzotriazole-5-carboxylate (150 mg) as a light-yellow oil.

LC-MS (ES, m/z): [M+1]⁺=255

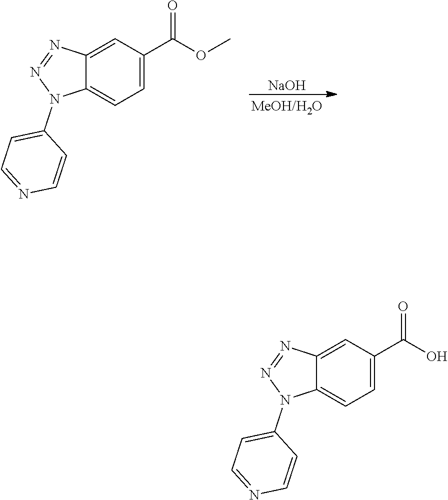 [SMILES: COC(=O)c1ccc2c(c1)nnn2-c1ccncc1 ; "NaOH" ; O=C(O)c1ccc2c(c1)nnn2-c1ccncc1]

To a stirred solution of methyl 1-(pyridin-4-yl)-1,2,3-benzotriazole-5-carboxylate (150 mg, 0.590 mmol, 1.0 equiv) in MeOH (5 mL) and H₂O (1 mL) were added sodium hydroxide (47 mg, 1.2 mmol, 2.0 equiv). The resulting mixture was stirred for 6 h at room temperature. The mixture was concentrated under vacuum and acidified to pH 3˜4 with 2M HCl (aq). The precipitated solids were collected by filtration and washed with water (3×5 mL). This resulted in 110 mg (crude). This crude product was purified by Prep-HPLC with the following conditions: mobile phase, A: 0.1% HCl in water; B: acetonitrile; Gradient: 24-95% B in 7.9 min. The fractions were collected and concentrated under vacuum to afford 1-(pyridin-4-yl)-1H-benzo[d][1,2,3]triazole-5-carboxylic acid (45 mg) as a white solid.

LC-MS (ES, m/z): [M+1]⁺=240

Acid 54: 1,4-dimethylphthalazine-6-carboxylic acid

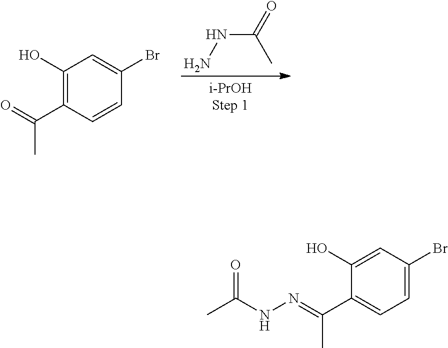

A solution of 2-hydroxy-4-bromo acetophenone (1 g, 4.7 mmol, 1 equiv) and acetohydrazide (0.34 g, 4.7 mmol, 1 equiv) in i-PrOH (20 mL) was stirred for 5 h at 100° C. The mixture was allowed to cool down to room temperature. The precipitated solids were collected by filtration and washed with PrOH (20 mL). This resulted in N′-[(1E)-1-(4-bromo-2-hydroxyphenyl)ethylidene]acetohydrazide (1.1 g, 87. %) as a yellow solid.

LC-MS: (ES, m/z): [M+H]⁺=271, 273

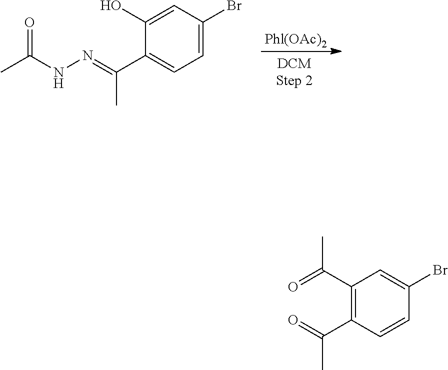

A solution of N′-[(1E)-1-(4-bromo-2-hydroxyphenyl)ethylidene]acetohydrazide (1.1 g, 4.0 mmol, 1 equiv) and (diacetoxyiodo)benzene (3.9 g, 12.2 mmol, 3 equiv) in DCM (30 mL) was stirred for 16 h at room temperature. The mixture was basified to pH=8 with NaHCO₃. The resulting mixture was extracted with DCM (30 mL×2). The combined organic layers were washed with NaCl (20 mL) and dried over anhydrous Na₂SO₄ and reduced under pressure. The residue was purified by silica gel column chromatography, eluted with 1/1 ethyl acetate/petroleum ether to afford 1-(2-acetyl-4-bromophenyl) ethanone (600 mg, 61%) as a brown oil.

LC-MS: (ES, m/z): [M+H]⁺=241,243

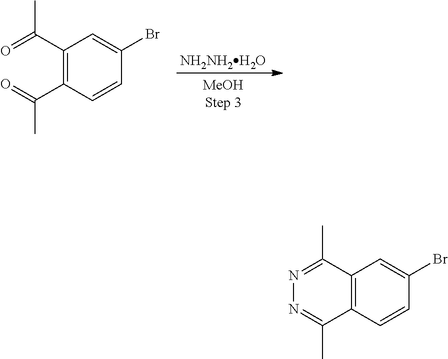

A solution of 1-(2-acetyl-5-bromophenyl) ethanone (0.6 g, 2.5 mmol, 1 equiv) and NH₂NH₂·H₂O (2.49 g, 49.8 mmol, 20 equiv) in methanol (20 mL) was stirred for 2 h at 60° C. . . . The mixture was allowed to cool down to room temperature. The resulting mixture was concentrated under reduced pressure. The crude product 6-bromo-1,4-dimethylphthalazine (0.5 g, 68%) was used in the next step directly without further purification.

LC-MS: (ES, m/z): [M+H]⁺=237,239

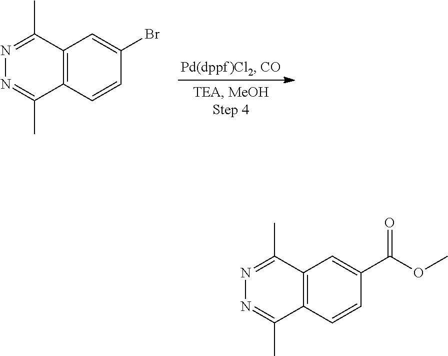 [SMILES: Cc1nnc(C)c2cc(Br)ccc12 ; COC(=O)c1ccc2c(C)nnc(C)c2c1]

To a solution of 6-bromo-1,4-dimethylphthalazine (0.5 g, 2.1 mmol, 1 equiv) in 20 mL MeOH was added triethylamine (0.64 g, 6.33 mmol, 3 equiv) and Pd(dppf)Cl₂ (0.17 g, 0.211 mmol, 0.1 equiv) in a pressure vessel. The mixture was purged with nitrogen for 3 mins and then was pressurized with carbon monoxide and heated at 120° C. for 4 h. The reaction mixture was cooled to room temperature. The residue was purified by silica gel column chromatography, eluted with petroleum ether/ethyl acetate (50/50) to afford methyl 1,4-dimethylphthalazine-6-carboxylate (300 mg, 66%) as a brown solid.

LC-MS: (ES, m/z): [M+H]⁺=217

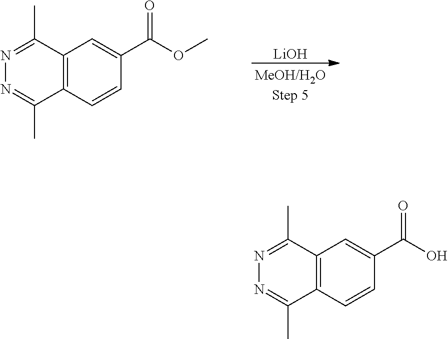 [SMILES: COC(=O)c1ccc2c(C)nnc(C)c2c1 ; "LiOH" ; Cc1nnc(C)c2cc(C(=O)O)ccc12]

To a stirred solution of methyl 1,4-dimethylphthalazine-6-carboxylate (300 mg, 1.4 mmol, 1 equiv) in MeOH (10 mL) H₂O (2 mL) was added lithium hydroxide (66.5 mg, 2.8 mmol, 2 equiv) in portions at room temperature. The resulting mixture was stirred for 2 h at room temperature. The resulting mixture was concentrated under reduced pressure. The mixture was acidified to pH 3. The precipitated solids were collected by filtration and washed with H₂O (5 mL) to afford 1,4-dimethylphthalazine-6-carboxylic acid (220 mg, 78%) as a brown solid.

LC-MS: (ES, m/z): [M+H]⁺=203

Acid 55: 1-methyl-2-oxoindoline-6-carboxylic acid

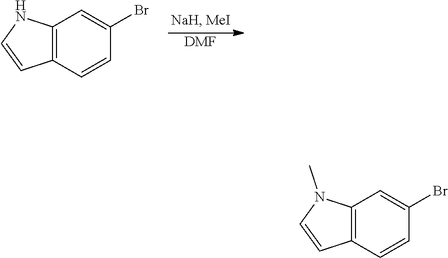

Into a 100 mL 3-necked round-bottom flask were added 6-bromo-1H-indole (2 g, 10.2 mmol, 1 equiv), DMF (40 mL) and NaH (0.37 g, 15.3 mmol, 1.5 equiv) at 0° C. The resulting mixture was stirred for 30 min at 0° C. under nitrogen atmosphere. To this stirred mixture was added methyl iodide (1.74 g, 12.2 mmol, 1.2 equiv) dropwise at 0° C. under nitrogen atmosphere. The resulting mixture was stirred for 3 h at 0° C. The reaction was quenched with water/ice at 0° C. The resulting mixture was extracted with EtOAc (2×100 mL). The combined organic layers were washed with brine (2×100 mL) and dried over anhydrous Na₂SO₄. After filtration, the filtrate was concentrated under reduced pressure. The residue was purified by silica gel column chromatography, eluted with petroleum ether/ethyl acetate (1:1) to afford 6-bromo-1-methylindole (1.8 g, 84%) as a brown oil.

LC-MS: (ES, m/z): [M+H]⁺=210

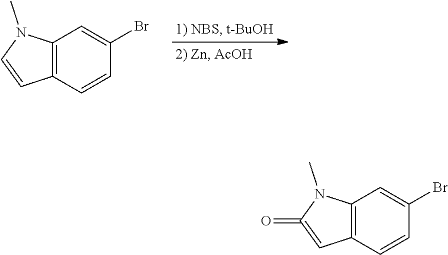

Into a 50 mL 3-necked round-bottom flask were added 6-bromo-1-methylindole (1.8 g, 8.6 mmol, 1 equiv), 2-methyl-2-propanol (20 mL) and N-bromosuccinimide (6.1 g, 34 mmol, 4 equiv) at room temperature. The resulting mixture was stirred for 1 h at 40° C. under nitrogen atmosphere. The mixture was allowed to cool down to room temperature. The resulting mixture was diluted with water (100 mL). The resulting mixture was extracted with EtOAc (2×100 mL). The combined organic layers were washed with brine (2×100 mL) and dried over anhydrous Na₂SO₄. After filtration, the filtrate was concentrated under reduced pressure. The crude product was diluted with acetic acid (30 mL). To the stirred solution was added zinc (2.8 g, 42.8 mmol, 5 equiv) at 0° C. The resulting mixture was stirred for 1 h at 0° C. under nitrogen atmosphere. The resulting mixture was concentrated under vacuum and diluted with water (100 mL). The resulting mixture was extracted with EtOAc (2×100 mL). The combined organic layers were washed with brine (2×100 mL), dried over anhydrous Na₂SO₄ and concentrated under reduced pressure. The residue was purified by silica gel column chromatography, eluted with petroleum ether/ethyl acetate (1:1) to afford 6-bromo-1-methyl-3H-indol-2-one (1.2 g, 62%) as a brown solid.

LC-MS: (ES, m/z): [M+H]⁺=226

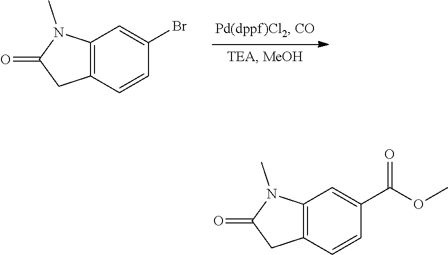

Into a 100 mL pressure vessel were added 6-bromo-1-methyl-3H-indol-2-one (1200 mg, 5.308 mmol, 1 equiv) MeOH (24 mL), Pd(dppf)Cl₂ (388.39 mg, 0.531 mmol, 0.10 equiv) and TEA (1611 mg, 15.924 mmol, 3 equiv) at room temperature. The resulting mixture was stirred for 5 h at 100° C. under carbon monoxide atmosphere. The mixture was allowed to cool down to room temperature. The resulting mixture was diluted with water (100 mL) and the mixture extracted with EtOAc (2×100 mL). The combined organic layers were washed with brine (2×100 mL) and dried over anhydrous Na₂SO₄. After filtration and concentration under reduced pressure, the residue was purified by silica gel column chromatography, eluting with petroleum ether/ethyl acetate (1:1) to afford methyl 1-methyl-2-oxo-3H-indole-6-carboxylate (500 mg, 46%) as a dark yellow solid.

LC-MS: (ES, m/z): [M+H]⁺=206

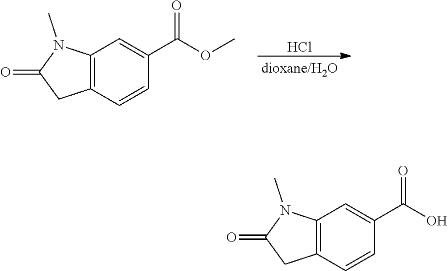

Into a 40 mL round-bottom flask was added methyl 1-methyl-2-oxo-3H-indole-6-carboxylate (300 mg, 1.5 mmol, 1 equiv), dioxane (6 mL), water (3 mL) and HCl (6M) (3 mL) at room temperature. The resulting mixture was stirred for 16 h at 100° C. under nitrogen atmosphere and cooled down to room temperature. The reaction mixture was diluted with water (10 mL) and extracted with DCM (2×20 mL). The combined organic layers were washed with brine (2×15 mL), dried over anhydrous Na₂SO₄ and concentrated under reduced pressure. The crude product was used in the next step directly without further purification. This resulted in 1-methyl-2-oxoindoline-6-carboxylic acid (200 mg, 72%) as a brown oil.

LC-MS: (ES, m/z): [M+H]⁺=192

Acid 56: 1-methyl-2-oxoindoline-5-carboxylic acid

Prepared as for Acid 55 using 5-bromo-1H-indole

Acid 57: 1-methyl-1,3-dihydrobenzo[c]isothiazole-6-carboxylic acid 2,2-dioxide

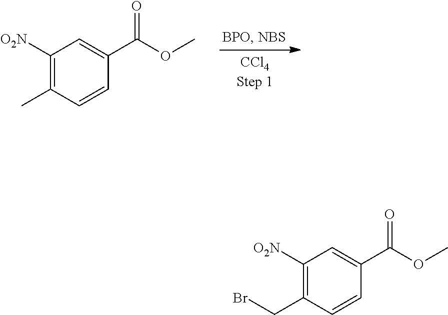

A solution of methyl 4-methyl-3-nitrobenzoate (6 g, 30.7 mmol, 1 equiv) in carbon tetrachloride (200 mL) was treated with NBS (6.0 g, 34 mmol, 1.1 equiv) at room temperature followed by the addition of benzoyl peroxide (0.79 g, 3.1 mmol, 0.1 equiv) in portions at room temperature. The resulting mixture was stirred for 16 h at 100° C. The mixture was allowed to cool down to room temperature and concentrated under reduced pressure. The residue was purified by silica gel column chromatography, eluting with petroleum ether/ethyl acetate=10/1 to afford methyl 4-(bromomethyl)-3-nitrobenzoate (5 g, 59%) as a yellow liquid.

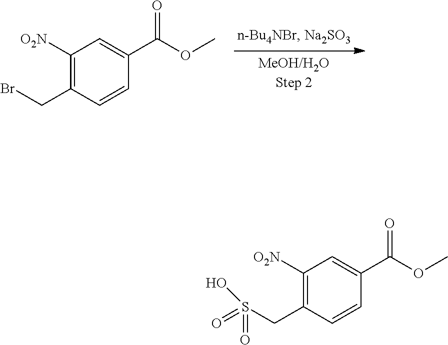 [SMILES: COC(=O)c1ccc(CBr)c([N+](=O)[O-])c1 ; COC(=O)c1ccc(CS(=O)(=O)O)c([N+](=O)[O-])c1]

To a stirred solution of methyl 4-(bromomethyl)-3-nitrobenzoate (5 g, 18.2 mmol, 1 equiv) and Na₂SO₃ (4.60 g, 36 mmol, 2 equiv) in H₂O (50 mL) and MeOH (100 mL) was added tetrabutylammonium bromide (5.88 g, 18.244 mmol, 1 equiv) in portions at room temperature. The resulting mixture was stirred for 3 h at 100° C. The mixture was allowed to cool down to room temperature and diluted with 50 mL water. The resulting mixture was extracted with ethyl acetate (100 mL×2). The combined organic layers were washed with NaCl (100 mL), dried over anhydrous Na₂SO₄ and concentrated under reduced pressure. The crude product [4-(methoxycarbonyl)-2-nitrophenyl]methanesulfonic acid (6 g) was used in the next step directly without further purification.

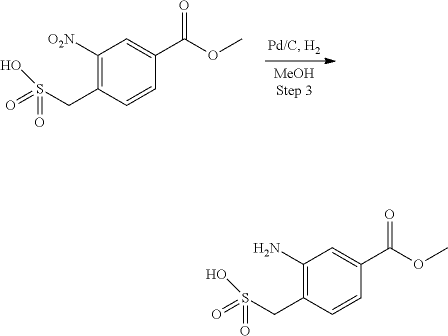 [SMILES: COC(=O)c1ccc(CS(=O)(=O)O)c([N+](=O)[O-])c1 ; COC(=O)c1ccc(CS(=O)(=O)O)c(N)c1]

To a solution of [4-(methoxycarbonyl)-2-nitrophenyl]methanesulfonic acid (6 g, 22 mmol, 1 equiv) in 100 mL MeOH was added Pd/C (10%, 2.3 g) in a pressure tank. The mixture was hydrogenated at room temperature under 30 psi of hydrogen pressure for 16 h, filtered through a Celite pad and concentrated under reduced pressure. This resulted in [2-amino-4-(methoxycarbonyl)phenyl]methanesulfonic acid (4.5 g, 84%) as a white solid.

LC-MS: (ES, m/z): [M+H]⁺=246

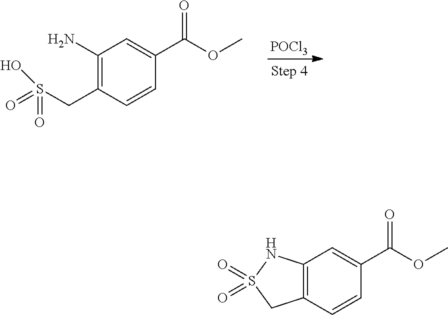

A solution of [2-amino-4-(methoxycarbonyl)phenyl]methanesulfonic acid (2 g, 5 mmol, 1 equiv, 60%) in phosphorus oxychloride (15 mL) was stirred for 1 h at 80° C. The mixture was allowed to cool down to room temperature and the resulting mixture concentrated under reduced pressure. The residue was purified by silica gel column chromatography, eluting with petroleum ether/ethyl acetate=1/1 to afford methyl 2,2-dioxo-1,3-dihydro-2lambda6,1-benzothiazole-6-carboxylate (300 mg, 27%) as a yellow solid.

LC-MS: (ES, m/z): [M+H]⁺=228

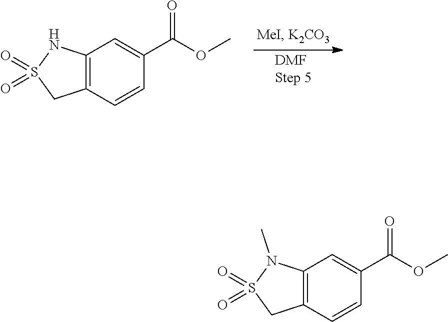 [SMILES: COC(=O)c1ccc2c(c1)NS(=O)(=O)C2 ; COC(=O)c1ccc2c(c1)N(C)S(=O)(=O)C2]

To a stirred solution of methyl 1,3-dihydrobenzo[c]isothiazole-6-carboxylate 2,2-dioxide (300 mg, 1.3 mmol, 1 equiv) and K₂CO₃ (365 mg, 2.6 mmol, 2 equiv) in dimethylformamide (10 mL) was added Mel (206 mg, 1.5 mmol, 1.1 equiv) dropwise at 0° C. The resulting mixture was quenched with water and extracted with ethyl acetate (10 mL×2). The combined organic layers were washed with brine (10 mL) and dried over anhydrous Na₂SO₄. The filtrate was concentrated under reduced pressure and the residue was purified by silica gel column chromatography, eluting with petroleum ether/ethyl acetate=1/1 to afford methyl 1-methyl-1,3-dihydrobenzo[c]isothiazole-6-carboxylate 2,2-dioxide (200 mg, 63%) as a brown solid.

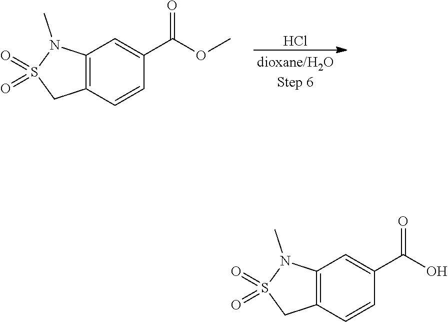 [SMILES: COC(=O)c1ccc2c(c1)N(C)S(=O)(=O)C2 ; Cl ; CN1c2cc(C(=O)O)ccc2CS1(=O)=O]

A solution of methyl 1-methyl-1,3-dihydrobenzo[c]isothiazole-6-carboxylate 2,2-dioxide (200 mg, 0.83 mmol, 1 equiv) and hydrogen chloride (1M in dioxane, 5 mL) in 1,4-dioxane was stirred for 5 h at 80° C. The resulting mixture was concentrated under reduced pressure to give the crude product 1-methyl-1,3-dihydrobenzo[c]isothiazole-6-carboxylic acid 2,2-dioxide (120 mg, 64%) which was used directly without purification.

LC-MS: (ES, m/z): [M−H]⁻=226

Acid 58: 1-methyl-2-oxo-1,2-dihydropyrazolo[1,5-a]pyridine-6-carboxylic acid

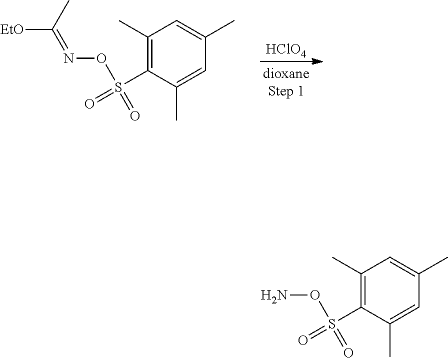

A solution of O-(mesitylsulfonyl) hydroxylamine (5 g, 17.5 mmol, 1 equiv) and HClO₄ (8.8 g, 87.6 mmol, 5 equiv) in 1,4-dioxane (20 ml) was stirred for 3 h at 0° C. under air atmosphere. The resulting mixture was concentrated under vacuum. The resulting mixture was diluted with water (50 mL) and the precipitated solids were collected by filtration and washed with diethyl ether (20 ml). This resulted in O-(mesitylsulfonyl) hydroxylamine (2.5 g, 31%) as a white solid.

LC-MS: (ES, m/z): [M+H]⁺=216

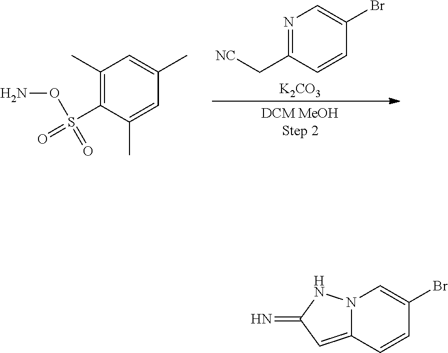

A solution of amino O-(mesitylsulfonyl) hydroxylamine (2.5 g, 5.3 mmol, 1 equiv) and 2-(5-bromopyridin-2-yl) acetonitrile (1.05 g, 5.3 mmol, 1 equiv) in DCM (30 ml) was stirred overnight at room temperature under nitrogen atmosphere. The resulting mixture was concentrated under vacuum. To the above mixture, K₂CO₃ (3.7 g, 26.7 mmol, 5 equiv) in MeOH (40 ml) was added and stirred for 6 h at room temperature under a nitrogen atmosphere. The resulting mixture was concentrated under vacuum and diluted with water (30 mL). The resulting mixture was extracted with EtOAc (3×30 mL). The combined organic layers were washed with water (2×20 ml), dried over anhydrous Na₂SO₄, filtered and concentrated under reduced pressure. The residue was purified by silica gel column chromatography, eluted with ethyl acetate/ethyl ether (8:1) to afford 6-bromopyrazolo[1,5-a]pyridin-2 (1H)-imine (0.75 g, 64%) as a yellow solid.

LC-MS: (ES, m/z): [M+H]⁺=212,214

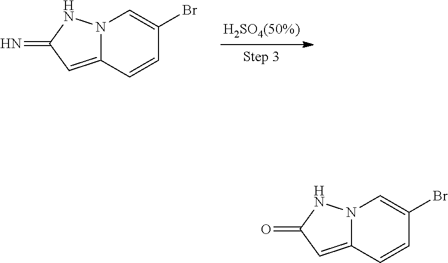 [SMILES: N=c1cc2ccc(Br)cn2[nH]1 ; O=c1cc2ccc(Br)cn2[nH]1]

A solution of 6-bromopyrazolo[1,5-a]pyridin-2 (1H)-one (700 mg, 3.3 mmol, 1 equiv) in 50% H₂SO₄ (10 ml) was stirred for 2 h at 100° C. under air atmosphere. The mixture was allowed to cool down to room temperature and quenched with water/ice (100 ml). The mixture was basified to pH 9 with saturated Na₂CO₃ (aq.) and extracted with EtOAc (3×60 mL). The combined organic layers were washed with water (2×50 mL), dried over anhydrous Na₂SO₄ and concentrated under reduced pressure. This resulted in 6-bromo-1H-pyrazolo[1,5-a]pyridin-2-one (560 mg, 72%) as a yellow solid.

LC-MS: (ES, m/z): [M+H]⁺=213,215

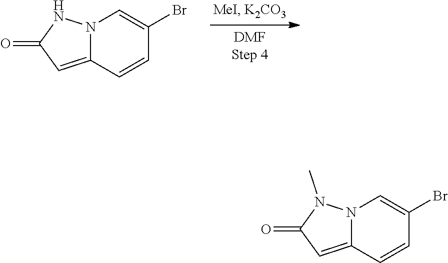

A solution of 6-bromo-1H-pyrazolo[1,5-a]pyridin-2-one (560 mg, 2.6 mmol, 1 equiv), methyl iodide (560 mg, 3.9 mmol, 1.5 equiv) and K₂CO₃ (732 mg, 5.3 mmol, 2.0 equiv) in DMF (10 ml) was stirred for 16 h at room temperature under nitrogen atmosphere. The resulting mixture was diluted with water (50 ml). The resulting mixture was extracted with EtOAc (3×30 ml). The combined organic layers were washed with water (2×30 ml), dried over anhydrous Na₂SO₄ and the filtrate concentrated under reduced pressure. The residue was purified by silica gel column chromatography, eluting with petroleum ether/ethyl acetate (4:1) to afford 6-bromo-1-methylpyrazolo[1,5-a]pyridin-2-one (420 mg, 70%) as a yellow solid.

LC-MS: (ES, m/z): [M+H]⁺=227, 229

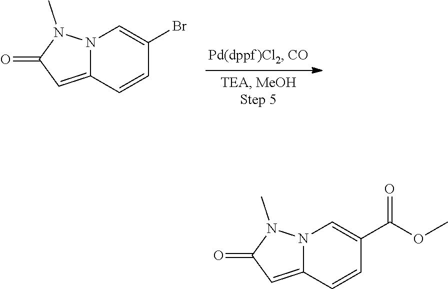

To a solution of 6-bromo-1-methylpyrazolo[1,5-a]pyridin-2-one (200 mg, 0.881 mmol, 1 equiv) and Et₃N (267 mg, 2.6 mmol, 3 equiv) in 20 mL MeOH was added Pd(dppf)Cl₂ (65 mg, 0.088 mmol, 0.1 equiv) in a pressure vessel. The mixture was purged with nitrogen for 2 mins and then was pressurized to 30 atm with carbon monoxide at 120° C. for 2 h. The reaction mixture was cooled to room temperature and filtered to remove insoluble solids. The resulting mixture was concentrated under vacuum. The residue was purified by silica gel column chromatography, eluting with petroleum ether/ethyl acetate (4:1) to afford methyl 1-methyl-2-oxopyrazolo[1,5-a]pyridine-6-carboxylate (120 mg, 66%) as a light yellow solid.

LC-MS−: (ES, m/z): [M+H]⁺=207

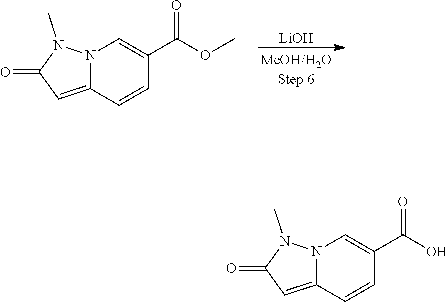

A solution of methyl 1-methyl-2-oxopyrazolo[1,5-a]pyridine-6-carboxylate (100 mg, 0.485 mmol, 1 equiv) and lithium hydroxide (23 mg, 0.97 mmol, 2 equiv) in MeOH (8 ml) and water (2 ml) was stirred for 2 h at 50° C. under air atmosphere. The reaction mixture was cooled to room temperature. The resulting mixture was concentrated under vacuum. The mixture was acidified to pH 4 with conc. HCl. The resulting mixture was concentrated under vacuum. This resulted in 1-methyl-2-oxo-1,2-dihydropyrazolo[1,5-a]pyridine-6-carboxylic acid (120 mg, crude) as a light yellow solid.

LC-MS: (ES, m/z): [M+H]⁺=193

The acids below were purchased from commercial suppliers:

| Structure | ID | CAS No |
|---|---|---|
| 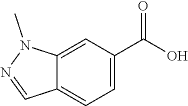 | A | 1031417-77-2 |
| 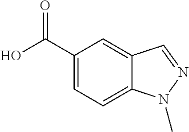 | B | 1176754-31-6 |
| 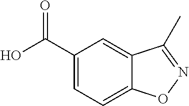 | C | 478169-74-3 |
| 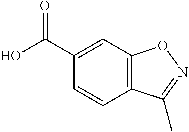 | D | 478169-72-1 |
| 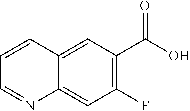 [SMILES: O=C(O)c1cc2cccnc2cc1F] | E | 1061650-21-2 |
| 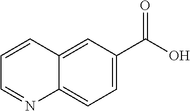 [SMILES: O=C(O)c1ccc2ncccc2c1] | F | 10349-57-2 |
| 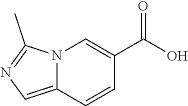 | G | 1260777-34-1 |
| 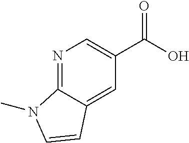 | H | 934568-20-4 |
| 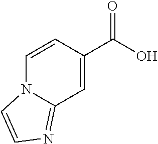 [SMILES: O=C(O)c1ccn2ccnc2c1] | I | 648423-85-2 |
| 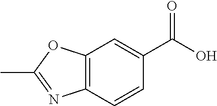 | J | 13452-14-7 |
| 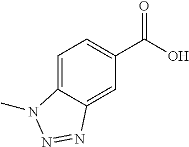 | K | 305381-67-3 |
| 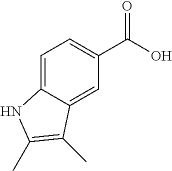 | L | 14844-73-6 |
| 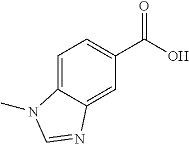 | M | 53484-17-6 |
| 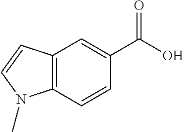 | N | 186129-25-9 |
| 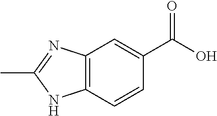 | O | 709-19-3 |
| 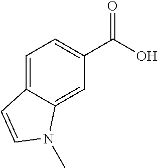 | P | 202745-73-1 |
| 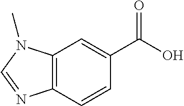 | Q | 53484-18-7 |
| 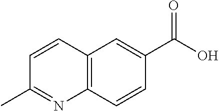 | R | 635-80-3 |

### Example 1: 2-methyl-N-{2-[(2R)-1-methylpyrrolidin-2-yl]-1H-pyrrolo[3,2-c]pyridin-6-yl}pyrazolo[1,5-a]pyridine-6-carboxamide

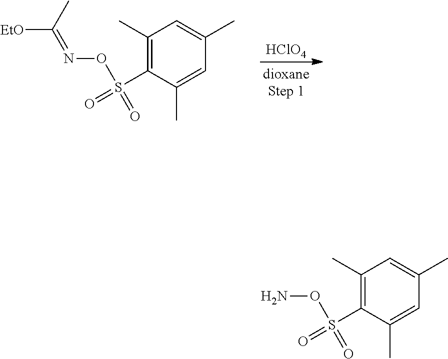

To a solution of (E)-(ethyl N-[(2,4,6-trimethylbenzenesulfonyl)oxy]ethanimidate) (5 g, 17.52 mmol, 1.00 equiv) in 100 mL 1,4-dioxane was added HClO₄ (3.77 g, 26.28 mmol, 1.5 equiv) In a 250 mL 3-necked round-bottom flask at 0˜5° C. The resulting mixture was stirred for 3 hr at 5° C. under nitrogen atmosphere. The reaction was monitored by LCMS. The reaction was quenched by the addition of water/ice (200 mL) at 5° C. The precipitated solids were collected by filtration and washed with PE (2×50 mL). This resulted in amino 2,4,6-trimethylbenzenesulfonate (1.89 g) as a white crude solid.

LC-MS (ES, m/z): [M+H]=216.

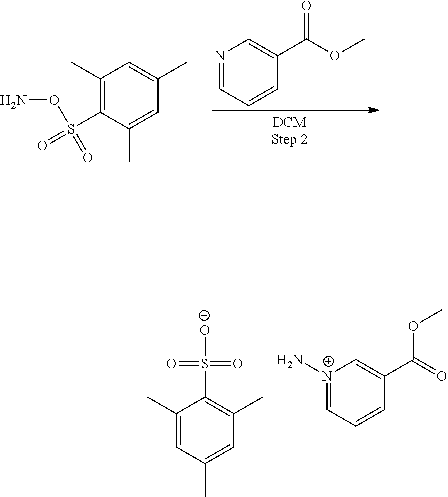

Into a 50 mL 3-necked round-bottom flask were added amino 2,4,6-trimethylbenzenesulfonate (1.8 g, 8.362 mmol, 1.00 equiv) and methyl nicotinate (1.61 g, 11.707 mmol, 1.4 equiv) in 18 mL DCM at room temperature. The resulting mixture was stirred for 1 hr at room temperature. The reaction was monitored by LCMS. The resulting mixture was concentrated under vacuum. This resulted in 1-amino-3-(methoxycarbonyl)pyridin-1-ium 2,4,6-trimethylbenzenesulfonate (1.9 g) as a yellow crude solid.

LC-MS (ES, m/z): [M−199]=153.

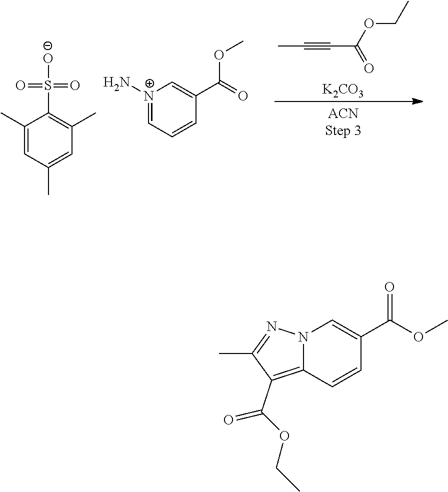

To a solution of but-2-ynoic acid ethyl ester (0.60 g, 5.39 mmol, 1.0 equiv) in 20 mL CH₃CN was added K₂CO₃ (2.98 g, 21.56 mmol, 4.0 equiv) and 1-amino-3-(methoxycarbonyl)pyridin-1-ium 2,4,6-trimethylbenzenesulfonate (1.9 g, 5.391 mmol, 1.00 equiv) in a 100 mL 3-necked round-bottom flask. The resulting mixture was stirred for 16 hr at room temperature. The reaction was monitored by LCMS. The resulting mixture was filtered. The filtrate was concentrated under reduced pressure. The residue was purified by silica gel column chromatography, eluted with PE/EA (5:1) to afford 3-ethyl 6-methyl 2-methylpyrazolo[1,5-a]pyridine-3,6-dicarboxylate (200 mg, 4% for 3 steps) as a yellow solid.

LC-MS (ES, m/z): [M+H]=263.

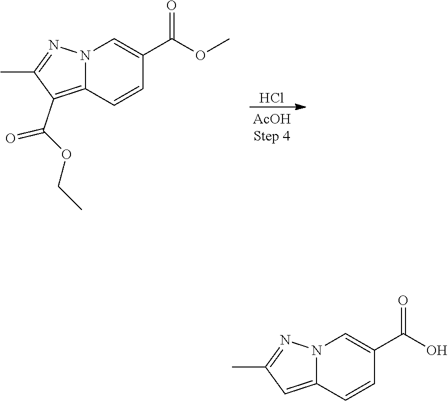

To a solution of 3-ethyl 6-methyl 2-methylpyrazolo[1,5-a]pyridine-3,6-dicarboxylate (180 mg, 0.686 mmol, 1.00 equiv) in 1 mL AcOH was added HCl (12M) (1 mL, 32.912 mmol, 47.95 equiv) into a 40 mL sealed tube. The resulting mixture was stirred for 20 hr at 100° C. The reaction was monitored by LCMS. The resulting mixture was concentrated under vacuum. This resulted in 2-methylpyrazolo[1,5-a]pyridine-6-carboxylic acid (84 mg, 69.5%) as a white solid.

LC-MS (ES, m/z): [M+H]=177

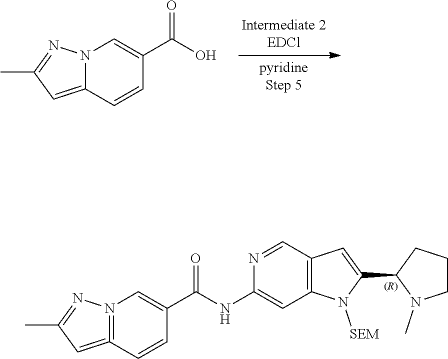

To a solution of 2-[(2R)-1-methylpyrrolidin-2-yl]-1-{[2-(trimethylsilyl)ethoxy]methyl}pyrrolo[3,2-c]pyridin-6-amine (70 mg, 0.202 mmol, 1.00 equiv) in 2 mL pyridine was added 2-methylpyrazolo[1,5-a]pyridine-6-carboxylic acid (35.59 mg, 0.202 mmol, 1 equiv) and EDCI (77.44 mg, 0.404 mmol, 2 equiv) in a 8 mL sealed tube. The resulting mixture was stirred for 16 hr at room temperature. The reaction was monitored by LCMS. The resulting mixture was concentrated under vacuum. This resulted in 2-methyl-N-{2-[(2R)-1-methylpyrrolidin-2-yl]-1-{[2-(trimethylsilyl)ethoxy]methyl}pyrrolo[3,2-c]pyridin-6-yl}pyrazolo[1,5-a]pyridine-6-carboxamide (120 mg) as a brown oil.

LC-MS (ES, m/z): [M+H]=505.

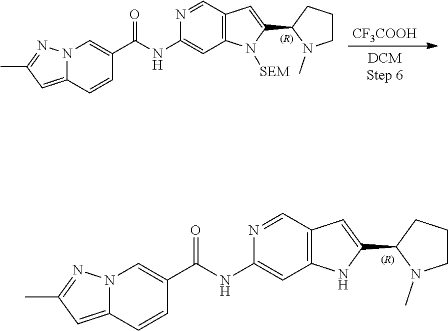

To a solution of 2-methyl-N-{2-[(2R)-1-methylpyrrolidin-2-yl]-1-{[2-(trimethylsilyl) ethoxy]methyl}pyrrolo[3,2-c]pyridin-6-yl}pyrazolo[1,5-a]pyridine-6-carboxamide (120 mg, 0.238 mmol, 1.00 equiv) in 5 mL DCM was added CF₃COOH (1 mL, 13.463 mmol, 56.62 equiv) in a 50 mL round-bottom flask. The resulting mixture was stirred for 16 hr at room temperature. The resulting mixture was concentrated under vacuum. The residue was basified to pH 9 with NH₄OH (aq). The crude product was purified by Prep-HPLC with the following conditions: Column, Sunfire Prep C18 OBD Column, 50*250 mm, 5 μm 10 nm; mobile phase, water (0.05% NH₃H₂O) and ACN (22% ACN up to 57% in 12 min; Detector, UV 254 nm. This afforded 2-methyl-N-{2-[(2R)-1-methylpyrrolidin-2-yl]-1H-pyrrolo[3,2-c]pyridin-6-yl}pyrazolo[1,5-a]pyridine-6-carboxamide (26.7 mg, 18% for two steps) as a brown solid.

LC-MS (ES, m/z): [M+H]=375.

¹H-NMR-(300 MHz, Methanol-d₄, ppm): δ 9.16 (s, 1H), 8.54 (s, 1H), 8.16 (s, 1H), 7.72 (dd, J=9.3, 1.6 Hz, 1H), 7.64 (d, J=9.3 Hz, 1H), 6.52 (d, J=9.5 Hz, 2H), 3.45 (t, J=7.9 Hz, 1H), 3.26-3.22 (m, 1H), 2.51 (s, 3H), 2.41 (q, J=8.9 Hz, 1H), 2.31 (s, 4H), 2.08-1.97 (m, 2H), 1.97-1.87 (m, 1H).

### Example 2: 1-methyl-N-(2-(1-methylpyrrolidin-2-yl)-1H-imidazo[4,5-c]pyridin-6-yl)-1H-indazole-5-carboxamide

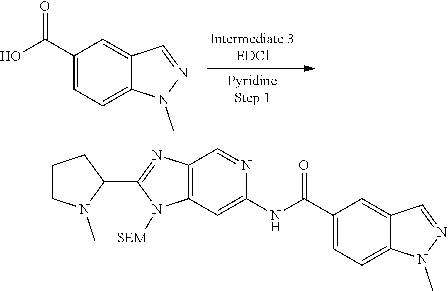

Into a 4-mL vial purged and maintained with an inert atmosphere of nitrogen, was placed 2-(1-methylpyrrolidin-2-yl)-1-[[2-(trimethylsilyl)ethoxy]methyl]imidazo[4,5-c]pyridin-6-amine (75.00 mg, 0.216 mmol, 1.00 equiv), 1-methylindazole-5-carboxylic acid (B, 41.82 mg, 0.237 mmol, 1.10 equiv), EDCI (62.06 mg, 0.324 mmol, 1.50 equiv), pyridine (2.00 mL). The resulting solution was stirred for 1 overnight at 25° C. The reaction was then quenched by the addition of 3 mL of water. The resulting solution was extracted with 3×3 mL of ethyl acetate and the organic layers combined. The resulting mixture was washed with 3×3 mL of brine. The resulting mixture was concentrated. This resulted in 160 mg (crude) of 1-methyl-N-[2-(1-methylpyrrolidin-2-yl)-1-[[2-(trimethylsilyl)ethoxy]methyl]imidazo[4,5-c]pyridin-6-yl]indazole-5-carboxamide as a brown oil.

LC-MS: (ES, m/z): [M+1]⁺=506.

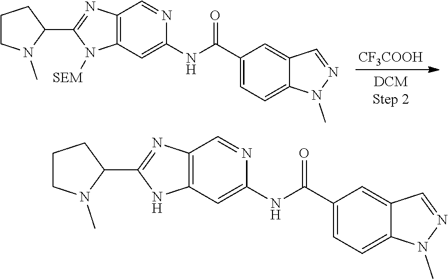

Into a 50-mL round-bottom flask purged and maintained with an inert atmosphere of nitrogen, was placed 1-methyl-N-[2-(1-methylpyrrolidin-2-yl)-1-[[2-(trimethylsilyl)ethoxy]methyl]imidazo [4,5-c]pyridin-6-yl]indazole-5-carboxamide (160.0 mg, 0.316 mmol, 1.00 equiv), CF₃COOH (3 mL), DCM (3.00 mL). The resulting solution was stirred for 20 hr at 25° C. The resulting mixture was concentrated. The crude product was purified by Prep-HPLC with the following conditions: XBridge Prep C18 OBD Column, 5 μm, 19*150 mm; mobile phase, water (0.05% NH₃·H₂O) and ACN (12% PhaseB up to 34% in 7 min; Detector, UV. 254 nm. This resulted in 25.6 mg (21.55%) of 1-methyl-N-[2-(1-methylpyrrolidin-2-yl)-1H-imidazo[4,5-c]pyridin-6-yl]indazole-5-carboxamide as a light yellow solid.

LC-MS: (ES, m/z): [M+1]⁺=376.

H-NMR (300 MHz, Methanol-d₄, ppm) δ 8.65 (d, J=1.1 Hz, 1H), 8.52 (dd, J=1.7, 0.8 Hz, 1H), 8.42 (d, J=1.0 Hz, 1H), 8.20 (d, J=0.9 Hz, 1H), 8.09 (dd, J=8.9, 1.7 Hz, 1H), 7.71 (dt, J=8.9, 1.0 Hz, 1H), 4.15 (s, 3H), 3.66 (s, 1H), 3.27 (d, J=8.0 Hz, 1H), 2.53-2.34 (m, 5H), 2.01 (dt, J=24.1, 9.5 Hz, 3H).

### Example 3: (R)—N-(2-(1-methylpyrrolidin-2-yl)-1H-benzo[d]imidazol-5-yl)-1-(pyridin-4-yl)-1H-indazole-5-carboxamide

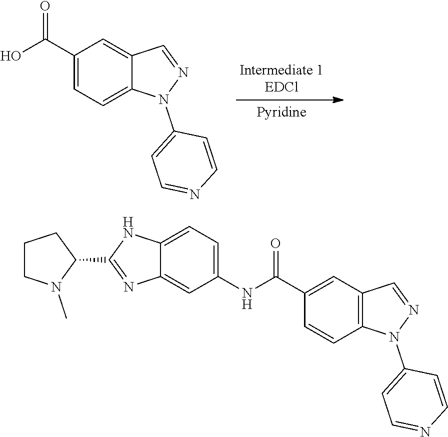

Into a 8-mL vial, was placed 1-(pyridin-4-yl) indazole-5-carboxylic acid, Acid 34 (60.00 mg, 0.251 mmol, 1.00 equiv), pyridine (1.20 mL), 2-[(2R)-1-methylpyrrolidin-2-yl]-1H-1,3-benzodiazol-5-amine (54.25 mg, 0.251 mmol, 1.00 equiv), EDCI (72.12 mg, 0.376 mmol, 1.50 equiv). The resulting solution was stirred for 16 hr at room temperature. The resulting mixture was concentrated under vacuum. The resulting solution was diluted with 20 mL of H₂O. The resulting solution was extracted with 3×10 mL of ethyl acetate and the organic layers combined. The resulting mixture was washed with 2×20 mL of brine. The mixture was dried over anhydrous sodium sulfate and concentrated under vacuum. The crude product (100 mg) was purified by Prep-HPLC with the following conditions: XBridge Prep C18 OBD Column, 5 μm, 19*150 mm; mobile phase, water (0.05% NH₃H₂O) and ACN (19% PhaseB up to 26% in 7 min; Detector, UV 254 nm. This resulted in 18.8 mg (17%) of N-[2-[(2R)-1-methylpyrrolidin-2-yl]-1H-1,3-benzodiazol-5-yl]-1-(pyridin-4-yl) indazole-5-carboxamide as a white solid.

LC-MS: (ES, m/z): [M+H]⁺=438.

¹H-NMR: (300 MHz, Methanol-d₄ ppm) 88.74-8.72 (m, 2H), 8.57-8.53 (m, 2H), 8.21 (d, J=1.3 Hz, 2H), 8.10 (d, J=1.9 Hz, 1H), 8.09-8.01 (m, 2H), 7.56 (d, J=8.7 Hz, 1H), 7.47 (dd, J=8.6, 2.0 Hz, 1H), 3.67-3.56 (m, 1H), 3.29 (t, J=7.7 Hz, 1H), 2.51-2.26 (m, 5H), 2.14-2.12 (m, 2H), 1.99-1.91 (m, 1H).

### Example 4: 3-(1-hydroxyethyl)-N-(2-((R)-1-methylpyrrolidin-2-yl)-1H-pyrrolo[3,2-c]pyridin-6-yl) imidazo[1,5-a]pyridine-7-carboxamide

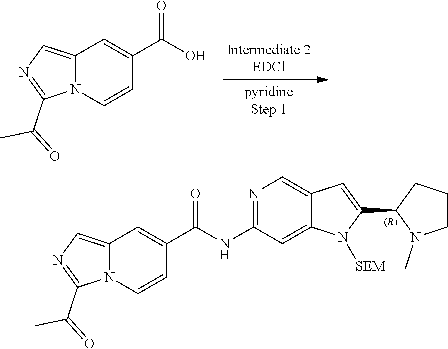

Into a 8-mL sealed tube, was placed 3-acetylimidazo[1,5-a]pyridine-7-carboxylic acid (Acid 2, 30.00 mg, 0.147 mmol, 1.00 equiv), 2-[(2R)-1-methylpyrrolidin-2-yl]-1-[[2-(trimethylsilyl)ethoxy]methyl]pyrrolo[3,2-c]pyridin-6-amine (50.92 mg, 0.147 mmol, 1.00 equiv), EDCI (56.33 mg, 0.294 mmol, 2.00 equiv), pyridine (1.00 mL). The resulting solution was stirred for 16 hr at room temperature. The resulting mixture was concentrated. This resulted in 60 mg (crude) of 3-acetyl-N-[2-[(2R)-1-methylpyrrolidin-2-yl]-1-[[2-(trimethylsilyl)ethoxy]methyl]pyrrolo[3,2-c]pyridin-6-yl]imidazo[1,5-a]pyridine-7-carboxamide as light brown oil.

LC-MS: (ES, m/z): [M+H]=533.

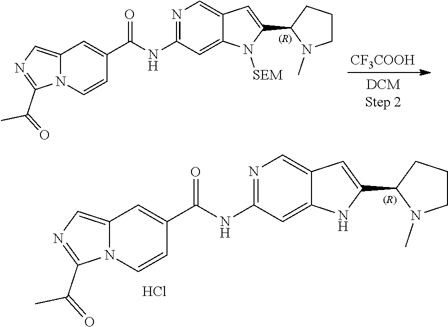

Into a 8-mL sealed tube, was placed 3-acetyl-N-[2-[(2R)-1-methylpyrrolidin-2-yl]-1-[[2-(trimethylsilyl)ethoxy]methyl]pyrrolo[3,2-c]pyridin-6-yl]imidazo[1,5-a]pyridine-7-carboxamide (60 mg, crude), CF₃COOH (1 mL), DCM (1 mL). The resulting solution was stirred for 16 hr at room temperature. The resulting mixture was concentrated. The crude product was purified by Prep-HPLC with the following conditions: Column, SunFire Prep C18 OBD Column, 19*150 mm; mobile phase, water (0.05% HCl) and ACN (5% Phase B up to 35% in 7 min; Detector, UV 254 nm. This resulted in 12 mg (19% for two steps) of 3-acetyl-N-[2-[(2R)-1-methylpyrrolidin-2-yl]-1H-pyrrolo[3,2-c]pyridin-6-yl]imidazo[1,5-a]pyridine-7-carboxamide hydrochloride as a white solid.

LC-MS: (ES, m/z): [M+H−HCl]=403

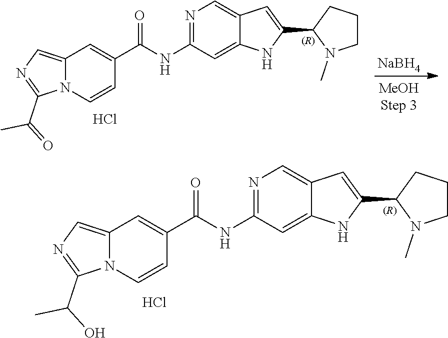

Into a 8-mL sealed tube, was placed 3-acetyl-N-[2-[(2R)-1-methylpyrrolidin-2-yl]-1H-pyrrolo[3,2-c]pyridin-6-yl]imidazo[1,5-a]pyridine-7-carboxamide hydrochloride (12 mg, 0.027 mmol, 1.00 equiv), MeOH (1 mL), NaBH₄ (5.17 mg, 0.135 mmol, 5.00 equiv). The resulting solution was stirred for 2 hr at room temperature. The reaction was then quenched by the addition of 1 mL of 1N HCl (aq). The crude product was purified by Prep-HPLC with the following conditions: Column, XBridge Shield RP18 OBD Column, 19*150 mm, 5 nm; mobile phase, water (0.05% NH₃H₂O) and ACN (19% Phase B up to 38% in 7 min; Detector, UV 254 nm. This resulted in 4 mg (36.2%) of 3-(1-hydroxyethyl)-N-[2-[(2R)-1-methylpyrrolidin-2-yl]-1H-pyrrolo[3,2-c]pyridin-6-yl]imidazo[1,5-a]pyridine-7-carboxamide as a light brown solid.

LC-MS: (ES, m/z): [M+H]=405.

¹H-NMR: (300 MHz, CD₃OD-d₄, ppm): δ 8.63 (s, 1H), 8.50 (d, J=7.6 Hz, 1H), 8.33 (d, J=1.8 Hz, 1H), 8.23 (s, 1H), 7.65 (s, 1H), 7.25 (dd, J=7.6, 1.8 Hz, 1H), 6.76 (s, 1H), 5.36 (q, J=6.6 Hz, 1H), 4.16 (s, 1H), 3.56 (s, 1H), 2.97 (d, J=9.9 Hz, 1H), 2.63 (s, 3H), 2.54-2.43 (m, 1H), 2.37 (d, J=8.3 Hz, 1H), 2.29-2.10 (m, 2H), 1.76 (d, J=6.6 Hz, 3H).

### Example 5: (R)-1-methyl-N-(2-(1-methylpyrrolidin-2-yl)-1H-pyrrolo[3,2-c]pyridin-6-yl)-1H-pyrazolo[4,3-c]pyridine-6-carboxamide

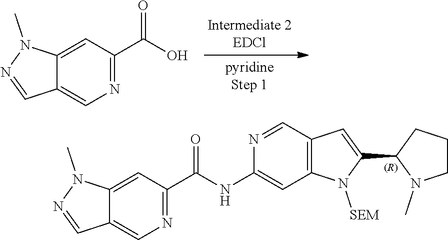

Into a 8-mL round-bottom flask purged and maintained with an inert atmosphere of nitrogen, was placed 1-methylpyrazolo[4,3-c]pyridine-6-carboxylic acid (Acid 3, 33.00 mg, 0.186 mmol, 1.00 equiv), 2-[(2R)-1-methylpyrrolidin-2-yl]-1-[[2-(trimethylsilyl)ethoxy]methyl]pyrrolo[3,2-c]pyridin-6-amine (64.55 mg, 0.186 mmol, 1.00 equiv), EDCI (71.42 mg, 0.372 mmol, 2.00 equiv), pyridine (2.00 mL). The resulting solution was stirred for 12 hr at 25° C. The reaction was then quenched by the addition of 8 mL of water/ice. The resulting solution was extracted with 3×3 mL of ethyl acetate and the organic layers combined. The resulting mixture was washed with 3×3 mL of brine. The resulting mixture was concentrated. This resulted in 45 mg (47.8%) of 1-methyl-N-[2-[(2R)-1-methylpyrrolidin-2-yl]-1-[[2-(trimethylsilyl)ethoxy]methyl]pyrrolo[3,2-c]pyridin-6-yl]pyrazolo[4,3-c]pyridine-6-carboxamide as a brown solid.

LC-MS: (ES, m/z): [M+1]⁺=506.

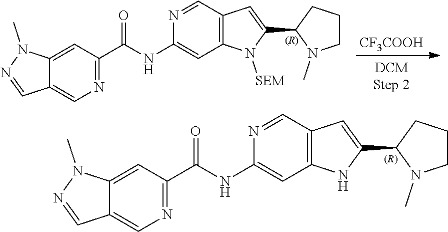

Into a 8-mL round-bottom flask purged and maintained with an inert atmosphere of nitrogen, was placed 1-methyl-N-[2-[(2R)-1-methylpyrrolidin-2-yl]-1-[[2-(trimethylsilyl)ethoxy]methyl]pyrrolo[3,2-c]pyridin-6-yl]pyrazolo[4,3-c]pyridine-6-carboxamide (45.00 mg, 0.089 mmol, 1.00 equiv), DCM (2 mL), CF₃COOH (2 mL). The resulting solution was stirred for 16 hr at 25° C. The resulting mixture was concentrated. The crude product was purified by Prep-HPLC with the following conditions: Column, XBridge Prep C18 OBD Column, 19*150 mm; mobile phase, water (0.05% NH₃·H₂O) and ACN (35% PhaseB up to 65% in 7 min; Detector, UV 254 nm. This resulted in 19.7 mg (59%) of 1-methyl-N-[2-[(2R)-1-methylpyrrolidin-2-yl]-1H-pyrrolo[3,2-c]pyridin-6-yl]pyrazolo[4,3-c]pyridine-6-carboxamide as an off-white solid.

LC-MS (ES, m/z): [M+1]⁺=376.

¹H-NMR (300 MHz, Methanol-d₄, ppm) δ 9.17 (d, J=1.1 Hz, 1H), 8.51 (d, J=1.0 Hz, 1H), 8.44 (dt, J=7.5, 1.1 Hz, 2H), 8.34 (d, J=1.1 Hz, 1H), 6.54 (d, J=1.0 Hz, 1H), 4.20 (s, 3H), 3.45 (t, J=7.9 Hz, 1H), 3.25 (t, J=7.7 Hz, 1H), 2.49-2.32 (m, 5H), 2.11-1.85 (m, 3H).

### Example 6: (R)—N-(2-(1-methylpyrrolidin-2-yl)-1H-pyrrolo[3,2-c]pyridin-6-yl)-3-(oxetan-3-yl) imidazo[1,5-a]pyridine-7-carboxamide

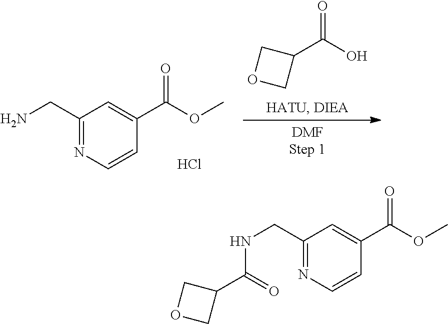

Into a 50-mL round-bottom flask purged and maintained with an inert atmosphere of nitrogen, was placed methyl 2-(aminomethyl)pyridine-4-carboxylate hydrochloride (750.0 mg, 3.7 mmol, 1.00 equiv), oxetane-3-carboxylic acid (453.42 mg, 4.44 mmol, 1.20 equiv), dimethylformamide (10.00 mL), HATU (1688.74 mg, 4.441 mmol, 1.20 equiv), DIEA (1913 mg, 14.805 mmol, 4.00 equiv). The resulting solution was stirred for 6 hr at room temperature. The reaction was then quenched by the addition of 30 mL of water/ice. The resulting solution was extracted with 4×10 mL of ethyl acetate and the organic layers combined and concentrated. This resulted in 400 mg (43%) of methyl 2-[(oxetan-3-ylformamido)methyl]pyridine-4-carboxylate as a brown solid.

LC-MS (ES, m/z): [M+1]⁺=251.

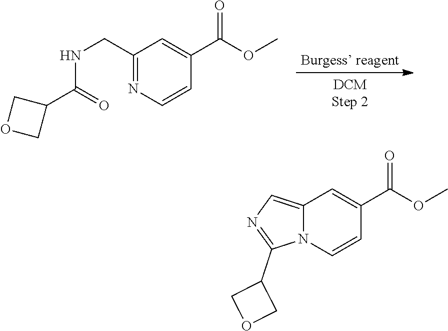 [SMILES: COC(=O)c1ccnc(CNC(=O)C2COC2)c1 ; COC(=O)c1ccn2c(C3COC3)ncc2c1]

Into a 50-mL round-bottom flask purged and maintained with an inert atmosphere of nitrogen, was placed methyl 2-[(oxetan-3-ylformamido)methyl]pyridine-4-carboxylate (400.0 mg, 1.6 mmol, 1.0 equiv), DCM (8.00 mL), Burgess reagent (1143 mg, 4.8 mmol, 3.0 equiv). The resulting solution was stirred for 4 hr at room temperature. The resulting mixture was concentrated. The residue was applied onto a silica gel column with THF/PE (1:3). This resulted in 140 mg (39%) of methyl (2Z)-2-(aminomethylidene)-1H-pyridine-4-carboxylate; oxetane as a light yellow solid.

LC-MS (ES, m/z): [M+1]⁺=233.

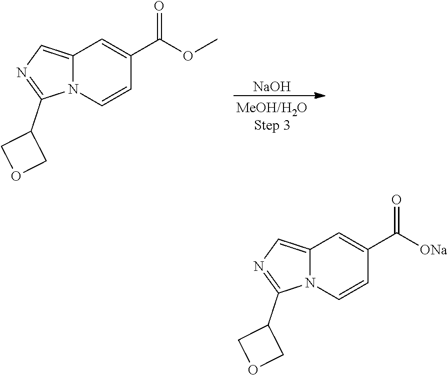

Into a 8-mL vial purged and maintained with an inert atmosphere of nitrogen, was placed methyl 3-(oxetan-3-yl) imidazo[1,5-a]pyridine-7-carboxylate (140.0 mg, 0.603 mmol, 1.00 equiv), sodium hydroxide (48.2 mg, 1.2 mmol, 2.00 equiv), MeOH (0.80 mL), H₂O (0.20 mL). The resulting solution was stirred for 16 hr at room temperature. The resulting mixture was concentrated. This resulted in 160 mg (crude) of sodium 3-(oxetan-3-yl) imidazo[1,5-a]pyridine-7-carboxylate as a light yellow solid.

LC-MS (ES, m/z): [M+1]⁺=219.

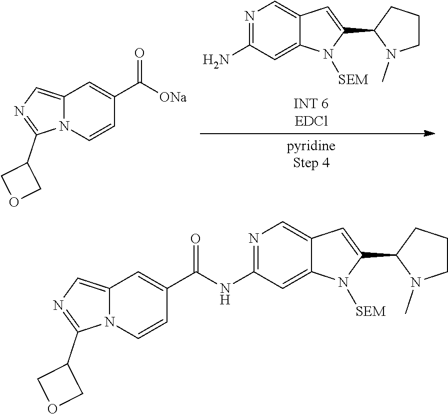

Into a 8-mL vial purged and maintained with an inert atmosphere of nitrogen, was placed sodium 3-(oxetan-3-yl) imidazo[1,5-a]pyridine-7-carboxylate (160.0 mg, 0.67 mmol, 1.00 equiv), 2-[(2R)-1-methylpyrrolidin-2-yl]-1-[[2-(trimethylsilyl)ethoxy]methyl]pyrrolo[3,2-c]pyridin-6-amine (92.34 mg, 0.27 mmol, 0.40 equiv), pyridine (2.00 mL), EDCI (255.40 mg, 1.33 mmol, 2.00 equiv). The resulting solution was stirred for 16 hr at room temperature. The reaction was then quenched by the addition of 10 mL of water/ice. The resulting solution was extracted with 3×4 mL of ethyl acetate and the organic layers combined. The resulting mixture was washed with 3×4 ml of brine. The resulting mixture was concentrated. This resulted in 120 mg (33%) of N-[2-[(2R)-1-methylpyrrolidin-2-yl]-1-[[2-(trimethylsilyl)ethoxy]methyl]pyrrolo[3,2-c]pyridin-6-yl]-3-(oxetan-3-yl) imidazo[1,5-a]pyridine-7-carboxamide as a brown solid.

LC-MS (ES, m/z): [M+1]⁺=547.

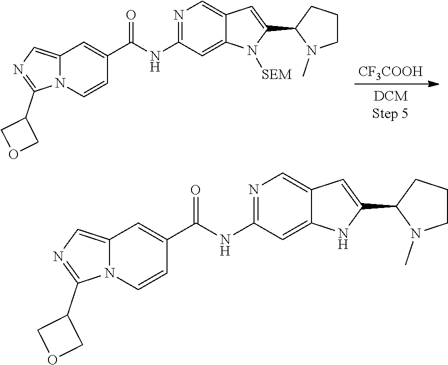

Into a 8-mL vial purged and maintained with an inert atmosphere of nitrogen, was placed N-{2-[(2R)-1-methylpyrrolidin-2-yl]-1-{[2-(trimethylsilyl)ethoxy]methyl}pyrrolo[3,2-c]pyridin-6-yl}-3-(oxetan-3-yl) imidazo[1,5-a]pyridine-7-carboxamide (120 mg, 0.219 mmol, 1.00 equiv), trifluoroacetic acid (2.00 mL), DCM (2.00 mL). The resulting solution was stirred for 16 hr at room temperature. The crude product was purified by Prep-HPLC with the following conditions: Column, XBridge Prep C18 OBD Column, 5 μm, 19*150 mm; mobile phase, water (0.05% NH₃·H₂O) and ACN (30% PhaseB up to 57% in 7 min; Detector, UV 254 nm. This resulted in N-{2-[(2R)-1-methylpyrrolidin-2-yl]-1H-pyrrolo[3,2-c]pyridin-6-yl}-3-(oxetan-3-yl) imidazo[1,5-a]pyridine-7-carboxamide (16 mg, 17.5%) as an off white solid.

LC-MS (ES, m/z): [M+1]⁺=417.

¹H-NMR (300 MHz, Methanol-d₄, ppm) δ 8.54 (s, 1H), 8.33 (t, J=1.5 Hz, 1H), 8.20-8.10 (m, 2H), 7.75 (d, J=0.9 Hz, 1H), 7.25 (dd, J=7.5, 1.8 Hz, 1H), 6.53 (d, J=1.0 Hz, 1H), 5.18 (dd, J=8.5, 5.7 Hz, 2H), 5.10 (dd, J=6.8, 5.7 Hz, 2H), 5.00-4.89 (m, 1H), 3.45 (t, J=7.9 Hz, 1H), 3.24 (t, J=7.9 Hz, 1H), 2.42 (q, J=8.8 Hz, 1H), 2.32 (s, 4H), 2.13-1.89 (m, 3H).

### Example 7: (R)-6-fluoro-N-(2-(1-methylpyrrolidin-2-yl)-1H-benzo[d]imidazol-5-yl)-1-(pyridin-4-yl)-1H-indazole-5-carboxamide

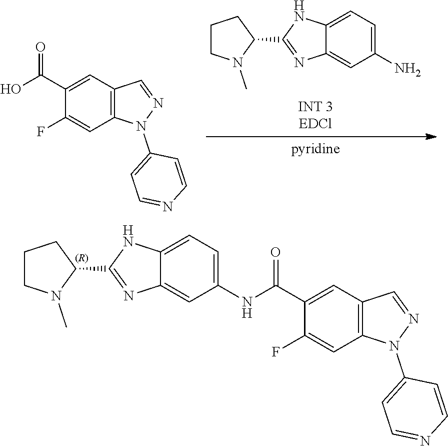

Into a 8-mL vial, was placed 2-[(2R)-1-methylpyrrolidin-2-yl]-1H-1,3-benzodiazol-5-amine (67.27 mg, 0.311 mmol, 1.00 equiv), pyridine (1.50 mL), 6-fluoro-1-(pyridin-4-yl) indazole-5-carboxylic acid (Prepared as for Acid 16 Step 2 using 6-fluoro-1H-indazole-5-carboxylic acid and 4-iodopyridine, 80.00 mg, 0.311 mmol, 1.00 equiv), EDCI (89.43 mg, 0.467 mmol, 1.50 equiv). The resulting solution was stirred for 4 hr at room temperature. The resulting mixture was concentrated under vacuum. The resulting solution was diluted with 10 mL of H₂O. The resulting solution was extracted with 3×10 mL of ethyl acetate and the organic layers combined. The resulting mixture was washed with 2×20 mL of brine. The mixture was dried over anhydrous sodium sulfate and concentrated under vacuum. The crude product (90 mg) was purified by Prep-HPLC with the following conditions: Column, XBridge Shield RP18 OBD Column, 5 μm, 19*150 mm; mobile phase, water (0.05% NH₃H₂O) and ACN (18% PhaseB up to 40% in 7 min; Detector, UV 254 nm. This resulted in 38.2 mg (27%) of 6-fluoro-N-[2-[(2R)-1-methylpyrrolidin-2-yl]-1H-1,3-benzodiazol-5-yl]-1-(pyridin-4-yl) indazole-5-carboxamide as a white solid.

LC-MS: (ES, m/z): [M+H]⁺=456.

¹H-NMR: (300 MHz, Methanol-d₄, ppm) δ 8.78-8.70 (m, 2H), 8.52 (s, 1H), 8.37 (d, J=6.8 Hz, 1H), 8.15 (d, J=1.9 Hz, 1H), 8.07-7.97 (m, 3H), 7.55 (d, J=8.7 Hz, 1H), 7.48-7.39 (m, 1H), 3.64 (t, J=7.7 Hz, 1H), 3.28-2.34 (m, 5H), 2.15-1.95 (m, 3H).

F-NMR: (300 MHz, Methanol-d₄, ppm) δ −114.420

### Example 8: 1-methyl-N-[2-[(2R)-1-methylpiperidin-2-yl]-1H-pyrrolo[3,2-c]pyridin-6-yl]indazole-6-carboxamide

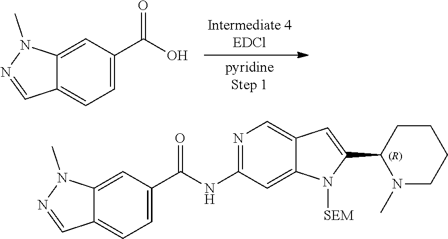

Into a 8-mL sealed tube, was placed 2-[(2R)-1-methylpiperidin-2-yl]-1-[[2-(trimethylsilyl)ethoxy]methyl]pyrrolo[3,2-c]pyridin-6-amine (44.00 mg, 0.074 mmol, 1.00 equiv, 61%), 1-methylindazole-6-carboxylic acid (A, 13.11 mg, 0.074 mmol, 1.00 equiv), EDCI (28.54 mg, 0.148 mmol, 2.00 equiv), pyridine (1.00 mL). The resulting solution was stirred for 16 hr at room temperature. The resulting mixture was concentrated. This resulted in 100 mg (crude) of 1-methyl-N-[2-[(2R)-1-methylpiperidin-2-yl]-1-[[2-(trimethylsilyl)ethoxy]methyl]pyrrolo[3,2-c]pyridin-6-yl]indazole-6-carboxamide as a brown solid.

LC-MS (ES, m/z): [M+H]=519.

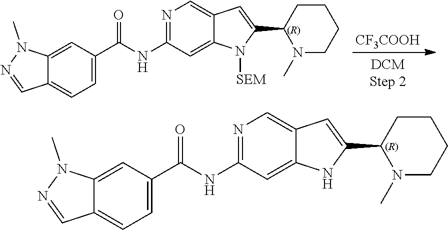

Into a 8-mL sealed tube, was placed 1-methyl-N-[2-[(2R)-1-methylpiperidin-2-yl]-1-[[2-(trimethylsilyl)ethoxy]methyl]pyrrolo[3,2-c]pyridin-6-yl]indazole-6-carboxamide (100.00 mg, crude), CF₃COOH (2.00 mL), DCM (2.00 mL). The resulting solution was stirred for 12 hr at room temperature. The resulting mixture was concentrated. The pH value of the solution was adjusted to 8 with NH₃. H₂O. The crude product was purified by Prep-HPLC with the following conditions: Column, XBridge Shield RP18 OBD Column, 19*150 mm, 5 um; mobile phase, water (0.05% NH₃H₂O) and ACN (25% PhaseB up to 55% in 7 min; Detector, UV 254 nm. This resulted in 20 mg of 1-methyl-N-[2-[(2R)-1-methylpiperidin-2-yl]-1H-pyrrolo[3,2-c]pyridin-6-yl]indazole-6-carboxamide as a light brown solid.

LC-MS (ES, m/z): [M+H]=389.

¹H-NMR (300 MHz, Methanol-d₄, ppm): δ 8.56 (d, J=1.0 Hz, 1H), 8.28 (s 1H), 8.24 (t, J=1.0 Hz, 1H), 8.12 (d, J=1.0 Hz, 1H), 7.93 (dd, J=8.5, 0.9 Hz, 1H), 7.79 (dd, J=8.5, 1.4 Hz, 1H), 6.53 (d, J=1.0 Hz, 1H), 4.20 (s, 3H), 3.19 (dd, J=9.2, 4.6 Hz, 1H), 3.09 (d, J=11.9 Hz, 1H), 2.30-2.21 (m, 1H), 2.14 (s, 3H), 1.90 (d, J=6.1 Hz, 3H), 1.79 (s, 3H), 1.49 (s, 1H).

### Example 9: 1-methyl-N-[2-(1-methylpiperidin-2-yl)-1H-1,3-benzodiazol-5-yl]indazole-5-carboxamide

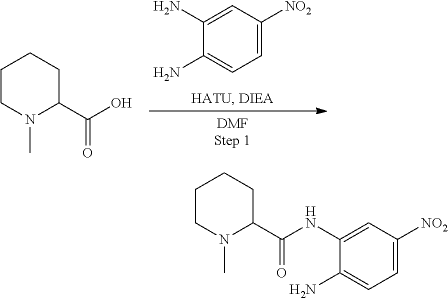

Into a 100-mL 3-necked round-bottom flask purged and maintained with an inert atmosphere of nitrogen, was placed 1-methylpiperidine-2-carboxylic acid (5.00 g, 34.92 mmol, 1.00 equiv), 2-amino-4-nitroaniline (5.88 g, 38.412 mmol, 1.10 equiv), DMF (50.00 mL), HATU (15.93 g, 41.904 mmol, 1.20 equiv), DIEA (18.05 g, 139.680 mmol, 4.00 equiv). The resulting solution was stirred for 1 hr at 25° C. The reaction was then quenched by the addition of 200 ml of water/ice. The resulting solution was extracted with 3×70 mL of ethyl acetate and the organic layers combined. The resulting mixture was washed with 3×70 mL of brine. The resulting mixture was concentrated. This resulted in 11 g (crude) of N-(2-amino-5-nitrophenyl)-1-methylpiperidine-2-carboxamide as a brown solid.

LC-MS (ES, m/z): [M+1]⁺=279.

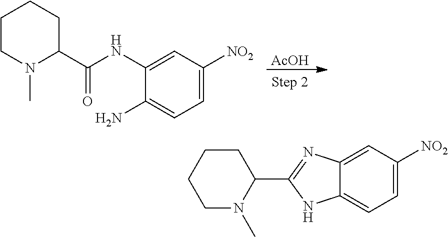

Into a 150-mL round-bottom flask purged and maintained with an inert atmosphere of nitrogen, was placed N-(2-amino-5-nitrophenyl)-1-methylpiperidine-2-carboxamide (11.00 g, 39.52 mmol, 1 equiv), acetic acid (150.00 mL). The resulting solution was stirred for 2 days at 90° C. The resulting mixture was concentrated. The residue was applied onto a silica gel column with ethyl acetate/petroleum ether (9:1). This resulted in 5 g (48.6%) of 2-(1-methylpiperidin-2-yl)-5-nitro-1H-1,3-benzodiazole as a brown solid.

LC-MS (ES, m/z): [M+1]⁺=261.

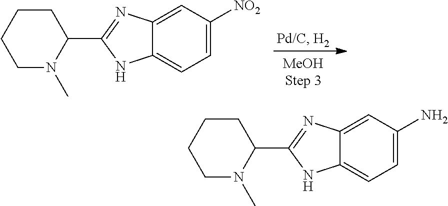 [SMILES: CN1CCCCC1c1nc2cc([N+](=O)[O-])ccc2[nH]1 ; CN1CCCCC1c1nc2cc(N)ccc2[nH]1]

Into a 50-mL round-bottom flask purged and maintained with an inert atmosphere of nitrogen, was placed 2-(1-methylpiperidin-2-yl)-5-nitro-1H-1,3-benzodiazole (500.0 mg, 1.92 mmol, 1.00 equiv), methanol (10.00 mL), Pd/C (41 mg, 0.20 equiv), H₂ (5 atm). The resulting solution was stirred for 1 overnight at 25° C. The solids were filtered out. The resulting mixture was concentrated. This resulted in 400 mg (90.4%) of 2-(1-methylpiperidin-2-yl)-1H-1,3-benzodiazol-5-amine as a brown solid.

LC-MS (ES, m/z): [M+1]⁺=231.

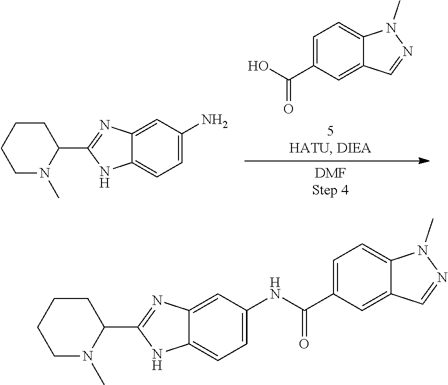 [SMILES: Cn1ncc2cc(C(=O)O)ccc21 ; CN1CCCCC1c1nc2cc(N)ccc2[nH]1 ; CN1CCCCC1c1nc2cc(NC(=O)c3ccc4c(cnn4C)c3)ccc2[nH]1]

Into a 8-mL vial purged and maintained with an inert atmosphere of nitrogen, was placed 2-(1-methylpiperidin-2-yl)-1H-1,3-benzodiazol-5-amine (100.00 mg, 0.434 mmol, 1.00 equiv), 1-methylindazole-5-carboxylic acid (B, 84.14 mg, 0.477 mmol, 1.10 equiv), DMF (2.00 mL), HATU (198.11 mg, 0.521 mmol, 1.20 equiv), DIEA (224.46 mg, 1.74 mmol, 4.00 equiv). The resulting solution was stirred for 2 hr at 25° C. The crude product was purified by Prep-HPLC with the following conditions: XBridge Prep C18 OBD Column, 5 μm, 19*150 mm; mobile phase, Water (0.05% NH₃·H₂O) and ACN (30% PhaseB up to 57% in 7 min; Detector, UV 254 nm. This resulted in 33.2 mg (19.7%) of 1-methyl-N-[2-(1-methylpiperidin-2-yl)-1H-1,3-benzodiazol-5-yl]indazole-5-carboxamide as an off-white solid.

LC-MS (ES, m/z): [M+1]⁺=389.

¹H-NMR (300 MHz, Methanol-da, ppm) δ 8.46 (dd, J=1.7, 0.8 Hz, 1H), 8.18 (d, J=0.9 Hz, 1H), 8.12-8.00 (m, 2H), 7.68 (dt, J=9.0, 1.0 Hz, 1H), 7.55 (d, J=8.6 Hz, 1H), 7.46 (d, J=8.7 Hz, 1H), 4.13 (s, 3H), 3.34-3.24 (m, 1H), 3.08 (d, J=11.3 Hz, 1H), 2.30-2.16 (m, 1H), 2.11 (s, 3H), 1.90 (dd, J=9.4, 4.9 Hz, 3H), 1.86-1.71 (m, 2H) 1.50 (s, 1H).

The following examples were prepared according to Example 3 using Intermediate 1 and the corresponding acid.

| Example | | | | |
|---|---|---|---|---|
| No. | Structure/Name | Acid | ¹H NMR | LCMS |
| 10 | 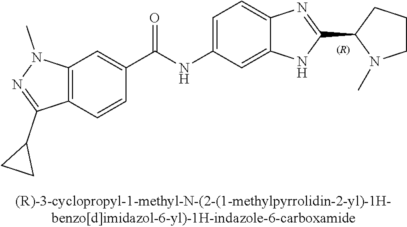 | 35 | ¹H-NMR: (300 MHz, Methanol-d₄, ppm) δ 8.16-8.07 (m, 2H), 7.90 (dd, J = 8.5, 0.9 Hz, 1H), 7.70 (dd, J = 8.5, 1.4 Hz, 1H), 7.55 (d, J = 8.6 Hz, 1H), 7.48 (dd, J = 8.7, 1.9 Hz, 1H), 4.06 (s, 3H), 3.62 (dd, J = 8.5, 6.9 Hz, 1H), 3.29-3.21 (m, 1H), 2.54-2.23 (m, 6H), 2.13-1.91 (m, 3H), 1.16-0.99 | LC-MS: (ES, m/z): [M + H] = 414 |
| | | | (m, 4H). | |
| 11 | 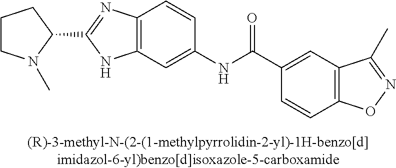 [SMILES: Cc1noc2ccc(C(=O)Nc3ccc4nc([C@H]5CCCN5C)[nH]c4c3)cc12] | C | ¹H-NMR (300 MHz, Methanol-d₄, ppm) δ 8.46 (dd, J = 1.8, 0.8 Hz, 1H), 8.24 (dd, J = 8.8, 1.8 Hz, 1H), 8.09 (d, J = 1.9 Hz, 1H), 7.72 (dd, J = 8.7, 0.8 Hz, 1H), 7.55 (dd, J = 8.7, 0.7 Hz, 1H), 7.47 (dd, J = 8.6, 2.0 Hz, 1H), 3.61 (dd, J = 8.5, | LC-MS: (ES, m/z): [M + 1]⁺ = 376 |
| | | | 7.0 Hz, 1H), 3.26 (t, | |
| | | | J = 7.7 Hz, 1H), 2.67 | |
| | | | (s, 3H), 2.54-2.29 (m, | |
| | | | 1H), 2.36-2.32 (m, | |
| | | | 4H), 2.15-1.87 (m, | |
| | | | 3H). | |
| 12 | 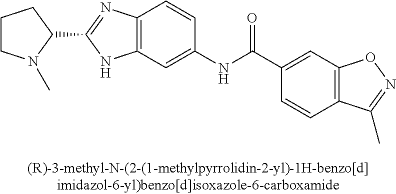 | D | ¹H-NMR (300 MHz, Methanol-d₄, ppm) δ 8.19 (t, J = 1.1 Hz, 1H), 8.11 (d, J = 1.9 Hz, 1H), 8.01-7.88 (m, 2H), 7.59-7.51 (m, 1H), 7.47 (dd, J = 8.6, 2.0 Hz, 1H), 3.62 (dd, J = 8.5, 6.9 Hz, 1H), 3.33-3.21 (m, 1H), 2.65 (s, 3H), 2.54-2.29 (m, 1H), | LC-MS: (ES, m/z): [M + 1]⁺ = 376 |
| | | | 2.36-2.32 (m, 4H), | |
| | | | 2.15-1.87 (m, 3H). | |
| 13 | 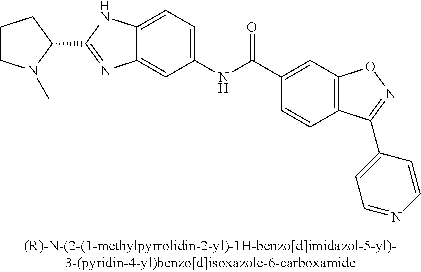 | 1 | ¹H-NMR(300 MHz, Methanol-d₄, ppm) δ 8.88-8.80 (m, 2H), 8.38 (d, J = 1.3 Hz, 1H), 8.31 (d, J = 8.4 Hz, 1H), 8.18-8.07 (m, 4H), 7.56 (d, J = 8.6 Hz, 1H), 7.53- 7.45 (m, 1H), 3.68- 3.57 (m, 1H), 3.26 (d, J = 8.2 Hz, 1H), 2.47- 2.30(m, 5H), 2.15- 1.88 (m, 3H). | LC-MS: (ES, m/z): [M + 1]⁺ = 418 |
| 14 | 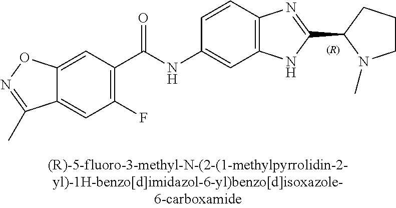 | 4 | ¹H-NMR (300 MHz, Methanol-d₄, ppm) δ 8.15 (d, J = 2.0 Hz, 1H), 7.97 (dd, J = 5.1, 1.5 Hz, 1H), 7.76- 7.67 (m, 1H), 7.55 (d, J = 8.5 Hz, 1H), 7.42 (d, J = 8.7 Hz, 1H), 3.63 (t, J = 7.5 Hz, 1H), 3.26 (d, J = 8.1 Hz, 1H), 2.63 (d, J = 1.5 Hz, 3H), 2.56- 2.34 (m, 5H), 2.15- | LC-MS-PH- PUK-BRM- 005-1238-0 (ES, m/z): [M + 1]⁺ = 394 |
| | | | 1.97 (m, 3H). | |
| 15 | 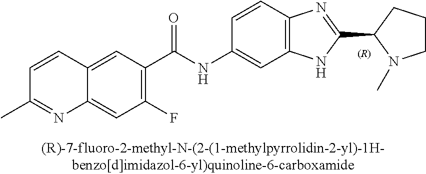 | 5 | ¹H-NMR (300 MHz, Methanol-d₄, ppm) δ 8.38 (dd, J = 8.1, 5.3 Hz, 2H), 8.16 (d, J = 1.9 Hz, 1H), 7.74 (d, J = 11.7 Hz, 1H), 7.53 (dd, J = 10.2, 8.6 Hz, 2H), 7.44 (dd, J = 8.6, 2.0 Hz, 1H), 3.67- 3.56 (m, 1H), 3.27 (s, 1H), 2.78 (s, 3H), | LC-MS (ES, m/z): [M + 1]⁺ = 418 |
| | | | 2.51-2.30 (m, 5H), | |
| | | | 2.17-1.87 (m, 3H). | |
| 16 | 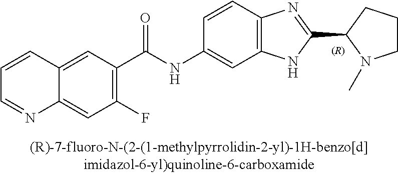 | E | ¹H-NMR (300 MHz, Methanol-d₄, ppm) δ 8.98 (dd, J = 4.4, 1.7 Hz, 1H), 8.58-8.49 (m, 1H), 8.43 (d, J = 7.6 Hz, 1H), 8.17 (d, J = 1.9 Hz, 1H), 7.84 (d, J = 11.4 Hz, 1H), 7.68-7.51 (m, 2H), 7.44 (dd, J = 8.7, 2.0 Hz, 1H), 3.62 (dd, | LC-MS: (ES, m/z): [M + 1]⁺ = 390 |
| | | | J = 8.5, 6.9 Hz, 1H), | |
| | | | 3.33-3.21 (m, 1H), | |
| | | | 2.54-2.31 (m, 5H), | |
| | | | 2.12-1.93 (m, 3H). | |
| 17 | 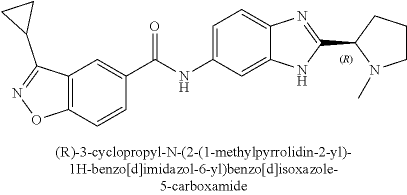 | 36 | ¹H-NMR (300 MHz, Methanol-d₄, ppm) δ 8.48 (d, J = 1.7 Hz, 1H), 8.23 (dd, J = 8.8, 1.8 Hz, 1H), 8.09 (d, J = 1.9 Hz, 1H), 7.71 (dd, J = 8.8, 0.8 Hz, 1H), 7.59-7.51 (m, 1H), 7.47 (dd, J = 8.6, 2.0 Hz, 1H), 3.62 (dd, J = 8.5, 7.0 Hz, 1H), 3.27 (t, J = 7.3 Hz, | LC-MS: (ES, m/z): [M + 1]⁺ = 402 |
| | | | 1H), 2.54-2.29 (m, | |
| | | | 3H), 2.36 (s, 3H), | |
| | | | 2.15-2.04 (m, 2H), | |
| | | | 2.08-1.90 (m, 1H), | |
| | | | 1.26 (d, J = 6.7 Hz, | |
| | | | 4H). | |
| 18 | 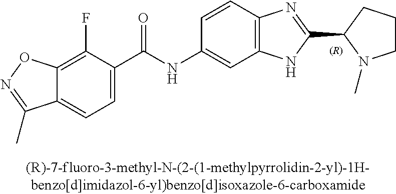 | 6 | ¹H-NMR (300 MHz, Methanol-d₄, ppm) δ 8.14 (d, J = 2.0 Hz, 1H), 7.76-7.62 (m, 2H), 7.55 (d, J = 8.7 Hz, 1H), 7.47-7.38 (m, 1H), 3.64 (t, J = 7.7 Hz, 1H), 3.28 (s, 1H), 2.65 (d, J = 1.5 Hz, 3H), 2.48 (q, J = 8.9 Hz, 1H), 2.38 (s, 4H), 2.08 (d, J = 8.7 | LC-MS: (ES, m/z): [M + 1]⁺ = 394 |
| | | | Hz, 2H), 1.97 (t, J = | |
| | | | 9.5 Hz, 1H). | |
| 19 | 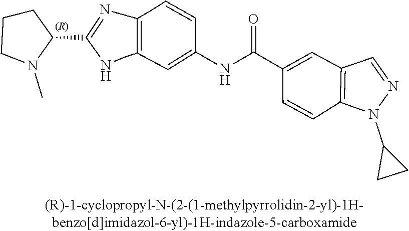 | 14 | ¹H-NMR14-PH- PUK-BRM-005- 1122-0: (300 MHz, Methanol-d₄, ppm) δ 8.45 (dd, J = 1.7, 0.8 Hz, 1H), 8.15 (d, J = 0.9 Hz, 1H), 8.07 (dd, J = 8.9, 1.7 Hz, 2H), 7.82 (d, J = 8.9 Hz, 1H), 7.54 (d, J = 8.4 Hz, 1H), 7.46 (dd, J = 8.7, 2.0 Hz, 1H), 3.74 (m, 1H), 3.67-3.56 (m, 1H), 3.27 (t, J = | LC-MS-PH- PUK-BRM- 005-1122-0: (ES, m/z): [M + H]⁺ = 401 |
| | | | 7.6 Hz, 1H), 2.54- | |
| | | | 2.29 (m, 5H), 2.19- | |
| | | | 1.87 (m, 3H), 1.24 | |
| | | | (m, 4H). | |
| 20 | 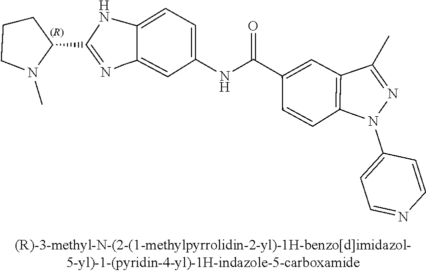 | 16 | ¹H-NMR (300 MHz, Methanol-d₄, ppm): δ 8.73-8.65 (m, 2H), 8.55 (t, J = 1.2 Hz, 1H), 8.22 (dd, J = 8.9, 1.7 Hz, 1H), 8.15 (d, J = 8.9 Hz, 1H), 8.10 (d, J = 1.9 Hz, 1H), 8.00 (d, J = 1.7 Hz, 1H), 7.98 (d, J = 1.7 Hz, 1H), 7.56 (d, J = 8.7 Hz, 1H), 7.49 (dd, J = 8.6, 1.9 Hz, 1H), 3.62 (dd, J = 8.5, 7.0 Hz, 1H), 3.26 (d, J = 7.7 Hz, 1H), 2.74 (s, 3H), 2.54-2.33 (s, | LC-MS (ES, m/z): [M + H] = 452 |
| | | | 5H), 2.15-1.92 (m, | |
| | | | 3H). | |
| 21 | 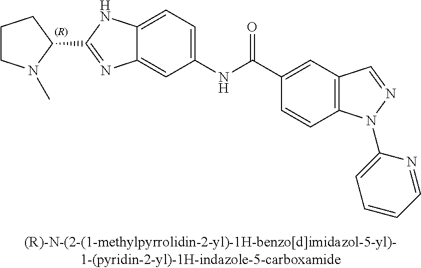 | 19 | ¹H-NMR (300 MHz, Methanol-d₄, ppm): δ 8.93 (d, J = 9 Hz, 1H), 8.60 (m, 1H), 8.52 (dd, J = 1.8, 0.8 Hz, 1H), 8.44 (d, J = 0.9 Hz, 1H), 8.20-8.06 (m, 3H), 8.05-7.93 (m, 1H), 7.56 (d, J = 8.6 Hz, 1H), 7.48 (dd, J = 8.7, 1.9 Hz, 1H), 7.32 (m, 1H), 3.67- 3.56 (m, 1H), 3.27 (m, 1H), 2.47 (m, 1H), 2.37 (s, 3H), 2.15-1.88 (m, 3H). | LC-MS(ES, m/z): 438[M + H]⁺ |
| 22 | 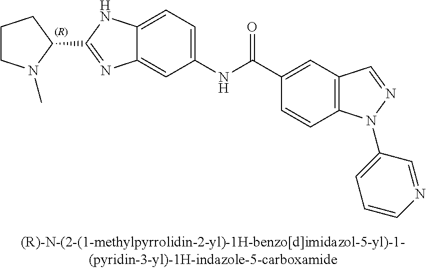 | 20 | ¹H-NMR (300 MHz, Methanol-d₄, ppm): δ 9.09 (dd, J = 2.6, 0.8 Hz, 1H), 8.68-8.55 (m, 2H), 8.52 (d, J = 0.9 Hz, 1H), 8.33 (m, 1H), 8.22-8.07 (m, 2H), 8.03-7.94 (m, 1H), 7.72 m, 1H), 7.56 (d, J = 8.7 Hz, 1H), 7.48 (dd, J = 8.6, 1.9 Hz, 1H), 3.67- 3.56 (m, 1H), 3.26 (d, J = 7.8 Hz, 1H), 2.47 (m, 1H), 2.37 (s, 3H), 2.06 (dd, J = 10.3, 6.9 Hz, 2H), 2.02-1.91 | LC-MS (ES, m/z): 438[M + H]⁺ |
| | | | (m, 1H). | |
| 23 | 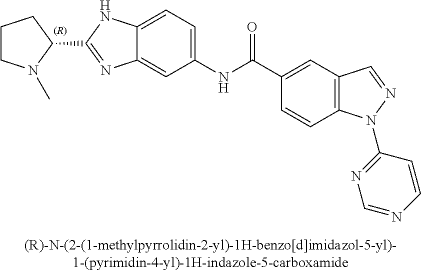 | 21 | ¹H-NMR (300 MHz, Methanol-d₄, ppm): δ 9.16 (d, J = 1.3 Hz, 1H), 9.03 (d, J = 8.9 Hz, 1H), 8.83 (d, J = 5.8 Hz, 1H), 8.60-8.51 (m, 2H), 8.23 (dd, J = 8.9, 1.8 Hz, 1H), 8.16 (dd, J = 5.9, 1.3 Hz, 1H), 8.11 (s, 1H), 7.56 (d, J = 8.6 Hz, 1H), 7.52-7.44 (m, 1H), 3.68-3.57 (m, 1H), 3.27 (s, 1H), 2.48 (m, 2H), 2.37 (s, 3H), 2.07-1.91 (m, 3H). | LC-MS (ES, m/z): 439[M + H]⁺ |
| 24 | 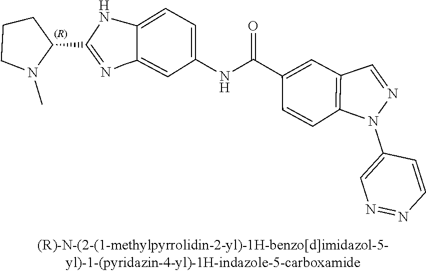 | 22 | ¹H-NMR: (300 MHz, Methanol-d₄, ppm) δ 9.97 (dd, J = 2.9, 1.0 Hz, 1H), 9.32 (dd, J = 5.9, 1.0 Hz, 1H), 8.62 (d, J = 8.9 Hz, 2H), 8.35-8.22 (m, 3H), 8.11 (s, 1H), 7.56- 7.48 (m, 2H), 3.63 (t, J = 7.7 Hz, 1H), 3.27 (s, 1H), 2.48-2.37 (m, 5H), 2.03-1.91 (m, 3H). | LC-MS: (ES, m/z): [M + H]⁺ = 439 |
| 25 | 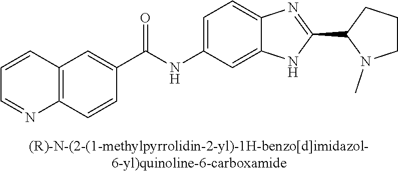 | F | ¹H-NMR (300 MHz, Methanol-d₄, ppm): δ 8.99 (dd, J = 4.4, 1.7 Hz, 1H), 8.62 (d, J = 2.0 Hz, 1H), 8.56 (d, J = 8.5 Hz, 1H), 8.33 (dd, J = 8.9, 2.0 Hz, 1H), 8.23-8.11 (m, 2H), 7.67 (dd, J = 8.4, 4.3 Hz, 1H), 7.61-7.46 (m, 2H), 3.70-3.59 | LC-MS (ES, m/z): 372[M + H]⁺ |
| | | | (m, 1H), 3.27 (d, J = | |
| | | | 7.3 Hz, 1H), 2.49- | |
| | | | 2.38 (m, 2H), 2.38 (s, | |
| | | | 3H), 2.11-1.95 (m, | |
| | | | 3H). | |
| 26 | 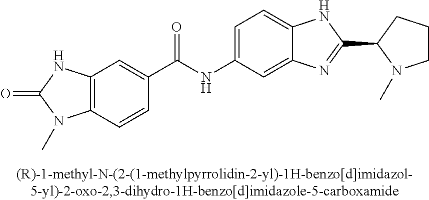 [SMILES: CN1CCC[C@@H]1c1nc2cc(NC(=O)c3ccc4c(c3)[nH]c(=O)n4C)ccc2[nH]1] | 37 | ¹H-NMR (300 MHz, Methanol-d₄, ppm): δ 8.04 (d, J = 1.9 Hz, 1H), 7.80 (dd, J = 8.3, 1.7 Hz, 1H), 7.71 (d, J = 1.7 Hz, 1H), 7.53 (d, J = 8.6 Hz, 1H), 7.43 (dd, J = 8.7, 2.0 Hz, 1H), 7.24 (d, J = 8.3 Hz, 1H), 3.61 (dd, J = 8.5, 7.0 Hz, 1H), 3.46 (s, 3H), 3.29- | LC-MS (ES, m/z): 391[M + H]⁺ |
| | | | 3.23 (m, 1H), 2.53- | |
| | | | 2.28 (m, 2H), 2.36 (s, | |
| | | | 3H), 2.14-1.86 (m, | |
| | | | 3H). | |
| 27 | 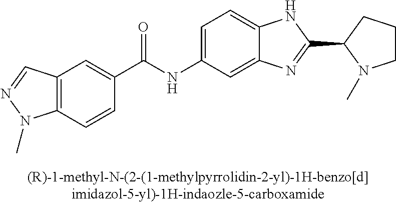 [SMILES: CN1CCC[C@@H]1c1nc2cc(NC(=O)c3ccc4c(cnn4C)c3)ccc2[nH]1] | B | ¹H NMR: (400 MHz, methanol-d₄), δ 8.44 (s, 1H), 8.18-8.13 (m, 2H), 8.03 (dd, J = 8.88, 1.63 Hz, 1H), 7.67 (d, J = 8.88 Hz, 1H), 7.57 (d, J = 8.63 Hz, 1H), 7.47 (dd, J = 8.69, 1.81 Hz, 1H), 4.12 (s, 3H), 4.08- 3.96 (m, 1H), 3.47 (br s, 1H), 2.87-2.71 (m, 1H), 2.60 (s, 3H), | LC-MS (ES, m/z): 375 [M + H]⁺ |
| | | | 2.54-2.41 (m, 1H), | |
| | | | 2.23-2.04 (m, 3H) | |

Prepared according to Example 1 Steps 5 and 6 using Intermediate 2 and the corresponding acid.

| Example | | | | |
|---|---|---|---|---|
| No. | Structure/Name | Acid | ¹H NMR | LCMS |
| 28 | 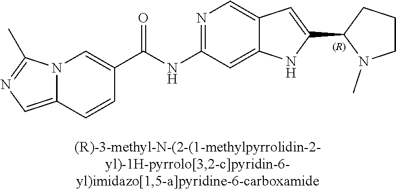 | G | ¹H-NMR: (300 MHz, Methanol-d₄, ppm) 88.78 (m, 1H), 8.55 (d, J = 1.0 Hz, 1H), 8.17 (t, J = 1.0 Hz, 1H), 7.61 (dd, J = 9.6, 1.1 Hz, 1H), 7.38 (d, J = 0.9 Hz, 1H), 7.29 (dd, J = | LC-MS: (ES, m/z): [M + H]⁺ = 375 |
| | | | 9.5, 1.5 Hz, | |
| | | | 1H), 6.54 (d, J = | |
| | | | 1.0 Hz, 1H), | |
| | | | 3.46 (t, J = 7.9 | |
| | | | Hz, 1H), 3.26 | |
| | | | (d, J = 7.9 Hz, | |
| | | | 1H), 2.77 (s, | |
| | | | 3H), 2.42-2.28 | |
| | | | (m, 5H), 2.11- | |
| | | | 1.91 (m, 3H). | |
| 29 | 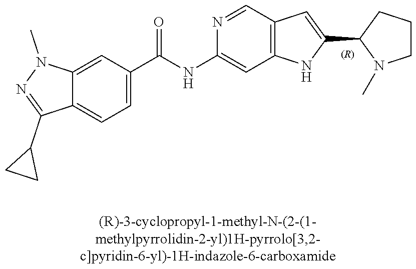 | 35 | ¹H-NMR(300 MHz, Methanol- d₄,ppm): δ 8.55 (d, J = 1.0 Hz, 1H), 8.23 (d, J = 1.0 Hz, 1H), 8.17 (t, J = 1.1 Hz, 1H), 7.92 (dd, J = 8.5, 0.9 Hz, 1H), 7.73 (dd, J = 8.5, 1.5 Hz, 1H), 6.54 (d, J = 1.0 Hz, 1H), 4.07 (s, 3H), | LC-MS: (ES, m/z): [M + H] = 415 |
| | | | 3.45 (t, J = 7.9 | |
| | | | Hz, 1H), 3.25 | |
| | | | (t, J = 8.0 Hz, | |
| | | | 1H), 2.43-2.25 | |
| | | | (m, 6H), 2.09- | |
| | | | 1.89 (m, 3H), | |
| | | | 1.13-1.02 (m, | |
| | | | 4H). | |
| 30 | 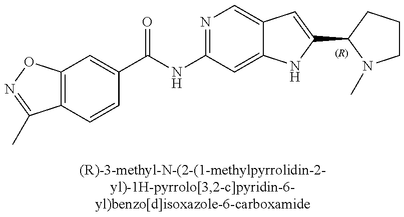 | D | ¹H-NMR (300 MHz, Methanol-d₄, ppm)δ 8.55 (d, J = 1.0 Hz, 1H), 8.22 (dt, J = 6.3, 1.1 Hz, 2H), 7.98 (qd, J = 8.2, 1.1 Hz, 2H), 6.54 (d, J = 1.0 Hz, 1H), 3.46 (t, J = 7.9 | LC-MS: (ES, m/z): [M + 1]⁺ = 376 |
| | | | Hz, 1H), 3.25 | |
| | | | (t, J = 7.8 Hz, | |
| | | | 1H), 2.66 (s, | |
| | | | 3H), 2.46- | |
| | | | 2.25(m, 5H), | |
| | | | 2.13 − 1.89 | |
| | | | (m, 3H). | |
| 31 | 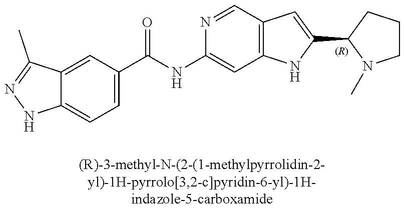 | 48 | ¹H-NMR (300 MHz, Methanol- d₄, ppm): δ 8.55 (d, J = 1.0 Hz, 1H), 8.50 (dd, J = 1.7, 0.8 Hz, 1H), 8.22 (t, J = 1.0 Hz, 1H), 8.05 (dd, J = 8.8, 1.7 Hz, 1H), 7.60 (dd, | LC-MS: (ES, m/z): [M + H] = 375 |
| | | | J = 8.8, 0.9 | |
| | | | Hz, 1H), 6.54 | |
| | | | (d, J = 1.0 Hz, | |
| | | | 1H), 3.46 (t, J = | |
| | | | 7.9 Hz, 1H), | |
| | | | 3.29-3.21 (m, | |
| | | | 1H), 2.67 (s, | |
| | | | 3H), 2.49-2.38 | |
| | | | (m, 1H), 2.32 | |
| | | | (s, 4H), 2.13- | |
| | | | 1.89 (m, 3H). | |
| 32 | 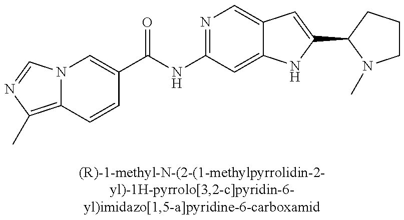 [SMILES: Cc1ncn2cc(C(=O)Nc3cc4[nH]c([C@H]5CCCN5C)cc4cn3)ccc12] | 7 | ¹H-NMR (300 MHz, Methanol-d₄, ppm)δ 8.88 (d, J = 1.4 Hz, 1H), 8.54 (d, J = 1.0 Hz, 1H), 8.40 (s, 1H), 8.16 (d, J = 1.0 Hz, 1H), 7.62 − 7.53 (m, 1H), 7.21 (dd, J = 9.6, 1.5 Hz, 1H), | LC-MS: (ES, m/z): [M + 1]⁺ = 375 |
| | | | 6.53 (d, J = | |
| | | | 1.0 Hz, 1H), | |
| | | | 3.46 (t, J = 7.9 | |
| | | | Hz, 1H), 3.25 | |
| | | | (t, J = 7.9 Hz, | |
| | | | 1H), 2.52 (s, | |
| | | | 3H), 2.41 (t, | |
| | | | J = 8.7 Hz, 1H), | |
| | | | 2.32 (s, 3H), | |
| | | | 2.06 − 1.88 | |
| | | | (m, 3H). | |
| 33 | 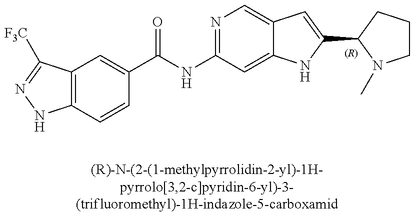 | 8 | ¹H-NMR (300 MHz, Methanol-d₄, ppm)δ 9.00 (s, 1H), 8.71 (s, 1H), 8.24 (dd, J = 9.0, 1.6 Hz, 1H), 7.92 − 7.83 (m, 2H), 7.33 (s, 1H), 3.93 (s, 1H), 3.42 (d, J = 10.7 Hz, | LC-MS: (ES, m/z): [M + 1]⁺ = 429 |
| | | | 1H), 3.02 (s, | |
| | | | 3H), 2.80 − | |
| | | | 2.66 (m, 1H), | |
| | | | 2.58 (s, 1H), | |
| | | | 2.40 (s, 2H). | |
| 34 | 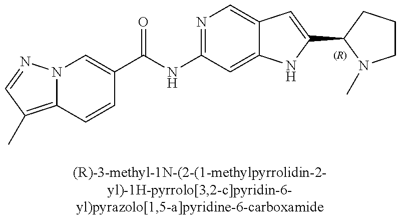 | 9 | ¹H-NMR (300 MHz, Methanol-d₄, ppm)δ 9.20 (s, 1H), 8.55 (s, 1H), 8.16 (s, 1H), 7.96 (s, 1H), 7.71 (s, 2 H), 6.53 (s, 1H), 3.45 (t, J = 7.8 Hz, 1H), 3.24 (s, 1H), 2.38 (s, 4H), 2.30 (d, J = 7.5 Hz, 4H), | LC-MS: (ES, m/z): [M + 1] = 375 |
| | | | 2.05 (s, 2H), | |
| | | | 1.96 (dd, J = | |
| | | | 20.5, 8.2 Hz, | |
| | | | 1H). | |
| 35 | 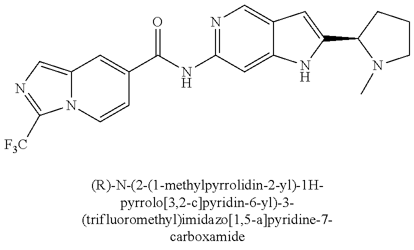 [SMILES: CN1CCC[C@@H]1c1cc2cnc(NC(=O)c3ccn4c(C(F)(F)F)ncc4c3)cc2[nH]1] | 10 | ¹H-NMR (300 MHz,CD₃OD- d4, ppm): δ 8.56 (d, J = 1.0 Hz, 1H), 8.53-8.44 (m, 2H), 8.18 (t, J = 1.0 Hz, 1H), 7.88 (d, J = 0.9 Hz, 1H), 7.54 (dd, J = 7.7, 1.6 Hz, 1H), 6.54 (d, J = 0.9 Hz, 1H), 3.45 (t, J = 8.0 Hz, 1H), 3.24 | LC-MS: (ES, m/z): [M + H] = 429 |
| | | | (td, J = 8.8, | |
| | | | 8.0, 2.2 Hz, | |
| | | | 1H), 2.42 (q, | |
| | | | J = 8.9 Hz, 1H), | |
| | | | 2.32 (s, 4H), | |
| | | | 2.14 − 1.85 | |
| | | | (m, 3H). | |
| 36 | 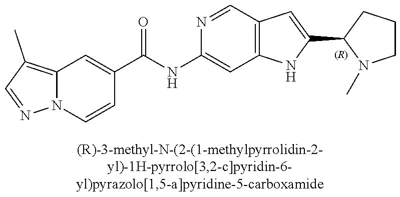 | 11 | ¹H-NMR (300 MHz, Methanol-d₄, ppm)δ 8.61 − 8.53 (m, 2H), 8.35 (dd, J = 2.1, 1.0 Hz, 1H), 8.20 (d, J = 1.0 Hz, 1H), 7.91 (s, 1H), 7.39 (dd, J = 7.3, 2.0 Hz, | LC-MS: (ES, m/z): [M + 1]⁺ = 375 |
| | | | 1H), 6.53 (s, | |
| | | | 1H), 3.45 (t, J = | |
| | | | 8.0 Hz, 1H), | |
| | | | 3.25 (s, 1H), | |
| | | | 2.44 (s, 3H), | |
| | | | 2.40 (t, J = 8.8 | |
| | | | Hz, 1H), 2.32 | |
| | | | (s, 4H), 2.04 | |
| | | | (s, 2H), 1.96 | |
| | | | (dd, J = 19.2, | |
| | | | 9.2 Hz, 1H). | |
| 37 | 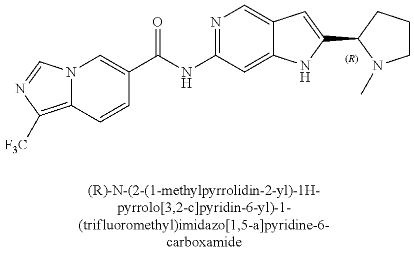 | 12 | ¹H-NMR (300 MHz, Methanol-d₄, ppm): δ 9.12 (t, J = 1.4 Hz, 1H), 8.62 (s, 1H), 8.56 (d, J = 1.0 Hz, 1H), 8.17 (d, J = 1.0 Hz, 1H), 7.79 (d, J = 9.7 Hz, 1H), 7.65 (dd, J = 9.7, 1.5 Hz, 1H), 6.54 (s, 1H), 3.45 (t, J = | LC-MS: (ES, m/z): [M + H] = 428 |
| | | | 7.9 Hz, 1H), | |
| | | | 3.25 (t, J = 8.1 | |
| | | | Hz, 1H), 2.19 − | |
| | | | 2.36 (m, | |
| | | | 1H), 2.32 (s, | |
| | | | 3H), 2.28 (d, | |
| | | | J = 8.2 Hz, 1H), | |
| | | | 2.13 − 1.89 | |
| | | | (m, 3H). | |
| 38 | 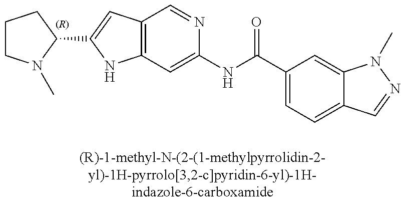 | A | ¹H-NMR (300 MHz, Methanol-d₄, ppm) δ 8.55 (s, 1H), 8.27 (s, 1H), 8.24(s, 1H), 8.11 (s, 1H), 7.91 (d, J = 8.4 Hz, 1H), 7.78 (d, J = 9 Hz, 1H), 6.53 (s, 1H), 4.19 (s, 3H), 3.45 | (ES, m/z): [M + 1] *= 375 |
| | | | (t, J = 7.5 Hz, | |
| | | | 1H), 3.24 (t, | |
| | | | J = 6.9 Hz, 1H), | |
| | | | 2.46-2.21 (m, | |
| | | | 2H), 2.32 (s, | |
| | | | 3H), 2.10-1.92 | |
| | | | (m, 3H). | |
| 39 | 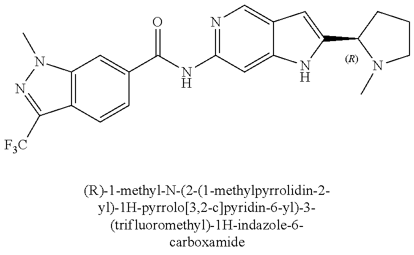 | 13 | ¹H-NMR: (300 MHz, Methanol-d₄, ppm): δ 8.56 (d, J = 0.9 Hz, 1H), 8.40 (d, J = 1.4 Hz, 1H), 8.23 (s, 1H), 7.95 (d, J = 1.5 Hz, 2H), 6.55 (s, 1H), 4.28 (s, 3H), 3.46 (t, J = 7.9 Hz, 1H), 3.24 (d, J = 8.1 Hz, 1H), 2.42 (q, | LC-MS: (ES, m/z): [M + H] = 443 |
| | | | J = 8.8 Hz, 2H), | |
| | | | 2.33 (s, 4H), | |
| | | | 2.12 − 1.93 | |
| | | | (m, 3H). | |
| 40 | 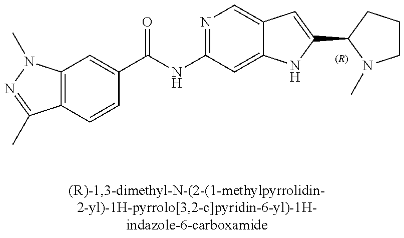 | 39 | ¹H-NMR: (300 MHz, Methanol-d₄, ppm) δ 8.55 (s, 1H), 8.21 (d, J = 14.7 Hz, 2H), 7.85 (d, J = 8.4 Hz, 1H), 7.75 (d, J = 8.5 Hz, 1H), 6.54 (s, 1H), 4.10 (s, 3H), 3.46 (t, J = 7.8 Hz, 1H), 3.25 (s, 1H), 2.60 | LC-MS: (ES, m/z): [M + H]⁺ = 389 |
| | | | (s, 3H), 2.46- | |
| | | | 2.26 (m, 5H), | |
| | | | 2.09-1.88 (m, | |
| | | | 3H). | |
| 41 | 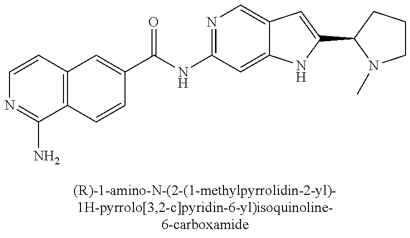 [SMILES: CN1CCC[C@@H]1c1cc2cnc(NC(=O)c3ccc4c(N)nccc4c3)cc2[nH]1] | 40 | ¹H-NMR: (300 MHz, DMSO-d6, ppm) δ 11.42 (s, 1H), 10.66 (s, 1H), 8.53 (s, 1H), 8.42 (d, J = 1.8 Hz, 1H), 8.34- 8.21 (m, 2H), 8.03 (dd, J = 8.7, 1.8 Hz, 1H), 7.88 (d, J = 5.8 Hz, 1H), 7.02 (d, J = | LC-MS: (ES, m/z): [M + H]⁺ = 387 |
| | | | 5.8 Hz, 1H), | |
| | | | 6.91 (s, 2H), | |
| | | | 6.41 (s, 1H), | |
| | | | 3.35 (s, 1H), | |
| | | | 3.15 (t, J = 8.0 | |
| | | | Hz, 1H), 2.31- | |
| | | | 2.11 (m, 5H), | |
| | | | 1.92-1.82 (m, | |
| | | | 3H). | |
| 42 | 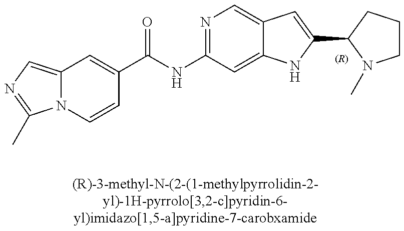 | 41 | ¹H-NMR: (300 MHz, Methanol-d₄, ppm) δ 8.54 (d, J = 1.0 Hz, 1H), 8.33- 8.26 (m, 1H), 8.17 (t, J = 1.0 Hz, 1H), 8.11 (m, 1H), 7.62 (d, J = 0.9 Hz, 1H), 7.25 (dd, J = 7.5, 1.8 Hz, 1H), 6.53 | LC-MS: (ES, m/z): [M + H]⁺ = 375 |
| | | | (d, J = 1.0 Hz, | |
| | | | 1H), 3.45 (t, J = | |
| | | | 7.9 Hz, 1H), | |
| | | | 3.24 (t, J = 7.7 | |
| | | | Hz, 1H), 2.71 | |
| | | | (s, 3H), 2.46- | |
| | | | 2.26 (m, 5H), | |
| | | | 2.14-1.98 (m, 3H). | |
| 43 | 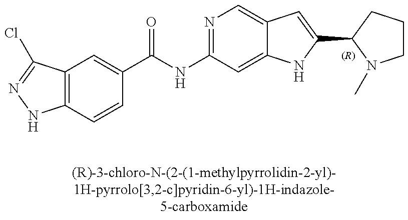 | 16 | ¹H-NMR: (300 MHz, Methanol-d₄, ppm) δ 8.55 (d, J = 1.0 Hz, 1H), 8.45 (dd, J = 1.7, 0.8 Hz, 1H), 8.21 (d, J = 1.0 Hz, 1H), 8.12 (dd, J = 8.9, 1.7 Hz, 1H), 7.67 (dd, J = 8.9, 0.8 Hz, 1H), | LC-MS: (ES, m/z): [M + H]⁺ = 395 |
| | | | 6.54 (d, J = | |
| | | | 1.0 Hz, 1H), | |
| | | | 3.46 (t, J = 7.9 | |
| | | | Hz, 1H), 3.25 | |
| | | | (t, J = 7.8 Hz, | |
| | | | 1H), 2.47- | |
| | | | 2.27 (m, 5H), | |
| | | | 2.12-1.97 (m, 3H). | |
| 44 | 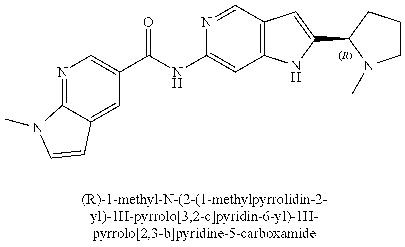 | H | ¹H-NMR: (300 MHz, Methanol-d₄, ppm) δ 8.92 (d, J = 2.1 Hz, 1H), 8.62 (d, J = 2.1 Hz, 1H), 8.53 (d, J = 1.0 Hz, 1H), 8.18 (t, J = 1.0 Hz, 1H), 7.50 (d, J = 3.5 Hz, 1H), 6.66 (d, J = 3.5 Hz, 1H), 6.51 (d, J = 1.0 Hz, 1H), | LC-MS: (ES, m/z): [M + H]⁺ = 375 |
| | | | 3.93 (s, 3H), | |
| | | | 3.44 (t, J = 7.9 | |
| | | | Hz, 1H), 3.29- | |
| | | | 3.18 (m, 1H), | |
| | | | 2.44-2.23 (m, | |
| | | | 5H), 2.07- | |
| | | | 1.88 (m, 3H). | |
| 45 | 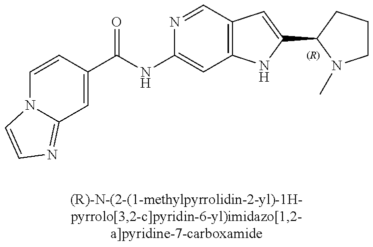 | I | ¹H-NMR: (300 MHz, Methanol-d₄, ppm) δ 8.63- 8.50 (m, 2H), 8.30-8.24 (m, 1H), 8.17 (t, J = 1.0 Hz, 1H), 8.00 (t, J = 1.0 Hz, 1H), 7.75 (d, J = 1.3 Hz, 1H), 7.49 (dd, J = 7.2, 1.8 Hz, 1H), 6.52 (d, J = 1.0 Hz, | LC-MS: (ES, m/z): [M + H]⁺ = 361 |
| | | | 1H), 3.44 (t, J = | |
| | | | 7.9 Hz, 1H), | |
| | | | 3.23 (t, J = 7.9 | |
| | | | Hz, 1H), 2.44- | |
| | | | 2.24 (m, 5H), | |
| | | | 2.09-1.90 (m, | |
| | | | 3H). | |
| 43 | 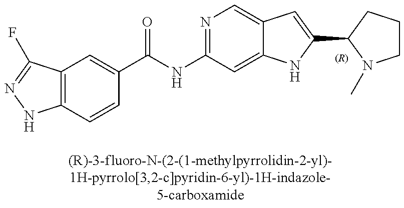 | 17 | ¹H-NMR: (300 MHz, Methanol-d₄, ppm) δ 8.52 (d, J = 1.0 Hz, 1H), 8.42 (s, 1H), 8.17 (t, J = 1.0 Hz, 1H), 8.08 (dd, J = 8.9, 1.7 Hz, 1H), 7.64- 7.54 (m, 1H), 6.51 (d, J = | LC-MS: (ES, m/z): [M + H]⁺ = 379 |
| | | | 0.9 Hz, 1H), | |
| | | | 3.46 (t, J = 7.9 | |
| | | | Hz, 1H), 3.25 | |
| | | | (t, J = 7.7 Hz, | |
| | | | 1H), 2.44-2.29 | |
| | | | (m, 5H), 2.09- | |
| | | | 1.86 (m, 3H). | |
| 47 | 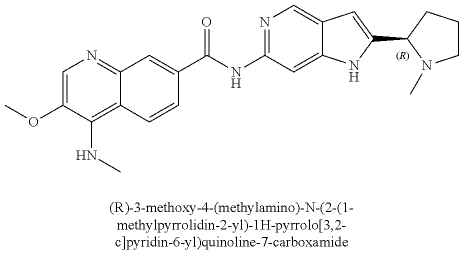 | 18 | ¹H-NMR (300 MHz, Methanol-d₄, ppm): δ 8.53 (s, 1H), 8.47 (s, 1H), 8.43 (d, J = 1.9 Hz, 1H), 8.29 (d, J = 9.0 Hz, 1H), 8.20 (s, 1H), 7.93 (dd, J = 8.9, 1.9 Hz, 1H), 6.51 (s, 1H), 3.98 (s, 3H), 3.43 (t, J = 8.0 Hz, 1H), | LC-MS (ES, m/z): [M + 1]⁺ = 431 |
| | | | 3.36 (s, 3H), | |
| | | | 3.24-3.20 (m, | |
| | | | 1H), 2.39 (q, J = | |
| | | | 8.9 Hz, 1H), | |
| | | | 2.30 (s, 3H), | |
| | | | 2.30-2.26 (m, | |
| | | | 1H), 2.07 − | |
| | | | 1.98 (m, 2H), | |
| | | | 1.97 − 1.88 | |
| | | | (m, 1H). | |
| 48 | 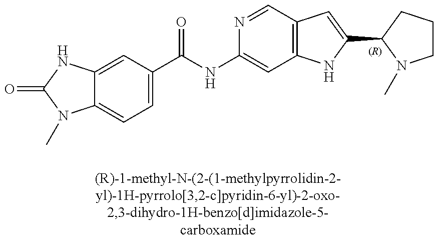 | 37 | H-NMR (300 MHz, DMSO- d₆, ppm): δ 11.34 (s, 1H), 11.11 (s, 1H), 10.32 (s, 1H), 8.49 (s, 1H), 8.18 (s, 1H), 7.85 (dd, J = 6.6, 1.5 Hz, 1H), 7.68 (d, J = 1.5 Hz, 1H), 7.19 (d, J = 8.1 Hz, 1H), 6.39 (s, 1H), | LC-MS (ES, m/z): 391[M + H]⁺ |
| | | | 3.29 (s, 3H), | |
| | | | 3.17-3.12 (m, | |
| | | | 1H), 3.24 (s, | |
| | | | 1H), 2.18 − | |
| | | | 2.10 (m, 2H), | |
| | | | 2.18 (s, 3H), | |
| | | | 1.93-1.78 (m, | |
| | | | 3H). | |
| 49 | 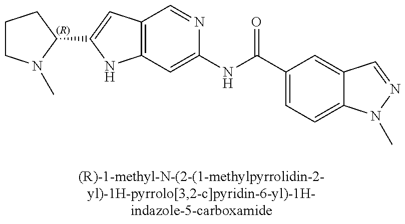 | B | ¹H-NMR (300 MHz, DMSO- d₆, ppm) δ 11.36 (s, 1H), 10.45 (s, 1H), 8.57 (d, J = 0.6 Hz, 1H), 8.51 (s, 1H), 8.22 − 8.21 (m, 2H), 8.09 (dd, J = 9.0, 1.8 Hz, 1H), 7.73 (d, J = 9.0, 1H), 6.40 | LC-MS (ES, m/z): [M + 1]⁺ =375 |
| | | | (s, 1H), 4.10 | |
| | | | (s, 3H), 3.35- | |
| | | | 3.32 (m, 1H), | |
| | | | 3.15 (t, J = 8.2 | |
| | | | Hz, 1H), 2.34 − | |
| | | | 2.06 (m, | |
| | | | 2H), 2.18 (s, | |
| | | | 3H), 1.96 − | |
| | | | 1.78 (m, 3H). | |
| 50 | 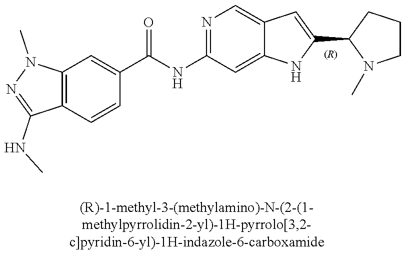 | 42 | ¹H-NMR (300 MHz, Methanol-d₄, ppm) δ 8.55 (d, J = 0.9 Hz, 1H), 8.23 (s, 1H), 7.98 (d, J = 1.2 Hz, 1H), 7.78 (d, J = 8.4 Hz, 1H), 7.56 (dd, J = 8.4, 1.5 Hz, 1H), 6.54 (d, J = 0.9 Hz, 1H), 3.92 (s, 3H), 3.46 (t, J = 7.8 | LC-MS (ES, m/z): [M + 1]⁺ = 404 |
| | | | Hz, 1H), 3.31 − | |
| | | | 3.17 (m, | |
| | | | 1H), 3.02 (s, | |
| | | | 3H), 2.50 − | |
| | | | 2.24 (m, 2H), | |
| | | | 2.32 (s, 3H), | |
| | | | 2.08 − 1.86 | |
| | | | (m, 3H). | |
| 51 | 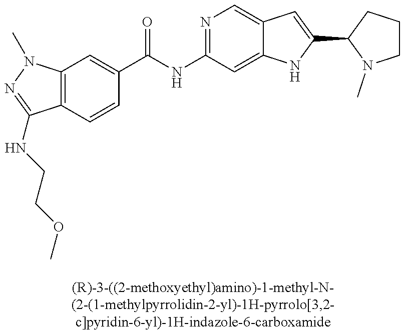 [SMILES: COCCNc1nn(C)c2cc(C(=O)Nc3cc4[nH]c([C@H]5CCCN5C)cc4cn3)ccc12] | 23 | ¹H-NMR (300 MHz, Methanol-d₄, ppm) δ 8.55 (s, 1H), 8.23 (s, 1H), 7.98 (s, 1H), 7.83 (d, J = 8.4 Hz, 1H), 7.57 (d, J = 8.4 Hz, 1H), 6.54 (s, 1H), 3.92 (d, J = 1.2 Hz, 3H), 3.70 (t, J = 5.4 Hz, 2H), 3.57 (t, J = 5.4 Hz, 2H), 3.48- 3.40 (m, 1H), 3.43 (s, 3H), 3.25 (t, J = 8.1 Hz, 1H), 2.49 − | LC-MS (ES, m/z): [M + 1]⁺ =448 |
| | | | 2.23 (m, | |
| | | | 2H), 2.32 (s, | |
| | | | 3H), 2.17 − | |
| | | | 1.85 (m, 3H). | |
| 52 | 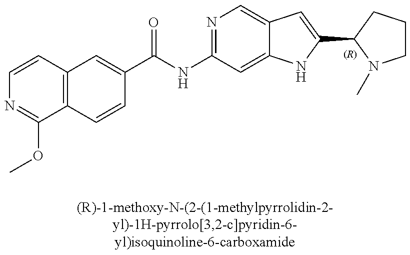 | 25 | ¹H-NMR(300 MHz, Methanol-d₄, ppm): δ 8.59 − 8.53 (m, 1H), 8.47 (d, J = 1.7 Hz, 1H), 8.40 (d, J = 8.7 Hz, 1H), 8.24 (s, 1H), 8.13 (dd, J = 8.7, 1.8 Hz, 1H), 8.08 (d, J = 5.9 Hz, 1H), 7.48 (d, J = 5.9 Hz, 1H), | LC-MS (ES, m/z): [M + H] = 402 |
| | | | 6.55 (d, J = | |
| | | | 0.9 Hz, 1H), | |
| | | | 4.17 (s, 3H), | |
| | | | 3.46 (t, J = 7.9 | |
| | | | Hz, 1H), 3.25 | |
| | | | (t, J = 7.7 Hz, | |
| | | | 1H), 2.42 (q, J = | |
| | | | 8.8 Hz, 1H), | |
| | | | 2.33 (s, 4H), | |
| | | | 2.12 − 1.89 | |
| | | | (m, 3H). | |
| 53 | 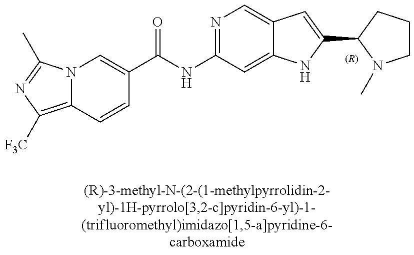 | 26 | ¹H-NMR (300 MHz, Methanol-d₄, ppm): δ 8.90 (s, 1H), 8.56 (s, 1H), 8.17 (s, 1H), 7.75 (d, J = 9.7 Hz, 1H), 7.62 (d, J = 9.6 Hz, 1H), 6.54 (s, 1H), 3.47 (t, J = 8.0 Hz, 1H), 3.30 − 3.21 (m, 1H), 2.81 (s, 3H), 2.43 (q, J = | LC-MS (ES, m/z): [M + H] = 443 |
| | | | 8.8 Hz, 1H), | |
| | | | 2.32 (s, 4H), | |
| | | | 2.14 − 2.02 | |
| | | | (m, 2H), 2.01 − | |
| | | | 1.90 (m, | |
| | | | 1H). | |
| 54 | 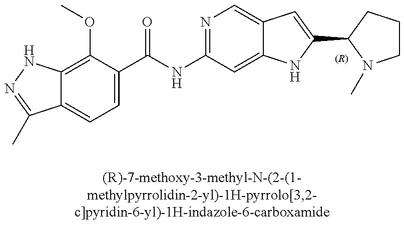 | 27 | ¹H-NMR (300 MHz, Methanol- d₄,ppm): δ 8.52 (d, J = 1.1 Hz, 1H), 8.41 (s, 1H), 7.80 (d, J = 8.5 Hz, 1H), 7.59 (d, J = 8.4 Hz, 1H), 6.54 (s, 1H), 4.24 (s, 2H), 3.47 (t, J = 7.8 | LC-MS (ES, m/z): [M + H] = 405 |
| | | | Hz, 1H), 3.24 | |
| | | | (d, J = 7.9 Hz, | |
| | | | 1H), 2.61 (s, | |
| | | | 3H), 2.48 − | |
| | | | 2.38 (m, 1H), | |
| | | | 2.33 (s, 3H), | |
| | | | 2.29 (d, J = | |
| | | | 8.3 Hz, 1H), | |
| | | | 2.14 − 1.91 | |
| | | | (m, 3H). | |
| 55 | 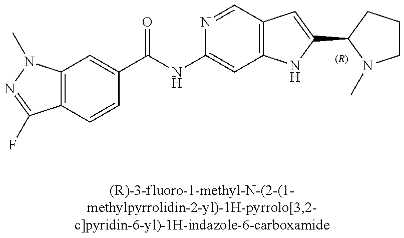 | 28 | ¹H-NMR: (300 MHz, Methanol-d₄, ppm) δ 8.54 (d, J = 1.0 Hz, 1H), 8.23- 8.15 (m, 2H), 7.85-7.71 (m, 2H), 6.52 (d, J = 1.0 Hz, 1H), 4.03 (d, J = 1.1 Hz, 3H), 3.43 (t, J = 7.9 Hz, 1H), 3.23 (t, J = 7.9 Hz, 1H), 2.47- | LC-MS: (ES, m/z): [M + H]⁺ = 393 |
| | | | 2.19 (m, 5H), | |
| | | | 2.04-1.95 (m, | |
| | | | 3H). | |
| 56 | 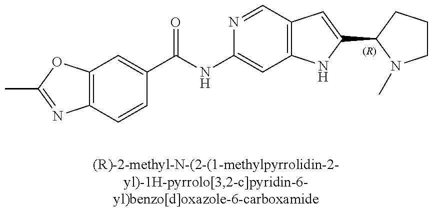 | J | ¹H-NMR (300 MHz, Methanol-d₄, ppm): δ 8.54 (s, 1H), 8.25 (d, J = 1.7 Hz, 1H), 8.20 (s, 1H), 8.05 (dd, J = 8.4, 1.7 Hz, 1H), 7.77 (d, J = 8.3 Hz, 1H), 6.53 (s, 1H), 3.46 (t, J = 7.9 Hz, 1H), | LC-MS (ES, m/z): [M + H] = 376 |
| | | | 3.29 − 3.20 | |
| | | | (m, 1H), 2.72 | |
| | | | (s, 3H), 2.42 | |
| | | | (q, J = 8.7 Hz, | |
| | | | 1H), 2.32 (s, | |
| | | | 4H), 2.07 − | |
| | | | 1.89 (m, 3H). | |
| 57 | 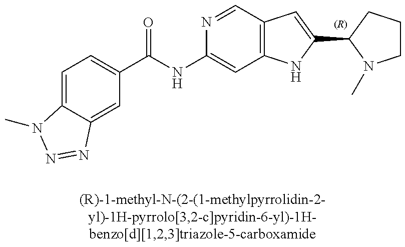 | K | ¹H-NMR (300 MHz, Methanol- d₄,ppm): δ 8.71 (dd, J = 1.6, 0.8 Hz, 1H), 8.56 (d, J = 1.0 Hz, 1H), 8.27 − 8.17 (m, 2H), 7.93 (dd, J = 8.8, 0.9 Hz, 1H), 6.54 (d, J = 1.0 Hz, 1H), 4.42 (s, 3H), | LC-MS (ES, m/z): [M + H] = 376 |
| | | | 3.46 (t, J = 7.9 | |
| | | | Hz, 1H), 3.29 − | |
| | | | 3.21 (m, | |
| | | | 1H), 2.42 (q, J = | |
| | | | 8.7 Hz, 1H), | |
| | | | 2.33 (s, 3H), | |
| | | | 2.29 (d, J = | |
| | | | 7.9 Hz, 1H), | |
| | | | 2.10 − 1.91 | |
| | | | (m, 3H) | |
| 58 | 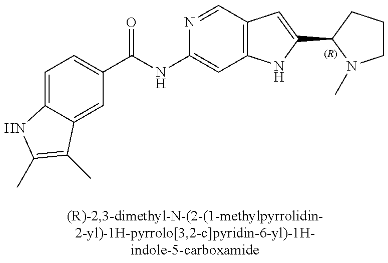 | L | ¹H-NMR (300 MHz, Methanol-d₄, ppm): δ 8.52 (d, J = 0.9 Hz, 1H), 8.25 (d, J = 1.0 Hz, 1H), 8.16 (d, J = 1.7 Hz, 1H), 7.72 (dd, J = 8.5, 1.8 Hz, 1H), 7.35 (d, J = 8.5 Hz, 1H), 6.52 (s, 1H), 3.45 (t, J = 8.0 Hz, 1H), 3.28 − | LC-MS (ES, m/z): [M + H] = 388 |
| | | | 3.20 (m, | |
| | | | 1H), 2.40 (s, | |
| | | | 4H), 2.31 (d, J = | |
| | | | 5.2 Hz, 7H), | |
| | | | 2.10 − 2.00 | |
| | | | (m, 2H), 1.98 − | |
| | | | 1.87 (m, | |
| | | | 1H). | |
| 59 | 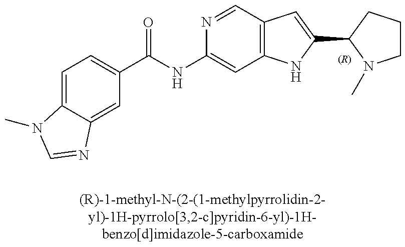 | M | ¹H-NMR (300 MHz, Methanol- d₄, ppm): δ 8.54 (d, J = 1.0 Hz, 1H), 8.41 (d, J = 1.6 Hz, 1H), 8.29 (s, 1H), 8.22 (t, J = 1.0 Hz, 1H), 8.05 (dd, J = 8.5, 1.7 Hz, 1H), 7.74 (d, J = 8.8 Hz, 1H), 6.54 (d, J = | LC-MS (ES, m/z): [M + H] = 375 |
| | | | 1.0 Hz, 1H), | |
| | | | 3.99 (s, 3H), | |
| | | | 3.45 (t, J = 7.9 | |
| | | | Hz, 1H), 3.25 | |
| | | | (t, J = 7.8 Hz, | |
| | | | 1H), 2.49 − | |
| | | | 2.37 (m, 1H), | |
| | | | 2.32 (s, 3H), | |
| | | | 2.26 (d, J = | |
| | | | 20.1 Hz, 1H), | |
| | | | 2.13 − 1.89 | |
| | | | (m, 3H). | |
| 60 | 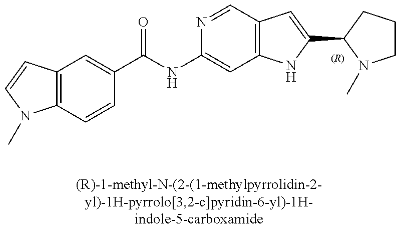 | N | ¹H-NMR (400 MHz, Methanol-d₄, ppm)δ 8.53 (d, J = 1.0 Hz, 1H), 8.31 (d, J = 1.8 Hz, 1H), 8.23 (d, J = 1.2 Hz, 1H), 7.87 (dd, J = 8.6, 1.8 Hz, 1H), 7.54 (d, J = 8.7 Hz, 1H), 7.32 (d, J = 3.2 Hz, 1H), | LC-MS (ES, m/z): [M + 1]⁺ = 374 |
| | | | 6.63 (d, J = | |
| | | | 3.1 Hz, 1H), | |
| | | | 6.53 (d, J = | |
| | | | 1.0 Hz, 1H), | |
| | | | 3.89 (s, 3H), | |
| | | | 3.45 (t, J = 8.0 | |
| | | | Hz, 1H), 3.24 | |
| | | | (t, J = 8.3 Hz, | |
| | | | 1H) , 2.42 (q, | |
| | | | J = 8.9 Hz, | |
| | | | 1H), 2.32 (s, | |
| | | | 3H), 2.36 − | |
| | | | 2.25 (m, 1H), | |
| | | | 2.05 (s, 2H), | |
| | | | 1.92 (dd, J = | |
| | | | 11.1, 6.7 Hz, | |
| | | | 1H) | |
| 61 | 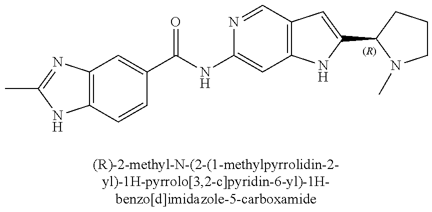 | O | ¹H-NMR (400 MHz, Methanol-d₄, ppm)δ 8.54 (d, J = 1.0 Hz, 1H), 8.22 (s, 2H), 7.90 (dd, J = 8.5, 1.7 Hz, 1H), 7.63 (d, J = 8.4 Hz, 1H), 6.53 (s, 1H), 3.45 (t, J = 8.0 Hz, 1H), | LC-MS (ES, m/z): [M + 1]⁺ = 375 |
| | | | 3.24 (s, 1H), | |
| | | | 2.64 (s, 3H), | |
| | | | 2.42 (q, J = | |
| | | | 8.8 Hz, 1H), | |
| | | | 2.32 (s, 4H), | |
| | | | 2.05 (s, 2H), | |
| | | | 1.99 − 1.90 | |
| | | | (m, 1H). | |
| 62 | 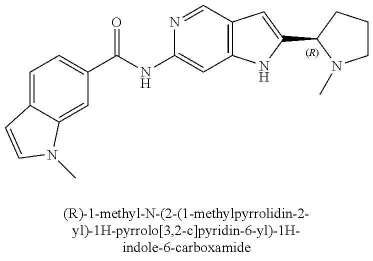 | P | ¹H-NMR (400 MHz, Methanol-d₄, ppm)δ 8.54 (d, J = 1.0 Hz, 1H), 8.25 (d, J = 1.0 Hz, 1H), 8.15 (d, J = 1.4 Hz, 1H), 7.76 − 7.66 (m, 2H), 7.40 (d, J = 3.1 Hz, 1H), 6.58- 6.50 (m, 2H),3.95 (s, 3H), 3.46 (t, J = | LC-MS (ES, m/z): [M + 1]⁺ = 374 |
| | | | 8.0 Hz, 1H), | |
| | | | 3.30-3.21 (m, | |
| | | | 1H), 2.48- | |
| | | | 2.26 (m, 1H), | |
| | | | 2.32 (s, 3H), | |
| | | | 2.11 − 2.00 | |
| | | | (m, 1H), 2.05 | |
| | | | (s, 2H), 1.33 | |
| | | | (d, J = 15.5 | |
| | | | Hz, 1H). | |
| 63 | 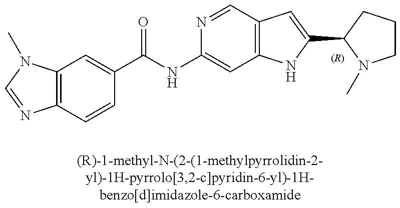 | Q | H-NMR (400 MHz, Methanol-d₄, ppm)δ 8.54 (d, J = 1.0 Hz, 1H), 8.22 (s, 2H), 7.90 (dd, J = 8.5, 1.7 Hz, 1H), 7.63 (d, J = 8.4 Hz, 1H), 6.53 (s, 1H), 3.45 (t, J = 8.0 Hz, 1H), | LC-MS (ES, m/z): [M + 1]⁺ = 375 |
| | | | 3.24 (s, 1H), | |
| | | | 2.64 (s, 3H), | |
| | | | 2.42 (q, J = | |
| | | | 8.8 Hz, 1H), | |
| | | | 2.32 (s, 4H), | |
| | | | 2.05 − 1.90 | |
| | | | (m, 1H). | |
| 64 | 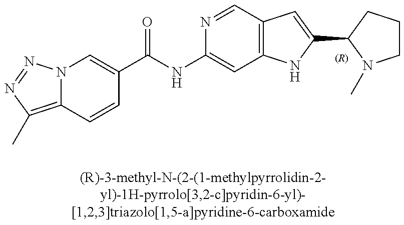 | 29 | ¹H-NMR (300 MHz, Methanol-d₄, ppm)δ 9.54 − 9.49 (m, 1H), 8.56 (d, J = 1.1 Hz, 1H), 8.18 (s, 1H), 7.97 (dd, J = 9.3, 1.2 Hz, 1H), 7.84 (dd, J = 9.3, 1.5 Hz, 1H), 6.54 (s, 1H), 3.45 | LC-MS (ES, m/z): [M + 1]⁺ = 376 |
| | | | (t, J = 7.9 Hz, | |
| | | | 1H), 3.25 (t, J = | |
| | | | 7.6 Hz, 1H), | |
| | | | 2.66 (d, J = | |
| | | | 1.2 Hz, 3H), | |
| | | | 2.42 (q, J = | |
| | | | 8.8 Hz, 1H), | |
| | | | 2.32-2.27 (m, | |
| | | | 4 H), 2.04 (s, | |
| | | | 2H), 1.96 (dd, | |
| | | | J = 19.2, 9.2 | |
| | | | Hz, 1H). | |
| 65 | 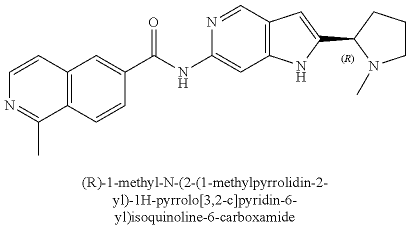 | 30 | ¹H-NMR2: (400 MHz, Methanol-d₄, ppm) δ 8.58 (dd, J = 4.8, 1.4 Hz, 2H), 8.42 (t, J = 7.4 Hz, 2H), 8.28- 8.20 (m, 2H), 7.86 (d, J = 5.9 Hz, 1H), 6.55 (s, 1H), 3.47 (d, J = 7.8 Hz, 1H), 3.26 (t, J = 7.8 | LC-MS: (ES, m/z): [M + H]⁺ = 386 |
| | | | Hz, 1H), 3.03 | |
| | | | (s, 3H), 2.47- | |
| | | | 2.28 (m, 5H), | |
| | | | 2.08-1.91 (m, | |
| | | | 3H). | |
| 66 | 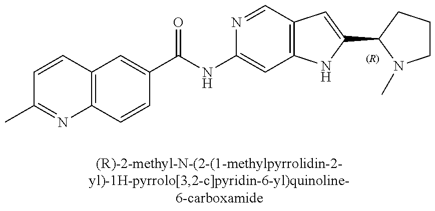 | R | ¹H-NMR (300 MHz, Methanol-d₄, ppm)δ 8.58 (dd, J = 14.7, 1.5 Hz, 2H), 8.43 (d, J = 8.5 Hz, 1H), 8.31 (dd, J = 8.8, 2.1 Hz, 1H), 8.24 (s, 1H), 8.10 (d, J = 8.8 Hz, 1H), | LC-MS (ES, m/z): [M + 1]⁺ = 386 |
| | | | 7.57 (d, J = | |
| | | | 8.5 Hz, 1H), | |
| | | | 6.54 (s, 1H), | |
| | | | 3.46 (t, J = 8.0 | |
| | | | Hz, 1H), 3.30 − | |
| | | | 3.20 (m, | |
| | | | 1H), 2.79 (s, | |
| | | | 3H), 2.42 (q, J = | |
| | | | 8.9 Hz, 1H), | |
| | | | 2.32 (s, 3H), | |
| | | | 2.05- 1.88 (m, | |
| | | | 3H). | |
| 67 | 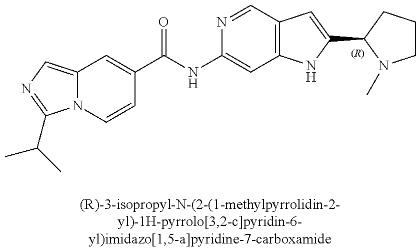 | 31 | ¹H-NMR (300 MHz, Methanol-d₄, ppm): δ 8.57 − 8.52 (m, 1H), 8.30 (t, J = 1.5 Hz, 1H), 8.22 (d, J = 7.6 Hz, 1H), 8.17 (s, 1H), 7.65 (s, 1H), 7.24 (dd, J = 7.5, 1.8 Hz, 1H), 6.53 (s, 1H), 3.56 (p, J = 6.9 Hz, 1H), 3.45 (t, J = 8.0 | LC-MS (ES, m/z): [M + H] = 403 |
| | | | Hz, 1H), 3.27 − | |
| | | | 3.22 (m, 1H), | |
| | | | 2.42 (q, J = 8.9 | |
| | | | Hz, 1H), 2.32 | |
| | | | (s, 4H), 2.11 − | |
| | | | 1.98 (m, 2H), | |
| | | | 1.98 − 1.87 (m, | |
| | | | 1H), 1.45 (d, J = | |
| | | | 6.9 Hz, 6H). | |
| 68 | 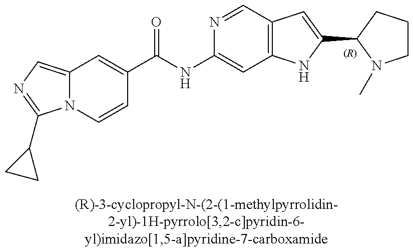 | 32 | ¹H-NMR (400 MHz, Methanol-d₄, ppm)δ 8.54 (s, 1H), 8.34 (d, J = 7.5 Hz, 1H), 8.27 (s, 1H), 8.17 (s, 1H), 7.56 (s, 1H), 7.25 (dd, J = 7.5, 1.8 Hz, 1H), 6.52 (s, 1H), 3.44 (t, J = 7.9 Hz, 1H), 3.28 − 3.20 (m, 1H), 2.41 | LC-MS (ES, m/z): [M + 1]⁺ = 401 |
| | | | (q, J = 8.8 Hz, | |
| | | | 1H), 2.31 − | |
| | | | 2.23 (m, 5H), | |
| | | | 2.03 (t, J = 8.5 | |
| | | | Hz, 2H), 1.95 | |
| | | | (dd, J = 19.1, | |
| | | | 8.9 Hz, 1H), | |
| | | | 1.17 (dt, J = | |
| | | | 8.4, 3.3 Hz, | |
| | | | 2H), 1.05 (dt, | |
| | | | J = 5.1, 3.1 | |
| | | | Hz, 2H). | |
| 69 | 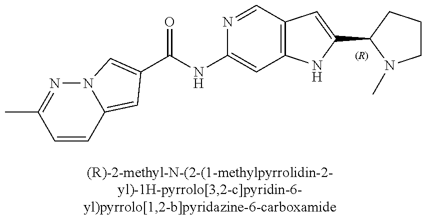 | 33 | ¹H-NMR (400 MHz, Methanol-d₄, ppm)δ 8.52 (d, J = 1.1 Hz, 1H), 8.27 (d, J = 1.9 Hz, 1H), 8.22 − 8.17 (m, 1H), 7.84 (d, J = 9.3 Hz, 1H), 7.02 (d, J = 1.9 Hz, 1H), 6.65 (d, J = 9.3 Hz, 1H), | LC-MS (ES, m/z): [M + 1]⁺ = 375 |
| | | | 6.54 − 6.49 | |
| | | | (m, 1H), 3.44 | |
| | | | (t, J = 8.0 Hz, | |
| | | | 1H), 3.24 (s, | |
| | | | 1H), 2.47 (s, | |
| | | | 3H), 2.41 (q, J = | |
| | | | 10.0, 9.0 Hz, | |
| | | | 1H), 2.31 (s, | |
| | | | 3H), 2.31 − | |
| | | | 2.24 (m, 1H), | |
| | | | 2.10 − 1.95 | |
| | | | (m, 2H), 1.93 | |
| | | | (s, 1H). | |
| 70 | 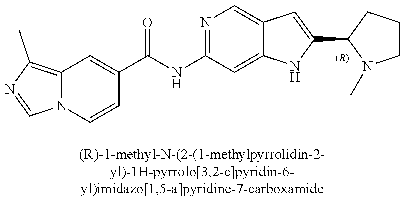 | 43 | ¹H-NMR (400 MHz, Methanol-d₄, ppm)δ 8.54 (s, 1H), 8.39 (s, 1H), 8.36 (s, 1H), 8.32 (s, 1H), 8.22 (d, J = 7.5 Hz, 1H), 8.17 (s, 1H), 7.17 (d, J = 7.5 Hz, 1H), 6.54 (s, 1H), | LC-MS (ES, m/z): [M + 1]⁺ = 375 |
| | | | 3.47 (t, J = 7.9 | |
| | | | Hz, 1H), 3.25 | |
| | | | (t, J = 8.4 Hz, | |
| | | | 1H), 2.60 (s, | |
| | | | 3H), 2.46 − | |
| | | | 2.37 (m, 1H), | |
| | | | 2.32 (s, 4H), | |
| | | | 2.05 (s, 2H), | |
| | | | 1.94 (d, J = | |
| | | | 7.0 Hz, 1H). | |
| 71 | 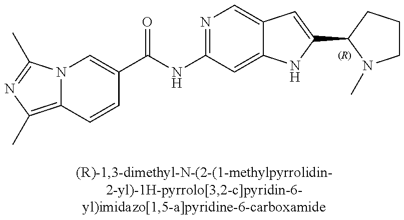 | 44 | ¹H-NMR (300 MHz, Methanol-d₄, ppm)δ 8.66 (s, 1H), 8.55 (s, 1H), 8.16 (s, 1H), 7.54 (d, J = 9.6 Hz, 1H), 7.16 (d, J = 9.6 Hz, 1H), 6.54 (s, 1H), 3.46 (t, J = 7.8 Hz, 1H), 3.25 (s, 1H), 2.72 | LC-MS (ES, m/z): [M + 1]⁺ = 389 |
| | | | (d, J = 1.5 Hz, | |
| | | | 3H), 2.52 − | |
| | | | 2.38 (m, 4H), | |
| | | | 2.32 (d, J = | |
| | | | 1.4 Hz, 4H), | |
| | | | 2.05 (s, 3H). | |
| 72 | 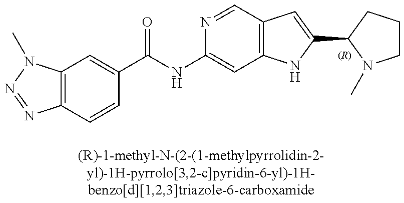 | 47 | ¹H-NMR: (300 MHz, Methanol-d₄, ppm) δ 8.54 (d, J = 1.0 Hz, 1H), 8.45 (t, J = 1.3 Hz, 1H), 8.21 (d, J = 1.0 Hz, 1H), 8.12 (dd, J = 8.8, 0.9 Hz, 1H), 8.03 (dd, J = 8.8, 1.5 | LC-MS: (ES, m/z): [M + H]⁺ =376 |
| | | | Hz, 1H), 6.52 | |
| | | | (d, J = 1.0 Hz, | |
| | | | 1H), 4.43 (s, | |
| | | | 3H), 3.44 (t, J = | |
| | | | 7.9 Hz, 1H), | |
| | | | 3.23 (t, J = 7.7 | |
| | | | Hz, 1H), 2.44- | |
| | | | 2.23 (m, 5H), | |
| | | | 2.09-1.92 (m, | |
| | | | 3H). | |

Prepared according to Example 1 Steps 5 and 6 using Intermediate 5 and the corresponding acid.

| Example | | | | |
|---|---|---|---|---|
| No. | Structure/Name | Acid | ¹H NMR | LCMS |
| 73 | 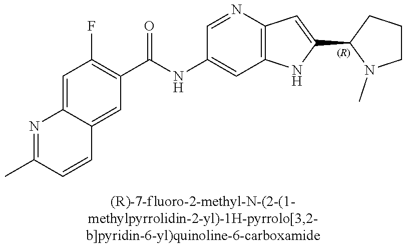 | 5 | ¹H-NMR (300 MHz, Methanol-d₄, ppm): δ 8.51- 8.33 (m, 4H), 7.75 (d, J = 11.7 Hz, 1H), 7.53 (d, J = 8.5 Hz, 1H), 6.55 (s, 1H), 3.50 (t, J = 7.8 Hz, 1H), 3.26-3.23 (m, 2H), 2.78 (s, 3H), 2.44 (q, J = 8.8 Hz, 1H), 2.34 (s, | LC-MS (ES, m/z): [M + H]⁺ = 404 |
| | | | 3H), 2.13- | |
| | | | 1.90 (m, 3H). | |
| 74 | 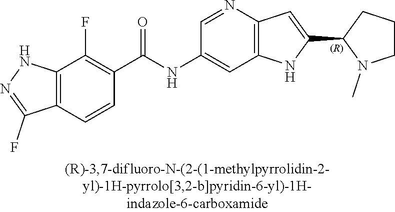 | 45 | ¹H-NMR (400 MHz, Methanol-d4, ppm) δ 8.43 (q, J = 2.3 Hz, 2H), 7.61 (d, J = 8.5 Hz, 1H), 7.47 (dd, J = 8.4, 5.4 Hz, 1H), 6.55 (s, 1H), 3.49 (t, J = 7.9 Hz, 1H), 3.25 (t, J = 7.7 | LC-MS (ES, m/z): [M + 1]⁺ = 397 |
| | | | Hz, 1H), 2.43 | |
| | | | (q, J = 8.9 Hz, | |
| | | | 1H), 2.33 (s, | |
| | | | 4H), 2.09- | |
| | | | 1.96 (m, 3H). | |
| 75 | 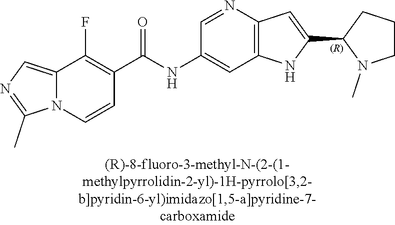 | 46 | ¹H-NMR (300 MHz, Methanol- d₄, ppm): δ 8.43 (d, J = 2.2 Hz, 1H), 8.37 (dd, J = 2.2, 0.9 Hz, 1H), 7.99 (dd, J = 7.4, 1.0 Hz, 1H), 7.71 (s, 1H), 7.12- 6.96 (m, 1H), 6.55 (d, J = 1.0 Hz, 1H), 3.51 | LC-MS (ES, m/z): [M + H]⁺ = 393 |
| | | | (t, J = 7.9 Hz, | |
| | | | 1H), 3.26 (t, J = | |
| | | | 8.0 Hz, 1H), | |
| | | | 2.73 (s, 3H), | |
| | | | 2.44 (q, J = 8.8 | |
| | | | Hz, 1H), 2.34 | |
| | | | (s, 4H), 2.14- | |
| | | | 1.90 (m, 3H). | |

Prepared according to Example 1 Steps 5 and 6 using Intermediate 6 and the corresponding acid.

| Example | | | | |
|---|---|---|---|---|
| No. | Structure/Name | Acid | ¹H NMR | LCMS |
| 76 | 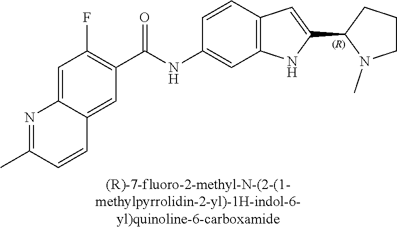 | 5 | ¹H-NMR (300 MHz, Methanol- d₄, ppm) δ 8.35 (t, J = 8.1 Hz, 2H), 8.00 (s, 1H), 7.71 (d, J = 11.7 Hz, 1H), 7.54-7.42 (m, 2H), 7.13 (dd, J = 8.4, 1.8 Hz, 1H), 6.38 (s, 1H), 3.44 (t, J = 8.1 Hz, 1H), 3.23 (t, J = 8.7 | LC-MS (ES, m/z): [M + 1]⁺ = 403 |
| | | | Hz, 1H), 2.75 (s, | |
| | | | 3H), 2.42 (q, J = | |
| | | | 8.7 Hz, 1H), | |
| | | | 2.31 (s, 3H), | |
| | | | 2.33-2.21 (m, | |
| | | | 1H), 2.16-1.86 | |
| | | | (m, 3H). | |

### Example 77: N-[2-[1-(dimethylamino)ethyl]-1H-pyrrolo[3,2-c]pyridin-6-yl]-1-methylindazole-6-carboxamide

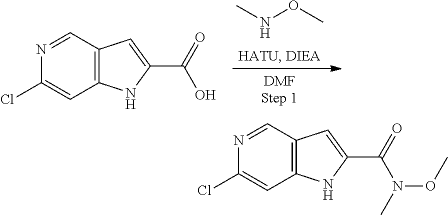

Into a 100-mL 3-necked round-bottom flask purged and maintained with an inert atmosphere of nitrogen, was placed N,O-dimethylhydroxylamine (1118.58 mg, 18.312 mmol, 1.2 equiv), DMF (30.00 mL), DIEA (5916.83 mg, 45.781 mmol, 3.0 equiv), HATU (6962.85 mg, 18.312 mmol, 1.2 equiv), 6-chloro-1H-pyrrolo[3,2-c]pyridine-2-carboxylic acid (3.00 g, 15.260 mmol, 1.00 equiv). The resulting solution was stirred for 16 hr at room temperature. The resulting solution was diluted with 50 mL of H₂O. The resulting solution was extracted with 3×50 mL of ethyl acetate and the organic layers combined. The resulting mixture was washed with 3×50 ml of brine. The resulting mixture was concentrated. The residue was applied onto a silica gel column with ethyl acetate/petroleum ether (1:1). This resulted in 1.6 g (43.75%) of 6-chloro-N-methoxy-N-methyl-1H-pyrrolo[3,2-c]pyridine-2-carboxamide as a light brown solid.

LC-MS (ES, m/z): [M+1]⁺=240.

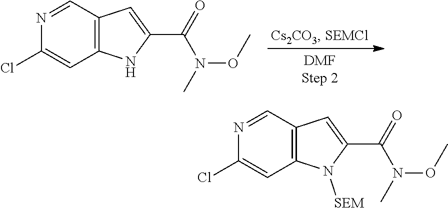 [SMILES: CON(C)C(=O)c1cc2cnc(Cl)cc2[nH]1 ; CON(C)C(=O)c1cc2cnc(Cl)cc2n1COCC[Si](C)(C)C]

Into a 50-mL 3-necked round-bottom flask purged and maintained with an inert atmosphere of nitrogen, was placed 6-chloro-N-methoxy-N-methyl-1H-pyrrolo[3,2-c]pyridine-2-carboxamide (1.60 g, 6.676 mmol, 1.00 equiv), DMF (16.00 mL), Cs₂CO₃ (6525.64 mg, 20.028 mmol, 3.00 equiv), SEMCl (1669.58 mg, 10.014 mmol, 1.50 equiv). The resulting solution was stirred for 6 hr at room temperature. The resulting solution was diluted with 20 mL of H₂O. The resulting solution was extracted with 3×20 mL of ethyl acetate and the organic layers combined and concentrated. The residue was applied onto a silica gel column with ethyl acetate/petroleum ether (1:3). This resulted in 1.5 g (60.74%) of 6-chloro-N-methoxy-N-methyl-1-[[2-(trimethylsilyl)ethoxy]methyl]pyrrolo[3,2-c]pyridine-2-carboxamide as a light brown solid.

LC-MS (ES, m/z): [M+1]⁺=370.

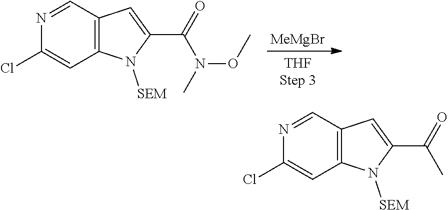 [SMILES: "MeMgBr" ; CON(C)C(=O)c1cc2cnc(Cl)cc2n1COCC[Si](C)(C)C ; CC(=O)c1cc2cnc(Cl)cc2n1COCC[Si](C)(C)C]

Into a 50-mL 3-necked round-bottom flask purged and maintained with an inert atmosphere of nitrogen, was placed 6-chloro-N-methoxy-N-methyl-1-[[2-(trimethylsilyl)ethoxy]methyl]pyrrolo[3,2-c]pyridine-2-carboxamide (1.50 g, 4.055 mmol, 1.00 equiv), THF (15.00 mL). This was followed by the addition of bromo(methyl)magnesium (725.29 mg, 6.082 mmol, 1.50 equiv) dropwise with stirring at −78° C. in 30 min. The resulting solution was stirred for 1 hr at −78° C. The resulting solution was diluted with 20 mL of H₂O. The resulting solution was extracted with 3×20 mL of ethyl acetate and the organic layers combined and concentrated. The residue was applied onto a silica gel column with ethyl acetate/petroleum ether (1:2). This resulted in 600 mg (45.55%) of 1-(6-chloro-1-[[2-(trimethylsilyl)ethoxy]methyl]pyrrolo[3,2-c]pyridin-2-yl) ethanone as a yellow solid.

LC-MS (ES, m/z): [M+1]⁺=325.

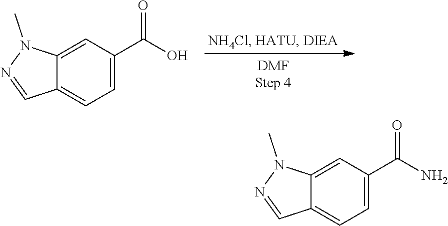

Into a 40-mL round-bottom flask, was placed NH₄Cl (166.99 mg, 3.122 mmol, 1.10 equiv), DMF (5.00 mL), HATU (1618.69 mg, 4.257 mmol, 1.50 equiv), DIEA (1100.41 mg, 8.514 mmol, 3.00 equiv), 1-methylindazole-6-carboxylic acid (A, 500.00 mg, 2.838 mmol, 1.00 equiv). The resulting solution was stirred for 12 hr at room temperature. The resulting solution was diluted with 20 mL of H₂O. The resulting solution was extracted with 3×20 mL of dichloromethane and the organic layers combined and concentrated. The residue was applied onto a silica gel column with dichloromethane/methanol (1:2). This resulted in 240 mg (48.27%) of 1-methylindazole-6-carboxamide as a white solid.

LC-MS (ES, m/z): [M+1]⁺=176.

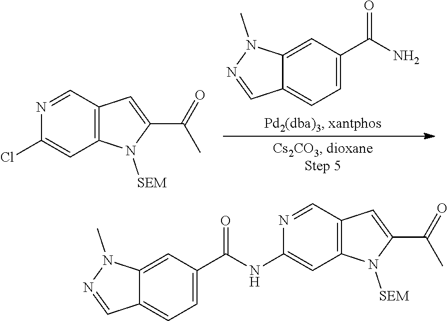

Into a 40-mL round-bottom flask, was placed 1-(6-chloro-1-[[2-(trimethylsilyl)ethoxy]methyl]pyrrolo[3,2-c]pyridin-2-yl) ethanone (500.00 mg, 1.539 mmol, 1.00 equiv), dioxane (5.00 mL), 1-methylindazole-6-carboxamide (269.62 mg, 1.539 mmol, 1.00 equiv), Pd₂(dba)₃ (70.47 mg, 0.077 mmol, 0.05 equiv), xantphos (44.53 mg, 0.077 mmol, 0.05 equiv), Cs₂CO₃ (1504.34 mg, 4.617 mmol, 3.00 equiv). The resulting solution was stirred for 5 hr at 110° C. The resulting solution was diluted with 20 mL of H₂O. The resulting solution was extracted with 3×20 mL of ethyl acetate and the organic layers combined. The resulting mixture was washed with 3×20 ml of brine. The resulting mixture was concentrated. The residue was applied onto a silica gel column with ethyl acetate/petroleum ether (1:1). This resulted in 300 mg (42.05%) of N-(2-acetyl-1-[[2-(trimethylsilyl)ethoxy]methyl]pyrrolo[3,2-c]pyridin-6-yl)-1-methylindazole-6-carboxamide as a white solid.

LC-MS (ES, m/z): [M+1]⁺=464.

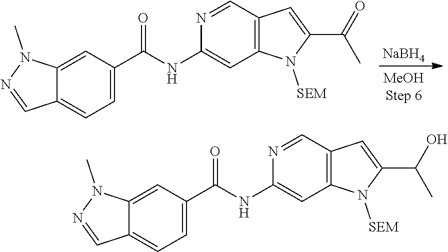

Into a 40-mL round-bottom flask, was placed N-(2-acetyl-1-[[2-(trimethylsilyl)ethoxy]methyl]pyrrolo[3,2-c]pyridin-6-yl)-1-methylindazole-6-carboxamide (200.00 mg, 0.431 mmol, 1.00 equiv), MeOH (4.00 mL), NaBH₄ (48.96 mg, 1.294 mmol, 3.00 equiv). The resulting solution was stirred for 3 hr at room temperature. The resulting solution was diluted with 20 mL of H₂O. The resulting solution was extracted with 3×20 mL of ethyl acetate and the organic layers combined and concentrated. This resulted in 100 mg (49.78%) of N-[2-(1-hydroxyethyl)-1-[[2-(trimethylsilyl)ethoxy]methyl]pyrrolo[3,2-c]pyridin-6-yl]-1-methylindazole-6-carboxamide as white oil.

LC-MS (ES, m/z): [M+1]⁺=466.

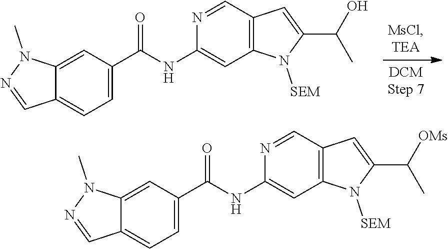

Into a 8-mL round-bottom flask, was placed N-[2-(1-hydroxyethyl)-1-[[2-(trimethylsilyl)ethoxy]methyl]pyrrolo[3,2-c]pyridin-6-yl]-1-methylindazole-6-carboxamide (100.00 mg, 0.215 mmol, 1.00 equiv), DCM (2.00 mL), TEA (65.20 mg, 0.644 mmol, 3.00 equiv), MsCl (29.52 mg, 0.258 mmol, 1.20 equiv). The resulting solution was stirred for 2 hr at room temperature. The resulting mixture was concentrated. This resulted in 100 mg (85.64%) of 1-[6-(1-methylindazole-6-amido)-1-[[2-(trimethylsilyl)ethoxy]methyl]pyrrolo[3,2-c]pyridin-2-ylethyl methanesulfonate as yellow oil.

LC-MS (ES, m/z): [M+1]⁺=544.

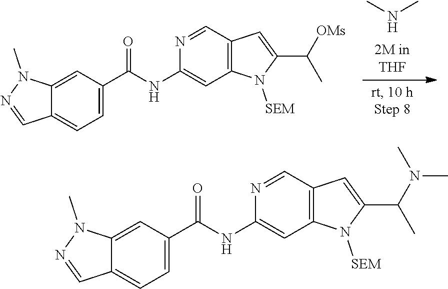

Into an 8-mL round-bottom flask, was placed 1-[6-(1-methylindazole-6-amido)-1-[[2-(trimethylsilyl)ethoxy]methyl]pyrrolo[3,2-c]pyridin-2-yl]ethyl methanesulfonate (100.00 mg, 0.184 mmol, 1.00 equiv), 2 M dimethylamine in THF (1.00 mL). The resulting solution was stirred for 10 hr at room temperature. The resulting mixture was concentrated. This resulted in 100 mg crude of N-[2-[1-(dimethylamino)ethyl]-1-[[2-(trimethylsilyl)ethoxy]methyl]pyrrolo[3,2-c]pyridin-6-yl]-1-methylindazole-6-carboxamide as yellow oil.

LC-MS (ES, m/z): [M+1]⁺=493.

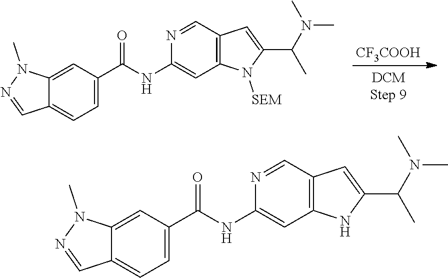 [SMILES: CC(c1cc2cnc(NC(=O)c3ccc4cnn(C)c4c3)cc2n1COCC[Si](C)(C)C)N(C)C ; "CF3COOH" ; CC(c1cc2cnc(NC(=O)c3ccc4cnn(C)c4c3)cc2[nH]1)N(C)C]

Into a 8-mL round-bottom flask, was placed N-[2-[1-(dimethylamino)ethyl]-1-[[2-(trimethylsilyl) ethoxy]methyl]pyrrolo[3,2-c]pyridin-6-yl]-1-methylindazole-6-carboxamide (100.00 mg, 0.203 mmol, 1.00 equiv), DCM (2.00 mL), CF₃COOH (99.48 mg, 1.015 mmol, 5.00 equiv). The resulting solution was stirred for 16 hr at room temperature. The resulting mixture was concentrated. The resulting solution was diluted with 10 mL of H₂O. The pH value of the solution was adjusted to 8-9 with NaHCO₃ (1 mol/L). The resulting solution was extracted with 3×10 mL of dichloromethane and the organic layers combined and concentrated. The crude product was purified by Prep-HPLC with the following conditions: Column: HPH C18, 50*3.0 mm, 2.6 μm; Mobile Phase A: Water/0.05% NH₃·H₂O, Mobile Phase B: ACN; Flow rate: 1.2 mL/min; Gradient: 5% B to 100% B in 1.1 min, hold 0.7 min, Detector, UV 254 nm. This resulted in 19 mg (25.8%) of N-[2-[1-(dimethylamino)ethyl]-1H-pyrrolo[3,2-c]pyridin-6-yl]-1-methylindazole-6-carboxamide as a white solid.

LC-MS (ES, m/z): [M+1]⁺=363.

¹H-NMR (300 MHz, Methanol-d₄, ppm): δ 8.58 (d, J=1.0 Hz, 1H), 8.26 (m, 2H), 8.12 (d, J=1.0 Hz, 1H), 7.92 (dd, J=8.5, 0.9 Hz, 1H), 7.79 (dd, J=8.5 Hz, 1H), 6.50 (m, 1H), 4.20 (s, 3H), 3.87 (m, 1H), 2.29 (s, 6H), 1.54 (d, J=6.9 Hz, 3H).

### Example 78: (R)-3-(difluoromethyl)-1-methyl-N-(2-(1-methylpyrrolidin-2-yl)-1H-pyrrolo[3,2-c]pyridin-6-yl)-1H-indazole-6-carboxamide

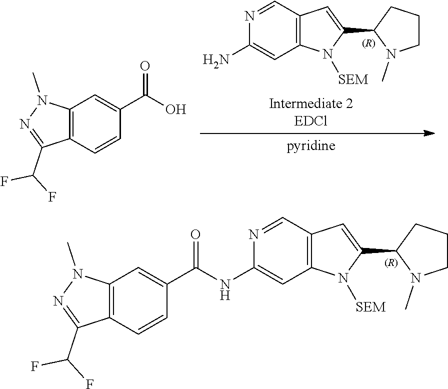

Into a 8-mL sealed tube, was placed 3-(difluoromethyl)-1-methylindazole-6-carboxylic acid (prepared according to WO2021127166, Acid AR, 52 mg, 0.23 mmol, 1.0 equiv), 2-[(2R)-1-methylpyrrolidin-2-yl]-1-[[2-(trimethylsilyl)ethoxy]methyl]pyrrolo[3,2-c]pyridin-6-amine (Intermediate 2, 79.7 mg, 0.23 mmol, 1.00 equiv), EDCI (88 mg, 0.46 mmol, 2.0 equiv) and pyridine (2.00 mL). The resulting solution was stirred for 12 h at room temperature. The resulting mixture was concentrated and extracted with 2×20 mL of ethyl acetate, the organic layers combined and concentrated. This resulted in 150 mg (crude) of 3-(difluoromethyl)-1-methyl-N-[2-[(2R)-1-methylpyrrolidin-2-yl]-1-[[2-(trimethylsilyl)ethoxy]methyl]pyrrolo[3,2-c]pyridin-6-yl]indazole-6-carboxamide as a brown solid.

LC-MS: (ES, m/z): [M+H]=555

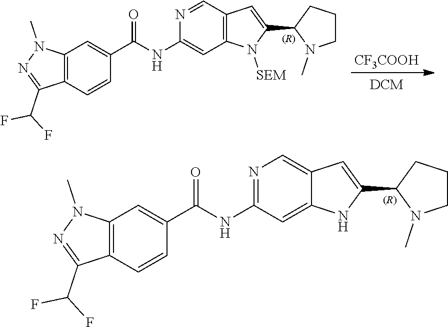

Into a 50-mL round-bottom flask, was placed 3-(difluoromethyl)-1-methyl-N-[2-[(2R)-1-methylpyrrolidin-2-yl]-1-[[2-(trimethylsilyl)ethoxy]methyl]pyrrolo[3,2-c]pyridin-6-yl]indazole-6-carboxamide (150.0 mg, crude), CF₃COOH (2.00 mL) and DCM (1.00 mL). The resulting solution was stirred for 16 h at room temperature and the mixture concentrated. The pH value of the solution was adjusted to 8 with NaHCO₃ (aq) solution and extracted with 2×20 mL of ethyl acetate, the organic layers combined and concentrated. The crude product (150 mg) was purified by Prep-HPLC eluting with 0.05% NH₃/H₂O and acetonitrile. Concentration of fractions gave 20.2 mg (20.8% over two steps) of (R)-3-(difluoromethyl)-1-methyl-N-(2-(1-methylpyrrolidin-2-yl)-1H-pyrrolo[3,2-c]pyridin-6-yl)-1H-indazole-6-carboxamide as a light brown solid.

LC-MS: (ES, m/z): [M+H]=425

¹H-NMR (300 MHz, Methanol-d₄, ppm): δ 8.56 (s, 1H), 8.34 (s, 1H), 8.24 (s, 1H), 8.03 (d, J=8.4 Hz, 1H), 7.89 (d, J=8.4 Hz, 1H), 7.11 (t, J=54.3 Hz, 1H), 6.55 (s, 1H), 4.23 (d, J=1.6 Hz, 3H), 3.47 (t, J=7.8 Hz, 1H), 3.27-3.22 (m, 1H), 2.55-2.26 (m, 5H), 2.10-1.93 (m, 3H).

¹⁹F-NMR (282 MHZ, Methanol-d₄, ppm): 8-119.83 (s, 2F)

### Example 79: (R)-1-(methylamino)-N-(2-(1-methylpyrrolidin-2-yl)-1H-pyrrolo[3,2-c]pyridin-6-yl) isoquinoline-6-carboxamide

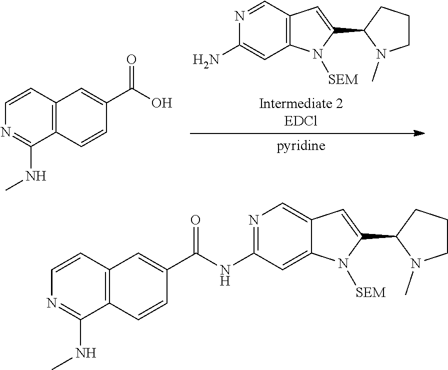 [SMILES: CNc1nccc2cc(C(=O)O)ccc12 ; CN1CCC[C@@H]1c1cc2cnc(N)cc2n1COCC[Si](C)(C)C ; CNc1nccc2cc(C(=O)Nc3cc4c(cn3)cc([C@H]3CCCN3C)n4COCC[Si](C)(C)C)ccc12]

Into a 8-mL vial purged and maintained with an inert atmosphere of nitrogen, was placed 1-(methylamino) isoquinoline-6-carboxylic acid (1374258-72-6, 160 mg, 0.79 mmol, 1.0 equiv), pyridine (3.50 mL), 2-[(2R)-1-methylpyrrolidin-2-yl]-1-[[2-(trimethylsilyl)ethoxy]methyl]pyrrolo[3,2-c]pyridin-6-amine (Intermediate 2, 137.1 mg, 0.4 mmol, 0.5 equiv) and EDCI (151.7 mg, 0.79 mmol, 1.0 equiv). The resulting solution was stirred for 16 h at room temperature. The resulting mixture was concentrated under vacuum and diluted with 20 mL of H₂O. The reaction mixture was extracted with 3×10 mL of ethyl acetate and the organic layers combined, washed with 2×20 mL of brine and dried over anhydrous sodium sulfate. Concentration in vacuo resulted in 120 mg (crude) of 1-(methylamino)-N-[2-[(2R)-1-methylpyrrolidin-2-yl]-1-[[2-(trimethylsilyl)ethoxy]methyl]pyrrolo[3,2-c]pyridin-6-yl]isoquinoline-6-carboxamide as a brown oil.

LC-MS: (ES, m/z): [M+H]⁺=531

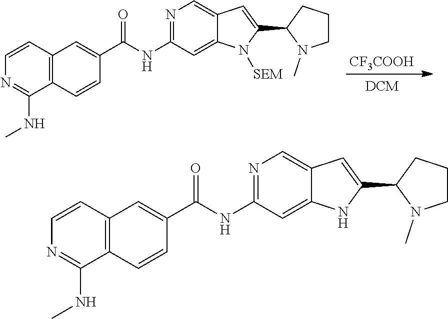

Into a 50-mL round-bottom flask, was placed 1-(methylamino)-N-[2-[(2R)-1-methylpyrrolidin-2-yl]-1-[[2-(trimethylsilyl)ethoxy]methyl]pyrrolo[3,2-c]pyridin-6-yl]isoquinoline-6-carboxamide (120.0 mg, 1 equiv), DCM (3.00 mL) and CF₃COOH (3.00 mL). The reaction mixture was stirred for 16 h at room temperature and concentrated under vacuum. The residue was diluted with 4 mL of DMF. The pH value of the solution was adjusted to 8 with NH₃/H₂O. The crude product was purified by Prep-HPLC eluting with 0.05% NH₃/H₂O and acetonitrile resulting in 34.8 mg of (R)-1-(methylamino)-N-(2-(1-methylpyrrolidin-2-yl)-1H-pyrrolo[3,2-c]pyridin-6-yl) isoquinoline-6-carboxamide as a white solid.

LC-MS: (ES, m/z): [M+H]⁺=401

¹H-NMR: (300 MHz, Methanol-d₄. ppm) δ 8.55 (s, 1H), 8.33 (d, J=1.8 Hz, 1H), 8.27-8.17 (m, 2H), 8.03 (dd, J=8.7, 1.8 Hz, 1H), 7.92 (d, J=6.0 Hz, 1H), 7.05 (d, J=6.0 Hz, 1H), 6.53 (s, 1H), 3.45 (t, J=7.8 Hz, 1H), 3.24 (t, J=7.8 Hz, 1H), 3.10 (s, 3H), 2.45-2.39 (m, 1H), 2.31 (s, 3H), 2.12-1.99 (m, 1H), 2.09-1.91 (m, 3H).

### Example 80: (R)-1-methyl-N-(2-(1-methylpiperidin-2-yl)-1H-pyrrolo[3,2-c]pyridin-6-yl) isoquinoline-6-carboxamide

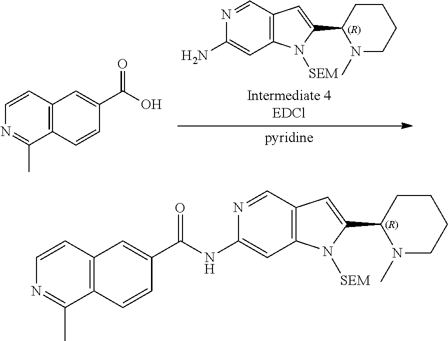

Into a 8-mL vial purged and maintained with an inert atmosphere of nitrogen, was placed 1-methylisoquinoline-6-carboxylic acid (858646-61-4, 30 mg, 0.16 mmol, 1.0 equiv), pyridine (3.00 mL), 2-[(2R)-1-methylpiperidin-2-yl]-1-{[2-(trimethylsilyl)ethoxy]methyl}pyrrolo[3,2-c]pyridin-6-amine (Intermediate 4, 57.8 mg, 0.16 mmol, 1 equiv) and EDCI (46 mg, 0.240 mmol, 1.5 equiv). The resulting solution was stirred for 12 h at room temperature and concentrated under vacuum. The residue was diluted with 20 mL of H₂O, extracted with 3×10 mL of ethyl acetate and the organic layers combined. The organic layer was washed with brine, dried over anhydrous sodium sulfate and concentrated under vacuum, resulting in 1-methyl-N-{2-[(2R)-1-methylpiperidin-2-yl]-1-{[2-(trimethylsilyl)ethoxy]methyl}pyrrolo[3,2-c]pyridin-6-yl}isoquinoline-6-carboxamide (70 mg, 83%) as brown oil.

LC-MS: (ES, m/z): [M+H]⁺=530

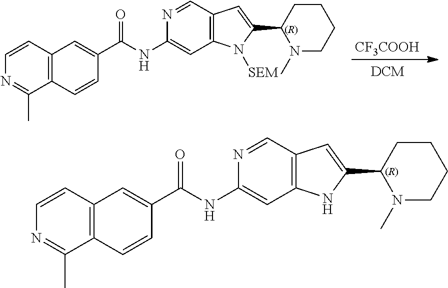

Into a 50-mL round-bottom flask, was placed 1-methyl-N-{2-[(2R)-1-methylpiperidin-2-yl]-1-{[2-(trimethylsilyl)ethoxy]methyl}pyrrolo[3,2-c]pyridin-6-yl}isoquinoline-6-carboxamide (70 mg, 0.132 mmol, 1.0 equiv), DCM (4.00 mL) and CF₃COOH (4.00 mL). The resulting solution was stirred for 12 h at room temperature and concentrated under vacuum. The residue was diluted with 4 mL of DMF. The pH of the solution was adjusted to 8 with NH₃/H₂O. The crude product (70 mg) was purified by Prep-HPLC eluting with 0.05% NH₃/H₂O and acetonitrile. This resulted in (R)-1-methyl-N-(2-(1-methylpiperidin-2-yl)-1H-pyrrolo[3,2-c]pyridin-6-yl) isoquinoline-6-carboxamide (29 mg, 55%) as a white solid.

LC-MS: (ES, m/z): [M+H]⁺=400

¹H-NMR: (300 MHz, Methanol-d₄ ppm) δ 8.55 (s, 2H), 8.50-8.40 (m, 2H), 8.23-8.21 (m, 2H), 7.84 (d, J=6.0 Hz, 1H), 6.51 (s, 1H), 3.21-3.17 (m, 1H), 3.16-3.13 (m, 1H), 3.00 (s, 3H), 2.26-2.23 (m, 1H), 2.11 (s, 3H), 1.89-1.76 (m, 5H), 1.55-1.41 (m, 1H).

### Example 81: (R)-3-fluoro-1-methyl-N-(2-(1-methylpiperidin-2-yl)-1H-pyrrolo[3,2-c]pyridin-6-yl)-1H-indazole-6-carboxamide

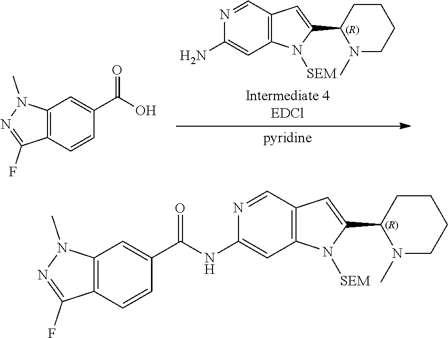

Into a 8-mL vial purged and maintained with an inert atmosphere of nitrogen, was placed 3-fluoro-1-methylindazole-6-carboxylic acid (Acid 28, 50 mg, 0.258 mmol, 1 equiv), pyridine (3.00 mL), 2-[(2R)-1-methylpiperidin-2-yl]-1-{[2-(trimethylsilyl)ethoxy]methyl}pyrrolo[3,2-c]pyridin-6-amine (Intermediate 4, 83.57 mg, 0.232 mmol, 0.9 equiv) and EDCI (148.10 mg, 0.774 mmol, 3 equiv). The resulting solution was stirred for 12 h at room temperature and concentrated under vacuum. The residue was diluted with 20 mL of H₂O and extracted with 3×10 mL of ethyl acetate. The organic layers were combined and washed with brine, dried over anhydrous sodium sulfate and concentrated under vacuum. This resulted in (R)-3-fluoro-1-methyl-N-(2-(1-methylpiperidin-2-yl)-1H-pyrrolo[3,2-c]pyridin-6-yl)-1H-indazole-6-carboxamide (80 mg, 58%) as brown oil.

LC-MS: (ES, m/z): [M+H]⁺=537

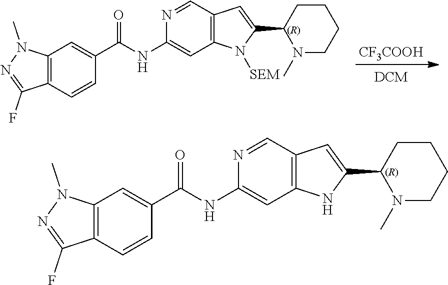

Into a 50-mL round-bottom flask, was placed 3-fluoro-1-methyl-N-{2-[(2R)-1-methylpiperidin-2-yl]-1-{[2-(trimethylsilyl)ethoxy]methyl}pyrrolo[3,2-c]pyridin-6-yl}indazole-6-carboxamide (80 mg, 0.149 mmol, 1 equiv), DCM (4.00 mL) and CF₃COOH (4.00 mL). The resulting solution was stirred for 12 h at room temperature and concentrated under vacuum. The residue was diluted with 4 mL of DMF and the pH of the solution was adjusted to 8 with NH₃/H₂O. The crude product (70 mg) was purified by Prep-HPLC eluting with 0.05% NH₃H₂O and acetonitrile. This resulted in 3-fluoro-1-methyl-N-{2-[(2R)-1-methylpiperidin-2-yl]-1H-pyrrolo[3,2-c]pyridin-6-yl}indazole-6-carboxamide (24.7 mg, 41%) as a white solid.

LC-MS: (ES, m/z): [M+H]⁺=407

¹H-NMR: (300 MHZ, Methanol-d₄. ppm) δ8.54 (s, 1H), 8.20 (s, 2H), 7.85-7.72 (m, 2H), 6.50 (s, 1H), 4.03 (s, 3H), 3.15 (dd, J=9.0, 4.2 Hz, 1H), 3.06 (d, J=11.1 Hz, 1H), 2.26-2.21 (m, 1H), 2.11 (s, 3H), 1.88-1.76 (m, 5H), 1.48-1.45 (m, 1H).

F-NMR: (282 MHZ, Methanol-d₄, ppm) δ −137.611

Prepared as for Example 79 using Intermediate 2 and corresponding acid:

| Example | | | | |
|---|---|---|---|---|
| No | Structure/Name | Acid | 1H NMR | LCMS |
| 82 | 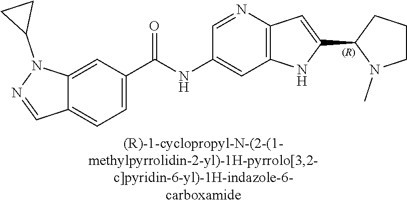 | 49 | ¹H-NMR: (300 MHz, Methanol- d₄, ppm) δ 8.54 (d, J = 0.6 Hz, 1H), 8.37 (s, 1H), 8.22 (s, 1H), 8.07 (d, J = 0.6 Hz, 1H), 7.89 (dd, J = 8.5, 0.9 Hz, 1H), 7.78 (dd, J = 8.5, 1.5 Hz, 1H), 6.53 (d, J = 0.9 | LC-MS: (ES, m/z): [M + H]⁺ = 401 |
| | | | Hz, 1H), 3.78- | |
| | | | 3.71 (m, 1H), | |
| | | | 3.45 (t, J = 7.8 | |
| | | | Hz, 1H), 3.23 (t, | |
| | | | J = 7.8 Hz, 1H), | |
| | | | 2.48-2.22 (m, | |
| | | | 5H), 2.12-1.87 | |
| | | | (m, 3H), 1.25 (d, | |
| | | | J = 5.4 Hz, 4H). | |
| 83 | 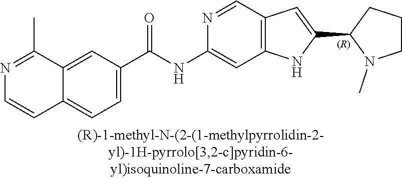 | 50 | ¹H-NMR (300 MHz, Methanol- d₄, ppm) δ 8.93 (s, 1H), 8.55 (s, 1H), 8.38 (d, J = 6.0 Hz, 1H), 8.31 (dd, J = 8.7, 1.8 Hz, 1H), 8.23 (s, 1H), 8.07 (d, J = 8.7 Hz, 1H), 7.76 (d, | LC-MS (ES, m/z): [M + 1]⁺ = 386 |
| | | | J = 6.0 Hz, 1H), | |
| | | | 6.53 (s, 1H), | |
| | | | 3.45 (t, J = 8.1 | |
| | | | Hz, 1H), 3.29- | |
| | | | 3.21 (m, 1H), | |
| | | | 3.07 (s, 3H), | |
| | | | 2.45-2.34 (m, | |
| | | | 1H), 2.31-2.45 | |
| | | | (m, 4H), 2.10- | |
| | | | 1.97 (m, 3H). | |
| 84 | 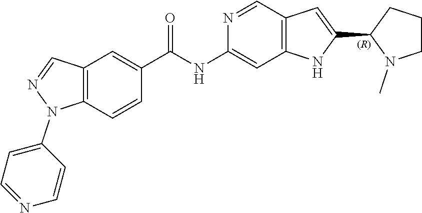 | 52 | ¹H-NMR: (300 MHz, CD₃OD, ppm): δ 8.72- 8.70 (m, 2H), 8.60 (s, 1H), 8.54 (s, 2H), 8.24-8.20 (m, 3H), 8.03-8.01 (m, 2H), 6.52 (s, 1H), 3.48- 3.42 (m, 1H), 3.30-3.21 (m, 1H), 2.43-2.36 | LC-MS (ES, m/z): [M + 1]⁺ = 438 |
| | | | (m, 1H), 2.32- | |
| | | | 2.25 (m, 4H), | |
| | | | 2.07-2.01 (m, | |
| | | | 2H), 1.98-1.92 | |
| | | | (m, 1H). | |
| 85 | 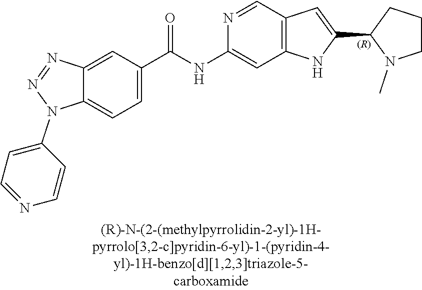 | 53 | ¹H-NMR (400 MHz, Methanol-d₄, ppm) δ 8.87- 8.85 (m, 3H), 8.54 (d, J = 0.8 Hz, 1H), 8.36 (dd, J = 8.8, 1.6 Hz, 1H), 8.26 (d, J = 8.8 Hz, 1H), 8.20 (s, 1H), 8.12 (dd, J = 4.8, 1.6 Hz, 2H), 6.53 (s, 1H), 3.48- 3.42 (m, 1H), 3.25-3.21 (m, 1H), 2.40 (q, J = | LC-MS (ES, m/z): [M + 1]⁺ = 389 |
| | | | 17.6, 8.8 Hz, | |
| | | | 1H), 2.32- | |
| | | | 2.25 (m, 4H), | |
| | | | 2.07-1.98 (m, | |
| | | | 2H), 1.95- | |
| | | | 1.91 (m, 1H). | |

Prepared as for Example 2 using Intermediate 4 and corresponding acid:

| Example | | | | |
|---|---|---|---|---|
| No | Structure/Name | Acid | ¹H NMR | LCMS |
| 86 | 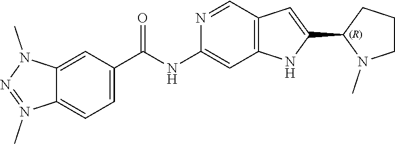 | 39 | ¹H-NMR: (300 MHz, Methanol-d₄, ppm) δ 8.56 (s, 1H), 8.23 (s, 1H), 8.19 (s, 1H), 7.86 (dd, J = 8.4, 0.9 Hz, 1H), 7.75 | LC-MS (ES, m/z): [M + H]⁺ = 403 |
| | | | (dd, J = 8.4, | |
| | | | 1.5 Hz, 1H), | |
| | | | 6.52 (d, J = | |
| | | | 0.9 Hz, 1H), | |
| | | | 4.11 (s, 3H), | |
| | | | 3.17 (dd, J = | |
| | | | 9.3, 4.5 Hz, | |
| | | | 1H), 3.08 (d, J = | |
| | | | 11.4 Hz, | |
| | | | 1H), 2.60 (s, | |
| | | | 3H), 2.32-2.17 | |
| | | | (m, 1H), 2.14 | |
| | | | (s, 3H), 1.94- | |
| | | | 1.78 (m, 5H), | |
| | | | 1.54-1.45 (m, | |
| | | | 1H). | |
| 87 | 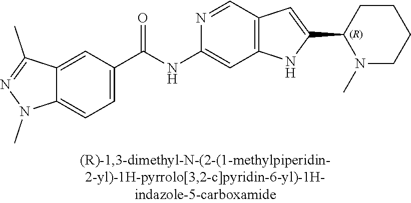 | 51 | ¹H-NMR: (300 MHz, Methanol-d₄, ppm) δ 8.55 (d, J = 0.9 Hz, 1H), 8.48 (s, 1H), 8.21 (s, 1H), 8.08 (dd, J = 9.0, 1.8 Hz, 1H), 7.63 (d, J = 9.0 Hz, 1H), 6.52 (s, 1H), 4.06 (s, | LC-MS: (ES, m/z): [M + H]⁺ = 403 |
| | | | 3H), 3.23-3.03 | |
| | | | (m, 2H), 2.65 | |
| | | | (s, 3H), 2.25- | |
| | | | 2.21 (m, 1H), | |
| | | | 2.14 (s, 3H), | |
| | | | 1.90-1.71 (m, | |
| | | | 5H), 1.54-1.51 | |
| | | | (m, 1H). | |
| 88 | 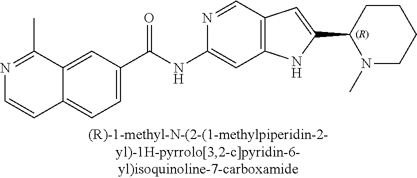 | 50 | ¹H-NMR (300 MHz, Methanol-d₄, ppm) δ 8.93 (s, 1H), 8.55 (s, 1H), 8.38 (d, J = 6.0 Hz, 1H), 8.32 (d, J = 8.7 Hz, 1H), 8.23 (s, 1H), 8.06 | LC-MS (ES, m/z): [M + 1]⁺ = 400 |
| | | | (d, J = 8.4 | |
| | | | Hz, 1H), 7.75 | |
| | | | (d, J = 5.7 | |
| | | | Hz, 1H), 6.51 | |
| | | | (s, 1H), 3.29- | |
| | | | 3.14 (m, | |
| | | | 1H), 3.07- | |
| | | | 3.05 (m, 4H), | |
| | | | 2.27-2.18 | |
| | | | (m, 1H), 2.12 | |
| | | | (s, 3H), 1.89- | |
| | | | 1.82 (m, | |
| | | | 3H), 1.76- | |
| | | | 1.73 (m, 2H), | |
| | | | 1.48-1.41 | |
| | | | (m, 1H). | |
| 89 | 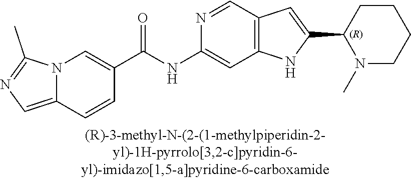 | G | ¹H-NMR: (300 MHz, Methanol-d₄, ppm) δ 8.78 (d, J = 0.9 Hz, 1H), 8.56 (d, J = 0.9 Hz, 1H), 8.17 (t, J = 0.9 Hz, 1H), 7.62 (dd, J = 9.6, 1.2 Hz, 1H), | LC-MS: (ES, m/z): [M + H]⁺ = 389 |
| | | | 7.38 (d, J = | |
| | | | 0.9 Hz, 1H), | |
| | | | 7.29 (dd, J = | |
| | | | 9.6, 1.5 Hz, | |
| | | | 1H), 6.52 (s, | |
| | | | 1H), 3.19 (dd, | |
| | | | J = 9.3, 4.5 | |
| | | | Hz, 1H), 3.09 | |
| | | | (d, J = 11.1 | |
| | | | Hz, 1H), 2.77 | |
| | | | (s, 3H), 2.30- | |
| | | | 2.24 (m, 1H), | |
| | | | 2.14 (s, 3H), | |
| | | | 1.91-1.75 (m, | |
| | | | 5H), 1.50-1.48 | |
| | | | (m, 1H). | |
| 90 | 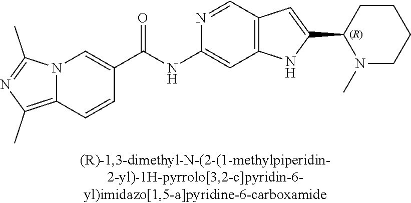 | 44 | ¹H-NMR: (400 MHz, CD₃OD- d₄, ppm): ) δ 8.64 (s, 1H), 8.53 (d, J = 0.8 Hz, 1H), 8.14 (s, 1H), 7.51 (dd, J = 9.6, 0.8 Hz, 1H), 7.14 (d, J = 9.6 Hz, 1H), 6.49 (s, 1H), 3.16-3.12 (m, | LC-MS: (ES, m/z): [M + H ]⁺ = 403 |
| | | | 1H), 3.07- | |
| | | | 3.04 (m, 1H), | |
| | | | 2.69 (s, 3H), | |
| | | | 2.46 (s, 3H), | |
| | | | 2.27-2.18 (m, | |
| | | | 1H), 2.11 (s, | |
| | | | 3H), 1.89- | |
| | | | 1.82 (m, 3H), | |
| | | | 1.80-1.73 (m, | |
| | | | 2H), 1.50 (m, | |
| | | | 1H). | |
| 91 | 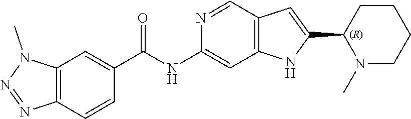 | 47 | ¹H-NMR: (400 MHz, CD₃OD- d₄, ppm): δ 8.55 (d, J = 0.8 Hz, 1H), 8.45 (s, 1H), 8.21 (s, 1H), 8.12 (dd, J = | LC-MS (ES, m/z): [M + H]⁺ = 390 |
| | (R)-1-methyl-N-(2-(1-methylpiperidin-2- | | 8.8, 0.8 Hz, | |
| | yl)-1H-pyrrolo[3,2-c]pyridin-6-yl)-1H- | | 1H), 8.04 (dd, | |
| | benzo[d][1,2,3]triazole-6-carboxamide | | J = 8.8, 1.6 | |
| | | | Hz, 1H), 6.50 | |
| | | | (d, J = 1.0 Hz, | |
| | | | 1H), 4.44 (s, | |
| | | | 3H), 3.18- | |
| | | | 3.12 (m, 1H), | |
| | | | 3.08-3.05 (d, | |
| | | | J = 11.7 Hz, | |
| | | | 1H), 2.26-2.20 | |
| | | | (m, 1H), 2.12 | |
| | | | (s, 3H), 1.88- | |
| | | | 1.81 (m, 3H), | |
| | | | 1.79-1.72 (m, | |
| | | | 2H), 1.51-1.49 | |
| | | | (m, 1H). | |
| 92 | 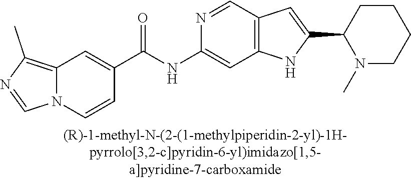 [SMILES: Cc1ncn2ccc(C(=O)Nc3cc4[nH]c([C@H]5CCCCN5C)cc4cn3)cc12] | 43 | ¹H-NMR (300 MHz, CD₃OD, ppm): δ 8.55 (d, J = 0.9 Hz, 1H), 8.37 (s, 1H), 8.33 (s, 1H), 8.23 (dd, J = 7.5, 0.9 Hz, 1H), 8.17 (s, 1H), 7.18 (dd, J = 7.5, | LC-MS: (ES, m/z): [M + H]⁺ = 389 |
| | | | 1.5 Hz, 1H), | |
| | | | 6.51 (s, 1H), | |
| | | | 3.20-3.15 (m, | |
| | | | 1H), 3.10- | |
| | | | 3.06 (m, 1H), | |
| | | | 2.61 (s, 3H), | |
| | | | 2.29-2.20 (m, | |
| | | | 1H), 2.13 (s, | |
| | | | 3H), 1.91- | |
| | | | 1.83 (m, 3H), | |
| | | | 1.78-1.75 | |
| | | | (m, 2H), 1.50- | |
| | | | 1.48 (m, 1H). | |
| 93 | 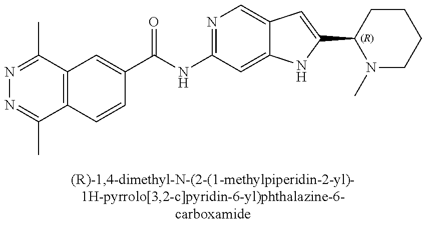 | 54 | ¹H-NMR (300 MHz, CD₃OD, ppm): δ 8.88 (s, 1H), 8.58- 8.56 (m, 2H), 8.42 (d, J = 8.7 Hz, 1H), 8.25 (s, 1H), 6.53 (s, 1H), 3.33-3.16 (m, 2H), 3.08 (s, 3H), 3.02 (s, 3H), 2.30- | LC-MS: (ES, m/z): [M + H]⁺ = 415 |
| | | | 2.20 (m, 1H), | |
| | | | 2.14 (s, 3H), | |
| | | | 1.92-1.79 | |
| | | | (m, 5H), 1.51- | |
| | | | 1.48 (m, | |
| | | | 1H). | |
| 94 | 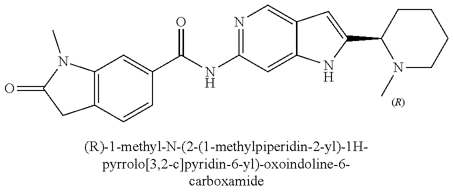 | 55 | ¹H-NMR: (300 MHz, DMSO- d₆, ppm) δ 11.44 (s, 1H), 10.47 (s, 1H), 8.51 (s, 1H), 8.21 (s, 1H), 7.72 (m, 2H), 7.38 (d, J = 8.1 Hz, 1H), 6.37 (s, 1H), | LC-MS: (ES, m/z): [M + H]⁺ = 404 |
| | | | 3.64 (s, 2H), | |
| | | | 3.21 (s, 3H), | |
| | | | 3.11-3.02 | |
| | | | (m, 1H), 2.98- | |
| | | | 2.88 (m, 1H), | |
| | | | 2.13-2.07 (m, | |
| | | | 1H), 1.97 (s, | |
| | | | 3H), 1.80- | |
| | | | 1.73 (m, 5H), | |
| | | | 1.46-1.27 (m, | |
| | | | 1H). | |
| 95 | 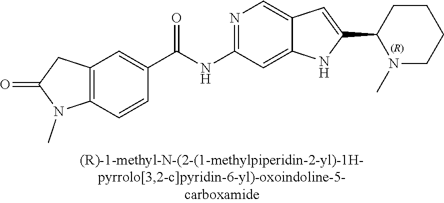 | 56 | ¹H-NMR: ¹H NMR (400 MHz, DMSO- d₆, ppm) δ 11.38 (s, 1H), 10.30 (s, 1H), 8.49 (s, 1H), 8.16 (s, 1H), 8.06 (s, 1H), 7.97 (s, 1H), 7.07 (d, J = 6.0 Hz, 1H), 6.35 (s, 1H), | LC-MS: (ES, m/z): [M + H]⁺ = 404 |
| | | | 3.63 (s, 2H), | |
| | | | 3.17 (s, 3H), | |
| | | | 3.09-3.00 | |
| | | | (m, 1H), 3.00- | |
| | | | 2.90 (m, 1H), | |
| | | | 2.13-2.03 (m, | |
| | | | 1H), 1.97 (s, | |
| | | | 3H), 1.84- | |
| | | | 1.70 (m, 3H), | |
| | | | 1.70-1.65 | |
| | | | (m, 2H), 1.45- | |
| | | | 1.25 (m, 1H). | |
| 96 | 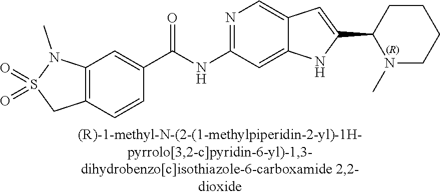 | 57 | ¹H-NMR (300 MHz, CD₃OD, ppm): δ 11.44 (s, 1H), 10.56 (s, 1H), 8.51 (s, 1H), 8.20 (s, 1H), 7.71-7.66 (m, 2H), 7.45 (d, J = 7.8 Hz, 1H), 6.37 (s, 1H), 4.76 (s, | LC-MS: (ES, m/z): [M + H]⁺ = 440 |
| | | | 2H), 3.15 (s, | |
| | | | 3H), 3.05 (d, J = | |
| | | | 6.9 Hz, 1H), | |
| | | | 2.96 (d, J = | |
| | | | 11.7 Hz, 1H), | |
| | | | 2.08 (m, 1H), | |
| | | | 1.97 (s, 3H), | |
| | | | 1.83-1.68 | |
| | | | (m, 3H), 1.63 | |
| | | | (s, 2H), 1.38- | |
| | | | 1.34 (m, 1H). | |
| 97 | 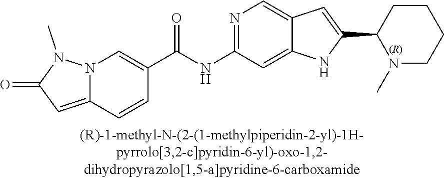 | 58 | ¹H-NMR (300 MHz, CD₃OD, ppm): δ 9.07 (dt, J = 1.8, 0.8 Hz, 1H), 8.54 (d, J = 0.9 Hz, 1H), 8.15 (s, 1H), 7.72 (dd, J = 9.3, 1.8 Hz, 1H), 7.52 (dd, | LC-MS: (ES, m/z): [M + H]⁺ = 405 |
| | | | J = 9.3, 0.9 | |
| | | | Hz, 1H), 6.51 | |
| | | | (d, J = 0.9 Hz, | |
| | | | 1H), 6.08 (s, | |
| | | | 1H), 4.04 (s, | |
| | | | 3H), 3.20- | |
| | | | 3.15 (m, 1H), | |
| | | | 3.08 (d, J = | |
| | | | 11.7 Hz, 1H), | |
| | | | 2.34-2.17 | |
| | | | (m, 1H), 2.13 | |
| | | | (s, 3H), 1.93- | |
| | | | 1.72 (m, 5H), | |
| | | | 1.53-1.51 | |
| | | | (m, 1H). | |
| 98 | 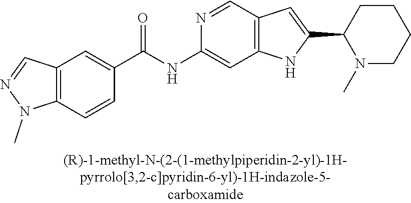 [SMILES: CN1CCCC[C@@H]1c1cc2cnc(NC(=O)c3ccc4c(cnn4C)c3)cc2[nH]1] | B | ¹H-NMR (300 MHz, CD₃OD, ppm): δ 8.53 (d, J = 0.9 Hz, 1H), 8.49 (d, J = 0.9 Hz, 1H), 8.19-8.18 (m, 2H), 8.06 (dd, J = 9.0, 1.8 Hz, 1H), 7.69 (d, J = 9.0 Hz, 1H), 6.50 (s, 1H), | LC-MS: (ES, m/z): [M + H]⁺ = 389 |
| | | | 4.13 (s, 3H), | |
| | | | 3.18-3.15 | |
| | | | (m, 1H), 3.08- | |
| | | | 3.05 (m, | |
| | | | 1H), 2.27-2.15 | |
| | | | (m, 1H), 2.12 | |
| | | | (s, 3H), 1.89- | |
| | | | 1.82 (m, 3H), | |
| | | | 1.80-1.76 (m, | |
| | | | 2H), 1.48-1.44 | |
| | | | (m, 1H). | |

### Example 99: (R)-1-methyl-N-(2-(1-methylpiperidin-2-yl)-1H-pyrrolo[3,2-c]pyridin-6-yl)-2-oxo-1,2-dihydroquinoline-6-carboxamide

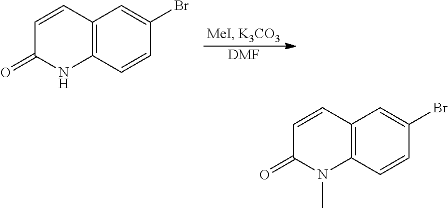

To a stirred mixture of 6-bromo-1H-quinolin-2-one (1 g, 4.46 mmol, 1 equiv) and K₂CO₃ (1.23 g, 8.93 mmol, 2 equiv) in DMF (20 mL) was added Mel (0.95 g, 6.7 mmol, 1.5 equiv) dropwise at room temperature. The resulting mixture was stirred for overnight at room temperature. The reaction was quenched by the addition of water (100 mL) at 0° C. and extracted with EtOAc (3×200 mL). The combined organic layers were washed with brine (1×200 mL) and dried over anhydrous Na₂SO₄. After filtration, the filtrate was concentrated under reduced pressure. The residue was purified by trituration with EtOAc (50 mL). This resulted in 6-bromo-1-methylquinolin-2-one (900 mg, 85%) as a light brown solid.

LC-MS: (ES, m/z): [M+H]⁺=238, 240

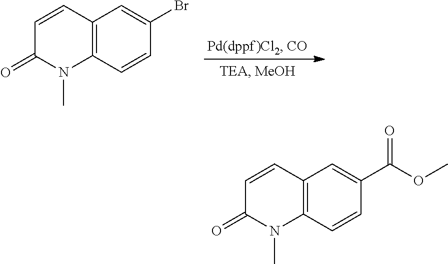 [SMILES: Cn1c(=O)ccc2cc(Br)ccc21 ; COC(=O)c1ccc2c(ccc(=O)n2C)c1]

Into a 30 mL pressure vessel were added 6-bromo-1-methylquinolin-2-one (900 mg, 3.78 mmol, 1 equiv), Pd(dppf)Cl₂ (138.3 mg, 0.189 mmol, 0.05 equiv), MeOH (20 mL, 494 mmol) and TEA (1530 mg, 15.12 mmol, 4 equiv) at room temperature. The resulting mixture was stirred for overnight at 120° C. under carbon monoxide atmosphere. The mixture was allowed to cool down to room temperature and concentrated under reduced pressure. The residue was purified by silica gel column chromatography, eluting with petroleum ether/ethyl acetate (1:2) to afford methyl 1-methyl-2-oxoquinoline-6-carboxylate (650 mg, 79%) as a brown solid.

LC-MS: (ES, m/z): [M+H]⁺=218

¹H-NMR: (400 MHZ, DMSO-d₆, ppm) δ 8.37 (d, J=2.0 Hz, 1H), 8.13 (dd, J=8.8, 2.0 Hz, 1H), 8.07 (d, J=9.6 Hz, 1H), 7.64 (d, J=8.8 Hz, 1H), 6.70 (d, J=9.6 Hz, 1H), 3.89 (s, 3H), 3.65 (s, 3H).

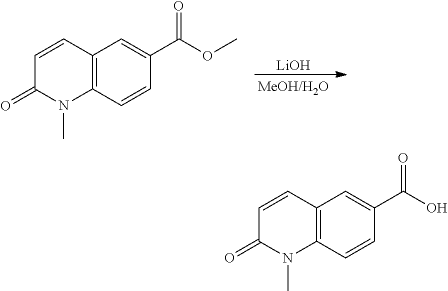

To a stirred solution of methyl 1-methyl-2-oxoquinoline-6-carboxylate (650 mg, 3.0 mmol, 1 equiv) in MeOH (10 mL) in THF (10 mL) was added a solution of LiOH (215 mg, 9 mmol, 3 equiv) in water (2 mL) at room temperature. The resulting mixture was stirred for overnight at room temperature, diluted with water (10 mL), then concentrated to about 10 mL under reduced pressure. The mixture was acidified to pH=3 with HCl (aq.). The precipitated solids were collected by filtration and washed with water (2×10 mL). The resulting solid was dried under infrared light. This resulted in 1-methyl-2-oxoquinoline-6-carboxylic acid (450 mg, 74%) as a light brown solid.

LC-MS: (ES, m/z): [M+H]⁺=204

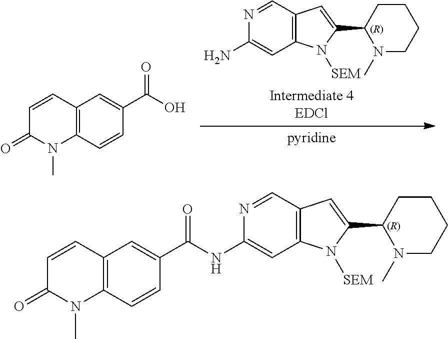

To a stirred solution 1-methyl-2-oxoquinoline-6-carboxylic acid (50.72 mg, 0.249 mmol, 1.5 equiv) and 2-[(2R)-1-methylpiperidin-2-yl]-1-{[2-(trimethylsilyl)ethoxy]methyl}pyrrolo[3,2-c]pyridin-6-amine (60 mg, 0.166 mmol, 1.00 equiv) in pyridine (2 mL) was added EDCI (63.80 mg, 0.332 mmol, 2 equiv) at room temperature. The resulting mixture was stirred for overnight at room temperature. The reaction was quenched by the addition of water (0.1 mL) at room temperature and concentrated under reduced pressure. The residue was dissolved in water (5 mL) and extracted with EtOAc (3×10 mL). The combined organic layers were washed with brine (1×20 mL), dried over anhydrous Na₂SO₄. After filtration, the filtrate was concentrated under reduced pressure. This resulted in 1-methyl-N-{2-[(2R)-1-methylpiperidin-2-yl]-1-{[2-(trimethylsilyl)ethoxy]methyl}pyrrolo[3,2-c]pyridin-6-yl}-2-oxoquinoline-6-carboxamide (90 mg, crude) as a brown oil. The crude product was used in the next step directly without further purification.

LC-MS: (ES, m/z): [M+H]⁺=546

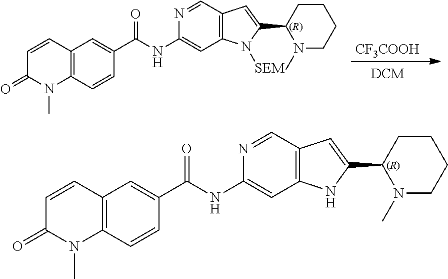

Into a 8 mL vial were added 1-methyl-N-{2-[(2R)-1-methylpiperidin-2-yl]-1-{[2-(trimethylsilyl)ethoxy]methyl}pyrrolo[3,2-c]pyridin-6-yl}-2-oxoquinoline-6-carboxamide (90 mg, crude), DCM (1.5 mL) and CF₃COOH (1.5 mL) at room temperature. The resulting mixture was stirred overnight at room temperature and concentrated under reduced pressure. The residue was dissolved in DMF (4 mL) and the mixture basified to pH 11 with ammonium hydroxide. The residue was purified by Prep-HPLC with the following conditions (Column: XBridge Shield RP18 OBD Column; Mobile Phase A: 0.05% NH₃/H₂O, Mobile Phase B: ACN; Flow rate: 20 mL/min; Gradient: 26% B to 50% B in 7 min; wave Length: 220 nm; RT1 (min): 6.7) to afford 1-methyl-N-{2-[(2R)-1-methylpiperidin-2-yl]-1H-pyrrolo[3,2-c]pyridin-6-yl}-2-oxoquinoline-6-carboxamide (19.6 mg) as a white solid.

LC-MS: (ES, m/z): [M+H]⁺=416

¹H-NMR (300 MHz, CD₃OD, ppm): δ 8.53 (s, 1H), 8.36 (d, J=2.1 Hz, 1H), 8.27 (dd, J=9.0, 2.1 Hz, 1H), 8.18 (s, 1H), 8.03 (d, J=9.3 Hz, 1H), 7.73 (d, J=9.0 Hz, 1H), 6.76 (d, J=9.6 Hz, 1H), 6.50 (s, 1H), 3.79 (s, 3H), 3.18-3.14 (m, 1H), 3.08-3.05 (m, 1H), 2.27-2.18 (m, 1H), 2.11 (s, 3H), 1.89-1.82 (m, 3H), 1.76-1.73 (m, 2H), 1.50-1.46 (m, 1H).

### Example 100: (R)-2-methyl-N-(2-(1-methylpiperidin-2-yl)-1H-pyrrolo[3,2-c]pyridin-6-yl)-1-oxo-1,2-dihydroisoquinoline-6-carboxamide

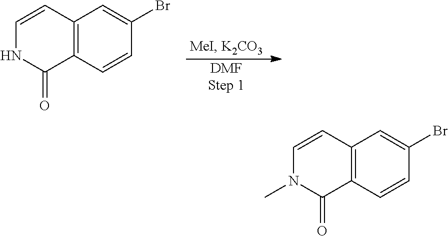

Into a 100-mL round-bottom flask, was placed methyl 6-bromo-2H-isoquinolin-1-one (1 g, 4.46 mmol, 1 equiv), DMF (20 mL), K₂CO₃ (1.85 g, 13.4 mmol, 3 equiv) and CH₃I (0.76 g, 5.4 mmol, 1.2 equiv). The resulting solution was stirred for 16 h at room temperature and diluted with 50 mL of H₂O. The reaction mixture was extracted with 3×20 mL of ethyl acetate and the combined organic layers were washed with brine, dried over anhydrous Na₂SO₄ and concentrated under reduced pressure. The residue was purified by silica gel column chromatography, eluting with ethyl acetate/petroleum ether (1:1) to afford 6-bromo-2-methylisoquinolin-1-one (900 mg, 85%) as a brown solid.

LC-MS: (ES, m/z): [M+H]⁺=238

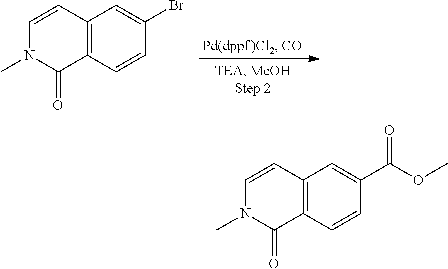

Into a 50-mL pressure vessel was placed 6-bromo-2-methylisoquinolin-1-one (900 mg, 3.78 mmol, 1 equiv), CH₃OH (5 mL), Pd(dppf)Cl₂ (276.6 mg, 0.378 mmol, 0.1 equiv), TEA (1530 mg, 15.12 mmol, 4 equiv) and CO (10 atm). The resulting solution was stirred for 16 h at 120° C. The reaction mixture was cooled and concentrated under vacuum. The residue was diluted with 60 mL of H₂O and extracted with 3×20 mL of ethyl acetate. The organic layers were combined and washed with 3×20 mL of brine, dried over anhydrous sodium sulfate and concentrated under vacuum. The residue was applied onto a silica gel column and eluted with ethyl acetate/petroleum ether (1:1). This resulted in methyl 2-methyl-1-oxoisoquinoline-6-carboxylate (750 mg, 91%) as an orange solid.

LC-MS: (ES, m/z): [M+H]⁺=218

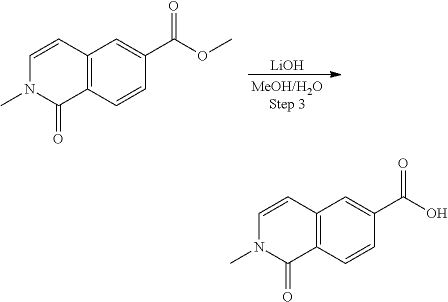

Into a 100-mL round-bottom flask, was placed methyl 2-methyl-1-oxoisoquinoline-6-carboxylate (750 mg, 3.45 mmol, 1 equiv), CH₃OH (15 mL), H₂O (5.0 mL), and lithium hydroxide (248 mg, 10.36 mmol, 3 equiv). The resulting solution was stirred for 16 h at room temperature. The resulting mixture was concentrated under vacuum and diluted with 20 mL of H₂O. The reaction mixture was extracted with 2×10 mL of ethyl acetate and the aqueous layers combined. The pH value of the solution was adjusted to 3 with HCl (3 mol/L). The solids were collected by filtration. This resulted in 2-methyl-1-oxoisoquinoline-6-carboxylic acid (700 mg, 99.8%) as an orange solid.

LC-MS: (ES, m/z): [M+H]⁺=204

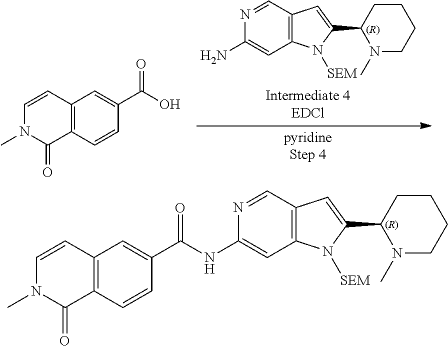

Into a 8-mL vial purged and maintained with an inert atmosphere of nitrogen, was placed 2-methyl-1-oxoisoquinoline-6-carboxylic acid (70 mg, 0.344 mmol, 1 equiv), pyridine (3.00 mL), 2-[(2R)-1-methylpiperidin-2-yl]-1-{[2-(trimethylsilyl)ethoxy]methyl}pyrrolo[3,2-c]pyridin-6-amine (124 mg, 0.344 mmol, 1 equiv) and EDCI (198 mg, 1.03 mmol, 3 equiv). The resulting solution was stirred for 16 h at room temperature and concentrated under vacuum. The residue was diluted with 20 mL of H₂O and extracted with 3×10 mL of ethyl acetate. The combined organic layers were washed with 2×10 mL of brine, dried over anhydrous sodium sulfate then concentrated under vacuum. This resulted in 2-methyl-N-{2-[(2R)-1-methylpiperidin-2-yl]-1-{[2-(trimethylsilyl)ethoxy]methyl}pyrrolo[3,2-c]pyridin-6-yl}-1-oxoisoquinoline-6-carboxamide (80 mg, crude) as brown oil.

LC-MS: (ES, m/z): [M+H]⁺=546

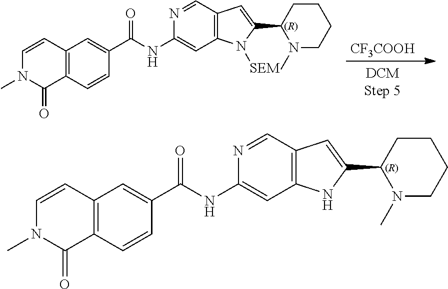

Into a 50-mL round-bottom flask, was placed 2-methyl-N-{2-[(2R)-1-methylpiperidin-2-yl]-1-{[2-(trimethylsilyl)ethoxy]methyl}pyrrolo[3,2-c]pyridin-6-yl}-1-oxoisoquinoline-6-carboxamide (80 mg, 0.147 mmol, 1 equiv), DCM (3.0 mL) and CF₃COOH (3.0 mL). The resulting solution was stirred for 16 h at room temperature and concentrated under vacuum. The residue was diluted with 4 mL of DMF and the pH of the solution was adjusted to 8 with NH₃·H₂O. The crude product (70 mg) was purified by Prep-HPLC with the following conditions: Column, XBridge Shield RP18 OBD Column; mobile phase A, 0.05% NH₃·H₂O) and acetonitrile (15% Phase B up to 31% in 7 min). This resulted in 2-methyl-N-{2-[(2R)-1-methylpiperidin-2-yl]-1H-pyrrolo[3,2-c]pyridin-6-yl}-1-oxoisoquinoline-6-carboxamide (36.5 mg, 60%) as a white solid.

LC-MS: (ES, m/z): [M+H]⁺=416

¹H-NMR: (300 MHz, Methanol-d₄ ppm) δ8.54 (d, J=0.6 Hz, 1H), 8.45 (d, J=8.4 Hz, 1H), 8.29-8.17 (m, 2H), 8.06 (dd, J=8.4, 1.8 Hz, 1H), 7.46 (d, J=7.2 Hz, 1H), 6.82 (d, J=7.2 Hz, 1H), 6.50 (s, 1H), 3.65 (s, 3H), 3.15 (dd, J=9.3, 4.5 Hz, 1H), 3.06 (d, J=11.4 Hz, 1H), 2.22 (dd, J=16.5, 10.2 Hz, 1H), 2.11 (s, 3H), 1.88-1.75 (m, 5H), 1.53-1.45 (m, 1H).

### Example 101: (R)-3-methyl-N-(2-(1-methylpiperidin-2-yl)-1H-pyrrolo[3,2-c]pyridin-6-yl)-4-oxo-3,4-dihydroquinazoline-7-carboxamide

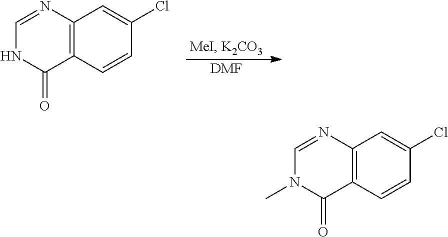

To a stirred mixture of 7-chloro-3H-quinazolin-4-one (1 g, 5.54 mmol, 1 equiv) and K₂CO₃ (2.30 g, 16.6 mmol, 3 equiv) in DMF (20 mL) was added Mel (1.18 g, 8.306 mmol, 1.5 equiv) dropwise at room temperature. The resulting mixture was stirred for overnight at room temperature. The reaction was quenched with water (100 mL) at 0° C. and extracted with EtOAc (3×30 mL). The combined organic layers were washed with brine (3×20 mL), dried over anhydrous Na₂SO₄ and concentrated under reduced pressure. The residue was purified by silica gel column chromatography, eluted with petroleum ether/ethyl acetate (3:1) to afford 7-chloro-3-methylquinazolin-4-one (900 mg, 84%) as a yellow solid.

LC-MS: (ES, m/z): [M+H]⁺=195,197

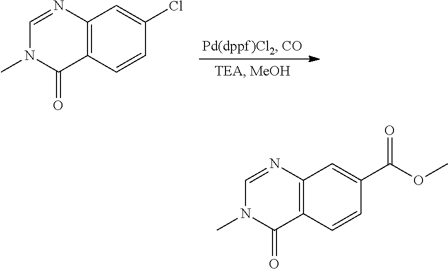

Into a 50 mL pressure tank reactor were added 7-chloro-3-methylquinazolin-4-one (900 mg, 4.624 mmol, 1 equiv), Pd(dppf)Cl₂ (169.18 mg, 0.231 mmol, 0.05 equiv), MeOH (20 mL) and TEA (1871.77 mg, 18.496 mmol, 4 equiv) at room temperature. The resulting mixture was stirred for 16 h at 140° C. under carbon monoxide atmosphere. The mixture was allowed to cool down to room temperature. The resulting mixture was concentrated under reduced pressure. The residue was purified by silica gel column chromatography, eluted with petroleum ether/THF (1:1) to afford methyl 3-methyl-4-oxoquinazoline-7-carboxylate (600 mg, 60%) as a light yellow solid.

LC-MS: (ES, m/z): [M+H]⁺=219

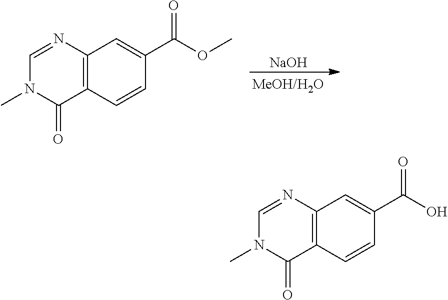

To a stirred solution of methyl 3-methyl-4-oxoquinazoline-7-carboxylate (600 mg, 2.75 mmol, 1 equiv) in MeOH (10 mL) was added a solution of NaOH (330 mg, 8.25 mmol, 3.00 equiv) in water (2 mL) at room temperature. The resulting mixture was stirred for overnight at room temperature and diluted with water (10 mL), then concentrated to ˜10 mL under reduced pressure. The resulting mixture was washed with 1×10 mL of DCM and the aqueous layer was acidified to pH 3 with HCl (aq.). The precipitated solids were collected by filtration and washed with water (2×10 mL). The resulting solid was dried under infrared light. This resulted in 3-methyl-4-oxoquinazoline-7-carboxylic acid (450 mg, 80%) as an off-white solid.

LC-MS: (ES, m/z): [M+H]⁺=205

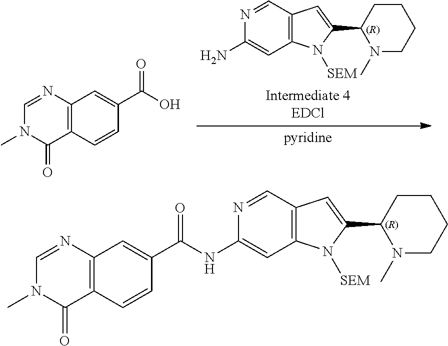

To a stirred solution of 3-methyl-4-oxoquinazoline-7-carboxylic acid (50.96 mg, 0.249 mmol, 1.5 equiv) and 2-[(2R)-1-methylpiperidin-2-yl]-1-{[2-(trimethylsilyl)ethoxy]methyl}pyrrolo[3,2-c]pyridin-6-amine (60 mg, 0.166 mmol, 1.00 equiv) in pyridine (2 mL) was added EDCI (63.80 mg, 0.332 mmol, 2 equiv) at room temperature. The resulting mixture was stirred for overnight at room temperature. The reaction was quenched by the addition of water (0.25 mL) at room temperature and concentrated under reduced pressure. The residue was dissolved in EtOAc (25 mL) and washed with 2×20 mL of water. The organic layer was dried over anhydrous Na₂SO₄ and concentrated under reduced pressure. This resulted in 3-methyl-N-{2-[(2R)-1-methylpiperidin-2-yl]-1-{[2-(trimethylsilyl)ethoxy]methyl}pyrrolo[3,2-c]pyridin-6-yl}-4-oxoquinazoline-7-carboxamide (100 mg, crude) as a brown oil. The crude product was used in the next step directly without further purification.

LC-MS: (ES, m/z): [M+H]⁺=547

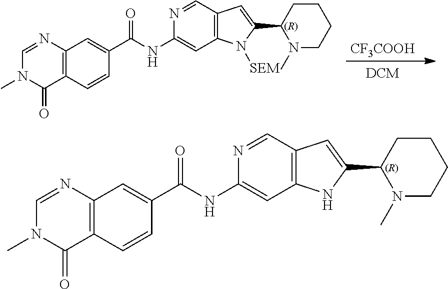

Into a 8 mL vial were added 3-methyl-N-{2-[(2R)-1-methylpiperidin-2-yl]-1-{[2-(trimethylsilyl)ethoxy]methyl}pyrrolo[3,2-c]pyridin-6-yl}-4-oxoquinazoline-7-carboxamide (100 mg, crude), DCM (1.5 mL) and CF₃COOH (1.5 mL) at room temperature. The resulting mixture was stirred overnight at room temperature and concentrated under vacuum. The residue was dissolved in DMF (3 mL). The mixture was basified to pH 11 with ammonium hydroxide. The resulting mixture was purified by Prep-HPLC with the following conditions (Column: XBridge Prep C18 OBD; Mobile Phase A: 0.05% NH₃·H₂O, Mobile Phase B: ACN; Flow rate: 60 mL/min; Gradient: 5% B to 50% B in 8 min). The collected solution was concentrated under vacuum to remove ACN and resulting solution was dried by lyophilization. This resulted in 3-methyl-N-{2-[(2R)-1-methylpiperidin-2-yl]-1H-pyrrolo[3,2-c]pyridin-6-yl}-4-oxoquinazoline-7-carboxamide (18.2 mg) as a white solid.

LC-MS: (ES, m/z): [M+H]⁺=417

¹H-NMR (300 MHz, CD₃OD. ppm): δ 8.54 (s, 1H), 8.40-8.37 (m, 2H), 8.28 (d, J=1.2 Hz, 1H), 8.19 (s, 1H), 8.09 (dd, J=8.4, 1.8 Hz, 1H), 6.50 (s, 1H), 3.63 (s, 3H), 3.19-3.14 (m, 1H), 3.06 (d, J=11.4 Hz, 1H), 2.27-2.19 (m, 1H), 2.12 (s, 3H), 1.89-1.82 (m, 3H), 1.76-1.73 (m, 2H), 1.48-1.46 (m, 1H).

### Example 102: (R)-3-methyl-N-(2-(1-methylpiperidin-2-yl)-1H-pyrrolo[3,2-c]pyridin-6-yl)-4-oxo-3,4-dihydroquinazoline-6-carboxamide

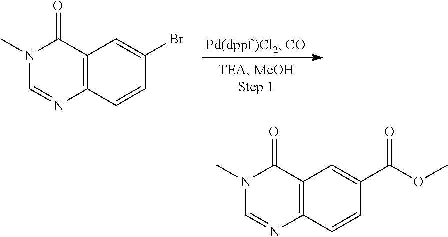

Into a 50-mL pressure seal-tube were added 6-bromo-3-methylquinazolin-4-one (1 g, 4.18 mmol, 1 equiv), TEA (1.27 g, 12.55 mmol, 3 equiv), MeOH (15 mL) and Pd(dppf)Cl₂ (0.31 g, 0.418 mmol, 0.1 equiv). The resulting mixture was stirred for 16 h at 120° C. under carbon monoxide atmosphere. The mixture was allowed to cool down to room temperature and filtered, the filter cake was washed with EtOAc (3×20 mL). The filtrate was concentrated under reduced pressure. The residue was purified by silica gel column chromatography, eluting with petroleum ether:ethyl acetate (1:2) to afford methyl 3-methyl-4-oxoquinazoline-6-carboxylate (810 mg, 89%) as a light yellow solid.

LC-MS (ES, m/z): [M+1]⁺=219

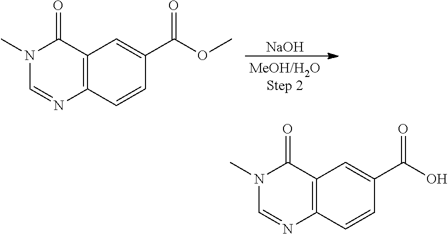

To a stirred solution of methyl 3-methyl-4-oxoquinazoline-6-carboxylate (810 mg, 3.71 mmol, 1 equiv) in MeOH (15 mL) and H₂O (5 mL) were added NaOH (296.9 mg, 7.42 mmol, 2 equiv). The resulting mixture was stirred for 4 h at room temperature. The mixture was concentrated under vacuum and basified to pH 3˜4 with 2M HCl (aq). The precipitated solids were collected by filtration and washed with water (3×5 mL). This resulted in 3-methyl-4-oxoquinazoline-6-carboxylic acid (600 mg, 71%) as an off-white solid.

LC-MS (ES, m/z): [M+1]⁺=205

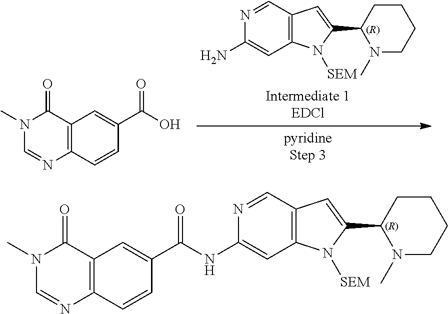

Into a 50-mL round-bottom-flask were added 3-methyl-4-oxoquinazoline-6-carboxylic acid (60 mg, 0.294 mmol, 1 equiv), 2-[(2R)-1-methylpiperidin-2-yl]-1-{[2-(trimethylsilyl)ethoxy]methyl}pyrrolo[3,2-c]pyridin-6-amine (105.96 mg, 0.294 mmol, 1 equiv), EDCI (225.33 mg, 1.176 mmol, 4 equiv) and pyridine (3 mL). The resulting mixture was stirred for 16 h at room temperature and concentrated under reduced pressure. The crude product 3-methyl-N-{2-[(2R)-1-methylpiperidin-2-yl]-1-{[2-(trimethylsilyl)ethoxy]methyl}pyrrolo[3,2-c]pyridin-6-yl}-4-oxoquinazoline-6-carboxamide (120 mg) was used in the next step directly without further purification.

LC-MS (ES, m/z): [M+1]⁺=547

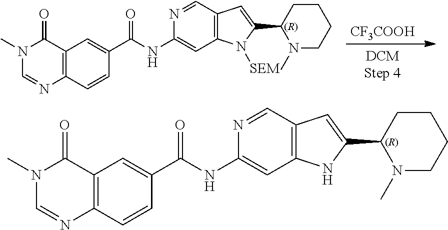

Into a 50-mL round-bottom-flask were added 3-methyl-N-{2-[(2R)-1-methylpiperidin-2-yl]-1-{[2-(trimethylsilyl)ethoxy]methyl}pyrrolo[3,2-c]pyridin-6-yl}-4-oxoquinazoline-6-carboxamide (120 mg, crude), DCM (1.5 mL) and trifluoroacetic acid (1.5 mL). The resulting mixture was stirred for 20 h at room temperature and concentrated under vacuum. The residue was diluted with DMF (3 mL) and NH₃·H₂O (˜10 drops) added then the mixture was stirred for other 4 h. The crude product was purified by Prep-HPLC with the following conditions (Column: XBridge Prep C18 OBD; Mobile Phase A: 0.05% NH₃·H₂O, Mobile Phase B: ACN; Flow rate: 60 mL/min; Gradient: 5% B to 50% B in 8 min. The fractions were concentrated under vacuum and dried by lyophilization. This resulted in 22.7 mg of 3-methyl-N-{2-[(2R)-1-methylpiperidin-2-yl]-1H-pyrrolo[3,2-c]pyridin-6-yl}-4-oxoquinazoline-6-carboxamide as a white solid.

LC-MS (ES, m/z): [M+1]⁺=417

¹H-NMR (400 MHZ, Methanol-d₄, ppm) δ 8.90 (d, J=2.0 Hz, 1H), 8.53 (s, 1H), 8.39-8.38 (m, 2H), 8.19 (s, 1H), 7.82 (d, J=8.4 Hz, 1H), 6.49-6.47 (m, 1H), 3.63 (s, 3H), 3.17-3.13 (m, 1H), 3.07-3.04 (m, 1H), 2.25-2.18 (m, 1H), 2.11 (s, 3H), 1.93-1.83 (m, 3H), 1.78-1.74 (m, 2H), 1.50-1.44 (m, 1H).

### Example 103: (R)-1,3-dimethyl-N-(2-(1-methylpiperidin-2-yl)-1H-pyrrolo[3,2-c]pyridin-6-yl)-4-oxo-3,4-dihydrophthalazine-6-carboxamide

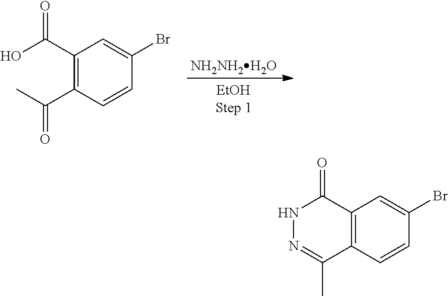

Into a 50-mL round-bottom-flask were added 2-acetyl-5-bromobenzoic acid (950 mg, 3.9 mmol, 1.0 equiv), hydrazine hydrate (85%) (587 mg, 11.73 mmol, 3.0 equiv) and EtOH (10 mL) at room temperature. The resulting mixture was stirred for 3 h at room temperature. The resulting mixture was concentrated under vacuum. The residue was purified by silica gel column chromatography, eluted with petroleum ether:ethyl acetate (1:2) to afford 7-bromo-4-methyl-2H-phthalazin-1-one (800 mg) as an off-white solid.

LC-MS (ES, m/z): [M+1]⁺=239

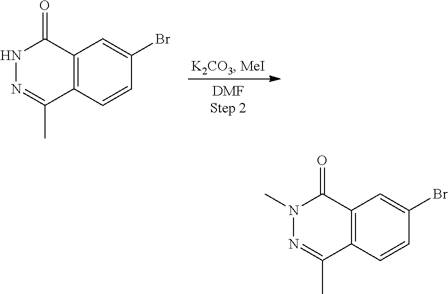

A solution of 7-bromo-4-methyl-2H-phthalazin-1-one (0.8 g, 3.35 mmol, 1 equiv) and K₂CO₃ (1.39 g, 10.04 mmol, 3.0 equiv) in DMF (10 mL) was treated with Mel (0.71 g, 5.02 mmol, 1.5 equiv) for 20 min at 0° C. dropwise. The mixture was stirred for 16 h at room temperature. The reaction was quenched with water (50 mL) at 0° C. and extracted with EtOAc (3×60 mL). The combined organic layers were washed with water (8×50 mL) and brine (60 mL), dried over anhydrous Na₂SO₄, then concentrated under reduced pressure. The residue was purified by silica gel column chromatography, eluting with petroleum ether:ethyl acetate (3:1) to afford 7-bromo-2,4-dimethylphthalazin-1-one (450 mg) as a yellow solid.

LC-MS (ES, m/z): [M+1]⁺=253

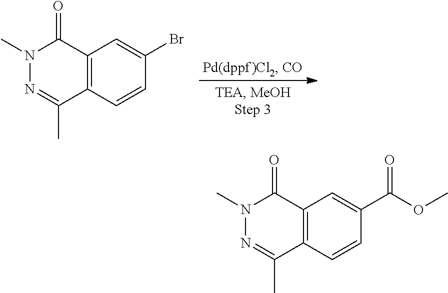

Into a 50-mL pressure sealed-tube were added 7-bromo-2,4-dimethylphthalazin-1-one (440 mg, 1.74 mmol, 1 equiv), TEA (703.68 mg, 6.95 mmol, 4.0 equiv), MeOH (10 mL) and Pd(dppf)Cl₂ (63.6 mg, 0.087 mmol, 0.05 equiv). The resulting mixture was stirred for 16 h at 120° C. under carbon monoxide atmosphere. The mixture was allowed to cool down to room temperature and filtered, the filter cake was washed with EtOAc (3×20 mL). The filtrate was concentrated under reduced pressure and the residue purified by silica gel column chromatography, eluting with petroleum ether:ethyl acetate (1:1) to afford methyl 1,3-dimethyl-4-oxophthalazine-6-carboxylate (310 mg) as a light yellow solid.

LC-MS (ES, m/z): [M+1]⁺=233

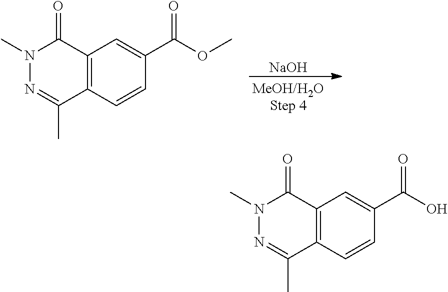

To a stirred solution of methyl 1,3-dimethyl-4-oxophthalazine-6-carboxylate (310 mg, 1.34 mmol, 1 equiv) in MeOH (10 mL) and H₂O (2 mL) were added NaOH (133.47 mg, 3.34 mmol, 2.5 equiv). The resulting mixture was stirred for 16 h at room temperature. The mixture was concentrated under vacuum and basified to pH 3˜4 with 2M HCl (aq). The precipitated solids were collected by filtration and washed with water (3×10 mL). This resulted in 1,3-dimethyl-4-oxophthalazine-6-carboxylic acid (220 mg) as an off-white solid.

LC-MS (ES, m/z): [M+1]⁺=219

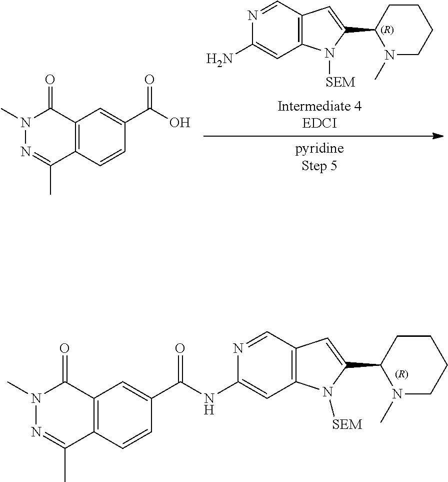

Into a 50-mL round-bottom-flask were added 1,3-dimethyl-4-oxophthalazine-6-carboxylic acid (60.0 mg, 0.275 mmol, 1 equiv), 2-[(2R)-1-methylpiperidin-2-yl]-1-{[2-(trimethylsilyl)ethoxy]methyl}pyrrolo[3,2-c]pyridin-6-amine (99.14 mg, 0.275 mmol, 1 equiv), EDCI (210.84 mg, 1.100 mmol, 4 equiv) and pyridine (3 mL). The resulting mixture was stirred for 16 h at room temperature and concentrated under reduced pressure. The crude product 1,3-dimethyl-N-{2-[(2R)-1-methylpiperidin-2-yl]-1-{[2-(trimethylsilyl)ethoxy]methyl}pyrrolo[3,2-c]pyridin-6-yl}-4-oxophthalazine-6-carboxamide (110 mg) was used in the next step directly without further purification.

LC-MS (ES, m/z): [M+1]⁺=561

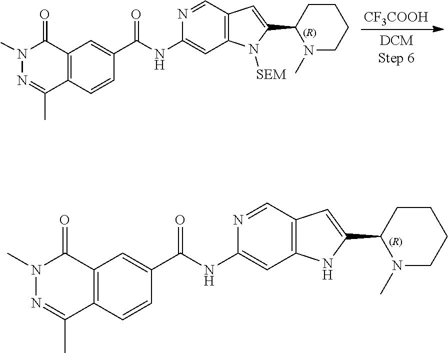

Into a 50-mL round-bottom-flask were added 1,3-dimethyl-N-{2-[(2R)-1-methylpiperidin-2-yl]-1-{[2-(trimethylsilyl)ethoxy]methyl}pyrrolo[3,2-c]pyridin-6-yl}-4-oxophthalazine-6-carboxamide (110 mg, crude), TFA (2 mL) and DCM (4 mL). The resulting mixture was stirred for 16 h at room temperature and concentrated under vacuum. The residue was diluted with DMF (3 mL), NH₃·H₂O (10 drops), then the mixture was stirred for other 4 h. The crude product was purified by Prep-HPLC with the following conditions (Column: XBridge Prep C18 OBD; Mobile Phase A: 0.05% NH₃·H₂O, Mobile Phase B: ACN; Flow rate: 60 mL/min; Gradient: 5% B to 50% B in 8 min. The fractions were concentrated and dried by lyophilization. This resulted in 15 mg of 1,3-dimethyl-N-{2-[(2R)-1-methylpiperidin-2-yl]-1H-pyrrolo[3,2-c]pyridin-6-yl}-4-oxophthalazine-6-carboxamide as a white solid.

LC-MS (ES, m/z): [M+1]⁺=431

¹H-NMR (400 MHZ, Methanol-d₄, ppm) δ 8.97 (s, 1H), 8.54 (s, 1H), 8.46 (dd, J=8.4, 2.0 Hz, 1H), 8.20 (s, 1H), 8.10 (d, J=8.4 Hz, 1H), 6.50 (s, 1H), 3.82 (s, 3H), 3.17-3.12 (m, 1H), 3.07-3.04 (m, 1H), 2.65 (s, 3H), 2.25-2.22 (m, 1H), 2.21 (s, 3H), 1.88-1.83 (m, 3H), 1.76-1.74 (m, 2H), 1.49-1.40 (m, 1H).

### Example 104: (R)-2,4-dimethyl-N-(2-(1-methylpiperidin-2-yl)-1H-pyrrolo[3,2-c]pyridin-6-yl)-1-oxo-1,2-dihydrophthalazine-6-carboxamide

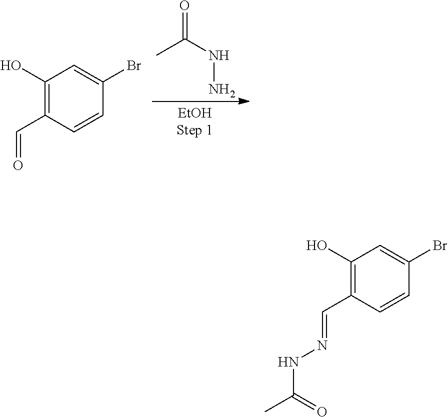

Into a 100-mL round-bottom-flask were added 4-bromo-2-hydroxybenzaldehyde (3.0 g, 14.92 mmol, 1 equiv) and acetohydrazide (2.21 g, 29.85 mmol, 2.0 equiv) in EtOH (30 mL) at room temperature. The resulting mixture was stirred for 16 h at reflux. The mixture was allowed to cool down to room temperature and concentrated under vacuum. The residue was purified by silica gel column chromatography, eluting with petroleum ether/ethyl acetate (1:1) to afford N′-[(1E)-(4-bromo-2-hydroxyphenyl)methylidene]acetohydrazide (2.7 g) as a white solid.

LC-MS (ES, m/z): [M+1]⁺=257

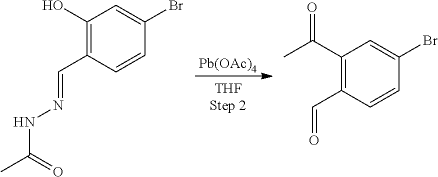

Into a 100-mL round-bottom-flask were added N′-[(1E)-(4-bromo-2-hydroxyphenyl)methylidene]acetohydrazide (2.7 g, 10.5 mmol, 1 equiv), Pb(OAc)₄ (9.31 g, 21.0 mmol, 2.0 equiv) in THF (35 mL) at room temperature. The resulting mixture was stirred for 4 h and the resulting mixture was diluted with water (60 mL) and extracted with EtOAc (3×30 mL). The combined organic layers were washed with brine (30 mL), dried over anhydrous Na₂SO₄, then concentrated under reduced pressure. The residue was purified by silica gel column chromatography, eluted with petroleum ether/ethyl acetate (1:1) to afford 2-acetyl-4-bromobenzaldehyde (2 g) as an off-white solid.

LC-MS (ES, m/z): [M+1]⁺=227

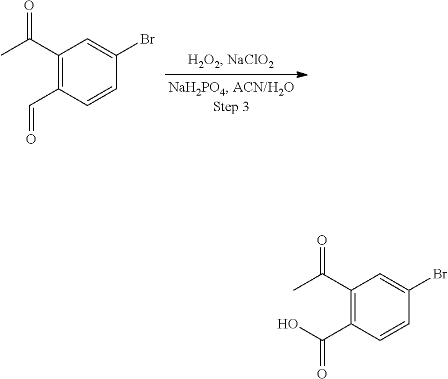

A solution of 2-acetyl-4-bromobenzaldehyde (1.9 g, 8.37 mmol, 1.0 equiv) in CH₃CN (30 mL) and H₂O (3 mL) was treated with H₂O₂ (2.5 mL) for 5 min at 0° C. followed by the addition of NaClO₂ (3.03 g, 33.47 mmol, 4.0 equiv) in portions at 0° C. The resulting mixture was stirred for 2 h at room temperature. The reaction was quenched by the addition of 2M HCl (20 mL) at 0° C. and extracted with EtOAc (3×50 mL). The combined organic layers were washed with NaHCO₃ (2×40 mL) and brine (50 mL), dried over anhydrous Na₂SO₄ and concentrated under reduced pressure. This resulted in 2-acetyl-4-bromobenzoic acid (1.3 g) as an off-white solid. The crude product was used in the next step directly without further purification.

LC-MS (ES, m/z): [M+1]⁺=243

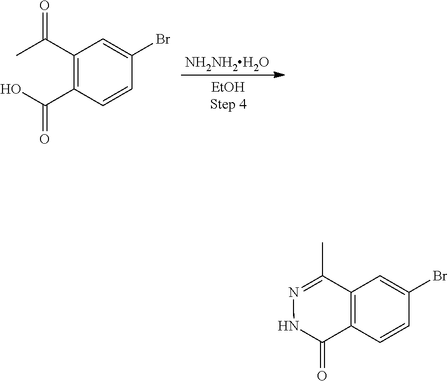

Into a 50-mL round-bottom-flask were added 2-acetyl-4-bromobenzoic acid (1.2 g, 4.94 mmol, 1 equiv), hydrazine hydrate (80%) (3 mL) and EtOH (15 mL) at room temperature. The resulting mixture was stirred for 3 h at room temperature and concentrated under reduced pressure. The residue was purified by silica gel column chromatography, eluted with petroleum ether/ethyl acetate (1:2) to afford 6-bromo-4-methyl-2H-phthalazin-1-one (0.9 g) as a light-yellow solid.

LC-MS (ES, m/z): [M+1]⁺=239

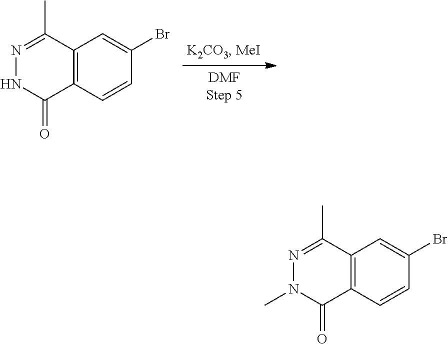

A solution of 6-bromo-4-methyl-2H-phthalazin-1-one (900 mg, 3.77 mmol, 1 equiv) and K₂CO₃ (1.56 g, 11.30 mmol, 3.0 equiv) in DMF (10 mL) was treated with Mel (1.07 g, 7.53 mmol, 2.0 equiv) for 20 min at 0° C. dropwise. The mixture was stirred for 16 h at room temperature. The reaction was quenched with water (50 mL) at 0° C. and extracted with EtOAc (3×60 mL). The combined organic layers were washed with water (8×50 mL) and brine (60 mL), dried over anhydrous Na₂SO₄ and concentrated under reduced pressure. The residue was purified by silica gel column chromatography, eluting with petroleum ether/ethyl acetate (3:1) to afford 6-bromo-2,4-dimethylphthalazin-1-one (550 mg) as a light-yellow solid.

LC-MS− (ES, m/z): [M+1]⁺=253

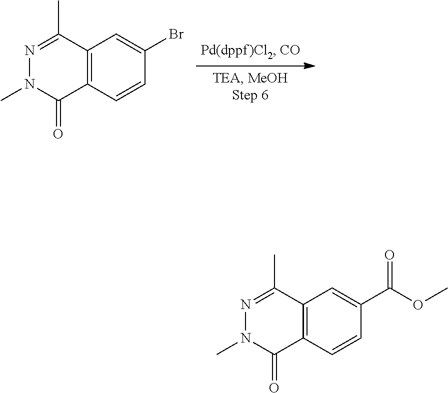

Into a 50-mL pressure seal-tube were added 6-bromo-2,4-dimethylphthalazin-1-one (550 mg, 2.17 mmol, 1 equiv), TEA (879.59 mg, 8.69 mmol, 4.0 equiv), MeOH (10 mL) and Pd (dppf)Cl₂ (79.50 mg, 0.109 mmol, 0.05 equiv). The resulting mixture was stirred for 16 h at 120° C. under carbon monoxide atmosphere and allowed to cool down to room temperature, filtered, and the filter cake washed with EtOAc (3×20 mL). The washings were concentrated under reduced pressure and the residue purified by silica gel column chromatography, eluting with petroleum ether/ethyl acetate (1:1) to afford methyl 2,4-dimethyl-1-oxophthalazine-6-carboxylate (400 mg) as a light yellow solid

LC-MS (ES, m/z): [M+1]⁺=233

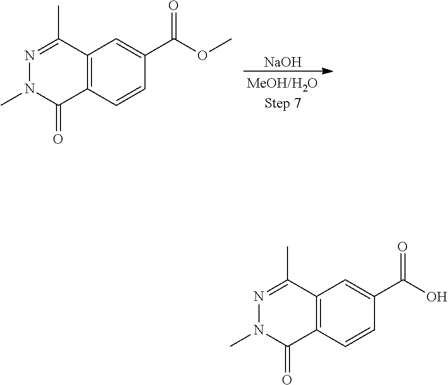

To a stirred solution of methyl 2,4-dimethyl-1-oxophthalazine-6-carboxylate (400 mg, 1.72 mmol, 1.0 equiv) in MeOH (6 mL) and H₂O (3 mL) was added NaOH (137.78 mg, 3.44 mmol, 2.0 equiv). The resulting mixture was stirred for 16 h at room temperature. The mixture was concentrated under vacuum and basified to pH 3˜4 with 2M HCl (aq). The precipitated solids were collected by filtration and washed with water (3×10 mL). This resulted in 2,4-dimethyl-1-oxophthalazine-6-carboxylic acid (370 mg) as an off-white solid.

LC-MS (ES, m/z): [M+1]⁺=219

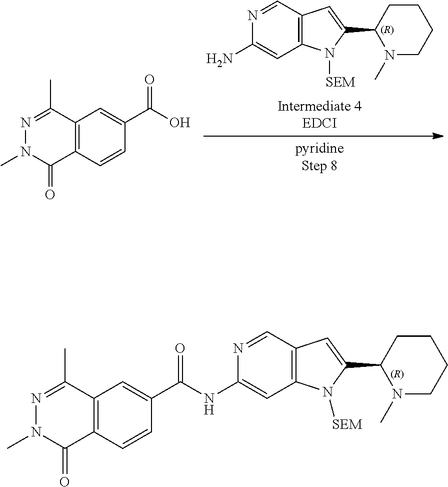

Into a 50-mL round-bottom-flask were added 2,4-dimethyl-1-oxophthalazine-6-carboxylic acid (60 mg, 0.275 mmol, 1 equiv), 2-[(2R)-1-methylpiperidin-2-yl]-1-{[2-(trimethylsilyl)ethoxy]methyl}pyrrolo[3,2-c]pyridin-6-amine (109.06 mg, 0.303 mmol, 1.1 equiv), EDCI (210.84 mg, 1.100 mmol, 4.0 equiv) and pyridine (3 mL). The resulting mixture was stirred for 16 h at room temperature. The mixture was concentrated under reduced pressure and the crude product 2,4-dimethyl-N-{2-[(2R)-1-methylpiperidin-2-yl]-1-{[2-(trimethylsilyl)ethoxy]methyl}pyrrolo [3,2-c]pyridin-6-yl}-1-oxophthalazine-6-carboxamide (110 mg) was used in the next step directly without further purification.

LC-MS (ES, m/z): [M+1]⁺=561

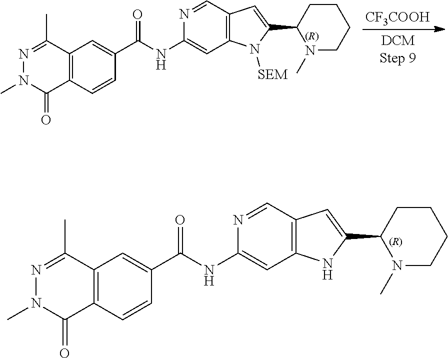

Into a 50-mL round-bottom-flask was added 2,4-dimethyl-N-{2-[(2R)-1-methylpiperidin-2-yl]-1-{[2-(trimethylsilyl)ethoxy]methyl}pyrrolo[3,2-c]pyridin-6-yl}-1-oxophthalazine-6-carboxamide (110 mg, crude), TFA (2 mL) and DCM (2 mL). The resulting mixture was stirred for 16 h at room temperature and concentrated under vacuum. The resulting mixture was diluted with DMF (3 mL) NH₃. H₂O (10 drops) added and the mixture was stirred for other 4 h. The crude product was purified by Prep-HPLC with the following conditions: Column: XBridge Prep C18 OBD 19*150 mm 5 um; Mobile Phase A: 0.05% NH₃·H₂O, Mobile Phase B: ACN; Flow rate: 60 mL/min; Gradient: 5% B to 50% B in 8 min. The fractions were concentrated and dried by lyophilization resulting in 15 mg of 2,4-dimethyl-N-{2-[(2R)-1-methylpiperidin-2-yl]-1H-pyrrolo[3,2-c]pyridin-6-yl}-1-oxophthalazine-6-carboxamide as a white solid.

LC-MS (ES, m/z): [M+1]⁺=431

¹H-NMR (400 MHZ, Methanol-d₄, ppm) δ 8.54 (s, 2H), 8.49 (d, J=8.0 Hz, 1H), 8.38 (dd, J=8.4, 1.6 Hz, 1H), 8.21 (s, 1H), 6.51 (s, 1H), 3.82-3.80 (m, 3H), 3.17-3.12 (m, 1H), 3.08-3.05 (m, 1H), 2.71 (s, 3H), 2.26-2.19 (m, 1H), 2.11 (s, 3H), 1.89-1.80 (m, 3H), 1.74-1.78 (m, 2H), 1.48-1.40 (m, 1H).

### Example 105: (R)-4-methyl-N-(2-(1-methylpiperidin-2-yl)-1H-pyrrolo[3,2-c]pyridin-6-yl)-3-oxo-3,4-dihydro-2H-benzo[b][1,4]oxazine-7-carboxamide

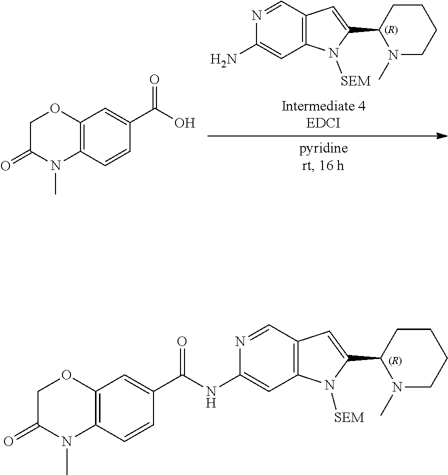

Into a 50-mL round-bottom-flask were added 4-methyl-3-oxo-2H-1,4-benzoxazine-7-carboxylic acid (Prepared according to WO2021127166, Acid CH, 60 mg, 0.290 mmol, 1 equiv), 2-[(2R)-1-methylpiperidin-2-yl]-1-{[2-(trimethylsilyl)ethoxy]methy 1}pyrrolo[3,2-c]pyridin-6-amine (104.42 mg, 0.290 mmol, 1 equiv), EDCI (222.06 mg, 1.160 mmol, 4 equiv) and pyridine (5 mL). The resulting mixture was stirred for 16 h at room temperature and concentrated under reduced pressure. The crude product 4-methyl-N-{2-[(2R)-1-methylpiperidin-2-yl]-1-{[2-(trimethylsilyl)ethoxy]methyl}pyrrolo[3,2-c]pyridin-6-yl}-3-oxo-2H-1,4-benzoxazine-7-carboxamide (110 mg, crude) was used in the next step directly without further purification.

LC-MS (ES, m/z): [M+1]⁺=550

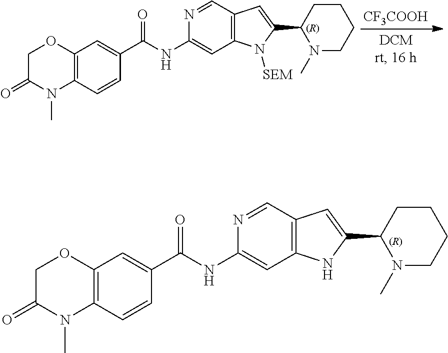

Into a 50-mL round-bottom-flask were added 4-methyl-N-{2-[(2R)-1-methylpiperidin-2-yl]-1-{[2-(trimethylsilyl)ethoxy]methyl}pyrrolo[3,2-c]pyridin-6-yl}-3-oxo-2H-1,4-benzoxazine-7-carboxamide (110 mg, crude), trifluoroacetic acid (1.5 mL) and DCM (1.5 mL). The resulting mixture was stirred for 20 h at room temperature and concentrated under vacuum. The resulting mixture was diluted with DMF (3 mL) and NH₃·H₂O (10 drops) added, then the mixture was stirred for other 4 h. The crude product was purified by Prep-HPLC with the following conditions: Column: XBridge Prep C18 OBD 19*150 mm 5 um; Mobile Phase A: 0.05% NH₃·H₂O, Mobile Phase B: ACN; Flow rate: 60 mL/min; Gradient: 5% B to 50% B in 8 min. The fractions were concentrated and dried by lyophilization. This resulted in 11.8 mg of 4-methyl-N-{2-[(2R)-1-methylpiperidin-2-yl]-1H-pyrrolo[3,2-c]pyridin-6-yl}-3-oxo-2H-1,4-benzoxazine-7-carboxamide as a white solid.

LC-MS (ES, m/z): [M+1]⁺=420

¹H-NMR (300 MHz, Methanol-d₄, ppm) δ 8.53 (d, J=0.9 Hz, 1H), 8.17 (s, 1H), 7.77 (dd, J=8.4, 2.1 Hz, 1H), 7.66 (d, J=1.8 Hz, 1H), 7.32 (d, J=8.7 Hz, 1H), 6.51 (d, J=0.6 Hz, 1H), 4.72 (s, 2H), 3.44 (s, 3H), 3.19-3.06 (m, 2H), 2.29-2.20 (m, 1H), 2.13 (s, 3H), 1.90-1.78 (m, 5H), 1.57-1.46 (m, 1H).

### Example 106

### FRET Assay:

Compounds of the invention were tested in a TR-FRET ENL Screening Assay. TR-FRET (time-resolved fluorescence energy transfer) can be used to quantify ENL YEATS domain binding to a crotonylated histone peptide (H3K9cr, aa1-20). Streptavidin-Europium (Eu) chelate binds the biotinylated peptide, while Anti-6×HIS ULight™ binds 6×HIS-ENL. When Eu chelate is excited at 320 nm, fluorescence resonance energy transfer (FRET) occurs if Eu and ULight are made proximal by ENL binding to the acyl-peptide. ULight emission (FRET) is measured at 665 nm and normalized to the Eu emission at 615 nm to reduce variability between wells.

### FRET Assay-Protocol

Compounds of the invention were dissolved in DMSO at a concentration of 3 mM with subsequent dilutions in assay buffer (50 mM HEPES PH7.0, 150 mM NaCl, 0.05% BSA, 0.2% Pluronic F-127) such that the assay contained 1% DMSO. In a white 384 shallow well Microplate (Proxiplate-384 Plus, PerkinElmer, 6008280), 150 nL of compound or vehicle (1% DMSO in assay buffer) for the high control (HC) wells and 5 μL of 30 nM ENL Protein (6×HIS ENL YEATS Domain, EpiCypher, 15-0069) were combined and incubated 15 minutes at RT. Low control (LC) wells received 5 μL of assay buffer instead of ENL protein. Then 5 μL of 15 nM H3K9cr peptide (H3 aa1-20, biotinylated; EpiCypher, 12-0099) in assay buffer was added and incubated 30 minutes at RT. Finally a 5 μL mix of 45 nM Anti-6HIS ULight (PerkinElmer, TRF0105) and 1.5 nM Streptavidin-Europium Chelate (PerkinElmer, AD0060) were added and incubated for a further 30 minutes at RT. The TR-FRET signal (665 nm signal/615 nm signal X 10,000) was measured using a PerkinElmer 2104 EnVision (Xenon Flash Lamp excitation, 320 nm±37.5 nm excitation filter, 407 nm cut off dichroic mirror, 615 nm±4.25 (Europium) nm and 665 nm±3.75 nM (ULight) emission filters). Compound concentration response curves were performed in duplicate over the concentration range of 0.15 nM-30 μM. The response at each compound concentration minus the LC value was converted to percent inhibition of the vehicle control group response (HC-LC). The relationship between the % inhibition and the compound concentration was analyzed using a four parameter logistic equation to estimate lower and upper asymptotes, the compound concentration producing 50% inhibition (IC50 value) and the slope at the mid-point location.

TABLE 1
FRET Assay Results

| Example | TR FRET ENL IC50 (μM) |
|---|---|
| 1 | 0.017 |
| 2 | 0.263 |
| 3 | 0.214 |
| 4 | 0.203 |
| 5 | 1.239 |
| 6 | 0.106 |
| 7 | 0.142 |
| 8 | 0.1 |
| 9 | 0.2 |
| 10 | 0.098 |
| 11 | 0.465 |
| 12 | 0.078 |
| 13 | 0.158 |
| 14 | 0.178 |
| 15 | 0.053 |
| 16 | 0.088 |
| 17 | 0.295 |
| 18 | 0.125 |
| 19 | 0.146 |
| 20 | 0.105 |
| 21 | 0.2 |
| 22 | 0.131 |
| 23 | 0.277 |
| 24 | 0.217 |
| 25 | 0.181 |
| 26 | 0.145 |
| 27 | 0.158 |
| 28 | 0.133 |
| 29 | 0.021 |
| 30 | 0.051 |
| 31 | 0.057 |
| 32 | 0.058 |
| 33 | 0.042 |
| 34 | 0.048 |
| 35 | 0.114 |
| 36 | 0.043 |
| 37 | 0.055 |
| 38 | 0.094 |
| 39 | 0.043 |
| 40 | 0.034 |
| 41 | 0.014 |
| 42 | 0.084 |
| 43 | 0.037 |
| 44 | 0.78 |
| 45 | 0.293 |
| 46 | 0.061 |
| 47 | 0.028 |
| 48 | 0.03 |
| 49 | 0.026 |
| 50 | 0.103 |
| 51 | 0.1 |
| 52 | 0.025 |
| 53 | 0.038 |
| 54 | 0.318 |
| 55 | 0.018 |
| 56 | 0.131 |
| 57 | 0.106 |
| 58 | 0.208 |
| 59 | 0.053 |
| 60 | 0.148 |
| 61 | 0.09 |
| 62 | 0.465 |
| 63 | 0.095 |
| 64 | 0.091 |
| 65 | 0.018 |
| 66 | 0.029 |
| 67 | 0.075 |
| 68 | 0.035 |
| 69 | 0.18 |
| 70 | 0.11 |
| 71 | 0.056 |
| 72 | 0.12 |
| 73 | 0.078 |
| 74 | 0.079 |
| 75 | 0.126 |
| 76 | 0.136 |
| 77 | 1.15 |
| 78 | 0.014 |
| 79 | 0.015 |
| 80 | 0.018 |
| 81 | 0.051 |
| 82 | 0.102 |
| 83 | 0.064 |
| 84 | 0.031 |
| 85 | 0.056 |
| 86 | 0.041 |
| 87 | 0.047 |
| 88 | 0.047 |
| 89 | 0.175 |
| 90 | 0.051 |
| 91 | 0.164 |
| 92 | 0.093 |
| 93 | 0.076 |
| 94 | 0.22 |
| 95 | 0.094 |
| 96 | 0.25 |
| 97 | 0.076 |
| 98 | 0.058 |
| 99 | 0.056 |
| 100 | 0.117 |
| 101 | 0.177 |
| 102 | 0.2 |
| 103 | 0.148 |
| 104 | 0.16 |
| 105 | 0.145 |

### Example 107

### Cell Assay:

Cell-based assays were used to assess the ability of test compounds to reduce cell viability in both MV4:11 (MLL-AF4 MLL) and K₅₆₂cells, which were cultured in Iscove's Modified Dulbecco's medium (Gibco, 12440061) containing 10% FBS. The assays were conducted over 12 days and the cells being split on days 4 and 8. Compound concentration response curves were performed in duplicate over the concentration range of 0.15 nM-30 μM. On day 0, the compounds or vehicle were plated in a 300 nL directly into 96 well cell culture plates (Corning, 3599) with 5000 cells/well in a volume of 100 μL. Blank wells received cell culture medium. Plates were incubated for 4 days at 37° C. with 5% CO₂. On days 4 and day 8 the cells were split and incubated for a further 4 days whilst an aliquot of cells were taken for the CTG readout. For the cell splitting, 270 nL of compounds or DMSO was added to a new 96 well cell culture plate to which 90 μL of medium plus 10 μL of cells from the original assay plate (after mixing) or 100 μL of medium (Blank wells) was added. This was repeated on day 8.

Cell viability was assessed using the CellTiter-Glo® homogeneous luminescent assay kit (Promega, G9243), according to the manufacturer's instructions. This quantifies ATP, which indicates the presence of metabolically active cells. On days 4, 8 and 12, 20 μl of the remaining cell suspension was aspirated into 384-well plate (Corning 3570) to which an equal volume CellTiter-Glo reagent was. Plates were incubated for 10 minute incubation at RT prior to recording the luminescence signal using EnVision plate reader (PE, 2104). The resulting data were analyzed as follows:

$$Inhibition ( \% ) = 100 \% \times ( L u {m}_{v e h i c l e} - L u {m}_{sample} ) / ( Lu {m}_{v e h i c l e} - {Lum}_{blank} )$$

where vehicle are cells treated with 0.3% DMSO, Blank is culture medium. IC50 determinations were calculated by fitting the curve using XLfit (v5.3.1.3): Y=Bottom+(Top−Bottom)/(1+10\hat{ }((LogIC50−X)*HillSlope)).

TABLE 2
Cell Assay

| | CTG_MV4; 11 |
|---|---|
| Example | 12 DAY_EC50 (μM) |
| 1 | 3.252 |
| 2 | 1.323 |
| 3 | 0.571 |
| 4 | 4.372 |
| 5 | |
| 6 | 0.033 |
| 7 | 0.101 |
| 8 | 0.123 |
| 9 | |
| 10 | 0.280 |
| 11 | |
| 12 | 0.201 |
| 13 | 0.359 |
| 14 | 4.643 |
| 15 | |
| 16 | 0.106 |
| 17 | 3.251 |
| 18 | |
| 19 | 0.031 |
| 20 | 0.337 |
| 21 | |
| 22 | |
| 23 | |
| 24 | |
| 25 | |
| 26 | 0.374 |
| 27 | 0.338 |
| 28 | 0.186 |
| 29 | 1.121 |
| 30 | 0.039 |
| 31 | 0.091 |
| 32 | 12.224 |
| 33 | 1.775 |
| 34 | 0.570 |
| 35 | 0.269 |
| 36 | 0.853 |
| 37 | 0.047 |
| 38 | 0.087 |
| 39 | 2.783 |
| 40 | 0.088 |
| 41 | 0.072 |
| 42 | 0.117 |
| 43 | 0.256 |
| 44 | |
| 45 | 6.946 |
| 46 | 0.257 |
| 47 | 0.745 |
| 48 | |
| 49 | 1.435 |
| 50 | 2.320 |
| 51 | 1.200 |
| 52 | |
| 53 | 5.294 |
| 54 | 0.621 |
| 55 | 0.688 |
| 56 | 4.780 |
| 57 | >30 |
| 58 | 8.579 |
| 59 | 15.824 |
| 60 | 0.298 |
| 61 | 2.859 |
| 62 | 0.597 |
| 63 | 7.020 |
| 64 | 0.773 |
| 65 | 0.054 |
| 66 | 1.630 |
| 67 | 0.038 |
| 68 | 0.048 |
| 69 | |
| 70 | 0.189 |
| 71 | 0.220 |
| 72 | 2.567 |
| 73 | 1.416 |
| 74 | 1.415 |
| 75 | 2.408 |
| 76 | 0.723 |
| 77 | |
| 78 | 0.167 |
| 79 | 0.446 |
| 80 | 0.187 |
| 81 | 0.292 |
| 82 | 0.409 |
| 83 | 0.229 |
| 84 | 0.134 |
| 85 | 0.488 |
| 86 | 0.180 |
| 87 | 0.172 |
| 88 | 0.069 |
| 89 | 0.252 |
| 90 | 0.144 |
| 91 | 0.215 |
| 92 | 0.100 |
| 93 | 0.146 |
| 94 | 0.368 |
| 95 | 0.451 |
| 96 | 0.319 |
| 97 | 0.146 |
| 98 | 0.440 |
| 99 | 0.293 |
| 100 | 0.202 |
| 101 | 0.426 |
| 102 | 0.289 |
| 103 | 0.292 |
| 104 | 0.492 |
| 105 | 0.416 |

1. A compound of Formula I: 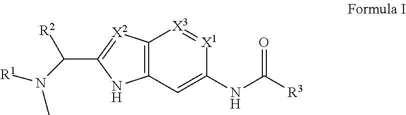
wherein:
X¹, X², and X³ are independently chosen from N and CH;
R¹ and R² are chosen from:
(a) R¹ and R² taken together form a pyrrolidine or piperidine; and
(b) R¹ and R² are methyl;
R³ is a fused bicycle selected from:
(a) a fused 5,6 bicyclic heterocycle, optionally substituted with one or more C₁-C₆ alkyl (methyl);
(b) a fused 6,5 bicyclic heterocycle, optionally substituted with one or more of the following: C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl; and
(c) a fused 6,6 bicyclic heterocycle, optionally substituted with one or more of the following: C₁-C₆ alkyl, C₁-C₆ alkoxy, halogen, and NHR⁵, wherein R⁵ is chosen from hydrogen and C₁-C₆ alkyl.

2. A compound according to claim 1 of Formula II: 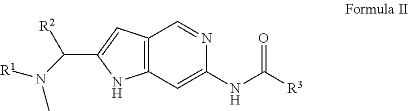
wherein R¹, R² and R³ are as defined above for Formula I.

3. A compound according to claim 1 of Formula II: 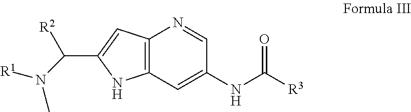
wherein R¹, R² and R³ are as defined above for Formula I.

4. A compound according to claim 1 of Formula IV: 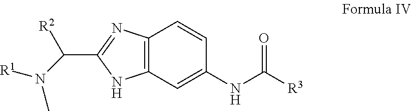
wherein R¹, R² and R³ are as defined above for Formula I.

5. A compound according to claim 1 of Formula V: 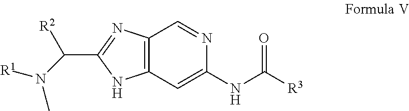
wherein R¹, R² and R³ are as defined above for Formula I.

6. A compound according to claim 1 of Formula VI 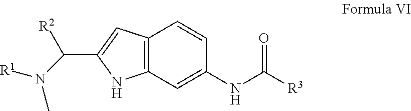
wherein R¹, R² and R³ are as defined above for Formula I.

7. A pharmaceutical composition comprising a compound of claim 1 and one or more pharmaceutically acceptable carriers.

8. The pharmaceutical composition of claim 7, further comprising one or more therapeutic agents.

9. The pharmaceutical composition of claim 8, wherein the one or more therapeutic agent is selected from the group consisting of Bcl-2 inhibitors, cyclin-dependent kinase 4 and 6 (CDK 4/6 inhibitors), DNA methyltransferase inhibitors, histone deacetylase (HDAC) inhibitors, mTOR inhibitors, mutant isocitrate dehydrogenase (IDH1 and IDH2) inhibitors, glucocorticoids, an epigenetic modulators and chemotherapeutic agents.

10. A method of treating an acute leukemia comprising administering a therapeutically effective amount of a compound of claim 1.

11. The method of claim 10, wherein the acute leukemia is acute lymphoblastic leukemia (ALL).

12. The method of claim 10, wherein the acute leukemia is acute myelogenous leukemia (AML).

13. The method of claim 12, wherein the AML is a subtype selected from the group consisting of acute myeloid leukemia, minimally differentiated (MO), acute myeloid leukemia without maturation (M1), acute myeloid leukemia with maturation (M2), acute myeloid leukemia with maturation with t (8;21), acute promyelocytic leukemia (M3), hypergranular type, microgranular type, acute myelomonocytic leukemia (M4), acute myelomonocytic leukemia with increased marrow eosinophils (M4E0), acute monocytic leukemia (M5), acute monoblastic leukemia (M5a), acute monocytic leukemia with maturation (M5b), erythroleukemia erythroid/myeloid (M6a), pure erythroid malignancy (M6b), acute megakaryoblastic leukemia (M7), acute megakaryoblastic leukemia associated with t (1;22), acute basophilic leukemia, acute myelofibrosis (acute myelodysplasia with myelofibrosis), acute leukemia and transient myeloproliferative disorder in Down's Syndrome, hypocellular acute myeloid leukemia, and myeloid sarcoma.

14. The method of claim 10, wherein the at least one compound is administered orally.

15. The method of claim 10, wherein the at least one compound is administered from one to four times per day.

16. A compound of Formula I: 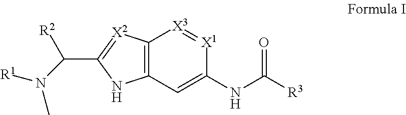
wherein:
X¹, X², and X³ are independently chosen from N and CH;
R¹ and R² are chosen from:
(a) R¹ and R² taken together form a pyrrolidine or piperidine; and
(b) R¹ and R² are methyl;
R³ is a fused bicycle selected from:
(a) a fused 5,6 bicyclic heterocycle, optionally substituted with one or more C₁-C₆ alkyl (methyl);
(b) a fused 6,5 bicyclic heterocycle, optionally substituted with one or more of the following: C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl; and
(c) a fused 6,6 bicyclic heterocycle, optionally substituted with one or more of the following: C₁-C₆ alkyl, C₁-C₆ alkoxy, halogen, oxo, and NHR⁵, wherein R⁵ is chosen from hydrogen and C₁-C₆ alkyl.

17. A compound according to claim 16 of Formula II: 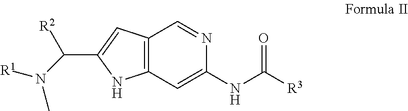
wherein R¹, R² and R³ are as defined above for Formula I.

18. A compound according to claim 17 of Formula IIa, IIb or IIc: 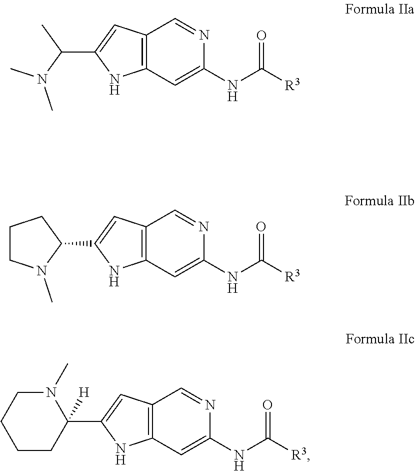
wherein R³ is defined as above for Formula I.

19. A compound according to claim 18, wherein R³ is a fused 6,5 bicyclic heterocycle selected from the following 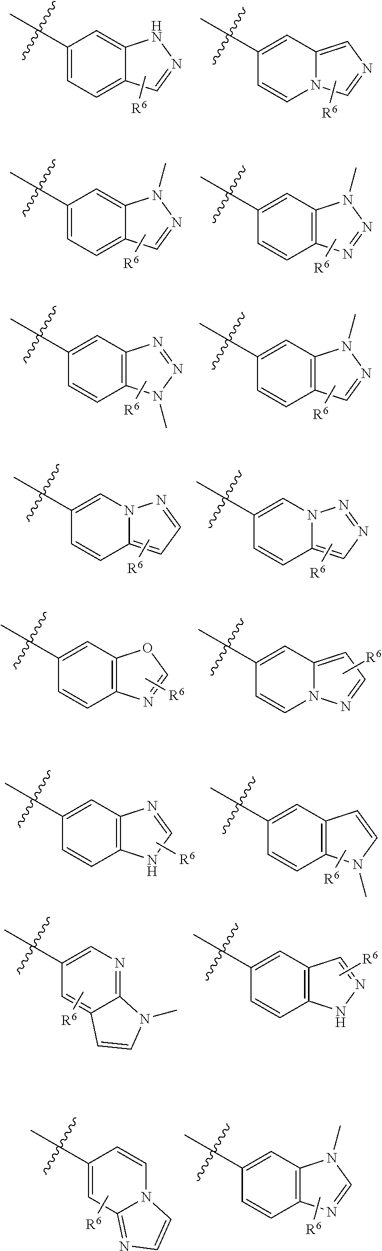 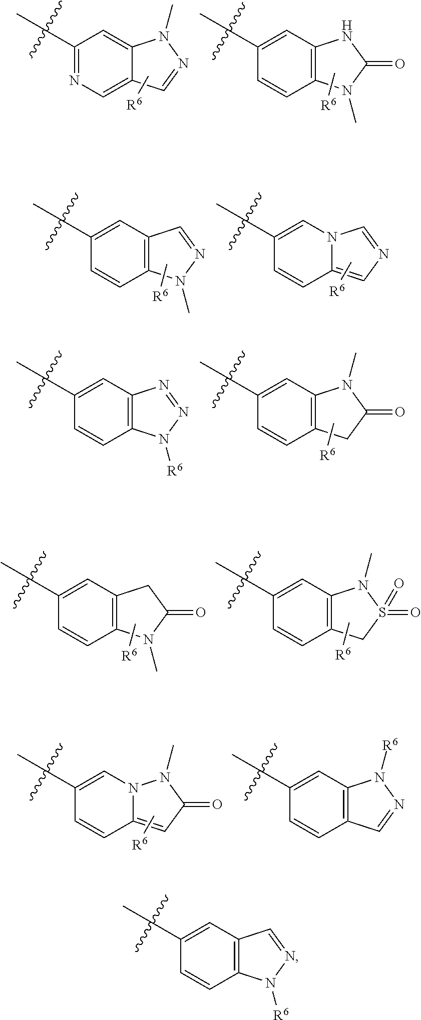
optionally substituted with one or more R⁶ selected from the group consisting of: C₁-C₆ alkyl, C₁-C₆ haloalkyl, C₃-C₈ carbocycle, C₁-C₆ oxaalkyl, C₁-C₆ alkoxy, oxo, halogen, heterocycle, and NHR⁴, where R⁴ is chosen from C₁-C₆ alkyl and C₁-C₆ oxaalkyl.

20. A compound according to claim 18, wherein R³ is a fused 6,6 bicyclic heterocycle selected from the following 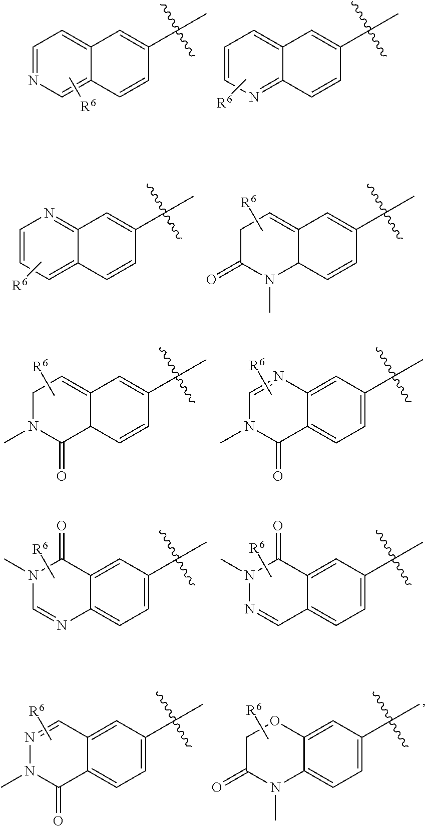
optionally substituted with one or more R⁶: C₁-C₆ alkyl, C₁-C₆ alkoxy, halogen, oxo, and NHR⁵, wherein R⁵ is chosen from hydrogen and C₁-C₆ alkyl.

21. A compound according to claim 16 of Formula III: 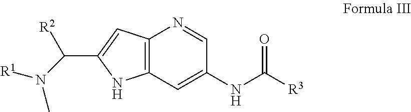
wherein R¹, R² and R³ are as defined above for Formula I.

22. A compound according to claim 16 of Formula IV: 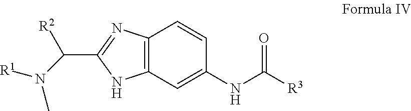
wherein R¹, R² and R³ are as defined above for Formula I.

23. A compound according to claim 16 of Formula V: 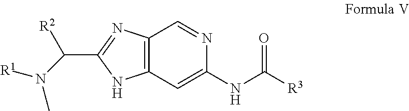
wherein R¹, R² and R³ are as defined above for Formula I.

24. A compound according to claim 16 of Formula VI 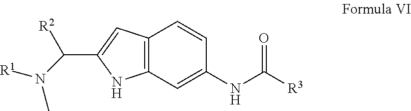
wherein R¹, R² and R³ are as defined above for Formula I.

25. A pharmaceutical composition comprising a compound of claim 16 and one or more pharmaceutically acceptable carriers.

26. The pharmaceutical composition of claim 25, further comprising one or more therapeutic agents.

27. The pharmaceutical composition of claim 26, wherein the one or more therapeutic agent is selected from the group consisting of Bcl-2 inhibitors, cyclin-dependent kinase 4 and 6 (CDK 4/6 inhibitors), DNA methyltransferase inhibitors, histone deacetylase (HDAC) inhibitors, mTOR inhibitors, mutant isocitrate dehydrogenase (IDH1 and IDH2) inhibitors, glucocorticoids, an epigenetic modulators and chemotherapeutic agents.

28. A method of treating an acute leukemia comprising administering a therapeutically effective amount of a compound of claim 16.

29. The method of claim 28, wherein the acute leukemia is acute lymphoblastic leukemia (ALL).

30. The method of claim 28, wherein the acute leukemia is acute myelogenous leukemia (AML).

31. The method of claim 30, wherein the AML is a subtype selected from the group consisting of acute myeloid leukemia, minimally differentiated (MO), acute myeloid leukemia without maturation (M1), acute myeloid leukemia with maturation (M2), acute myeloid leukemia with maturation with t (8;21), acute promyelocytic leukemia (M3), hypergranular type, microgranular type, acute myelomonocytic leukemia (M4), acute myelomonocytic leukemia with increased marrow eosinophils (M4E0), acute monocytic leukemia (M5), acute monoblastic leukemia (M5a), acute monocytic leukemia with maturation (M5b), erythroleukemia erythroid/myeloid (M6a), pure erythroid malignancy (M6b), acute megakaryoblastic leukemia (M7), acute megakaryoblastic leukemia associated with t (1;22), acute basophilic leukemia, acute myelofibrosis (acute myelodysplasia with myelofibrosis), acute leukemia and transient myeloproliferative disorder in Down's Syndrome, hypocellular acute myeloid leukemia, and myeloid sarcoma.

32. The method of claim 28, wherein the at least one compound is administered orally.

33. The method of claim 28, wherein the at least one compound is administered from one to four times per day.
